(19) United States
(12) Patent Application Publication
(10) Pub. No.: US 2022/0064735 A1
(43) Pub. Date: Mar. 3, 2022

(54) Synaptojanin 2 (SYNJ2) Variants And Uses Thereof

(71) Applicant: Regeneron Pharmaceuticals, Inc., Tarrytown, NY (US)

(72) Inventors: Kavita Praveen, Tarrytown, NY (US); Giovanni Coppola, Tarrytown, NY (US); Manuel Allen Revez Ferreira, Tarrytown, NY (US); Lauren Gurski, Tarrytown, NY (US); Aris Baras, Tarrytown, NY (US); Meghan Drummond Samuelson, Tarrytown, NY (US); Goncalo Abecasis, Tarrytown, NY (US)

(21) Appl. No.: 17/525,336

(22) Filed: Nov. 12, 2021

Related U.S. Application Data
(63) Continuation of application No. 16/881,167, filed on May 22, 2020.
(60) Provisional application No. 62/851,296, filed on May 22, 2019.

(51) Int. Cl. C12Q 1/6883

(52) U.S. Cl. CPC C12Q 1/6883; C12Q 2600/156; C12Q 2600/106

(57) ABSTRACT

The present disclosure provides methods of treating patients having hearing loss, methods of identifying subjects having an increased risk of developing hearing loss, methods of detecting human Synaptojanin-2 (SYNJ2) variant nucleic acid molecules and variant polypeptides, and SYNJ2 variant nucleic acid molecules and variant polypeptides.

### REFERENCE TO SEQUENCE LISTING

This application includes a Sequence Listing submitted electronically as a text file named 18923802701SEQ, created on May 16, 2020, with a size of 508 kilobytes. The Sequence Listing is incorporated herein by reference.

### FIELD

The present disclosure relates generally to the treatment of patients having hearing loss, methods of identifying subjects having an increased risk of developing hearing loss, methods of detecting SYNJ2 variant nucleic acid molecules and variant polypeptides, and SYNJ2 variant nucleic acid molecules and SYNJ2 variant polypeptides.

### BACKGROUND

Hearing impairment is the most common sensory defect in humans, affecting approximately 15% of Americans. Studies have shown that approximately 1 in 1000 infants are born with a level of deafness that will affect their linguistic development and speech perception (McHugh and Friedman, Anat. Rec. A Discov. Mol. Cell Evol. Biol., 2006, 288, 370-381). Hearing loss usually has a negative impact on education, social interactions and career opportunities and is, therefore, associated with major social and financial costs. The impact of hearing loss upon an individual can be quite significant (Yoshinaga-Itano, Otolaryngol Clin. North Am., 1999, 32, 1089-1102; Mohr and Feldman, Int. J. Technol. Assess. Health Care, 2000, 16, 1120-35).

The degree of hearing loss varies from mild to profound deafness. It can be stable or the hearing loss can progress with time. The deafness can be present at birth or develop late in life. By far the largest group of people with a hearing loss are amongst the elderly, many of whom develop an age-related hearing loss or presbyacusis (Petit, Trends Mol. Med., 2006, 12, 57-64). Both genetic and environmental factors can cause deafness. Genetic factors are the underlying aetiology of deafness in the majority of children and young people with a hearing loss. Genetic factors play a significant role in age-related hearing loss. Hearing loss can be associated with other clinical features (syndromic hearing loss) or isolated (non-syndromic hearing loss) (Petersen and Willems, Clin. Genet., 2006, 69, 371-392).

Synaptojanins belong to a family of inositol polyphosphate phosphatases characterized by the presence of an N-terminal phosphatidylinositol polyphosphate 3′ and 4′ phosphatase domain similar to the yeast Sac1 domain and a central inositol polyphosphate 5′ phosphatase (IPP5Pase) domain. Mammals possess two paralogs, Synaptojanin-1 (SYNJ1) and Synaptojanin-2 (SYNJ2), encoding proteins that are approximately 55% identical over their Sad-like and IPP5Pase domains but with divergent carboxyl termini.

SYNJ2 is believed to function in recycling neurotransmitter vesicles and is implicated in spermatogenesis. SYNJ2 may also mediate the inhibitory effect of Rac1 on endocytosis. Defects in Synj1 also cause slowed endocytosis, depletion of synaptic vesicles, accumulation of CCVs and aggregation of cortical actin at neuromuscular junction synapses in C.e legans, Drosophila and zebrafish.

### SUMMARY

The present disclosure provides novel nucleic acid molecules (i.e., genomic DNA, mRNA, and cDNA) encoding SYNJ2 variant polypeptides, and SYNJ2 variant polypeptides, that have been demonstrated herein to be associated with hearing loss and the risk of developing hearing loss.

The present disclosure also provides methods for identifying a human subject having an increased risk for developing hearing loss, wherein the method comprises: determining or having determined the presence or absence of a Synaptojanin-2 predicted loss-of-function variant nucleic acid molecule encoding a human SYNJ2 polypeptide in a biological sample obtained from the subject; wherein: when the human subject is SYNJ2 reference, then the human subject does not have an increased risk for developing hearing loss; and when the human subject is heterozygous for a SYNJ2 predicted loss-of-function variant or homozygous for a SYNJ2 predicted loss-of-function variant, then the human subject has an increased risk for developing hearing loss.

The present disclosure also provides methods for treating a patient with a therapeutic agent that treats or inhibits hearing loss, wherein the patient is suffering from hearing loss, the method comprising the steps of: determining whether the patient has a Synaptojanin-2 predicted loss-of-function variant nucleic acid molecule encoding a human SYNJ2 polypeptide by: obtaining or having obtained a biological sample from the patient; and performing or having performed a genotyping assay on the biological sample to determine if the patient has a genotype comprising the SYNJ2 predicted loss-of-function variant nucleic acid molecule; and when the patient is SYNJ2 reference, then administering or continuing to administer to the patient the therapeutic agent that treats or inhibits hearing loss in a standard dosage amount; and when the patient is heterozygous or homozygous for a SYNJ2 predicted loss-of-function variant, then administering or continuing to administer to the patient the therapeutic agent that treats or inhibits hearing loss in an amount that is the same as or greater than a standard dosage amount; wherein the presence of a genotype having the SYNJ2 predicted loss-of-function variant nucleic acid molecule encoding the human SYNJ2 polypeptide indicates the patient has an increased risk of developing hearing loss.

The present disclosure also provides methods for detecting a human Synaptojanin-2 (SYNJ2) variant nucleic acid molecule in a human subject comprising assaying a sample obtained from the human subject to determine whether a genomic nucleic acid molecule in the sample comprises a nucleotide sequence comprising: a thymine at a position corresponding to position 89,742 according to SEQ ID NO:2, or the complement thereof; or a thymine at a position corresponding to position 99,219 according to SEQ ID NO:3, or the complement thereof.

The present disclosure also provides methods for detecting a human Synaptojanin-2 (SYNJ2) variant nucleic acid molecule in a human subject comprising assaying a sample obtained from the human subject to determine whether an mRNA molecule in the sample comprises a nucleotide sequence comprising a uracil at a position corresponding to position 2,050 according to SEQ ID NO:5, or the complement thereof.

The present disclosure also provides methods for detecting a human Synaptojanin-2 (SYNJ2) variant nucleic acid molecule in a human subject comprising assaying a sample obtained from the human subject to determine whether a cDNA molecule produced from an mRNA molecule in the sample comprises a nucleotide sequence comprising a thymine at a position corresponding to position 2,050 according to SEQ ID NO:7, or the complement thereof.

The present disclosure also provides methods for detecting the presence of a human Synaptojanin-2 Thr656Met or rs2256014 variant polypeptide, comprising performing an assay on a sample obtained from a human subject to determine whether a SYNJ2 protein in the sample comprises a methionine at a position corresponding to position 656 according to SEQ ID NO:9.

The present disclosure also provides isolated alteration-specific probes or alteration-specific primers comprising at least about 15 nucleotides, wherein the alteration-specific probes or alteration-specific primers comprise a nucleotide sequence which is complementary to a portion of a nucleotide sequence encoding a human Synaptojanin-2 polypeptide, wherein the portion comprises a position corresponding to: position 89,742 according to SEQ ID NO:2, or the complement thereof; position 2,050 according to SEQ ID NO:5, or the complement thereof; position 2,050 according to SEQ ID NO:7, or the complement thereof; or position 99,219 according to SEQ ID NO:3, or the complement thereof.

The present disclosure also provides molecular complexes comprising an alteration-specific primer or an alteration-specific probe hybridized to a genomic nucleic acid molecule comprising a nucleotide sequence encoding a human Synaptojanin-2 polypeptide, wherein the alteration-specific primer or the alteration-specific probe is hybridized to: a thymine at a position corresponding to position 89,742 according to SEQ ID NO:2, or the complement thereof; or a thymine at a position corresponding to position 99,219 according to SEQ ID NO:3, or the complement thereof.

The present disclosure also provides molecular complexes comprising an alteration-specific primer or an alteration-specific probe hybridized to an mRNA molecule comprising a nucleotide sequence encoding a human Synaptojanin-2 polypeptide, wherein the alteration-specific primer or the alteration-specific probe is hybridized to a uracil at a position corresponding to position 2,050 according to SEQ ID NO:5, or the complement thereof.

The present disclosure also provides molecular complexes comprising an alteration-specific primer or an alteration-specific probe hybridized to a cDNA molecule comprising a nucleotide sequence encoding a human Synaptojanin-2 polypeptide, wherein the alteration-specific primer or the alteration-specific probe is hybridized to a thymine at a position corresponding to position 2,050 according to SEQ ID NO:7, or the complement thereof.

The present disclosure also provides therapeutic agents that treat or inhibit hearing loss for use in the treatment of hearing loss in a human subject having: i) a genomic nucleic acid molecule having a nucleotide sequence encoding a human Synaptojanin-2 polypeptide, wherein the nucleotide sequence comprises: a thymine at a position corresponding to position 89,742 according to SEQ ID NO:2, or the complement thereof; or a thymine at a position corresponding to position 99,219 according to SEQ ID NO:3, or the complement thereof; ii) an mRNA molecule having a nucleotide sequence encoding a human SYNJ2 polypeptide, wherein the nucleotide sequence comprises a uracil at a position corresponding to position 2,050 according to SEQ ID NO:5, or the complement thereof; iii) a cDNA molecule having a nucleotide sequence encoding a human SYNJ2 polypeptide, wherein the nucleotide sequence comprises a thymine at a position corresponding to position 2,050 according to SEQ ID NO:7, or the complement thereof.

### Description

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the terms “subject” and “patient” are used interchangeably. A subject may include any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human.

A rare and a common variant in the SYNJ2 gene associated with an increased risk of developing hearing loss and an increased mean speech reception threshold (SRT) in human subjects have been identified in accordance with the present disclosure. For example, a genetic alteration that changes the cytosine nucleotide of position 89,742 in the human SYNJ2 reference (see, SEQ ID NO:1) to thymine, or the adenine nucleotide of position 114,390 in the human SYNJ2 reference (see, SEQ ID NO:1) to guanine, or the guanine nucleotide of position 99,219 in the human SYNJ2 reference (see, SEQ ID NO:1) to thymine, or the thymine nucleotide of position 95,136 in the human SYNJ2 reference (see, SEQ ID NO:1) to cytosine has been observed to indicate that the human having such an alteration may have an increased risk of developing hearing loss. Altogether, the genetic analyses described herein indicate that the SYNJ2 gene variants described herein associate with an increased risk of developing hearing loss and with an increased mean SRT. Therefore, human subjects that have a SYNJ2 variant nucleic acid molecule or polypeptide that associates with an increased risk of developing hearing loss and/or increased mean SRT may be treated such that hearing loss is prevented, the symptoms thereof are reduced, and/or development of symptoms is repressed. Accordingly, the present disclosure provides methods of leveraging the identification of such SYNJ2 variants in subjects to identify or stratify risk in such subjects of developing hearing loss and/or increased mean SRT, or to diagnose subjects as having an increased risk of developing hearing loss and/or increased mean SRT such that subjects at risk or subjects with active disease may be treated. Additionally, the present disclosure provides isolated SYNJ2 variant genomic nucleic acid molecules, variant mRNA molecules, and variant cDNA molecules. Also provided herein are SYNJ2 loss-of-function variant nucleic acid molecules discovered to be associated with an increased risk of developing hearing loss and/or increased mean SRT.

For purposes of the present disclosure, any particular human can be categorized as having one of three SYNJ2 genotypes: i) SYNJ2 reference; ii) heterozygous for a SYNJ2 predicted loss-of-function variant; or iii) homozygous for a SYNJ2 predicted loss-of-function variant. A human is SYNJ2 reference when the human does not have a copy of a SYNJ2 predicted loss-of-function variant nucleic acid molecule. A human is heterozygous for a SYNJ2 predicted loss-of-function variant when the human has a single copy of a SYNJ2 predicted loss-of-function variant nucleic acid molecule. A SYNJ2 predicted loss-of-function variant nucleic acid molecule is any SYNJ2 nucleic acid molecule (such as, a genomic nucleic acid molecule, an mRNA molecule, or a cDNA molecule) encoding a SYNJ2 polypeptide having a partial loss-of-function, a complete loss-of-function, a predicted partial loss-of-function, or a predicted complete loss-of-function. A human who has a SYNJ2 polypeptide having a partial loss-of-function (or predicted partial loss-of-function) is hypomorphic for SYNJ2. The SYNJ2 predicted loss-of-function variant nucleic acid molecule can be any nucleic acid molecule encoding SYNJ2 Thr656Met or rs2256014. In some embodiments, the SYNJ2 predicted loss-of-function variant nucleic acid molecule encodes SYNJ2 Thr656Met. A human is homozygous for a SYNJ2 predicted loss-of-function variant when the human has two copies of a SYNJ2 predicted loss-of-function variant nucleic acid molecule.

For human subjects or patients that are genotyped or determined to be heterozygous or homozygous for a SYNJ2 predicted loss-of-function variant nucleic acid molecule, such human subjects or patients have an increased risk of developing hearing loss and/or have an increased mean SRT. For human subjects or patients that are genotyped or determined to be heterozygous or homozygous for a SYNJ2 predicted loss-of-function variant nucleic acid molecule, such human subjects or patients can be treated with an agent effective to treat hearing loss and/or have an increased mean SRT.

The present disclosure provides methods of treating a patient with a therapeutic agent that treats or inhibits hearing loss, wherein the patient is suffering from hearing loss, the method comprising the steps of: determining whether the patient has a SYNJ2 predicted loss-of-function variant nucleic acid molecule encoding a human SYNJ2 polypeptide by: obtaining or having obtained a biological sample from the patient; and performing or having performed a genotyping assay on the biological sample to determine if the patient has a genotype comprising the SYNJ2 predicted loss-of-function variant nucleic acid molecule; and when the patient is SYNJ2 reference, then administering or continuing to administer to the patient the therapeutic agent that treats or inhibits hearing loss in a standard dosage amount; and when the patient is heterozygous or homozygous for a SYNJ2 predicted loss-of-function variant, then administering or continuing to administer to the patient the therapeutic agent that treats or inhibits hearing loss in an amount that is the same as or greater than a standard dosage amount; wherein the presence of a genotype having the SYNJ2 predicted loss-of-function variant nucleic acid molecule encoding the human SYNJ2 polypeptide indicates the patient has an increased risk of developing hearing loss; provided that the SYNJ2 predicted loss-of-function variant is not Asn538Lys. In some embodiments, the patient is heterozygous for a SYNJ2 predicted loss-of-function variant. In some embodiments, the patient is homozygous for a SYNJ2 predicted loss-of-function variant.

In some embodiments, the treatment methods further comprise detecting the presence or absence of a SYNJ2 predicted loss-of-function variant nucleic acid molecule encoding a human SYNJ2 polypeptide in a biological sample from the patient. As used throughout the present disclosure, a “SYNJ2 predicted loss-of-function variant nucleic acid molecule” is any SYNJ2 nucleic acid molecule (such as, for example, genomic nucleic acid molecule, mRNA molecule, or cDNA molecule) encoding a SYNJ2 polypeptide having a partial loss-of-function, a complete loss-of-function, a predicted partial loss-of-function, or a predicted complete loss-of-function; provided that the SYNJ2 predicted loss-of-function variant is not Asn538Lys.

In any of the embodiments described herein, the SYNJ2 predicted loss-of-function variant nucleic acid molecule can be any SYNJ2 nucleic acid molecule (such as, for example, genomic nucleic acid molecule, mRNA molecule, or cDNA molecule) encoding a SYNJ2 polypeptide having a partial loss-of-function, a complete loss-of-function, a predicted partial loss-of-function, or a predicted complete loss-of-function; provided that the SYNJ2 predicted loss-of-function variant is not Asn538Lys. For example, the SYNJ2 predicted loss-of-function variant nucleic acid molecule can be any nucleic acid molecule encoding SYNJ2 Thr656Met or rs2256014. In some embodiments, the SYNJ2 predicted loss-of-function variant nucleic acid molecule encodes SYNJ2 Thr656Met.

Detecting the presence or absence of a SYNJ2 predicted loss-of-function variant nucleic acid molecule in a biological sample from a patient and/or determining whether a patient has a SYNJ2 predicted loss-of-function variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the human subject.

The present disclosure also provides methods of treating a patient with a therapeutic agent that treats or inhibits hearing loss, wherein the patient is suffering from hearing loss, the method comprising the steps of: determining whether the patient has a SYNJ2 predicted loss-of-function polypeptide by: obtaining or having obtained a biological sample from the patient; and performing or having performed an assay on the biological sample to determine if the patient has a SYNJ2 predicted loss-of-function polypeptide; and when the patient does not have a SYNJ2 predicted loss-of-function polypeptide, then administering or continuing to administer to the patient the therapeutic agent that treats or inhibits hearing loss in a standard dosage amount; and when the patient has a SYNJ2 predicted loss-of-function polypeptide, then administering or continuing to administer to the patient the therapeutic agent that treats or inhibits hearing loss in an amount that is the same as or greater than a standard dosage amount; wherein the presence of a SYNJ2 predicted loss-of-function polypeptide indicates the patient has an increased risk of developing hearing loss. In some embodiments, the patient has a SYNJ2 predicted loss-of-function polypeptide. In some embodiments, the patient does not have a SYNJ2 predicted loss-of-function polypeptide.

In any of the embodiments described herein, the SYNJ2 predicted loss-of-function polypeptide can be any SYNJ2 polypeptide having a partial loss-of-function, a complete loss-of-function, a predicted partial loss-of-function, or a predicted complete loss-of-function; provided that the SYNJ2 predicted loss-of-function variant is not Asn538Lys. In any of the embodiments described herein, the SYNJ2 predicted loss-of-function polypeptide can be any of the SYNJ2 polypeptides described herein including, for example, SYNJ2 Thr656Met or rs2256014. In some embodiments, the SYNJ2 predicted loss-of-function polypeptide is SYNJ2 Thr656Met.

Detecting the presence or absence of a SYNJ2 predicted loss-of-function polypeptide in a biological sample from a patient and/or determining whether a patient has a SYNJ2 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the human subject.

In any of the embodiments described herein, the hearing loss is increased speech reception threshold and/or increased speech-in-noise threshold. In any of the embodiments described herein, the hearing loss is increased pure-tone threshold, bone conduction threshold, increased auditory brainstem response or increased distortion product otoacoustic emission (DPOAE). In any of the embodiments described herein, the hearing loss is decreased word recognition score.

Symptoms of hearing loss include, but are not limited to, muffling of speech and other sounds, difficulty understanding words, difficulty hearing high-pitched sounds, ringing in the ears, and hypersensitivity to certain sounds.

Examples of therapeutic agents that treat or inhibit hearing loss include, but are not limited to: antioxidants, calcium-channel blockers, anti-inflammatory drugs (such as steroids), apoptosis inhibitors, D-methionine, combination of ebselen and allopurinol, resveratrol, neurotrophic factors (such as T-817MA), caspase inhibitors (such as z-DEVD-fmk), and copper transport inhibitors (such as cimetidine and copper sulphate).

In some embodiments, the dose of the therapeutic agents that treat or inhibit hearing loss can be increased by about 10%, by about 20%, by about 30%, by about 40%, by about 50%, by about 60%, by about 70%, by about 80%, or by about 90% for patients or human subjects that are heterozygous or homozygous for a SYNJ2 predicted loss-of-function variant (i.e., a greater amount than the standard dosage amount) compared to patients or human subjects that are SYNJ2 reference (who may receive a standard dosage amount). In some embodiments, the dose of the therapeutic agents that treat or inhibit the hearing loss can be increased by about 10%, by about 20%, by about 30%, by about 40%, or by about 50%. In addition, the dose of therapeutic agents that treat or inhibit the hearing loss in patients or human subjects that are heterozygous or homozygous for a SYNJ2 predicted loss-of-function variant can be administered more frequently compared to patients or human subjects that are SYNJ2 reference.

In some embodiments, the dose of the therapeutic agents that treat or inhibit hearing loss can be increased by about 10%, by about 20%, by about 30%, by about 40%, by about 50%, by about 60%, by about 70%, by about 80%, or by about 90% for patients or human subjects that are homozygous for a SYNJ2 predicted loss-of-function variant compared to patients or human subjects that are heterozygous for a SYNJ2 predicted loss-of-function variant. In some embodiments, the dose of the therapeutic agents that treat or inhibit hearing loss can be increased by about 10%, by about 20%, by about 30%, by about 40%, or by about 50%. In addition, the dose of therapeutic agents that treat or inhibit hearing loss in patients or human subjects that are homozygous for a SYNJ2 predicted loss-of-function variant can be administered more frequently compared to patients or human subjects that are heterozygous for a SYNJ2 predicted loss-of-function variant.

Administration of the therapeutic agents that treat or inhibit hearing loss can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a patient can receive therapy for a prolonged period of time such as, for example, 6 months, 1 year, or more.

Administration of the therapeutic agents that treat or inhibit hearing loss can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in hearing loss, a decrease/reduction in the severity of hearing loss (such as, for example, a reduction or inhibition of development or hearing loss), a decrease/reduction in symptoms and hearing loss-related effects, delaying the onset of symptoms and hearing loss-related effects, reducing the severity of symptoms of hearing loss-related effects, reducing the severity of an acute episode, reducing the number of symptoms and hearing loss-related effects, reducing the latency of symptoms and hearing loss-related effects, an amelioration of symptoms and hearing loss-related effects, reducing secondary symptoms, reducing secondary infections, preventing relapse to hearing loss, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of hearing loss development/progression (such as, for example, a complete or partial avoidance/inhibition or a delay) following administration of a therapeutic protocol. Treatment of hearing loss encompasses the treatment of patients already diagnosed as having any form of hearing loss at any clinical stage or manifestation, the delay of the onset or evolution or aggravation or deterioration of the symptoms or signs of hearing loss, and/or preventing and/or reducing the severity of hearing loss.

The present disclosure also provides methods of identifying a human subject having an increased risk for developing hearing loss, wherein the method comprises: determining or having determined in a biological sample obtained from the subject the presence or absence of a SYNJ2 predicted loss-of-function variant nucleic acid molecule (such as a genomic nucleic acid molecule, mRNA molecule, and/or cDNA molecule) encoding a human SYNJ2 polypeptide; wherein: i) when the human subject lacks a SYNJ2 predicted loss-of-function variant nucleic acid molecule (i.e., the human subject is genotypically categorized as a SYNJ2 reference), then the human subject does not have an increased risk for developing hearing loss; and ii) when the human subject has a SYNJ2 predicted loss-of-function variant nucleic acid molecule (i.e., the human subject is heterozygous for a SYNJ2 predicted loss-of-function variant or homozygous for a SYNJ2 predicted loss-of-function variant), then the human subject has an increased risk for developing hearing loss.

In any of the embodiments described herein, the SYNJ2 predicted loss-of-function variant nucleic acid molecule can be any SYNJ2 nucleic acid molecule (such as, for example, genomic nucleic acid molecule, mRNA molecule, or cDNA molecule) encoding a SYNJ2 polypeptide having a partial loss-of-function, a complete loss-of-function, a predicted partial loss-of-function, or a predicted complete loss-of-function; provided that the SYNJ2 predicted loss-of-function variant is not Asn538Lys. For example, the SYNJ2 predicted loss-of-function variant nucleic acid molecule can be any nucleic acid molecule encoding SYNJ2 Thr656Met or rs2256014. In some embodiments, the SYNJ2 predicted loss-of-function variant nucleic acid molecule encodes SYNJ2 Thr656Met.

Determining whether a human subject has a SYNJ2 predicted loss-of-function variant nucleic acid molecule in a biological sample from a patient and/or determining whether a patient has a SYNJ2 predicted loss-of-function variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the human subject.

In any of the embodiments described herein, the hearing loss is increased speech reception threshold and/or increased speech-in-noise threshold. In any of the embodiments described herein, the hearing loss is increased pure-tone threshold, bone conduction threshold, increased auditory brainstem response or increased distortion product otoacoustic emission (DPOAE). In any of the embodiments described herein, the hearing loss is decreased word recognition score.

In some embodiments, when a human subject is identified as having an increased risk of developing hearing loss, the human subject is treated with a therapeutic agent that treats or inhibits hearing loss, as described herein. In some embodiments, when the patient is heterozygous or homozygous for a SYNJ2 predicted loss-of-function variant, the patient is administered the therapeutic agent that treats or inhibits hearing loss in a dosage amount that is the same as or greater than the standard dosage amount. In some embodiments, when the patient is homozygous for a SYNJ2 predicted loss-of-function variant, the patient is administered the therapeutic agent that treats or inhibits hearing loss in a dosage amount that is the same as or greater than the dosage amount administered to a patient that is heterozygous for a SYNJ2 predicted loss-of-function variant. In some embodiments, the patient is heterozygous for a SYNJ2 predicted loss-of-function variant. In some embodiments, the patient is homozygous for a SYNJ2 predicted loss-of-function variant.

The present disclosure also provides methods of detecting the presence or absence of a SYNJ2 predicted loss-of-function variant genomic nucleic acid molecule in a biological sample from a subject, a SYNJ2 predicted loss-of-function variant mRNA molecule in a biological sample from a subject, and/or a SYNJ2 predicted loss-of-function variant cDNA molecule produced from an mRNA molecule in a biological sample from a subject. It is understood that gene sequences within a population and mRNA molecules encoded by such genes can vary due to polymorphisms such as single-nucleotide polymorphisms. The sequences provided herein for the SYNJ2 variant genomic nucleic acid molecule, SYNJ2 variant mRNA molecule, and SYNJ2 variant cDNA molecule are only exemplary sequences. Other sequences for the SYNJ2 variant genomic nucleic acid molecule, variant mRNA molecule, and variant cDNA molecule are also possible.

The biological sample can be derived from any cell, tissue, or biological fluid from the subject. The sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The sample used in the methods disclosed herein will vary based on the assay format, nature of the detection method, and the tissues, cells, or extracts that are used as the sample. A biological sample can be processed differently depending on the assay being employed. For example, when detecting any SYNJ2 variant nucleic acid molecule, preliminary processing designed to isolate or enrich the sample for the genomic DNA can be employed. A variety of techniques may be used for this purpose. When detecting the level of any SYNJ2 variant mRNA, different techniques can be used enrich the biological sample with mRNA. Various methods to detect the presence or level of an mRNA or the presence of a particular variant genomic DNA locus can be used.

In some embodiments, detecting a human SYNJ2 predicted loss-of-function variant nucleic acid molecule in a human subject comprises assaying or genotyping a biological sample obtained from the human subject to determine whether a SYNJ2 genomic nucleic acid molecule and/or a SYNJ2 mRNA molecule in the biological sample, and/or a SYNJ2 cDNA molecule produced from an mRNA moleucle in the biological sample, comprises one or more variations that cause a loss-of-function (partial or complete) or are predicted to cause a loss-of-function (partial or complete).

In some embodiments, the methods of detecting the presence or absence of a SYNJ2 predicted loss-of-function variant nucleic acid molecule (such as, for example, a genomic nucleic acid molecule, an mRNA molecule, and/or a cDNA molecule produced from an mRNA molecule) in a human subject, comprise: performing an assay on a biological sample obtained from the human subject, which assay determines whether a nucleic acid molecule in the biological sample comprises a particular nucleotide sequence. In some embodiments, the nucleotide sequence comprises: a thymine at a position corresponding to position 89,742 according to SEQ ID NO:2 (for genomic nucleic acid molecules); a uracil at a position corresponding to position 2,050 according to SEQ ID NO:5 (for mRNA molecules); or a thymine at a position corresponding to position 2,050 according to SEQ ID NO:7 (for cDNA molecules obtained from mRNA molecules).

In some embodiments, the nucleotide sequence comprises a thymine at a position corresponding to position 99,219 according to SEQ ID NO:3 (for genomic nucleic acid molecules).

In some embodiments, the method of detecting a human SYNJ2 variant nucleic acid molecule in a human subject comprising assaying a sample obtained from the human subject to determine whether a genomic nucleic acid molecule in the sample comprises a nucleotide sequence comprising: a thymine at a position corresponding to position 89,742 according to SEQ ID NO:2, or the complement thereof; or a thymine at a position corresponding to position 99,219 according to SEQ ID NO:3, or the complement thereof.

In some embodiments, the method of detecting a human SYNJ2 variant nucleic acid molecule in a human subject comprising assaying a sample obtained from the human subject to determine whether an mRNA molecule in the sample comprises a nucleotide sequence comprising a uracil at a position corresponding to position 2,050 according to SEQ ID NO:5, or the complement thereof.

In some embodiments, the method of detecting a human SYNJ2 variant nucleic acid molecule in a human subject comprising assaying a sample obtained from the human subject to determine whether a cDNA molecule produced from an mRNA molecule in the sample comprises a nucleotide sequence comprising a thymine at a position corresponding to position 2,050 according to SEQ ID NO:7, or the complement thereof.

In some embodiments, the biological sample comprises a cell or cell lysate. Such methods can further comprise, for example, obtaining a biological sample from the subject comprising a SYNJ2 genomic nucleic acid molecule or mRNA molecule, and if mRNA, optionally reverse transcribing the mRNA into cDNA. Such assays can comprise, for example determining the identity of these positions of the particular SYNJ2 nucleic acid molecule. In some embodiments, the method is an in vitro method.

In some embodiments, the determining step, detecting step, or genotyping assay comprises sequencing at least a portion of the nucleotide sequence of the SYNJ2 genomic nucleic acid molecule, the SYNJ2 mRNA molecule, or the SYNJ2 cDNA molecule in the biological sample, wherein the sequenced portion comprises one or more variations that cause a loss-of-function (partial or complete) or are predicted to cause a loss-of-function (partial or complete).

In some embodiments, the determining step, detecting step, or genotyping assay comprises sequencing at least a portion of: the nucleotide sequence of the SYNJ2 genomic nucleic acid molecule in the biological sample, wherein the sequenced portion comprises a position corresponding to position 89,742 according to SEQ ID NO:2, or the complement thereof; the nucleotide sequence of the SYNJ2 mRNA molecule in the biological sample, wherein the sequenced portion comprises a position corresponding to position 2,050 according to SEQ ID NO:5, or the complement thereof; the nucleotide sequence of the SYNJ2 cDNA molecule in the biological sample, wherein the sequenced portion comprises a position corresponding to position 2,050 according to SEQ ID NO:7, or the complement thereof; or the nucleotide sequence of the SYNJ2 genomic nucleic acid molecule in the biological sample, wherein the sequenced portion comprises a position corresponding to position 99,219 according to SEQ ID NO:3, or the complement thereof. When the sequenced portion of the SYNJ2 genomic nucleic acid molecule in the biological sample comprises: a thymine at a position corresponding to position 89,742 according to SEQ ID NO:2; or a thymine at a position corresponding to position 99,219 according to SEQ ID NO:3, then the SYNJ2 genomic nucleic acid molecule in the biological sample is a SYNJ2 predicted loss-of-function variant genomic nucleic acid molecule. When the sequenced portion of the SYNJ2 mRNA molecule in the biological sample comprises: a uracil at a position corresponding to position 2,050 according to SEQ ID NO:5, then the SYNJ2 mRNA molecule in the biological sample is a SYNJ2 predicted loss-of-function variant mRNA molecule. When the sequenced portion of the SYNJ2 cDNA molecule in the biological sample comprises: a thymine at a position corresponding to position 2,050 according to SEQ ID NO:7, then the SYNJ2 cDNA molecule in the biological sample is a SYNJ2 predicted loss-of-function variant cDNA molecule.

In some embodiments, the assay comprises sequencing at least a portion of the genomic nucleic acid molecule, wherein the sequenced portion comprises: a thymine at a position corresponding to position 89,742 according to SEQ ID NO:2, or the complement thereof; or a thymine at a position corresponding to position 99,219 according to SEQ ID NO:3, or the complement thereof.

In some embodiments, the assay comprises sequencing at least a portion of the mRNA molecule, wherein the sequenced portion comprises a uracil at a position corresponding to position 2,050 according to SEQ ID NO:5, or the complement thereof.

In some embodiments, the assay comprises sequencing at least a portion of the cNDA molecule, wherein the sequenced portion comprises a thymine at a position corresponding to position 2,050 according to SEQ ID NO:7, or the complement thereof.

In some embodiments, the determining step, detecting step, or genotyping assay comprises: a) contacting the biological sample with a primer hybridizing to a portion of the nucleotide sequence of the SYNJ2: genomic nucleic acid molecule that is proximate to a position corresponding to position 89,742 according to SEQ ID NO:2; mRNA molecule that is proximate to a position corresponding to position 2,050 according to SEQ ID NO:5; or cDNA molecule that is proximate to a position corresponding to position 2,050 according to SEQ ID NO:7; b) extending the primer at least through the position of the nucleotide sequence of the SYNJ2: genomic nucleic acid molecule corresponding to position 89,742 according to SEQ ID NO:2; mRNA molecule corresponding to position 2,050 according to SEQ ID NO:5; or cDNA molecule corresponding to position 2,050 according to SEQ ID NO:7; and c) determining whether the extension product of the primer comprises: a thymine at a position corresponding to position 89,742 according to SEQ ID NO:2; a uracil at a position corresponding to position 2,050 according to SEQ ID NO:5; or a thymine at a position corresponding to position 2,050 according to SEQ ID NO:7.

In some embodiments, the determining step, detecting step, or genotyping assay comprises: a) contacting the biological sample with a primer hybridizing to a portion of the nucleotide sequence of the SYNJ2: genomic nucleic acid molecule that is proximate to a position corresponding to position 99,219 according to SEQ ID NO:3; b) extending the primer at least through the position of the nucleotide sequence of the SYNJ2: genomic nucleic acid molecule corresponding to position 99,219 according to SEQ ID NO:3; and c) determining whether the extension product of the primer comprises: a thymine at a position corresponding to position 99,219 according to SEQ ID NO:3.

In some embodiments, the assay comprises: a) contacting the sample with a primer hybridizing to a portion of the nucleotide sequence of the SYNJ2 genomic nucleic acid molecule that is proximate to: a position corresponding to position 89,742 according to SEQ ID NO:2; or a position corresponding to position 99,219 according to SEQ ID NO:3; b) extending the primer at least through the position of the nucleotide sequence of the SYNJ2 genomic nucleic acid molecule corresponding to: position 89,742 according to SEQ ID NO:2; or to position 99,219 according to SEQ ID NO:3; c) determining whether the extension product of the primer comprises: a thymine at a position corresponding to position 89,742 according to SEQ ID NO:2; or a thymine at a position corresponding to position 99,219 according to SEQ ID NO:3.

In some embodiments, the assay comprises: a) contacting the sample with a primer hybridizing to a portion of the nucleotide sequence of the SYNJ2 mRNA molecule that is proximate to a position corresponding to position 2,050 according to SEQ ID NO:5; b) extending the primer at least through the position of the nucleotide sequence of the SYNJ2 mRNA molecule corresponding to position 2,050 according to SEQ ID NO:5; c) determining whether the extension product of the primer comprises a uracil at a position corresponding to position 2,050 according to SEQ ID NO:5.

In some embodiments, the assay comprises: a) contacting the sample with a primer hybridizing to a portion of the nucleotide sequence of the SYNJ2 cDNA molecule that is proximate to a position corresponding to position 2,050 according to SEQ ID NO:7; b) extending the primer at least through the position of the nucleotide sequence of the SYNJ2 cDNA molecule corresponding to position 2,050 according to SEQ ID NO:7; and c) determining whether the extension product of the primer comprises a thymine at a position corresponding to position 2,050 according to SEQ ID NO:7.

In some embodiments, the assay comprises sequencing the entire nucleic acid molecule. In some embodiments, only a SYNJ2 genomic nucleic acid molecule is analyzed. In some embodiments, only a SYNJ2 mRNA is analyzed. In some embodiments, only a SYNJ2 cDNA obtained from SYNJ2 mRNA is analyzed.

In some embodiments, the determining step, detecting step, or genotyping assay comprises: a) amplifying at least a portion of the nucleic acid molecule; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to: the nucleic acid sequence of the amplified nucleic acid molecule comprising a thymine at a position corresponding to position 89,742 according to SEQ ID NO:2, or the complement thereof; the nucleic acid sequence of the amplified nucleic acid molecule comprising a uracil at a position corresponding to position 2,050 according to SEQ ID NO:5, or the complement thereof; the nucleic acid sequence of the amplified nucleic acid molecule comprising a thymine at a position corresponding to position 2,050 according to SEQ ID NO:7, or the complement thereof; or the nucleic acid sequence of the amplified nucleic acid molecule comprising a thymine at a position corresponding to position 99,219 according to SEQ ID NO:3, or the complement thereof; and d) detecting the detectable label.

In some embodiments, the assay comprises: a) amplifying at least a portion of the genomic nucleic acid molecule; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: a thymine at a position corresponding to position 89,742 according to SEQ ID NO:2, or the complement thereof; or a thymine at a position corresponding to position 99,219 according to SEQ ID NO:3, or the complement thereof; and d) detecting the detectable label.

In some embodiments, the assay comprises: a) amplifying at least a portion of the mRNA molecule; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a uracil at a position corresponding to position 2,050 according to SEQ ID NO:5, or the complement thereof; and d) detecting the detectable label.

In some embodiments, the assay comprises: a) amplifying at least a portion of the cDNA molecule; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a thymine at a position corresponding to position 2,050 according to SEQ ID NO:7, or the complement thereof; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or genotyping assay comprises: contacting the nucleic acid molecule with an alteration-specific probe comprising a detectable label, wherein the alteration-specific probe comprises a nucleotide sequence which hybridizes under stringent conditions to: the nucleic acid sequence of the amplified nucleic acid molecule comprising a thymine at a position corresponding to position 89,742 according to SEQ ID NO:2, or the complement thereof; the nucleic acid sequence of the amplified nucleic acid molecule comprising a uracil at a position corresponding to position 2,050 according to SEQ ID NO:5, or the complement thereof; the nucleic acid sequence of the amplified nucleic acid molecule comprising a thymine at a position corresponding to position 2,050 according to SEQ ID NO:7, or the complement thereof; or the nucleic acid sequence of the amplified nucleic acid molecule comprising a thymine at a position corresponding to position 99,219 according to SEQ ID NO:3, or the complement thereof; and detecting the detectable label.

In some embodiments, the assay comprises: contacting the genomic nucleic acid molecule with an alteration-specific probe comprising a detectable label, wherein the alteration-specific probe comprises a nucleotide sequence which hybridizes under stringent conditions to the nucleic acid sequence of the amplified nucleic acid molecule comprising: a thymine at a position corresponding to position 89,742 according to SEQ ID NO:2, or the complement thereof; or a thymine at a position corresponding to position 99,219 according to SEQ ID NO:3, or the complement thereof; and detecting the detectable label.

In some embodiments, the assay comprises: contacting the mRNA molecule with an alteration-specific probe comprising a detectable label, wherein the alteration-specific probe comprises a nucleotide sequence which hybridizes under stringent conditions to the nucleic acid sequence of the amplified nucleic acid molecule comprising a uracil at a position corresponding to position 2,050 according to SEQ ID NO:5, or the complement thereof; and detecting the detectable label.

In some embodiments, the assay comprises: contacting the cDNA molecule with an alteration-specific probe comprising a detectable label, wherein the alteration-specific probe comprises a nucleotide sequence which hybridizes under stringent conditions to the nucleic acid sequence of the amplified nucleic acid molecule comprising a thymine at a position corresponding to position 2,050 according to SEQ ID NO:7, or the complement thereof; and detecting the detectable label.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the human subject.

The SYNJ2 predicted loss-of-function variant nucleic acid molecule can be any SYNJ2 nucleic acid molecule (such as, for example, genomic nucleic acid molecule, mRNA molecule, or cDNA molecule) encoding a SYNJ2 polypeptide having a partial loss-of-function, a complete loss-of-function, a predicted partial loss-of-function, or a predicted complete loss-of-function; provided that the SYNJ2 predicted loss-of-function variant is not Asn538Lys. For example, the SYNJ2 predicted loss-of-function variant nucleic acid molecule can be any nucleic acid molecule encoding SYNJ2 Thr656Met or rs2256014. In some embodiments, the SYNJ2 predicted loss-of-function variant nucleic acid molecule encodes SYNJ2 Thr656Met.

In some embodiments, the assay comprises contacting the biological sample with a primer or probe, such as an alteration-specific primer or alteration-specific probe, that specifically hybridizes to a SYNJ2 variant genomic sequence, variant mRNA sequence, or variant cDNA sequence and not the corresponding SYNJ2 reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a SYNJ2 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether a SYNJ2 nucleic acid molecule (genomic nucleic acid molecule, mRNA molecule, or cDNA molecule), or complement thereof, within a biological sample comprises a nucleotide sequence comprising a thymine at a position corresponding to position 89,742 according to SEQ ID NO:2 (genomic nucleic acid molecule), a uracil at a position corresponding to position 2,050 according to SEQ ID NO:5 (mRNA molecule), or a thymine at a position corresponding to position 2,050 according to SEQ ID NO:7 (cDNA molecule), the biological sample can be subjected to an amplification method using a primer pair that includes a first primer derived from the 5′ flanking sequence adjacent to a thymine at a position corresponding to position 89,742 according to SEQ ID NO:2, a uracil at a position corresponding to position 2,050 according to SEQ ID NO:5, or a thymine at a position corresponding to position 2,050 according to SEQ ID NO:7, and a second primer derived from the 3′ flanking sequence adjacent to a thymine at a position corresponding to position 89,742 according to SEQ ID NO:2, a uracil at a position corresponding to position 2,050 according to SEQ ID NO:5, or a thymine at a position corresponding to position 2,050 according to SEQ ID NO:7, to produce an amplicon that is indicative of the presence of the SNP at positions encoding a thymine at a position corresponding to position 89,742 according to SEQ ID NO:2, a uracil at a position corresponding to position 2,050 according to SEQ ID NO:5, or a thymine at a position corresponding to position 2,050 according to SEQ ID NO:7. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a thymine at a position corresponding to position 89,742 according to SEQ ID NO:2, or a uracil at a position corresponding to position 2,050 according to SEQ ID NO:5, or a thymine at a position corresponding to position 2,050 according to SEQ ID NO:7, and at least 1, 2, 3, 4, 5, 6, 7, 8, 9, 10, or more nucleotides on each side of positions comprising a thymine at a position corresponding to position 89,742 according to SEQ ID NO:2, or a uracil at a position corresponding to position 2,050 according to SEQ ID NO:5, or a thymine at a position corresponding to position 2,050 according to SEQ ID NO:7.

In some embodiments, to determine whether a SYNJ2 nucleic acid molecule (genomic nucleic acid molecule, mRNA molecule, or cDNA molecule), or complement thereof, within a biological sample comprises a nucleotide sequence comprising a thymine at a position corresponding to position 99,219 according to SEQ ID NO:3 (genomic nucleic acid molecule), the biological sample can be subjected to an amplification method using a primer pair that includes a first primer derived from the 5′ flanking sequence adjacent to a thymine at a position corresponding to position 99,219 according to SEQ ID NO:3, and a second primer derived from the 3′ flanking sequence adjacent to a thymine at a position corresponding to position 99,219 according to SEQ ID NO:3 to produce an amplicon that is indicative of the presence of the SNP at positions encoding a thymine at a position corresponding to position 99,219 according to SEQ ID NO:3. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a thymine at a position corresponding to position 99,219 according to SEQ ID NO:3 and at least 1, 2, 3, 4, 5, 6, 7, 8, 9, 10, or more nucleotides on each side of positions comprising a thymine at a position corresponding to position 99,219 according to SEQ ID NO:3.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2× SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of a human SYNJ2 predicted loss-of-function polypeptide comprising performing an assay on a sample obtained from a human subject to determine whether a SYNJ2 polypeptide in the subject contains one or more variations that causes the polypeptide to have a loss-of-function (partial or complete) or predicted loss-of-function (partial or complete); provided that the SYNJ2 predicted loss-of-function variant is not Asn538Lys. The SYNJ2 predicted loss-of-function polypeptide can be any of the SYNJ2 truncated variant polypeptides described herein. In some embodiments, the methods detect the presence of SYNJ2 Thr656Met, Thr419Met, Glu1468Gly, Glu1231Gly, rs2256014, or rs6899791. In some embodiments, the methods detect the presence of SYNJ2 Thr656Met or Thr419Met.

In some embodiments, the methods comprise performing an assay on a sample obtained from a human subject to determine whether a SYNJ2 polypeptide in the sample comprises a methionine at a position corresponding to position 656 according to SEQ ID NO:9. In some embodiments, the detecting step comprises sequencing at least a portion of the polypeptide that comprises a position corresponding to position 656 according to SEQ ID NO:8 or SEQ ID NO:9. In some embodiments, the detecting step comprises sequencing the entire polypeptide.

In some embodiments, the detecting step comprises an immunoassay for detecting the presence of a polypeptide that comprises a position corresponding to position 656 according to SEQ ID NO:8 or SEQ ID NO:9.

In some embodiments, when the human subject does not have a SYNJ2 predicted loss-of-function polypeptide, then the human subject does not have an increased risk for developing hearing loss; and when the human subject has a SYNJ2 predicted loss-of-function polypeptide, then the human subject has an increased risk for developing hearing loss.

The present disclosure also provides isolated nucleic acid molecules that hybridize to SYNJ2 variant genomic nucleic acid molecules, SYNJ2 variant mRNA molecules, and/or SYNJ2 variant cDNA molecules (such as any of the genomic variant nucleic acid molecules, mRNA variant molecules, and cDNA variant molecules disclosed herein. In some embodiments, the isolated nucleic acid molecules hybridize to a portion of the SYNJ2 nucleic acid molecule that includes: a position corresponding to position 89,742 according to SEQ ID NO:2; a position corresponding to position 2,050 according to SEQ ID NO:5; or a position corresponding to position 2,050 according to SEQ ID NO:7. In some embodiments, the isolated nucleic acid molecules hybridize to a portion of the SYNJ2 nucleic acid molecule that includes a position corresponding to position 99,219 according to SEQ ID NO:3.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to SYNJ2 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to SYNJ2 variant genomic nucleic acid molecules, SYNJ2 variant mRNA molecules, and/or SYNJ2 variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human SYNJ2 polypeptide, wherein the portion comprises a position corresponding to: position 89,742 according to SEQ ID NO:2, or the complement thereof; position 2,050 according to SEQ ID NO:5, or the complement thereof; or position 2,050 according to SEQ ID NO:7, or the complement thereof. In some embodiments, the alteration-specific probe or alteration-specific primer comprises a nucleotide sequence which is complementary to a portion of a nucleotide sequence comprising positions corresponding to: positions 89,741-89,743 according to SEQ ID NO:2, or the complement thereof; positions 2,049-2,051 according to SEQ ID NO:5, or the complement thereof; or positions 2,049-2,051 according to SEQ ID NO:7, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human SYNJ2 polypeptide, wherein the portion comprises a position corresponding to position 99,219 according to SEQ ID NO:3, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the SYNJ2 variant genomic nucleic acid molecules, SYNJ2 variant mRNA molecules, and/or SYNJ2 variant cDNA molecules disclosed herein. The primers described herein can be used to amplify SYNJ2 variant genomic nucleic acid molecules, SYNJ2 variant mRNA molecules, or SYNJ2 variant cDNA molecules, or a fragment thereof.

The present disclosure also provides pairs of primers comprising any of the primers described above. If one of the primers' 3′-ends hybridizes to a cytosine at a position coresponding to position 89,742 according to SEQ ID NO:1 (rather than thymine) in a particular SYNJ2 nucleic acid molecule, then the presence of the amplified fragment would indicate the presence of a SYNJ2 reference genomic nucleic acid molecule. Conversely, if one of the primers' 3′-ends hybridizes to a thymine at a position corresponding to position 89,742 according to SEQ ID NO:2 (rather than cytosine) in a particular SYNJ2 nucleic acid molecule, then the presence of the amplified fragment would indicate the presence of the SYNJ2 variant genomic nucleic acid molecule. In some embodiments, the nucleotide of the primer complementary to the thymine at a position corresponding to position 89,742 according to SEQ ID NO:2 can be at the 3′ end of the primer. In addition, if one of the primers' 3′-ends hybridizes to a cytosine at a position corresponding to position 2,050 according to SEQ ID NO:4 (rather than uracil) in a particular SYNJ2 nucleic acid molecule, then the presence of the amplified fragment would indicate the presence of a SYNJ2 reference mRNA molecule. Conversely, if one of the primers' 3′-ends hybridizes to a uracil at a position corresponding to position 2,050 according to SEQ ID NO:5 (rather than cytosine) in a particular SYNJ2 mRNA molecule, then the presence of the amplified fragment would indicate the presence of the SYNJ2 variant mRNA molecule. In some embodiments, the nucleotide of the primer complementary to the uracil at a position corresponding to position 2,050 according to SEQ ID NO:5 can be at the 3′ end of the primer. In addition, if one of the primers' 3′-ends hybridizes to a cytosine at a position corresponding to position 2,050 according to SEQ ID NO:6 (rather than thymine) in a particular SYNJ2 nucleic acid molecule, then the presence of the amplified fragment would indicate the presence of a SYNJ2 reference cDNA molecule. Conversely, if one of the primers' 3′-ends hybridizes to a thymine at a position corresponding to position 2,050 according to SEQ ID NO:7 (rather than cytosine) in a particular SYNJ2 cDNA molecule, then the presence of the amplified fragment would indicate the presence of the SYNJ2 variant cDNA molecule. In some embodiments, the nucleotide of the primer complementary to the thymine at a position corresponding to position 2,050 according to SEQ ID NO:7 can be at the 3′ end of the primer.

If one of the primers' 3′-ends hybridizes to a guanine at a position coresponding to position 99,219 according to SEQ ID NO:1 (rather than thymine) in a particular SYNJ2 nucleic acid molecule, then the presence of the amplified fragment would indicate the presence of a SYNJ2 reference genomic nucleic acid molecule. Conversely, if one of the primers' 3′-ends hybridizes to a thymine at a position corresponding to position 99,219 according to SEQ ID NO:3 (rather than guanine) in a particular SYNJ2 nucleic acid molecule, then the presence of the amplified fragment would indicate the presence of the SYNJ2 variant genomic nucleic acid molecule. In some embodiments, the nucleotide of the primer complementary to the thymine at a position corresponding to position 99,219 according to SEQ ID NO:3 can be at the 3′ end of the primer.

In the context of the disclosure “specifically hybridizes” means that the probe or primer (such as, for example, the alteration-specific probe or alteration-specific primer) does not hybridize to a nucleic acid sequence encoding a SYNJ2 reference genomic nucleic acid molecule, a SYNJ2 reference mRNA molecule, and/or a SYNJ2 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The present disclosure also provides molecular complexes comprising or consisting of any of the Synaptojanin-2 nucleic acid molecules (genomic nucleic acid molecules, mRNA molecules, or cDNA molecules), or complement thereof, described herein and any of the alteration-specific primers or alteration-specific probes described herein. In some embodiments, the SYNJ2 nucleic acid molecules (genomic nucleic acid molecules, mRNA molecules, or cDNA molecules), or complement thereof, in the molecular complexes are single-stranded. In some embodiments, the SYNJ2 nucleic acid molecule is any of the genomic nucleic acid molecules described herein. In some embodiments, the SYNJ2 nucleic acid molecule is any of the mRNA molecules described herein. In some embodiments, the SYNJ2 nucleic acid molecule is any of the cDNA molecules described herein. In some embodiments, the molecular complex comprises or consists of any of the SYNJ2 nucleic acid molecules (genomic nucleic acid molecules, mRNA molecules, or cDNA molecules), or complement thereof, described herein and any of the alteration-specific primers described herein. In some embodiments, the molecular complex comprises or consists of any of the SYNJ2 nucleic acid molecules (genomic nucleic acid molecules, mRNA molecules, or cDNA molecules), or complement thereof, described herein and any of the alteration-specific probes described herein.

In some embodiments, the molecular complex comprises or consists of an alteration-specific primer or an alteration-specific probe hybridized to a genomic nucleic acid molecule comprising a nucleotide sequence encoding a human Synaptojanin-2 polypeptide, wherein the alteration-specific primer or the alteration-specific probe is hybridized to: a thymine at a position corresponding to position 89,742 according to SEQ ID NO:2, or the complement thereof; or a thymine at a position corresponding to position 99,219 according to SEQ ID NO:3, or the complement thereof.

In some embodiments, the molecular complex comprises or consists of an alteration-specific primer or an alteration-specific probe that is hybridized to an ATG codon at positions corresponding to positions 89,741-89,743 according to SEQ ID NO:2.

In some embodiments, the molecular complex comprises or consists of a genomic nucleic acid molecule that comprises or consists of a nucleotide sequence according to SEQ ID NO:2 or SEQ ID NO:3.

In some embodiments, the molecular complex comprises or consists of an alteration-specific primer or an alteration-specific probe hybridized to an mRNA molecule comprising a nucleotide sequence encoding a human Synaptojanin-2 polypeptide, wherein the alteration-specific primer or the alteration-specific probe is hybridized to a uracil at a position corresponding to position 2,050 according to SEQ ID NO:5, or the complement thereof.

In some embodiments, the molecular complex comprises or consists of an alteration-specific primer or an alteration-specific probe that is hybridized to an AUG codon at positions corresponding to positions 2,049-2,051 according to SEQ ID NO:5.

In some embodiments, the molecular complex comprises or consists of an mRNA molecule that comprises or consists of a nucleotide sequencde according to SEQ ID NO:5.

In some embodiments, the molecular complex comprises or consists of an alteration-specific primer or an alteration-specific probe hybridized to a cDNA molecule comprising a nucleotide sequence encoding a human Synaptojanin-2 polypeptide, wherein the alteration-specific primer or the alteration-specific probe is hybridized to a thymine at a position corresponding to position 2,050 according to SEQ ID NO:7, or the complement thereof.

In some embodiments, the molecular complex comprises or consists of an alteration-specific primer or an alteration-specific probe that is hybridized to an ATG codon at positions corresponding to positions 2,049-2,051 according to SEQ ID NO:7.

In some embodiments, the molecular complex comprises or consists of a cDNA molecule that comprises or consists of a nucleotide sequence according to SEQ ID NO:7.

In some embodiments, the molecular complex comprises an alteration-specific probe or an alteration-specific primer comprising a label. In some embodiments, the label is a fluorescent label, a radiolabel, or biotin. In some embodiments, the molecular complex further comprises a non-human polymerase.

The nucleotide sequence of a SYNJ2 reference genomic nucleic acid molecule is set forth in SEQ ID NO:1. Referring to SEQ ID NO:1, position 89,742 is a cytosine. Referring to SEQ ID NO:1, position 114,390 is an adenine. Referring to SEQ ID NO:1, position 99,219 is a guanine. Referring to SEQ ID NO:1, position 95,136 is a thymine.

A variant genomic nucleic acid molecule of SYNJ2 exists, wherein the cytosine at position 89,742 is replaced with thymine (resulting from SNP designated r5146694394). The nucleotide sequence of this SYNJ2 variant genomic nucleic acid molecule is set forth in SEQ ID NO:2. The nucleotide sequence comprises an ATG codon at positions corresponding to positions 89,741-89,743 according to SEQ ID NO:2.

Another variant genomic nucleic acid molecule of SYNJ2 exists, wherein the guanine at position 99,219 is replaced with thymine (resulting from SNP designated r52256014). The nucleotide sequence of this SYNJ2 variant genomic nucleic acid molecule is set forth in SEQ ID NO:3.

The nucleotide sequence of a first SYNJ2 reference mRNA isoform molecule (GenBank Accession Number AF318616) is set forth in SEQ ID NO:4. Referring to SEQ ID NO:4, position 2,050 is a cytosine. Referring to SEQ ID NO:4, position 4,486 is an adenine.

A variant mRNA molecule of SYNJ2 exists, wherein the cytosine at position 2,050 is replaced with uracil. The nucleotide sequence of this SYNJ2 variant mRNA molecule is set forth in SEQ ID NO:5. The nucleotide sequence comprises an AUG codon at positions corresponding to positions 2,049-2,051 according to SEQ ID NO:5.

The nucleotide sequence of a first SYNJ2 reference cDNA isoform molecule (GenBank Accession Number AF318616) is set forth in SEQ ID NO:6. Referring to SEQ ID NO:6, position 2,050 is a cytosine. Referring to SEQ ID NO:6, position 4,486 is an adenine.

A variant cDNA molecule of SYNJ2 exists, wherein the cytosine at position 2,050 is replaced with thymine. The nucleotide sequence of this SYNJ2 variant cDNA molecule is set forth in SEQ ID NO:7. The nucleotide sequence comprises an ATG codon at positions corresponding to positions 2,049-2,051 according to SEQ ID NO:7.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

The present disclosure also provides fragments of any of the isolated genomic nucleic acid molecules, mRNA molecules, or cDNA molecules disclosed herein. In some embodiments, the fragments comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 contiguous residues of any of the nucleic acid molecules disclosed herein, or any complement thereof. In some embodiments, the fragments comprise or consist of at least about 20, at least about 25, at least about 30, or at least about 35 contiguous residues of any of the nucleic acid molecules disclosed herein, or any complement thereof. In this regard, the longer fragments are preferred over the shorter ones. Such fragments may be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6XHis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10. Other modifications at the 2′ position include, but are not limited to, C₁₋₁₀oalkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO2, N3, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:4, or SEQ ID NO:6). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, a nucleic acid molecule comprising a nucleotide sequence encoding a human SYNJ2 polypeptide, wherein the nucleotide sequence comprises a thymine at a position corresponding to position 89,742 according to SEQ ID NO:2 means that if the nucleotide sequence of the SYNJ2 genomic nucleic acid molecule is aligned to the sequence of SEQ ID NO:2, the SYNJ2 sequence has a thymine residue at the position that corresponds to position 89,742 of SEQ ID NO:2. The same applies for mRNA molecules comprising a nucleotide sequence encoding a human SYNJ2 polypeptide, wherein the nucleotide sequence comprises a uracil at a position corresponding to position 2,050 according to SEQ ID NO:5, and cDNA molecules comprising a nucleotide sequence encoding a human SYNJ2 polypeptide, wherein the nucleotide sequence comprises a thymine at a position corresponding to position 2,050 according to SEQ ID NO:7. In other words, these phrases refer to a nucleic acid molecule encoding a SYNJ2 polypeptide, wherein the genomic nucleic acid molecule has a nucleotide sequence that comprises a thymine residue that is homologous to the thymine residue at position 89,742 of SEQ ID NO:2 (or wherein the mRNA molecule has a nucleotide sequence that comprises a uracil residue that is homologous to the uracil residue at position 2,050 of SEQ ID NO:5, or wherein the cDNA molecule has a nucleotide sequence that comprises a thymine residue that is homologous to the thymine residue at position 2,050 of SEQ ID NO:7). Herein, such a sequence is also referred to as “SYNJ2 sequence with the Thr656Met alteration” or “SYNJ2 sequence with the Thr656Met variation” referring to genomic nucleic acid molecules (or “SYNJ2 sequence with the C2,050U alteration” or “SYNJ2 sequence with the C2,050U variation” referring to mRNA molecules, and “SYNJ2 sequence with the C2,050T alteration” or “SYNJ2 sequence with the C2,050T variation” referring to cDNA molecules).

As described herein, a position within a SYNJ2 genomic nucleic acid molecule that corresponds to position 89,742 according to SEQ ID NO:2, for example, can be identified by performing a sequence alignment between the nucleotide sequence of a particular SYNJ2 nucleic acid molecule and the nucleotide sequence of SEQ ID NO:2. A variety of computational algorithms exist that can be used for performing a sequence alignment to identify a nucleotide position that corresponds to, for example, position 89,742 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequence of a SYNJ2 reference polypeptide is set forth in SEQ ID NO:8 (UniProt Accession Number 015056-1). Referring to SEQ ID NO:8, the SYNJ2 reference polypeptide is 1,496 amino acids in length. Referring to SEQ ID NO:8, position 656 is threonine. Referring to SEQ ID NO:8, position 1,468 is glutamic acid.

A SYNJ2 variant polypeptide exists (Thr656Met), the amino acid sequence of which is set forth in SEQ ID NO:9. Referring to SEQ ID NO:9, the SYNJ2 variant polypeptide is 1,496 amino acids in length. Referring to SEQ ID NO:9, position 656 is methionine.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or inhibit hearing loss for use in the treatment of hearing loss (or for use in the preparation of a medicament for treating hearing loss) in a human subject, wherein the human subject has any of the genomic nucleic acid molecules, mRNA molecules, and/or cDNA molecules encoding a human SYNJ2 polypeptide described herein. The therapeutic agents that treat or inhibit hearing loss can be any of the therapeutic agents that treat or inhibit hearing loss described herein.

In some embodiments, the human subject has: a genomic nucleic acid molecule having a nucleotide sequence encoding a human SYNJ2 polypeptide, wherein the nucleotide sequence comprises a thymine at a position corresponding to position 89,742 according to SEQ ID NO:2, or the complement thereof; an mRNA molecule having a nucleotide sequence encoding a human SYNJ2 polypeptide, wherein the nucleotide sequence comprises a uracil at a position corresponding to position 2,050 according to SEQ ID NO:5, or the complement thereof; a cDNA molecule having a nucleotide sequence encoding a human SYNJ2 polypeptide, wherein the nucleotide sequence comprises a thymine at a position corresponding to position 2,050 according to SEQ ID NO:7, or the complement thereof; or a SYNJ2 polypeptide that comprises a methionine at a position corresponding to position 656 according to SEQ ID NO:9.

In some embodiments, the human subject has a genomic nucleic acid molecule having a nucleotide sequence encoding a human SYNJ2 polypeptide, wherein the nucleotide sequence comprises a thymine at a position corresponding to position 99,219 according to SEQ ID NO:3.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Genomic Data Analyses

Genotype and exome sequence analysis of the UK Biobank 500 k (genotype) and 150 k (exome sequence) dataset identified that two variants, including a rare missense variant, in SYNJ2 significantly associate with increased risk for hearing loss (see, Table 1 and Table 2), and also with increased mean speech reception threshold (SRT) (see, Table 4), wherein speech reception threshold is defined as the minimum intensity (dB) at which a person can recognize 50% of spoken words (individuals with hearing problems will have an increased mean SRT). Further, association analyses of one of the associated variants in SYNJ2 (p.Thr656Met) with hearing loss in the Geisinger 90 k cohort showed a trend towards increased risk of hearing loss in variant carriers, consistent with results from UK Biobank, although this result was not statistically significant (see, Table 4).

TABLE 1
Association of variants in SYNJ2 with increased risk for self-reported
hearing loss in UKB (500 k imputed/genotyped) dataset

| Name | repEffect | hgvsc | hgvsp | | OR_95_CI | Pval |
|---|---|---|---|---|---|---|
| 6:158071628:C:T | missense | c.1967C > T | p.Thr656Met | | 1.3873 | 1.37E−12 |
| | | | | | (1.2672, 1.5188) | |
| 6:158081105:G:T | splice_region | c.2568-4G > T | NA | | 1.0345 | 1.87E−08 |
| | | | | | (1.0223, 1.0468) | |
| Name | repEffect | hgvsc | Hgvsp | AAF | Ncase_RR_RA_AA | Nctrl_RR_RA_AA |
| 6:158071628:C:T | missense | c.1967C > T | p.Thr656Met | 0.0048 | 100,633 99,857\|770\|6 | 250,910 |
| | | | | | | 249,326\|1,583\|1 |
| 6:158081105:G:T | splice_region | c.2568-4G > T | NA | 0.5137 | 101,158 | 25,1941 |
| | | | | | 23,362\|50,363\|27,433 | 60,151\|126,059\|65,731 |

TABLE 2
Association of SYNJ2 Thr656Met variant with self-reported hearing loss in UKB 150 k exome dataset

| | cDNA; | | OR | | | Case_N | Control_N |
|---|---|---|---|---|---|---|---|
| Position | Amino acid | Cohort | (LCI, UCI) | Pvalue | AAF | RR\|RA\|AA | RR\|RA\|AA |
| 6:158071628:C:T | c.1967C > T; | UKB 150 | 1.343 | 3.8E−08 | 0.0053 | 35,860 | 106,734 |
| | p.Thr656Met | exome | (1.209, 1.492) | | | 35,396\|457\|6 | 105,685\|1,039\|1 |

TABLE 3
Association of SYNJ2 Thr656Met with an increase in mean speech
reception threshold (SRT) in UKB 500 k imputed/genotyped and 150 k exome datasets

| | | | Effect in | | | |
|---|---|---|---|---|---|---|
| | | | standard | | | |
| | | | deviation | | | |
| | | | units | | | Sample N |
| Position | Variant | Cohort | (LCI, UCI) | Pvalue | AAF | RR\|RA\|AA |
| 6:158071628:C:T | c.1967C > T; | UKB | 0.144 | 3.59E−07 | 0.0048 | 145,877 |
| | p.Thr656Met | 500K | (0.088, 0.199) | | | 144,916\|958\|3 |
| | | Imputed | | | | |
| | | UKB | 0.15 | 1.3E−04 | 0.0053 | 63,117 |
| | | 150K | (0.072, 0.22) | | | 62,449\|657\|3 |
| | | exome | | | | |

TABLE 4
Association of SYNJ2 Thr656Met with ICD10 codes for hearing loss in GHS90k exome meta analysis dataset

| | | | | | | Cases | Controls |
|---|---|---|---|---|---|---|---|
| Phenotype | Cohort | Variant | OR (CI) | Pvalue | AAF | N RR\|RA\|AA | N RR\|RA\|AA |
| ICD10 3D: | GHS 90 k | c.1967C > T; | 1.236 | | 0.0060 | 3,673 | 75,437 |
| Conductive and | Meta | p.Thr656Met | (0.926, 1.648) | 1.50E−01 | | 3,621\|52\|0 | 74,546\|889\|2 |
| sensorineural | | | | | | | |
| hearing loss | | | | | | | |
| ICD10 3D: | GHS 90 k | c.1967C > T; | 1.080 | 5.48E−01 | 0.0060 | 5,478 | 72,729 |
| Other and | Meta | p.Thr656Met | (0.840, 1.390) | | | 5,410\|67\|1 | 71,866\|861\|2 |
| unspecified | | | | | | | |
| hearing loss | | | | | | | |

### Example 2

### Detection

The presence of a certain genetic variant in a subject can indicate that the subject has an increased risk of having or developing hearing loss. A sample, such as a blood sample, can be obtained from a subject. Nucleic acids can be isolated from the sample using common nucleic acid extraction kits. After isolating the nucleic acid from the sample obtained from the subject, the nucleic acid is sequenced to determine if there is a genetic variant present. The sequence of the nucleic acid can be compared to a control sequence (wild type sequence). Finding a difference between the nucleic acid obtained from the sample obtained from the subject and the control sequence indicates the presence of a genetic variant. These steps can be performed as described in the examples above and throughout the present disclosure. The presence of one or more genetic variants is indicative of the subject's increased risk for having or developing early-onset inflammatory bowel disease.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of identifying a human subject having an increased risk for developing hearing loss, wherein the method comprises:
determining or having determined the presence or absence of an Synaptojanin-2 (SYNJ2) rs2256014 predicted loss-of-function variant nucleic acid molecule in a biological sample obtained from the subject;
wherein:
when the human subject is SYNJ2 reference, then the human subject does not have an increased risk for developing hearing loss; and
when the human subject is heterozygous or homozygous for SYNJ2 rs2256014, then the human subject has an increased risk for developing hearing loss.

2-6. (canceled)

7. The method according to claim 1, wherein the determining step is carried out in vitro.

8. The method according to claim 1, wherein the determining step comprises sequencing at least a portion of the nucleotide sequence of the SYNJ2 genomic nucleic acid molecule in the biological sample, wherein the sequenced portion comprises: a position corresponding to position 99,219 according to SEQ ID NO:3, or the complement thereof; wherein when the sequenced portion of the SYNJ2 genomic nucleic acid molecule in the biological sample comprises a thymine at a position corresponding to position 99,219 according to SEQ ID NO:3, then the SYNJ2 genomic nucleic acid molecule in the biological sample is a SYNJ2 predicted loss-of-function variant genomic nucleic acid molecule.

9-10. (canceled)

11. The method according to claim 1, wherein the determining step comprises:
a) contacting the biological sample with a primer hybridizing to a portion of the nucleotide sequence of the SYNJ2 genomic nucleic acid molecule that is proximate to a position corresponding to position 99,219 according to SEQ ID NO:3;
b) extending the primer at least through the position of the nucleotide sequence of the SYNJ2 genomic nucleic acid molecule corresponding to position 99,219 according to SEQ ID NO:3; and
c) determining whether the extension product of the primer comprises a thymine at a position corresponding to position 99,219 according to SEQ ID NO:3.

12-13. (canceled)

14. The method according to claim 8, wherein the determining step comprises sequencing the entire nucleic acid molecule.

15. The method according to claim 1, wherein the determining step comprises:
a) amplifying at least a portion of the genomic nucleic acid molecule that encodes the human SYNJ2 polypeptide, wherein the portion comprises a thymine at a position corresponding to position 99,219 according to SEQ ID NO:3, or the complement thereof;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a thymine at a position corresponding to position 99,219 according to SEQ ID NO:3, or the complement thereof; and
d) detecting the detectable label.

16-18. (canceled)

19. The method according to claim 15, wherein the detecting step comprises:
contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a thymine at a position corresponding to position 99,219 according to SEQ ID NO:3, or the complement thereof; and
detecting the detectable label.

20-21. (canceled)

22. The method according to claim 1, wherein when the human subject is heterozygous or homozygous for SYNJ2 rs2256014, the human subject is further administered a therapeutic agent that treats or inhibits the hearing loss.

23. A method of treating a patient with a therapeutic agent that treats or inhibits hearing loss, wherein the patient is suffering from hearing loss, the method comprising the steps of:
determining whether the patient has a Synaptojanin-2 (SYNJ2) rs2256014 predicted loss-of-function variant nucleic acid molecule by:
obtaining or having obtained a biological sample from the patient; and
performing or having performed a genotyping assay on the biological sample to determine if the patient has a genotype comprising SYNJ2 rs2256014; and
when the patient is SYNJ2 reference, then administering or continuing to administer to the patient the therapeutic agent that treats or inhibits hearing loss in a standard dosage amount; and
when the patient is heterozygous or homozygous for SYNJ2 rs2256014, then administering or continuing to administer to the patient the therapeutic agent that treats or inhibits hearing loss in an amount that is the same as or greater than a standard dosage amount;
wherein the presence of a genotype having SYNJ2 rs2256014 indicates the patient has an increased risk of developing hearing loss.

24-28. (canceled)

29. The method according to claim 23, wherein the genotyping assay comprises sequencing at least a portion of the nucleotide sequence of the SYNJ2 genomic nucleic acid molecule in the biological sample, wherein the sequenced portion comprises a position corresponding to position 99,219 according to SEQ ID NO:3, or the complement thereof; wherein when the sequenced portion of the SYNJ2 genomic nucleic acid molecule in the biological sample comprises a thymine at a position corresponding to position 99,219 according to SEQ ID NO:3; then the SYNJ2 genomic nucleic acid molecule in the biological sample is a SYNJ2 predicted loss-of-function variant genomic nucleic acid molecule.

30-31. (canceled)

32. The method according to claim 23, wherein the genotyping assay comprises:
a) contacting the biological sample with a primer hybridizing to a portion of the nucleotide sequence of the SYNJ2 genomic nucleic acid molecule that is proximate to a position corresponding to position 99,219 according to SEQ ID NO:3;
b) extending the primer at least through the position of the nucleotide sequence of the SYNJ2 genomic nucleic acid molecule corresponding to position 99,219 according to SEQ ID NO:3; and
c) determining whether the extension product of the primer comprises a thymine at a position corresponding to position 99,219 according to SEQ ID NO:3.

33-34. (canceled)

35. The method according to claim 29, wherein the detecting step comprises sequencing the entire nucleic acid molecule.

36. The method according to claim 23, wherein the genotyping assay comprises:
a) amplifying at least a portion of the nucleic acid molecule that encodes the human SYNJ2 polypeptide, wherein the portion comprises a thymine at a position corresponding to position 99,219 according to SEQ ID NO:3, or the complement thereof;
b) labeling the amplified nucleic acid molecule with a detectable label;
c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a thymine at a position corresponding to position 99,219 according to SEQ ID NO:3, or the complement thereof; and
d) detecting the detectable label.

37-39. (canceled)

40. The method according to claim 36, wherein the genotyping assay comprises:
contacting the genomic nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a thymine at a position corresponding to position 99,219 according to SEQ ID NO:3, or the complement thereof; and
detecting the detectable label.

41-42. (canceled)

43. The method according to claim 23, wherein the nucleic acid molecule is present within a cell obtained from the human subject.

44-65. (canceled)

66. An isolated alteration-specific probe or alteration-specific primer comprising at least about 15 nucleotides, wherein the alteration-specific probe or alteration-specific primer comprises a nucleotide sequence which is complementary to a portion pf human Synaptojanin-2 (SYNJ2) rs2256014 variant nucleic acid molecule, wherein the portion comprises a position corresponding to position 99,219 according to SEQ ID NO:3, or the complement thereof.

67-72. (canceled)

73. The alteration-specific probe or alteration-specific primer according to claim 66, wherein the alteration-specific probe or alteration-specific primer comprises DNA.

74. The alteration-specific probe or alteration-specific primer according to claim 66, wherein the alteration-specific probe or alteration-specific primer comprises RNA.

75. The alteration-specific probe or alteration-specific primer according to claim 66, wherein the alteration-specific probe or alteration-specific primer comprises a label.

76. The alteration-specific probe or alteration-specific primer according to claim 75, wherein the label is a fluorescent label, a radiolabel, or biotin.

77. A support comprising a substrate to which an alteration-specific probe or alteration-specific primer according to claim 66 is attached.

78. The support according to claim 77, wherein the support is a microarray.

79. A molecular complex comprising an alteration-specific primer or an alteration-specific probe hybridized to a SYNJ2 rs2256014 genomic nucleic acid molecule, wherein the alteration-specific primer or the alteration-specific probe is hybridized to thymine at a position corresponding to position 99,219 according to SEQ ID NO:3, or the complement thereof.

80-86. (canceled)

87. The molecular complex according to claim 79, wherein the alteration-specific probe or alteration-specific primer comprises a label.

88. The molecular complex according to claim 87, wherein the label is a fluorescent label, a radiolabel, or biotin.

89. The molecular complex according to claim 79, further comprising a non-human polymerase.

90. (canceled)


SEQUENCE LISTING (9 sequences)

SEQ ID NO: 1 - DNA, 117290 bp, homo sapien
gcggggagctgtcgcgggcagcgcgccctcgggaggacgtggccccggcccccgcccgcagtgggcccgaccctcatggccctgagcaaagggctgcggctgctggggcgcctgggggccgagggggactgtagcgtgctgctggaggcgcgcggccgcgacgactgcctgctgttcgaggccggcacggtggccacgctgggtgagtccgggccgggggcagcgacgcccggaggagagggcgcccgcattcgcccagcctcgggaagacgggtacccccccttcccgaggggatcgggcggcgctgggactgccggggcgtaggggtcgcgcgcagaggggtggctgtttgaatgaagtggggctggggactcgagagagcactctggctgggccggtggcgcacggaggccggccgcccctccacggggatctccggcccgcgccgccagaggatatggttgctggatagccggctggggcgctttgcgtattcatgaggatcccggggtgttgacttagccggcctgaccctgtgcctggcgccttttcttttgcacctgttgatgaaaccagtgattgccctttggtttgtgttggcggctccactcctggaggggatggggacaaaagccccaaaacataggctctggagaaaccgctgatggcagctttgtcccacgtaaatacttggtcgagaagcatcggtgctgcaatttatagaacttgtaaacaaatataccatgacctttatgctgtcttagatcctgccaagccagcgaatttcacctatggacttcagaagccttgaaactggcattgtgctatgggctttagtacgtgatctcacttaattctcacaattctactaaaaagctattattatccccattttgcagatgagcaaactgaggcctaaagaggctaagttgcttgccaaggtgacaccatccacctgacttttatgtggctgtttctgtaatgtcattcaatgcgtggcttcctggtatcgctaacctgggtcccttgttttgtgctaaccattataaggaaaacctgggtagcaccaaagcacgtgaaaccaggtctgattgttagacttctaaatgtggacacccgagagtaagctcccaccactaggcgtccagctgtaacagcttgccggtattcctagctaaacaaggtgcagcccggggtcactcgtacttgaggtggtcggcaaatgcagcaggctgctttgtagatgcctctagccttcttttcacatttgtatgcatcgactcccgttcggatgtatgtcttggaaggactatctctcctatttctgattccctaatcatttcattaacatgcatttatccagcttgttctcagtcacttgattactgaaacaatgtctccagtagtctcagcatcaaaagtaggcagtgaattgtagattaagttattttcagattattctaatttgtgctgtcctgaagagtctaagtttggttatttggtgtggccttgaaagaaaaccacagaaatttccattccattttagatgtctacttcataccacatttatcggaagcgaaagagtaatttcatggtacaaaactactgtttcatcaaatagattaagatgtccttcagttggattacggcaacattaagccaagttcatacacattcaagaattctttgaagcagcttagaaaaaagagattttattttttcaaaagtgaagatttaaaaatgattaatgacagtaatgtagtgcatattttccattaggagctgaagtttgggctgagatttacatcacggtggtgattacagtataccagattttagaacagagaatgtaagaaaggaattggctcgtcttcaactcatgtgcagtccacatttgtcatttaacctagcattcatttttcagtagaaaactttaaaaaaaaattttttttttttttgaaacagaggcttcttctgttgcctaggctggcatgcagtggcacagtcacagcccactgcagcctcaacttcccaggctcaagcgatcctcctgcctcagcctcctgagtagctgggactataggcacatgccaccatgcctggctaattttttctctttcttttcttttctttaaatggggtcttactatgttgcccatgctgttctcgaactcctgggctcaggtgatctttccgccttggtctcccaaagcgttgggattacaggcatgaactacttactgcgtctggcctgaaaacttaattttttaaagtctgttttgtttaagtaaaatgaaactatttttaaaaagcagctgtagcatttggtggctttgctggtccagagaatgtttgtaggagagagccttatgttactagaaagttgtttaaaactttcaacatatcctaaatgcatcactattggataaaatggctctgaacttgaaattcccatctgtgttccaaagggctagctagctgttttactttcctttcttggagaaagtgattattttttttcttttttttgagatggagcttcactcttgttgcctgggctgagtgaagtggtgcgatctcggctgactgcaacaccgccacccccacctgggttcaagtgattctcctgcctcagcctccccagtagctaggattacaggcacacgccaccatgcctggctaatttttgtatttttagtagagacggggttgtgccattttggccaggctggtttctaactcctgaccttaggtgatccacctgctttggcctcccaaagtgctaggattacaggcgtgagccaccatgcctgacccgagaaggtgaatttttacaagaaggctgcaatctctacttttgtatgggacaggacacttgtggcagctggcgtagctggtggtctaatcttgacctttcaccctttccttgaagttccgagtcgggggtagcttccttttctcttcagctgtggttttcctgtgatgggtccctcccagttagtaatgagaggctgagagacaagttgtggtaactgcttcgtgggtagtggtctttcccggcccctgagtcattctaacttccatcctgctccgctgcggatgcacatagctgctttcctaggggcgagaggagatgaggggactgggcctgatgaccacaggcgctgccccttcatagcacattttattcctgaaggatcctaactggaaaagtacctctcgccgaaggggctgggtggttccttatcattttcaggcaaggcctggagacaggggcttgaactgggctgtgatttttaggaaaaacggatggtccatgattcgttgattgctggactccttccatctgaacctgtcttcacctgtcttcacctgtcctgctctaaagtgaactcttcacactagggccgtctggcctgctagcaggattgttgaaggaattattccaaattcctgcttctaaaagaattaggttgtgaatctgaatctgtcttttatgtagatcagtgggagcaaaaattccagaggcagaaaagccaaatgtattggcactgatttaaaaggcttttatgaagtctcgggaacctgactggagggtagttggccttctcagcacaggtgggaagccccgtagccggtaccagcatccctggccaacaatccacaattagcaaacagatgttcccttatttgcacatgggacaaaaacctactcctgtctctgtctcttgtaaacttagggaaaagtgcttattgttaatggattttgtgtattttcagagtaagcgacagtgttccaaagttcaaggttctccaacagcccccaggaacggtggcctgagggtgtccctgtgtttacagtgcgggctgtgtgtgagtgaagtcgtgcctcctgccgcccagccgggctttgctgtgaacaggcagcagggagggagggatggggcaggagacttgagagaattaattaagttaattaatgggtgtccctaaattactttgagcttctggggggaaaggttctgtgtaaacaccaggcccccaggttctcccagctgctgttgtgcgaggccacgtctcgagtttagctttgtttgaattggtgagatggccccagggcagagcaggactcagtctgaactgttgcctagatcactttctcaggcattatggccagatttatttggtcacggactggaattttgatctgctagagaggaccacagagaatcaaataagtcaccattttataaatgctcctggacaaagttgaaaccacaggtaggaccaggcaagggttccggggcccctgacgtgggaaactagagcagcgacccgggaccacagtggtctgtctgcctccaggactggtttgcctcctctcctagccggaggctgtctcagcgtagtgtttacaaaatgcaaactgggatggtgtgtgcttctcccgcgggttttgtttgtttgtttgagacggagtctctttgcccagctggagtacaatggcgcaatcttggctcactgcaacccccacctcccaggttcaagggattctcctgcctcagcctcctgagtagctgggactacaggcatgcaccaccacgcccggctaatatttgtatttttagtagagaaagggtttcaccatgttggtcaggctggtctcaaactcctggccccaaatgatccgcccacctcagcctctcaaagtgctggggttacaggcaatgagccaccacgcccggccttccccgtgatttttgtagctcacctgtaagatcgtgatttcaaatcttaccaggctcttgttttggctgtgtggctcaaaaacttggcttttagcctttaattgtgaaaacgttcaaacacaaaagtagagggaaaaatgaatcccctgtctacctgccattcggcattgatagttatcaatatattgccatttttatttcacttgtccctgtattttttatgaaaacgtgaatctcctaaaatttagtttgtttaaagccaggcaagattctaatttgttgggttggcttttttttttttcttgcatttagatatagaagttcaatttttttattttattgtaaattgacaaattattatcgttttttgagatggagtcttgctctgtcgcccagctgggagtgctgtggcacaatcttggctcactgcaacctcttcctcccaggttcaagtgattctctggccttagccttccgagtagctgggactataggcgtgtgctaccatgcccggctaatttttgtatttttagtagagatggcatttcaccatgttggccaggctggtatcgaactcctgacctcaggtgatccacccgcttcggcctcccaaagtgctgggattataggcgtgagccactgtgctaggccaacagattaggatgattatatattatggggtacaaagtgatcatatatgtgtacaatgtggaatgattaaatcaagatgattaacgtagtcatcacctcaaatacttctttgtttatttatttatcttttttgaggtggagtcttgctctctcacccaggctggagtgcagtggcaccgatctcggctcactgtaacctccgcctcccaggttcaagcgattctcctgcctcagcctcctgagtagctgggactacaggcgcgtgccatcacgtccggctaatttttgtatttttagtagagatggggtttcgccgtgttggccaagctggtctcgaactcccgacttcaagtgatccgcccgcctcggcctcccaaagttctgggattacagacatgagccactgcacctgaccacttattgtttgtagggagaacatttgaaatttactctcctagtgatttcaaaatatacattattattaactatagttaccatgctatgcaagttcgaaatttacttggcagatttcgtgtggccagcagcagaaatggtggcatgaactgattctgagacattggccaagacacatttttaaaagcaaagtgaccacttgggaagggtcgaccacagcctggaagaacagggagcgctctgctggtgagctcgggaggacaaggtctgggccctttgacggcactgcctctagccccgggcaagagagggccgagggccatgtccagtggcctctatgctcccctttctggatgacattcctgctgctcaagaccctggagtgtgctaagcacatagtgcagcctgctcagccccctcccagggcctcaggcagactgtgcagccgtatgtccaccccaggcccgggcgttggggtggcaagggcatgttacaggtggaacgggacacacactagctgggactctgtgtgtgtatgcacgaggctccctgcaggggggcatgaagcccaggttgggggaagtcggggaagagaaggtggccaggctggggccgccctccccactggctacataatgaacctggccttccaggtagtcacggtggcatacttttcgaggtaggaggatagaacctattttatttaactgttttactttgttagcttgatttaaaactgaacagctgggcatacaggatgcagcactcatgagtactctggccccagaccctacagatattcaggcagacctgtcctgggcacctgtcccagcccgtgtctctctgctgtgtctctcttctggagcgttcctggggtgctcagggtcccctttgtgctttttggctgctcagtttctgttgctgggctccttcggccttcccactgaggatggggaatatattctgttttctttccacttgaattgtccttaaagccatgggtaaactggttcaggctcagacttgggaatttgatcggagtccctttagcatttctgggcagggcttgttggtgtcgtggccagctgtcctcgaggtgaaaaccaaattgggttgggagggtgcacatgacacttaaagcccccagagctgcggtgccatcaaccacacactgtggacaggaggctgtcagtgggcaggctgggtccaccttcaaagtccatacgaaacagcccagttcagggtactgcttcttttcgagatctcacgggcaggagccttctggcactgttcatgtaaatgtaagtcattctttctttctggaggaaaatttagacaggtgaatgaaaaccggagtgcttaatccgctattctacatgcagaagtttatctcaggaaacagtcccagatcactagcccattttgcttcaaggatgtttattgcaagctgttttgagtttttcactggttaaataaactttggttcaacttgatcataatagctgacatttgtggagtgtttaccacctaccaggtgctgatccaagtggcttacatttattaacttacttcatccttatgtcaaggcaagtactagtaatctccattttacacttggggaaactgaggcacaagaggcctaagaacttgcctgaggctccacctccagtggaaggctccagagaacaatatgttcagtaaaatctcattcttatgtaaaaaaatgaatatatatatatatatatatatatatatatatatatatatggaaaaagactccctttgtcctgttgtctctggcttatcttggttttcatctgacatacacttttgatcgaggaggttcccacttggggaaacagccagcccctttctgagcagagggtgtggcactacaaaggcgaaacatgatctctgggagaaacatgcttgaagcccacgctcctgggggcagagctgagcctccacacggatgtccccagcactgggagcagaggctgatgcggccccaaggtgctgaggaaagcgccccccagtgcaaaggatgctacaaggccccaggcttctcaaggaacatgaggcctggcaaacccagaacgttagactgggtcggctccaagatgcccctgggtttgagctggagaagtgtctgcgggagggtgctggggctcctgcagagcgtgtcttcctgaccctgtgggcttttctggggggggctaggaggctctgccaagttggtggatggaactgtgccttgccctggggggcactgggcaggagccagcctcggagcgtgagtctggtgctgcgccaggaggctagatgccccggccaggtcctgtgggctgcgccgtctgtgcccttggcccgcgaggctggcaggctgccactgcttgtaaagcctcctggttgaaaaggtgactcggagcagcggattcactgggagcctgtcagccacagaggcagcttgtgcttgtgccccaggcccaggccgcaggaggtgggatcaagttccacggctccagtgtgaaggtctgagcttcattctaaggcaggtttttgaggacatggggctctcgggcacctggttgtattcctgcatcctgcattcctgcccactctctcatgccttcttctccttctccttggcgtgccccaagccagccttccatctcagatggagttgtatgtttctagtgagcccacacaccccctgaggacagggcctcacccccgcacgcctgcttcttcatcacagctgctaggaaatgaagatgcatgtccccaggggcctttgaagtaggatgtggtctccctgagcagaactctgactctaaattctctcctggctctggcgttggtgtgggacttgggttgcgggagggggcaggttgttagcggctgctgctcagacccagcggaggaccgggctgctccaggctcccggcaagatgtagcaaaatggccaaatttaaggtgagcacgggctcccgtgcagtttctcacccctgctgctggctgcctgtctgtgaatgctaaggcgaggggtggttctacattgacttcttactgactggtgggagtaacccggaagaggcaaatgacttccttccctttcctcggctctgaacctagaatgttccagtgataccacctccaaactcttcacctgtgtcctttcccacctctctgccctgaggacagggaactaagtggctgctcacggtgggtaaaactgaggcttacaagacaccttgggcaatgggacagccagaccccagccctcagcacctgcctctcccatcccaagccccagtctctttttcagctgcaaagtcaatacctttgctttcagagagggagcatgtgccacgccaggagaggctttgggtgtatcacagatggagactgcctagcttaactgcgcagtctgaatgattctaaggaggggaagagagaacgcctctggagcctggcacacacatttgccggagaaagtggtgagtgcgttgtgaccaggtgagggacgaaccttaaggagattggctgagaacacctcccctcctcaccctaagtcaacaaacccgggacagcagtgcttccaaaaccttacttgttcacctcacctggagattcttgtggcaaatcgggttctgcctcgttggctctggggtgggccttgagagcctgccgttctaacaagctcccaggtgatgctgatccacgaccagctttgagtaccaggatctcgcatcagcttgggagggaggagacctgaatcctagccctggctccttaggctcttctttccagtgggggaatttagttaattgctccgggccccaatatcttaatctgaaaacaaatgaatgacaacctagttgcctttcctactctgagattccatgcgtcttaaaagaaaaaagttttaaaactaaacatgtcactgtcaccatcgctacttcacacacctaaagttataaaccactcagaaatgcaaatcttaggagatgtgatctggcaaggcaaaatcgaagatggctttgaggtctcagctaaacctagaaatctgatgctctgcattttttatgggatgcttatgaattaatgaaaatacatgagattatctctccttgagtctaccctttcctacaaccagcaaaaacttataaaaagcaattgaagcttactttgggaatgccaagggattgattaaaatatggactgtgctgattccatggctggtttagcagattcttgttaaaagccatgactttttaaaatttttatttttttgtgggtacatagtaggtatatatatttatgggatacatgagatattttgatacaggcatgtaatatgtaataaccacatcatggaaaatggggcatccatcccctcacacatttatcttttgtgttacaaaccaattatactcttttagttattttaaaatgtacagttaaattatatagtcaccatgttgtgctatcaagtaccagttcttattcattccttttatttttatttgttttactcattaaccatccccacctcctccctagacccccgccgctacccttcctagcctctggtaaccatccttctattctctatgtccatgaattcaattttgatttttagatcccacaaataagtgagaatatgcaatgtttgtctttctgtgcctggcttgtttcttttttttttttttttttttttccttttttttgagacagagtcttgctctgtcacacaggctatagtgcagtggtgcgatctcggctcactgcaacctccgccttctgggttcaacctcccaagtagctgggattacaggcacccaccaccacacccagctaatttttgtatttttagtagagacagggtttcaccatgttgtccaggctggtctcgaactcctgacctctggtgatccacccgcctcggcctcccaaagtgctgggattacaggtgtgagcaggtgtgagcaactgcactcggctggctggcttgtttcacttaacataatgacctccagttccatccatgttgttgcagataacaggatctcattcttttttatggataaatagtactccactgtgtatatgtaccacattttctttatccattcatctgttggtggacactgaagttgcttccaaatcttggctattgtgaacagtgctgcaactaacatgggagtgcagacatctgtttgatatactgatttcctttcttttgggtagatacccagcagtgggattgctggatcatatgatagctctacttttagttttttgaggaacctccaaactgttctccatagtggttgtacgcatatagtacatatagtacataacatatataacatatgtatactacataacgtatatatagtatataacatatatagtggttgtactaatttacattcccaccaactgtgtacaagggtttccttttctccacatccttgccagcatttgttattgcctgacttttggatgtaagccattgtaattggggtaagatgatctctcagtgtagttttgatttgcctttttctgacgatcagtgatgttgggcaccttttcatatgcctgtttaccatttgtatttcttcttttgagaaatgtacattcaaatcttttgctcattttttggatcggattattagatttttttcctgtagaattgtttgagctccttatatattctggttattagtcccttgtcagatgagtagtttgcaaatatttcctcccattctgtgggctgtctcttcactttgttgattgtttcctttgctgtgcaaaagctttttaacttaatatgatctcttttgttcatttttgctttggttgcctgtgcttgtggggtattgctcaatacatttttgcccaggtcaatgtcctggaggtctttcccaatgttttactgtagtagcttcatagtttgagggccatggcattcttaaaaacataactttcattcttgcatagctttttttttttttttttttttttttaagtaacctggactgtcatttggctagggaggcgggagtttgtgtgtctggaaatgtgtgggtttttttttttttttttttttttttttttttttttgggacggagtctcgctctgtctcccaggctagagggcagtgcagctacctcggctcactgcaagctccgcctccctggttcacaccattctcctgcctcagcctcccgagtagctgggactacaggcgcccgccaccacccctggctaattttttgtattttttagtagagacggggtttcaccatgttagtcaggatggtctcgatctcctgacctcttgatccacctgcctcggacttcaaaagtgctgggattacattacaggtgtgagccaccacacccagccagtgtgtggtcttttttatctgtctgtttaaactgtctgttttatatgttgcatgaaccccgctttaccatgaatcttatcaatgactgataaatattgtctattggaaacattctctttaagaggaggtgaccgttctctgcagctcctgctgattcagcaaagctgaggtcaggtggagccagctgggcagagatgagtgattagaaggctgtcattggcagtgacagtccacgtgctccagggcagaacagtgacactggctgtcacagtctgtttcaacaatggaggttgtttgtttttatttaaatgatgtgaataaagaaaggaatctttgaaaactaaggctggagctgcccaggcctgagccaggcccttgtaacaaggcccttctgggtgggggatctacctctcttttcacctggaccacctggcggtgggtggcgagctcagaaccacagctgggtggaaaatgggagggccagcagcgagctgcaggaatggaattccatgatggcatctcatgtctccagagtggacagacagaagcttgtgttttcatttcaaagggcatctgccacgccctccaggcagaccacataaatggcatttttgggtgaggcctgagggattctgtctggtggtatacactgattttaaccagaagcaatagatttgggctctttttgacaggggaagatagatgagagatgatttccatgaggatgttgaaaatattggcaattctgaagaccccagggagtcccagggggttcctggccaggagcgcccctccattgccctgcaccaccttgctttccaggctgtggctctgtgtgtctgaatctacggcatgggggcacttccttctaattcacagaagaagctatatgggccccagactgcccatcctgctaaaactggcaaaggccttttcccagcctgagcagtagtgattcagccaactgcagaagtggtatttggagctcacccttaaaacataaagcaagttatgttcagactatatacaggcaatgagaaaatatgatgaaatatgactgcgtatgaatagccccagacttctagggcctttgagtgtgtctgtcttgggcaacatcttctccttttagatttcaggctcagtttactgtcaggaaaattaagttgctgagagcgtctcagaattttggcttaaaagagaatatacacctggaacaaagacttgaggggtgctcctattaaaaaaaacaaacagtgtgtgagcttcaaaggagcaccttgaaactccagttccatcacatccactctgctgggccctcctagggcagctctctgtcttgtgcctgccttctcttctgaacactcctaaggtattttgagtgcggaggataggatcatacactcaggaaagccgagagctagatcctgaagcatctgtgcgggaaatgcccatctgtgcctgaaatgagcatctgcgagtgaagtgatcccatgtctgggatctgtttggcatgcgccaggagacagacggacacatgcatgcacacacacacacagcggggacggggagttggtgaaataagagtgggaaaatgttgacacctccgtgagccgggtgacaagctgcttggtggttcactagacagggagttataaattggccgcttttcaacatcgacagataaaaatcaatagccttcccataatgccatcattaatcaattagagtctgtaatgggaaaagcagatgccactcccccacagtttctgggaataaacaaagtgtataaggcctaaaagaagaaagctatagaacttgactgaagcccacatctgtggaaaggcatactatgttcttggagtgggaacatcaacactatagagatggcagttcctctccagttaatctggaagtgcaagtatcgatgagtaaatccaattaaattccaacaagagcctggtctaaaatgtaacaggctgattttacagtatcgctggactccccctacatccaaacttattcttaccttcattctcccattttggttaatggcagcaccatccaggcaactgctggtaataaaagcctgtgagtcattctgacgccccttccttcctcagcctcatccaacctaccagccagtagtgaatcccaccccaaaacagatggtgaatcccaccgagtcccccggtcatcgctgctacatcgagagtgcagccactgccccatgtgctttcctggtctgcttggcagcctttgaactagtctcctggctcttcccctgctccccgtatagggatctgcttgaagctcacacagccctgatcctctgcccctgcctctttctccaccctcatctcccaccatttcctgggctcactgcattcagcagcctgacttgttcctgctcagaatcttcacacccacctgcatgcactccctcccccaggtgatatggtttggatctatgtccccacccgaagtcatgttgaaatgtgatccccagtgctggagatggggcccagtgggaggtgattgggccatggtggtggtttctcatgaatggtcagcaccatcccccctggggttatcattgaaacagtgagttgccacaagatctgattgtttaaaagtgtgtggcacccaccccaccctcttggtcctgctcctgccatgtaagttgcctattcccactttgccttccaccatgagtaaaagctccccgaggcctcccaagaagcagatgctgccatgcttcccgtatagcctacagatccatgagccaattaaatctcttttctttataaattacccagtctcaggcatttctttatagcagtgcgagaacagactagtacaccaagggttggcctggctgctgctgcctccgtctccccacctaccccacccccccccacccagcttcgtgactgcagtaagaccttcctgacctctgtggctggaataccctgcagcccccacacaccaactgtctctgccccatgcctggtttcctttcttcttggcgcttatcactgaaattaccttatgtgtgtgcttacgtgggtgtcttctgttgtccactgcaaaaatgcattgtgggttgaattgtgtgccccagaaaaatacattgctgtcctaacccgggataccttgcaatcttatttggaaatagggtctttgcagatgcaatgaagatgtaagttaagatgggatcgtactggtatagggtgggcctttaatccaatgtgactggcgtgcttataagaagaaaagggacacaggcagacgcacgcagaggggagaatgtcatgtgaaggcatacagggaagacagccatatgacaatggaggcagagatgggagtgacactgccgcaagacaaggaatgcctgggctaccggaagctgggaaaagcaagggaagatgctcctacccacgccttgatcttggatttctggcctccagaactgtgagacaataaatttgctgttgtttcaagccccccgttctgtggttctttgttacagcaacccaggcaactaatacaggggcattgtgtatctcgttcagtaccttaccttaacactgcctcgtgtggaaatgaggcttggcaaacactgttgaatgacgaagcattggcaaaaaaaatagccaagattttgaaaaggatccacaaaggcagactgatttaccaggtagcacaacatattataaagccctaaccatcgtggaggctgtcagcacaaatcaacacagtagagtggaaggctcagaacagacttgtgtgagcagtgttactttgtttcttataaaggtgatgacttcagtcggtcagagaaaggtaagggtggatactcagcacctgctgttggtgtggttgatcagtgcagtgcctaagaggcccctgctctattcaactggatattcacgcaggcattcttggccggtctgctcagagaggaataggctggaaaccttggctggccagcaggatgctgcctgctgctctgcttccaggggagcgaggggtcttcctggcagtttttgcaggtggctgcacacacaggtgtccttctggtcactagctcctcccccagctgaaagccctgggtccccaaggagccagaatcccttataaaattaccagccttacagagccctgggttcacacaggggacattccatgttacaagagttaccacacctaaggaagccccatgcctggcttcttcaccgagtggttataattcacttatgggtcttccctgtccagaggcaatttgccaatcaaggtcatttttaccatgagcaatgttgtctagtaacagagttgacgttccatcttgattggggttcccagggaaccagagctagataattaaacattaaattctcatacagaagggaagaaagattttgctaagcctgtgaccaaaactatccatatggacactaagtgagagtcctatgttttatcatacattaaaataaatgcagaggtatttaagtgtaaaaaaacaactttcaaagtattagaaaatataggctatgctttcacagtgttacggcatggaaagatgttctaaggaaagcatcaaactcagaagtcataaaggaaataaacctgacagatatgggaaatatttttattgaagtgtaacatacatagagaaaaatgaacactcgaagcgttgagctccatgaatcttcacccggtgaacacacccacatagccagcacgcagatctggagagagaacgtttgcatcccaggcccctgcctcccattccccctgtgtccgaatgatagatttgactacataaaaatggaaaactccaatatatcaaaagacaccataaacgtagtgaaaagacaagcaacagcccagaaggaaagtttgtgcagccttttgtaacatagaaaaaggccaaagatggatgatcatagcatataaaagaactcctatagatcatgaagaaggaaggacaaatagttcaacagagaaattggcaaaggatataatgagcaattctccaaaggaaaaatacaaatggccaaaacaatggagccagtgtggccggctctgcccagcacggccatttgcacatggtcactgttgggtgggtgagggctgcttgccccgtctccagccccactctcttgccatctccccctgtgattcaggcatggagctcaggccccaggtggcaggcagcgcactctgctcagttaccccagctcctggcttcatggccatgccctggcacctgtctccctccccttagcctgctgacctttttcccatgggatgtttggttgtctctgtacaggctctgagccagccctatgggcttccagacaagcccagattgagcctgccctctagggagcacccctgtgtagcttccagctgagaccttgagacacaggcccagggagccaagctcagggctgcgaacatccaggtggggctcaggtgctagaggcggacaacagcctggtgcaggacccagagtggcaagaggtagtaagggggccatggtgttcagaaattggggcaggtggtgggtaaggcatcctgcttctaaaggccagggtttgggagggcaaggaagcccctggcaggagtcagggcaggcatcccgggcaggtagtgtttgttctgtgaagcaggcttccccaaggaggggccctagagcagaaagctgtgatgtgcaggtgactgggagacctgttctctttcgagccgtgcagctccggttagtggatgacagatttcaagccctgctcaggccagtgtttttcacacatgtttgacctttggttgacagcacacagcacaacagcttatggtccaggagtgcacctcggcatgcagtttgggaatcctggcgatgatgtgattaagattctggggccaggttctcaccaagccaaaaggctttttttttttttttttttttttttttttttttttttttgaggcaggagctaattctaggtacagaaaaccattggtaaagtggaggaagctgtttccagatccacggaatgacggtaactagcgtcacttgagaaggtttcttctgcccttttgtaaaaaatagcatgatgttttctcctagagacagacagggcctctgtacgtaagagcagacatgtatggagaaggccggagagtcacaaaccatatgtgcctgagatgtgccacctgtggggctgtgaataaggaagcagaatcagtgtgtttactcagagctggatttggctgtttgttccttcatctttcttggaccacatgtcctggtgttcagaggctcctgagagcctggggctcagtaggccgtctcatagcccctctaacccaggtgagaaaacaggtcccagtgggtccctaacccatgctcattttctcaccaaacaacaacaaataaatcagaaagtcctgaaaaatgaattctggcctgttttctggattctggattgaaatatggagtaggagctgacttttagtaagttgatcaactctccccgccgaccccctagccttctggaactgtcttcagtaaacacaacccccgccacccctgacctacccgcaccgcactgtggtcggcctcttctttgaacacttcccttacagcacgcactccccccaagctgggccttctcttgagaccactctgctgtccttgggagtgatggtggaggcggggtagacaccctgtccctgtctcgtggtgctcctttgggccacagctcctcttccctccgtaggaggaaccatctggaatccatcttcctccttgaccacacctctccattcattaaggagcagagttccctgctcactgggtctctctacccctcctctgtccctgtctgggtgaggtcacagagagcctcacccgcctgctctgttggcctctcacttgccgtcctctctgccccgttccctctcctgcacacctgtccgcggtatccccagctcttagtgactctagccctctcccgccctcctctgcaccctgcatgcacttgtgttgctgaatgtggccggagaaaaacacagccatcttgactgttctgtgagacttcatagatttccagggctgtgtggaatcaaaaaatcttcctgcatttccccagccatctggcctccaggatgactatttcacatctcctcaagcctttcacatctcatccccctcacacctgatgattttgcttcctatttcactgggaaaatgaaggtaactgagaactttcttttttttttttttttttttttgagatggagcctggctcttgttccccaggctggagtgcaatggcacgatctcggctcactgcaacctccacctcccgggttcaagcgatttctcctgcctcagcctcccaagtagctgggactacaggcgcccgccaccacgcccggctaattgtcttgtatttttagtagggacggggtttcatcatgttggccaggctggtctcgatcttctgacctcgtgagccgcccacctcggcctcccaaagtgctgggattacaggcatgagccaccgcgcccagccaaaggtagctaagaactttcaacagcccccacacctactccctggctctggtacagtggacagcctcccaggcttgtcccccctcccagtgaccttgacccctctcatcttttaagacctcgctcccgcagtttccccctttccttcctaactcggtcatttgcaccattataccaacaggctgtcttagctcacattaagaaaccctctcttgaccctggcctccacctcatctcttggttttcccatgtgacgaggttgtctacacaccttccctcttcccagtctcccttgaagtgactccagccaggctgtgtcctcccactcttctgaaacctttcctcaaggcctccaggaccttctggctgctcagtcttccatccaacagcatcccacagatgaagcattttcttcgcttggcttgtcttacgctctgttctcatcatattgcttcctcactgcctgtttttctccagagcctagaaccatgcctggcacacagtgggcatctagaccctttgctgagtggtgaaattgaagtgtgacagggtgcgtggcttccctgctcaggctccctcagttcccaaagctgtgctccttcctggatctcatgtagcacaaggaggcatacgtgggtcgcaccacatggcttctagaggagtatgtgtgaatgtgttcatgttcatgtggaaatagggaggcggccctgaggtgggaggagccttcccaagaggaatcacctgagagaggaagaagagctggggaggaactgaggaactggtttggcaggttcaggaagaagtgagtgaagaaactggaaaagtagccaggatcagaacctgcagccgcaggacctaatttgttaggacctaaattaggggtgattgtgcaaggcagtgcagcatggaagccccactccaggacttccctggtggctaggaaacattgctccacccctccctcttgtcaaaagagaaggtgcacacacttggagagaaatgggtatttttgtgcttctttcttgttccctgttgggtaggagggttggggctaccaggcccagtgaaaacacagcccagccctgcctgttctgcctcatcctgaagtgtaaggaactgcccattgtgctgggccctgtcactggctctgcccgaggcatatgtggcactgggcctggctttttaaggtgcaaactgttttcctggatttcttatttttaagggaaaaaactcaactacaaagactcagtggccagtcctgtgctcaagccctcttgttaggggagctggcctccctctttggccacagctcagtgcagggaggggtgtggaaagtgctgggaaagggggaggacccaccctccccacagatgggtttctggctctgactgcgggaaaccaaactttcttgtattttctctggctgggttgtgatgtgggcatggcgttggtgggaggtagtgcatttgctgagggtgatgtgttctgagtgtcccctcactccgtggctctttcatgtggcctggactttttggtgagggagcagcagtgggaggacaggagagtcctttagcgggctgatgtgtgtctgccagttggctttgtcttggctcgcagcttctgtttcagacagatgttttgatccccctttgcttagcaaccagctgcagccttgctctgtcttgggaaaaccactgtctttctgaagagctgcttcagctccaaaactgataactctggaaggagcagccaccagccagtcagaactgagctccgaattaacacatgaaggagactttggcctcttgagaccctgaaatacaagaagatggtttcatgtcatgtccaagaatgtggccacaaatgctacctcagtctcgggctgtctgcattgttaaatgacctcattatttttctgacctgtgggccgggcttcgttggcatttccttaaggcgtgttattgtggaaaatattattcaagaaagaagagcatgtggtttgcttggttactgagctggactggctctgggtgaccagagctctcacgcagagccgtgcacggctgctcccactgagaccagcctctgcctcgggatggtctgacccacggagctctgttttgggacccagaatccatgagccacatttgctgtgctgtgggacaaaccacagtttccttgggagcctctcaagtggtggctgtttctcttccacacccttggaccactggagctggagacggggagttccccagcctcctccaaggataagcctattccttaagcctccacaagccccatcattatttatttttatcataagccatgttgttcgagtagcatagacaactttctcaggttaccagggaaacaggactagaaagtcaacccacgatcaaattaccttgccgaaagggaaccagctcagttaacccttgaccccacacgccattgtcagaggcgtatgaaccagagcgactccatcttgaataggggctgggtcaaatggatctgagacctgctgggctgcattcccaggaggttaggcattcttagccacaggatgagataagaggtcagcacaagatacaggtcataaaggccttactgctaaaacaggttgtggtacagaagccggccaaaacccaccaaaaccaagatggcaatgaaagtgacgtctggccatactcactgctcattatatgctaattataatgtattagcatgctaagagacactcgcaccagcaccatgacagtttacaaatgccatggcaaaatcaggaagttaccctatatcctctaaaaaggggaagaaccctcagttcctgggaattgcctacccctttcccagaaatgaataatccaccccttatttagcatataatccagaaataatcataaaaatgggcaaccagcagctcatactgctgccctgcctatagagtagccattcttttattcctttactttcctaataaacttgctttcacttcatggacttgctctgaattctctcttgcatgagatctaagaactctctctcggggtctggatcgggacccctttccagtaacaccacgagactgcgtccctcactgcccctcgtttcggcagttgcatggttccttggtgagttttccctctttctcgctgtcatttctcactatgagtctggcgcttgttcttggtgtcttcagtggccacgtcgttgatccttcggaggaccttcgccccctctcctccagtgagtgaccccacccctgcagccactcgctcccatgccacgcccctgacccctccagcatctgtctcaaacatcccactcttctttctccactttcaccttgtcactcatcctgcaattcccagtcccagtgactattttttttttttttttttgagacagtcttgctctgtcgcccaggctggagtgcagtggcgcgatctcggctcactgcaagctccgcctcccaggttcacgccattctcctgcctcagcctccctagtagctgggactacaggtgcccgcaacgacgcccggctaattttttgtatttttaatagagatggggtttcaccatgttagccaggatggtctcaatctcctgacctcgtgatccacccaccttggccttccaaagtgctgggattagaggcgtgagccaccacgcccggcccccagtgatctttgaccccactgggatggcgacccttagaacccatggccttctcatagcccctcgtctgtctcacaccttccctctttgccatgtcagtgaccagccctgccttgcactcaccctccccacccgcccttctcttgctgggtcctccccacctggtgaaacctcagccctggttacatcctgcactccacgcctgcaccagccgctgtgcagtagaagtttctggagatgccccactaccacacgggcagagcgtaccatcaggtcacaacccctgagccaaggtgggcctttcacttgcgtttctgtgctctgtccactcccccaccctcccagatggcaactccaccctcccctttcctcagctctgctgtgcctcctccccatcctcactttcagctgatggcctcgtcctctcattccaccccaaaatttgaagccactggaagagaattcccgctggctccctgcaccaccactacccgcccaccatgtctgtcccccttcccacctgctccccgcagtgcactgtctacgtttcaggccagtgctacctgtgtgcagatgcccatggcctgctccagggcatggctttcggagctccctgctcctgccacatccgtcctgacctctctcttggctgcattggtcctgacctctctctcagctgcatcaatcctcgcctctctcctggctcatttctgccagcacaaaacttggtataaatgcccctggcctaaggaaatcttttgacccactttcctctccagcagcctctccatttctctgcttgccacacgcacacatgcacatatgcacacgcacacacatgcgcacacacgtatgcacacacgcacgatccatgcacacaatgcatgcacacacaggaaattcctcagagaatcatctgttgccactgtgtccattttctctctccctccccattcaggtttttgcccctcaactttactgaaactgttcctctctgagctgccaaagtctgactaaagtgacaaattctcagtcctcaaattatagatgcagaagcagcctctgacctgctgactgctcttgcttccttgaaacgctttcttctcctggccgccaggatgccacgcactgctgggttttcttgtatcttcttggtcacttctcagtccaccctgaccttcaaacatgctgcacccagaagtcggtgccctccactcactccctaggggatcccctccaacctcatgattattttattttattgctgggattacaggtgcacgccacactcccagctaatttttgttgttgtttgagacggagtctcactctgtcgccctggctggagtgcagtggtgcgatctcggctcactgcaaccttcgcctcctgggttcaagtgattctcctgcctcagcctcccaagtagctgtgattacaggcgcatgccaccacacccagctaaattttgtatttttagtggaggtggggtttcaccatgctggccaggctggtctcgaactcctaaacctcaggtgatgcactcacctcagcctcccaaagtgctgggattacaggcatgagccatggcaccgatctgtttatttttgagacagtgtctcactctgtcaaccaggctggattgcagtggcgcaatcatggctaactgcagcctccacctcccaggttcagatgatcctctcacctcagcctcccaagaagctgagaccacaagtgtgcacaccttgcccgactaatattttttattatatttttagagatggagtctccctatgttgtccaggttgttctcgaactcctgagctcaagtgatcctcccatctcagcctcccaaagtgctgggattagaggcaggagcccccacacccggccccatgattgtaaataactctgtctgctgggacgcctgggggatctttccaaccagatttctctgctggactccagacttgcagagctctgtcgtcagcctccacgcagggctgcaacagagccacggcatgtctggtcccagcagatgctctgcccagacctgtcctctcactctttgctgtcacgccagggccactccatcccttccattgttcaggcaatgaacatggcatcatccttgaaccctgtctttctctcatatcccataaccaatctgtccatggttccaccttcaaaatatacccagatatgtgatgatgttgaggaattgctggtaacttttttagatgtgataatggtattgtggttgtgttttgtttttgtttttgttttagacggagtttcgctcttgtcacgcaggctggagtgcagtggcgtgaccatggctccctgcaacctccgcctcccaggtttaagcaattttcctgcctcagcctcctgagtagctggagttacaggcatgcaccacctcacctggctaatttttatatttttagtagagactaaaaacagggtagtggtgtgatcacagctcactgtagtcttgacctcttgggctcaagcaatcctcctgtctcagcctcccaaatagctgggaccacaggctcccaccaccacgcctggctaatttctttaaaaatttttatagggatggagtctcactgtgttgcccagtcttgtcttgaactcctgggctcaaccagtcctcctgccctagcctcctaaagtgctgggattacagggagggagataccatgcctagctagtttcatttgttatctgcctggtgtgtgtagacatctgctttgtcacctccattgtggagagcaacatggggtttcaccatgttggccaggctggtcttgaactcctgacctcaggtgatccattccccttggcctcccaaagtgctgggattacaggcatgagccaccacgtctggccatggttgtgttttttaaaagagctcttatcttttccagagtcttactgaaatatttacagatgaaataatgtgatgcctggaatttactcacaataacctagggcagtgcttctcaagctttggcatgcattcaagtcccctggagggctcattaaaacagctttctaggtcttgagcccagagcttctgattcacaaggcctggggtgggccccgtaacttgtatttctaatgcttggtgctgctggtgtgaggagcccattttgagttgagctgatctaaggtataggagagtgagtagcctgggcgtgggctgggagaaggtggagttggtgggtacgtggtaatgaaggtacaagggtttattatgtcaccctacttttctttggaaagttccgcaactaaaaagcacgtgtgtatgtgtgtctgtctgtctgtctgtctccagaactcttcagctcttccattgctgcgccctggctcaggctagagtcctctcttgcccaggtggcgctagcacctcctaactggtctccttccttctctccttgttcctctagagtcagttctcaacagagcagccaggatgatccccagacccctgcagtggcattcagagtgacacccaaaacctttctgtgacctcaaacttctgtgcagtgtgttccccacgccacttcctccatgaaccttcagtcacactgcccgcctcgctggggcttgcactggtcaggagtgcttctgccgcaggacctttgcacgtgcccttctgggcctacagtgctcctccactctccatgcagccatctccctggcttccttcaggtccgcattcagatgtcacccttgcaatgaggccttctcagacaacaccatctaaaatgtcacttccccttccccactattcataccctactccctgctcactttttctgctccgcgcctgtcactgtctatattgcactgattgatcttatctatcctggcaggaagcttgagcagcgcccgcctcttcctggctgtgcctcggcagtgcccccaccccagcctcccaccccatcttggtctgcaccccctgccatcactcatccattcattcctcctgttcctcttggccactgccaaatgctggatggtttccatctgtttggttcactgcagtgtctctgtccctggaagggtgccagccacagaaggcacgaagatgtgtccacagagccggcggctaaaaatctggcaagaggagtcgaggaagggggctgaggaggtagaagggagggctttcctcaaaaggccagaaattcgtggccagccaaaggggaagcaaagacattctcgtgtaggaaaacttacaggaaatgcaactaaaagcgtgggagacaggaagtctgtgtatttctgagattatcaagaaaggggggatggaagttgcaaatgggcaggccaaattcagtctgcagaggtgtgcttccagcccacgcagtgcttaaaataaatggagatggttgccagcactggaaggtctggagtgccctgtctcctggaaacactgcagcctggcaccactgggctggtgagcatgactggcacctgcagcagctgcccaccagctaatggcaggtgtgtgttctctgctcacccaaagcccttccagcagcttcattttgttgttgttgtttatttgtttttgagacagggtagtggtacaatcacagctcactgcagtcttgacctcctgggctcaagcaatcctgctatctcagcctcccaagtagctgggaccacaggttcccaccaccatgcctggttactttttaaaagaatttttgtagggatggagtctccctgtgttgcccaggctggtcttgaactcctgggctgaagcaatcctcctgccttggcctcccaaagtgctaggattacaggtgcgagccaccatgcctggctagcttcatttgctaccttcctggtgtgtgtagacatctgtgttgtcacccccattttggaaagcaaaaaggatttcttcttccttcaaacataattgagaaaatacagttgtttgggggcaagtagggtgcggggtgactctggaaccggcctcctgggaagccaggcctctgggagtgctggaggtgggcagtgaggctgccaggcccctgccttctggggtggggtgcatgctcctgatacatcagaagggtcgggggaggacttggaggtgaaggcactgcaggaagagaggaagcttactggagagacggtcaatggggcagtcaggctttaaaaggacgggcccaccaagtgaaaagatgaccaagtgtttgggctgaattgtgtcctccaaaaggatatgttgaagcctaacccccgggacctcagaaggggatcttatttggagatagggttgttgcagttgtaacgctgagatgagattatactggagtagggtgggcccttaatccagtgtgactgctgttctaagaagatgtacatgtgacaatggagggacatgtggggagaggatggccacgtgatgatgggggggacatgttgggagaggatggccacgtgacaacagggggatgtcaggagaggatggcctcgtgacaacgggggaatgccaaaagaggatggcctcgtgatgacaggggacacatggggagaggatggccttgggacgacaggacatgtggggaggggatggccacgtgatgatgggaggacatgtggggagagaatggccacataatggtggggggacacatggagagagaatggccacataatggtggggggacacgtgaggagaggatggccacatgacaattgggggacaccacgcggggagaggatgaccacataacaacgaggggacatattgggagagaattgccacgcgacgacggaggaagggactggagtgaggaagtcataagccaaggaacgccaaggacagaacaagcagcaaacacaagacacagggccaggtaaggaggtccccggctccaggtatcagaggagccctgctgatgcctggatttcagctctctggcctctggacccagaagagaatgaagttctgctgcctggggcctcccagtttgtttacagtgagtctgttcatggtggagaaacatcttctcatgatgttcctgtccaccatggtcaaggcctgttttgagctcctgcattgtataagtcagaggtaaagctgtttttctcaaggggtcaataattgacagtattggaatgggacaaggtggtcctgttgggttttgtttgtttgtttttaaaatatgaggatagaaagtcgtgtttttacttgtgcttaattttcccagtggccgggatgtgaagtccacgcctgtcatctgtaatgcaccagggcaaggggtcggttttgtttgcatagcttctcccagggtcatcttcccacccctcaagtgtggatttggccacaacactttgtttagcaatttcctgtcattttgttttatttggtcaggcttatttgcatagttgaatgaatgcacaaaggcctccttgtttcgtggttcacaaagtcagaactggatcataccagggcatacgggttctgtttcaaggctttgtaaagctgctagggttgaaataggtatttttggtgtcggaattattttattgcatttatcagtttatctactgagatggggcactgagaactgctgatttacctctctgcagccctgtctgcagtacctgaaggctgggggaagacatttggcaatgcacacagcctttgtttctgcctgtgaagttggggtttcataggccacctcccaggagggccctgtaggtgcatgacagagtccgagtgtctcaccagtgggtttgtcattgcacccaagcagatgctgggggagaggatgcctcttccttctcctcacccttcatcagtgtgtgagcagaggcaggagacctgtgggctggaaggcccacatcctccttcccaggctgtgaatgtggcacttacatggcaggaggactttgcagatgtgatcaggaccttgagaggagacattatcctggatgatctggatgagtccatcgcactcacagggtccttaaaagctggaagagggaggccaggcagggagagggaaggaaatgtgaggatggaagagggatccgattgccgcgattgctgaatttgaagaggaaggaaggagccgagagccaaggaatccaggcagcctctgggagctgcaagagacaaggaaatggattctgtcctgcagccttcggagcagatgcagacctgccgacaccttcctttcagctcagcgaaatccatttcagaattttgagtgcagagctgtaaggtcatgagtctgtgctgcttaagccactgtgtttgtagtcatttgttgcggtagtaaaggagactaatatgctattttgccaccattctgagcttttgaggcaggctgatggaaaaggtgggacagtggcttcccagtacttttctaagtgatcctgagaacctaatatcccaggcacttcatattctgtaactctccaccctgctgccagcagagtctgcttagtcctcaagacgaggggcttctgctgctctcagaggttgatgatttcattgattatctttaagttactctgcactctcccttccccagagaaaacccagtcaaattcaagtgagcattggggaaaatattcattcattaaactggttcttaacctttttgggggcaccgacctcttgagattctgttgaaaatgggattttagcccccataaaaatacatatcaaaaatttgcataaattttggccaggcacagtggctcatgcctgtaatctcagcactttggaaggccgagacgggcagatcacttgaggtcgggagttcaagaccagcctggcaacatggcgaaatgccgtccctactaaaactacaaaaaatagctgggtatggtggcgcatgcctgtaatcccagctgcttgagtggctgaggtggaagaatagcttgaacccaggaggtggaggttacagtgagctgagattgcaccattgtactccagcctgggcaacacagcgagactgtcaaaaaaaaaattgcatacaattttgagaacaattttgtatacagattcccccaggagctaccccttgaagctccaatggctgccaggggcttcctctgtcaccccaggtgaagagcccttgtacagattaattcacattaaggctgatgaatggctgttttcctagtaagtctcctcgctgtttgccaggtccttaaaccatgttcagtggatcctcattatttgtggattccatatttgtaaattcatctgctctctaaaatttgtttgtcaccccaagatcagaactcatagacaccagcagagtggcaaaaatgtgttacccgatgagccttttcccagctgaaaaggatgctctgccatcttgtttcagcctcttggtgtaaacgagtgtggttttcatgatttgtttagtgccatgttttccatgttttgtgctttttcttcgtggtttcactgtttgaaagtgctgtctagtgtttccaagtgcaagaaggcagtgatgtgccttatggggaaaatatgtgtgtttgataaactgtattcaggtttgtgttctagtgccactggttgagtgttaacgaatcaacactgtacagatgattcctgacttacgatggttccttccagtatgatttttcaacttaacaacggtgtgaaagtgatatgcattcagtagaaaccatctttcaaatacccatgggaccattctgtttttcactttctgttcaatagtctatacatttcataagatattcaacactctttcagaaagtaggctttgtatttcatgatttcgcccagctgtaggctaacatgtgttctggataggtggaaggcaagctgagacatacctctatggtgtttggtaggttaggtgtattgaatgcattgtcaacttaggatattttcaacctatcctgggtttatcaggacatgaccccattgtaagttgaggaccatctgtttattcagtaaggtgtcttttaaatagaaaaacacataaaacaaggttagtattgattgattgacaaaaatgttgcaacctgaggctttcaggaacctaactctgcatttcctttaggagtgctgcttgcttcagtatctgctggtaactctatggaacatagctactgtgaataatgggaattgggtgtattttcaagttgttggcctctccagctacgctggtaatcagtcctgctctttaaatcacatcttaccatcagtcctgcctagagaaatggaggaagggaggtctctggtcttacggtctgtcccgactctctgccccaccagctatgtcccgagaaccagcctggacagagccatctgattggtgggtggtagcaggtcatgttgtgaggccctggtgcctgccttctgtcctcctgtgctcgctagaggagcatgaggcaggagcagccagtccatgctggttcctgagcgaggcagctctagggctgccagcacccactcctggggggctgagggtaggaagagcttcagaggggaggatgggactgtctgactatatttccaatcccagttgggacttgtggtctgatgaggacaagtgcctttgagggggatggggatggttgagaggaaccaggaaatcaaatgtgtcctggaaaacatggcatggtggatcttttgagttggggtgttgggatgtgtgtcagctaaaacaaggttcacctgcttcaaatgttcatccaagctttctacacttgcctctctgagtcaacatctttagagacagcaggaagtgatgaaagtcagttcacagtccatctgctctgagggcttggtatgaggggctgatgttttctgtcattttctcctttctaacatttcactttgggatcttagttcctggttgaaattccttaactgtgggagtcgaggcagcctaagaatccttttcggctgcttaagacaatgaggtagagagggaaccagctgccccttttcttacctccatgatgatgtagaaatggttccatccagcccaagggttcttctggggcccaaagctccttaagggctctgtgcataggatttgaggattagttttcttgggtggggagaaaagtccatctttatttgtataaatcctacctgaaatgcagcatttcctctaattaggaaggcaggcaacaaaaccagtatgttagctatgtctgtgactctgtcaccaatgtgggttagccgtatctctgaccctgtcaccaattgcaattacagatgtctccacatcacattagtgttgtttcagatatcttgaagtagcatttgttcccattactagttttaaattaaggtggttaatagaccccactggattttgcgatttaatatgctgataaggcacatatattgctatactataacattttttaaaaattttgataactagatttcaataagattggtatttgtgatcatatttattttcttgatttttataacagctttattgagatataatttctgtaccatacaactcattaaagtgtacaactccatgtttctaaaatatattttcaggattgtacaaccatcaccaccaccatcaccataatctaattttagaatatttttgtcccccttaaaagaagcttcgtactcattagcaattatctacattccccctttccccactctcagcccctggcaaccacaacatctactttctgtctctagaaatgtgcctgttctggacgtttcatagaaatggaatcttgtttttgagacagagtctcactctgtcacccaggctggagtgcagtggcgtaatttcggctcactgcaacctccacctcctgggttcaagccatccccctgcctcagcctcccgagtagctgggattacaggcgtgtgccaccacgcccagctaatttttgtatttttagtagagatagggttccgccacgttgcccaggctggtctcgaacttctgacctcaggtgatccgcctgccttcgcctcccaaagtgctggggttacaggttacaaagtaagccactgtgcccggccaaaatggaatctttctgaggtcttttgtgactggcttttttcatgtagcataatgttttcaaggttcatctatctttgtattttgttctgtgcacttaaaacattattctgagaaggggtctaaaggcctgccccgtcgtcaccagaagggcctgtggcatcattggagaggttagggctctccctaaccattagggctccctgcgcctaatggttcttccctctgtcagttttgcattaaaactagtacatgccttggcttgagcatggacaatcactgccaaagacccggcaggcaaggcacgcagctttccttgggatgggttggacccaggccctgggcactcagaggtactcaggcacctctgcccagtctccccaagttaccacctccacaggtgccttggagtgagtgggcatctccaggatgtagcctctagtggatgatacccgtggacagggacagcaaggtagagtaatagggccaggccccaggtgcctgcccccagctggacgccagaaccccaggaccccagctcctccagcccccaggaagctgtcagcacttccagagagcaagctgttggtgggaaggtctgaatccttgcagctctgtgattcgcgaatcataaaacctccacacttggtgtttgtgggccgagctattgtctttgtgttttctttttggagtgggtgggaagccagcttggcaagccgggtgtgaatgaacaggaatgagcaggagacgctcgctgatgccttctgtgatgtgtttcttccccagctccagaagaaaaggaagtcattaaaggacagtatggcaagctcacggacgcgtacggctgcctgggggagctgaggctgaaatctggtgagtagccgctcgctggaggagcaggcgccaggctccccggtgggcaggagcctctgtgtcggaaggggcctcagtgcaggcattctgtttgaccgctcttctctctctcttcttttttttttttttttttttttctccgagatagagttttgctcttgttgcccaggctgcagtgcaatggcgcgatctcggctcaccacaacctctgcctcctgggttcaagcaattctcctgcctcagcctcctgaatagctgggactacaggcatgcgccaccatgcccaactaattttttgtatttttagtagagacggggtttttccatgttggtcaggctggtctgaaactccgacctcaggtggtccacctgcctcagcctcccaaagtgctgggattacagccatgagccgccgtgcctggccgacctctcttctcttttaagatgtttctttatgcctcgggacctgctcctcctgcaggaacctgcaggtggggaggtcaaggtgagtgttgcctggttgtcacctgcctgtgctggggagcagggtggggttcccggcacccccaccctgtgccagagaagctcgtcttcattgcttgccggcgagggcctaagtgtttcgaaagtctggtctggggcttctgcccagggcattgtccgcagtactctagaagatgtgccaaggcgggtgggtgaagacagggaccagggcagatgcccgggggcacaggggtgggctgggagttgtcagcaggagaaaaaagccaccgatgtgtgactgtcagtcagtgtggggtgggagggagaggaagggacagtgtgatctgacttgcgaagggagagaacttccaggaagacgtgggcatctctgctcactctgagagactcgggggtgagccggtggtggccggctgtcgtgggtggtcacagccaaactcagctgttcctggagctcaggagccctggggctttaggttctaccacctgtttccttccttctctctcaggggcccccttgccatctgtgacctggctgtggacatctcttgtgacttctggggccgagacaagagcccagtcgcattcctgattctcagtcccacctggctgagatgggaatggctgtatgggttgtctgaattcgggctttccctgagccaagcgcacacctcccttccctgctcacagcagctgttttcagagactgaggatgtttaaagctgccagaaaagcacggttacgttggccattccttttctgggtgttttctgtcatggaatgtttttccctgaactgtgacgattacagtgatcatttccaaatagcatttaggaaaaatacacagtacccacatgacaaagtgcatttatgaaatgtctgtttcgtgccaagagctagccgtacattcacttatttaatcctaaaaataggctaccgtgataatgcatctttgtccccgtttacagtaacgggaacaggctcagaagcttcaagtaacttgccttaggtcagaaacttagttggggtttgaagcgcctccaaacgcttgctttttctgtggaaatgtgcgggctgccgagggttttctgcaggactttccgctctgcagcgatcttcctgaagttgcaggaaatccctgtggcccttgccttccatggtgatgaccgtgaggatggtggcctgcacctgcatgggactcacttaggaacttgttaacacggctcccgaggccacctgtgctgttgtcaccctgccttgcaggtgtggcagggatgccgcgttccatccatgctgatgcccttttttccacatggtagtatctgggcccccaggatgcctcacagtcttggcttccttcagttatagttggcagctgcttttggtccttacatgaatgactcattttgcatccgacagggactgggattcgacgaattacagtaaccggctcaaggtcacgccattctcaagtggtgccctgggttttaaactcatgccttgcggcaccagggactgccttcaaccaccaggctgttctcctttctgaaagggacaggtggctgggctcacaagggggacaggaagtgcagtaccttcccccatcccaggggaaaagtgttttttcccagggctgtgatctaagatcagctgtgggaaattggtcctaaccaggaagggtggcagccccaaagatgtcttattagagcgagccagggtgtctgcgggggtcagcagggtctaatggggcagggagtgctcagagaggagaccacctcccagggctgccctaggcgaggcctcctccctgccttcagccagtccccgccaccccacaccccacagaagggaatgaggcttcttgtggaccatgcgagaccagccctgctccgaggctttgggtgcccccaacccctgcccacctgtgagagtcaccccccggagtgggttggagtttctctgagtgtggtgtgtatgagtttgactttggcgatgtcaggataatttggtctgttttcaacacattctggaaagcaccctgagcatctgaaggatggaaggattgggctttaaatcaagttttttcagtccccattggttatcttaaggggcgtttgactttaacttgattttctttttcccctgtaagtccctgacccagaactgtgatctattttcttttgtgttttcagctgttgagcatgttgggcagcacggagtgtagctacaactgtgtgaccccacctgtgtgactgactccacctgtgtgactccagtcgtgtgaccaactacaactgtgtaactgactccaattgtgtgactaactccacctgtgtgactctgcgtgactccaactgtgactccatctgtatgactccaattgtgtgacccccacctgtgtgactctgactgtgtgacccccacctgcgtgactctgactgtgtgacccccacctgcgtgactctgactgtgtgacccccacctgcgtgactccgactgtgtgacccccacctgtgtgactccaactgtggactgactctaactatgtgactgactccaactgtgtgactaactccacctgtatgactccaactatatgattgacttcaaccgtgtgaccccacctgtgtgactccaattgtatgactgactctaactgtgtgactccaactgtgactccatctgtatgactgacctcaactgtgtgaccccacctgtgtgactccaactgtgtactgactctgtgtgactgactccacctgtatgactccaactatatgactgacttcaaccgtgtgaccccacctgtgtgactccagttgtgtgactgatttcactgcgtggctaactccactgttgcctgactccagctgcgtgacttcagctgtgtgactccatgtatgtgaccctagctgtgtgactccaactttcagttgccacgaagacaaccagctgagtcaggagtgggcctccctttcaatgtttttagctaagcatctctgcgcaaagaaagaggcttccccacaggctcttaatttgggacaagttttcccttagtttgggaaaaatttcaatctacctaaattagccttttacattagaaatcgagaaatatgcacaaaaattcataaaagcctttttccaggttccaaggtttttgtagaaagcccaaagttcccctttccttcctgaaatgccagcaggaaggggttggagctcccgtgattagccagggagtcaggcttcctgaagtttacacaatacaaagcatattaagtgagaatgtagcagaaaaaagaacttatcccaatgattatagcaatttaaattctcttgaacccaagctctcaaggcctacctgttttatagcctcaagggactctactcctatcccagactataatgaattgtgtctcctggagttgtgcagtacgcaacctgtgaagctgtacacgacagccctataaattctctagcagacagccctcctagattctagttcccacttcacagctgaactgcatttaatctctgtaggactgttttcctgtctgtaaacctgggaaaactgcctcatctctatgttgcctagttctcaggaggtccacatgggccatgcacatggtagccatgaagagtgtggagtgctcctccaacaccaggcgggctggccacaggcagtttcttagagtgtggcctggggacgctgccaccactgagaagctctgctgtctctcctctgtccctgggagggctggagggaggagggtgctgagtgagagggacctggcctgcagagggctgaggacgaggtgtgagctggaaggccaggctgtgtggcctcggggtgtggactcctggtggggggctcctctgcacaggctagcagagggctggtttcatgggcagtcacttcagaaggaggattccctggaggggagtttgaacccttagtcttttgggatgcagtgttctctagcaagtcttgaagcgaactggcctttgtcggtcttagattaatttaattcctgtgcccagtggtgggatattaccaagagagttgacacccagctccctgagggggaagctctgagatgatgcagcatttgccagtttccaaggtgtaaattcacgggccagggctgatttcaggttcccagcgtggagctgaggaaggatctgcaccaggggtgctcactgcgggtgttcactgctgtggccgccgctgcctacacctccctcatgttctgcagaagggaggcgggagaaggccctgagaccatgccgcatgggcacacagtgcttagggttctgaacctgcgggtggcgggggaggctcaagggtcatctggccgaagcttcccccttcacaggtgggaaagcctagacatctttagtcctttgtttggagctttaagtctgagggtgtctgtgtgagcagagggacagataggatggctggagggaccccagggttgggattggcacagaccaggctgggaaggtgaactcagagctgagacaacacaaggaaatgccagcccagagccttgtgggaggcctggggtggtcccggtaaacggggccttgggtctgcaagccacgtatcatgactccaacagccagctttcctggcctgataacaaattgcctaggacctgccactggctcttgcctccctccacctcactctggcagctccctctgacccatgcatggctttgctttggccagagccctggaaccaaagccctttacctggaggggacattgagactattggcgcatcctcctgggaggcccagagagggtaagagccttgcctgaggccacacaggccactggaggtgtgcagagcctggagccccactcttctgagttttgtttttccacttgctatgagatccttgatggtttcagggccctggtctctccctcagctgaggactgccaagaaccccgcaagcctagttagcttcttggctgtcagagaattgatcctgcctcacaaaagcaaatcctgtgctctaggtgagcatcgggaggggcctcacacacctggggcaggtggggaccagtggcttgtaccacactgacggtggcccgggagtcagcgaccccccacagcctggtgctaggctcaagccaggacactttccttaaaacccatagtacaggccaggcatggtggctcatacctgtaatcctagcacctcagggggccgaggcaggaggatcacttgagcctagaagttcaagaccagcctgggctatgtagcaagactatgtctctacatagtgatggtatctacagcaccatgccaggagcggtggtgcacgcctgtagtcccagctacttgggaggctgacatgggaggatcgctggaacccagaaaggggaagttgcagtgtactgagatcaagtcactgcactccagcctgggtgacagtgcaagaacctgtctctaaatttaaaaaaaaaaaaaaaagcaaacaaacaaaaaacccatggtatacagagaacacagtcaggcagtcaggctgtcaatgagattaggcaggacacagtgaggctgggattgaaggaggaggaagccatcagtgctgggtcaggtaggcagggcggggccttcccggagaggcagcgctccacacagcggtcctgaggtttgggtaaggctcttccacagaggcctctgtggttacacccgtcccaccaactgtctgggacacacaggcaggggcagttctaaggaaaagtgtccaggtccttggcttttttctatcatggagcacctgtgatggaggacagggttcttattcccctctgaggtctccctgcttccatgttagggtagagggtaggcccttctctcttctggaagctcaccgtgtgccgtgctccaggaagtaactccccctaggattccagacaaagggacagttcaaaagcttgtcaacagcattgcaataaggtcaggactaggtagcaatctgtttgcaagtcaggtgatgtccaattgtttgcttcccccataacggaaggagaatttaattggggtatcagctgacacattagtaaccattttatcagcaggttattgtcatttcttagcgctaactcagaaggactttgaagaatcgagtgacatttctacaatgaagccctttccattcccatctatttattaatgtgaacaggatcttttagtgtttgcgtctacaaaaatgaaaactagaattagaattggtgcagaacctactttcattccttcaataaatatttattcatctcattaagagatgcctttccaacacattatgactttttgtatcaaatgattatttgtcggccaggcacggtggctcatgcctgtaatcccagcactttgggaggccaaggtgggcggaccactagaggtcaggagttctagaccagcccagtcaacatggtgaaaccccatctctactaaaaatacaaaaattagctgggcatgggggctcatgcctgtaatcccggctactcaagaggctgaggcagtaaacctgaaaggcgaaagttgaacccgggagacagaggttgcagtgagcccagattgcgccactgcactctagcctgggcgacagagcaagactgtcacctcccagagaccccacctcctaaaaccatcgccttgggtgttcatacttcagcatattaatttgggggtgcacacattcagccaatagcaggtacactggttgaccacactcttaaagaggacagccaaaaaaaattatttggcagtgttttatttacattgtattgtttacattgtttgaccagttaggtatcactggaacgataaaccagaacgctctctttttaaacacttggagttgaggaacacacggctgcaggggctgatggggtgatcagaacaggacgtagaagcaccagatatgctacattaggatggaattctgaagaggggggactggaaatatgagtgaaagttccatgtggtaagaaagagcatgctcctgcaatcctgtaaataatagatggcaatgagtatcacgttactgtgtagattccatgggatactgacaagaggctcccagagttttagttttacgtgtcaatatttagaatacactgcacaatatattatttacaggcatttcaactaccgatgaaaaccttcaatggcagttttaaaatgggcagggaacgctgtctgagtccagtgtgttgctgtgacaaagttccgtagtagacggggtggcttatcagcaacagacatttatttcgcgcagttctggaggctggaagtccgagaccagcggcccgcagattggccggctgtgaggacctgcttcttgctccatagatgctgccttttctctgcatctgacatggtgggagaggtgcaggagatctctgggcacaaaccccattcatgagggctccactgccataacccagtcacctcccaaatacccccaccacctcaaaccatcaccttgggtgttcgtacttcagcatattaacttgggggtgcacacattcagtcaatagcaggtacactggttgaccacactcttttaaagggtacagcaggccaggtgcagtggttcacacctgtaatcccagcactttgggaggctgaggcaggtgaatcacctgaggtcaggagttcgagaccagcctggccaacatggtaacaccccatctctaccaaaaatacaaaaaattagccggtataatcccagcaactcgggaggctgaggcaagagaattacttgaacccaggaggcggaggtagcagtgagctgagattgcaccactccactccagcctgggcaacaagagtgaaactccatctcaaaaaaaaatagccgggcgtggtggctcacgcctgtaatcccagcactttgggaggccgaagcaggcagatcacctgaggtcgggagtttgagaccagcctgacctacatggagaaatcccatctctactaaaaatataaaattagctgggcgtggtgacgcatgcttgtaatctcagttactccagagggtgaggcaggagaatcactagaacctgggaggcagaggttgcggtgagccgagatcacaccattgcactccagcttgggcaacaagagtaaaactctgtctcaaaaaataaataaataaataatacatgcatacatacatacatacgtacatacatacatacatacagggtacagcaaactaatccgattggaaactgccggactttggggctagatggagcatgggcttaaagtgaaacctacattttggctccaacacagactagctgtgtgcttgggcgggtgattcagcttccctgggggacagtcatgcgtgtgtgaaatggcagtggtagccctgagacagcagcatgggtaagcaggctggcgacgtcatctgcaaggaggtgtaattaggagcaccagctctggggccagacttggcttgagaaacttgcccaggcactgtagctagaaagtggatttggatcccagctccccgctctgtcaccttgggcaggctgcttaacctctctgtgcgtctgtttccctccctgtgagatggggctggtaagggcgccccttgcggtgggcgggaggatggagtccgcgcctgtacagctcccctgcctccactgcgctcaccagataccggctctcctcactcccctgtaccactcccgaccccctcctccctggacccaggagccccagtgctctgtggcaatgttaagagcctctcctggctgctacagtgctcctctccccagcctcccccaggccgtggtcctgcccaggaaatgcacctgggactgttgccaggcactcaggccccccacacggaggctgttgaggaagtggtgctagaaatcagctcatgactctcacggagtgatgtcatgcaaaattttccatagctctggcagccacaggggccatcctcctgcctctccgagggcacttctgctgaactgtgcttttccctgcactgtgtcagaagaggcaggtgggtgccgggagcgctctgtggccaaggctctgatcacaagctgccaaccccggaaccccacctatcctacttgagaagctgaactctgacccctgtccctgtccccagtgtgataacttcctttgttcttaggtggttttgaggtggtttcctgaggatgtgtctggggagatgtgatgggatcaaccccctgccctgctggcagccccaccccatggcatagcagcaggcccctgggagccctgagcgctcattaaaggaacgcactttaatgacagccacacgccaccctgccacaagcagcgctcttctccctatgaagtgtggtgaggaaattggggtgagagggtggtggaactggttgttttgggggtctcttcctggagtgtggagagatcagaaccatctccagaccatggctgtagacagcggcccttgatcattcacagaagatctcgctgggcggatccttcagacctcagcggggtgggaagagtgtccttgggactagagccctgccagaccccaaaggccggtttctacccgatgatctcttgggccccgtcccgagagtgaggcacggcgccaccaggcacccccgatacgcagcgtgttgtgaagtgcccttgtgagcagcgggggatagggaacagaaaaccagctgtcagcgacagcgaggtggtgccccagagagggctgcctcattcgcctggctgcagggtccgaggtcgtcttggtagcatttggcaggaatttatttaggaagcattcagcaaaccgagtgtgtgtggtgctgggagatgacgacgaaatggccctgcccccaaggaagcccctcccctgggggcacaggtggggagggaggtcatgagctttgtgtgtgtgacaactgctgatgggaatgctattggcacgtacgaggggtgcctgtccgtgtcttgggagagcaaggaaggcttcccggaggagttgctgccaagttggcccagaaggatgaggaggagttcttctggtgaagtggggagggcagaggcaggatggtccctacggagcatgtggggacgctcgtgttcttccagaggatggaatgctgggcccgaagcctgaagtattgttagggtagagacaggcgggaggtgcgtggagaaggggtcatggaggtcttgttgggcagatggcggtgggacgtcccaggcagggggagcatcttgggcaagagcccagggtgcgaggtcatgcttatggcacattggaccagctggaggacagccagctcaggagagcagccggctggggtgtctgcccacgtgtggggcagggatgggctgcagcggggagggatttgggagcttctgtcgtagtcctgcagggagtgggagctgtgcaagaaggaagggcggagcggctgtgatccaatgactttgggaaggccactcctgtgatggagtgggggaagacagaccccaggccaggggtcctgagtcaaggcagtggctgaggaggcactggggaaggtgatttggagaggctttgggggtaggtcctgggggtctggaagggctctaggccaggaattttcagactcctgggggcatcagaatcacctggagggcttgttaaagcaaagcttgcccccagcctcaggcagctgggcggcaggaatctgcatgtctaacaggctcccaggggatgctgacttgctggccagggtcccacactttgagaagccccgcctgaagcttacaggacagggcaggttcttgagccattgagttgggcaatgccatgggaagttgcaggtgcacagacgttgccttcctggctgcggttgaacctgggctccatttgtccccttggagctccaggcggccgggccgacttcgaccctgagctctcctgtctgatttcctttccaggtggcacgtctctgagcttcctggtgttggtgacaggctgcacatctgtgggcagaattccagatgctgaaatctacaaaatcactgccactgacttttaccctcttcaggaagaggccaaggaggaggaacgcctcatagctttgaagaaaatcctcagctcgggggtgttctatttctcatggccaaacgatgggtctcgctttgacctgactgtccgcacgcagaagcagggggatgacagctctgaatgggggaactccttcttctggtgaggccctgggtcccctgcagagggtgggccctgggtctcctgcagagggtgggccctggttggcatctgaggccttgacctccacttgcaccacccccgggttatggggcacctctggagagttaggacccaggcctgctctcagcacaatgagaggaaaactaaccctgaactccctgccaccccccacccctggcccctggaatgtgttcagtgtagatttggaaaagaaacctgcgccaggcatggtgcctcatgcctgtaatcccagcactttgggaggctgaggcaggtggatcacttgagttaaggagtttgagacaagcctggccaacatggtgaaagcccatctctactaaaaatacaaaaaaaaaaaaaaaaaaaaattagctgggcgtggtggcaggtgcctgtaatcccagctactcgggaggctgaggcaggagaatcgcttaaacccaggaaacggaggttgctgtgagccaagatggtgctactacactccaccctgggtgacagagcaagactctgtctcaaagaaaaagaaagaaagaaaagaaaccctgttccataagtaaaaaacataagagccgacgtctgctgagtgcttcctcagtcccgggtgccgagctcagtgcttctgcctggattcactctttcagtcttctcaatcatggttacctccagtgtgcagccgtttaccgctgatgtttataaagcactgtgttcgggtcaccgtgctaagatctctctgctggtaataactcatcattattataaaaacctttataataattatgcggaggctctgttaccccattttacaggtagagagactgaggcacgaatagtcgagggaattttgccaaactccacacacttactaagtggcagagccaggatttgaacccaggctttctgggtccaggtcccaagctggtaatcacagtgctaaaccagtcccatcaatggggaagctgagtcaccctgcccaaagggacttgtagcaatgcactaagactagtcccggagcccaggcagaggcggctggatttctgtatgtttaagatctcctggtgctttttcactccacagcaggacagagacaagagtggacaaccctgtaaatacataaattatctattgccctttctggacataatgctgtttctgtcagattcgcactgatgcctagagatgccaggtagaagcaaaagtgaggaaagtcctgccatgggagtcgttccaatgaacctcatctaatgtaatgttttagaatccagtgtctgccttttgaagctatatcagttttcccagctctgagacgggactcgtgaggtttatttctgacctgtttgtgcctctctccctttgccagaggcagccacaggttttcatctggccttggtgctttggattccccagaagtgaattcctgatttctgccggaagaaggggaccctccccacatccctgtgtccatcccaacgctccccacccccacatcctctttggttctctggtattgtggatatttgggggcagtaatgtgcccagacttcaaaatgcacgtgaatcaaggctgggcatggtagctcacgcctgcaatcccagcactttgggaggccaaggcaggcggatcacctgaggtcagcagtttgagaccagcctggccaacatggtgaaaccctgtctctactaaagatacaaagaaaattagccaggtgtggtggcgcacacctgtaatcccagctacatgggaggctgaggcagaagaatcgtttgaacccaggaggctgagattgcagtgagccgagatcgcaccattgcactccagcctgggctacagggtgagactctgtctcaaaataaataaataaaataaaataaataaaatcgtgaatctcatgctgatctcattaaaaatgcagattctgggatggcaggtctgggctgggcccaggattctgcatttctaataagctgccagtgctgcttgtctgcagacggcaggttgagtagcagcctctggggatggcatttctgtgaatcgctggttacattttgcagggcccagcatgtgccccgcgagctgttacatgttcccctaaaggcagccaagcttaagcacagaggggctgtgtattatgaggtatctttgtgtggagtccgtgaaactattttaaaccagaactgtcaggggagcagaggtgagagctctcagcggcagtgagggtccttggaacccctaggggatccttggggtatctgaattgcaccagcacctttgcaagttctcttccctatgccagctctgtggctggaggtgtgaggggcactaacaaagcgaggcaaaggggattcacgaggtggaggctgagaggagctttggaaggaggagccccatggtgtccctttctgaagggagaggaggaagtaactctggaggtcgccaggctcctcagctgtaacacgccagggcgtggtgtttaggctcagggtggagcccccagaactgaccacaactgagtcaccacgggcagtgtggttctgggcttgatcgcctgaacccccactgttgtggcgcagcgtgaggtctggagacccacagcaggagtcagatggggtcaacgctcagcgtggctggcacataaggcagagtgagcagcagggtgaagtcatctttctgggctggtaattgatcacactgacaattgcctcccagtacaatcaggtcctgggaggaaactctttgggggtcctggatatgagtcatcctcctgccagagggccaggcagtgtcactagaaacagggctgatttaagggttgtccacttggcaaaaggtttcttctcctgagccctggtgatagtggccatagctgtgtagctggcagactctgaaatattctggaggctttgccttgatgccacatgcagatttcatatatgtgtttcggaagctggcttatcatctgaatttgagagagtgatttggaagagggacatgtgtttttgtacagcaatgcttcagcagcatttttcaagcagaggagcgctgtggctgtgcgacctcagatggggtcagacgcgggctgtgctccctgtgggggtgcatgtgactgtgcgacctcagacggggtcagacgcgggctgtgctccctgtgggggtgcatgtggctttggtcacagtcggggaggctggcctgagagcaggaagcttggagaagccccctgggacccatcagagctcacagcacagatttccagcaatgagaaaatgctgctgctgtctttgaggaaggctcaagggcaggaattgtgttgccttttttaaacaagtagggcactgtgatctagattcactcagtgccctgtaagagggcatctcctgatgatgggtcctcctccctgagagtgagtatgggccctcaggatggtaccactcaggtcccctgggtgctaaagcaagggggccaggccagctcagggatgagagaactacgtggtcgttcgcaggtgcacatggaaacgtagggcctcatgaagaaaggctcggcgtgaagagcgcgggctccaagctccatgacgccactggctggctctgtggcattagacaagcacttcacttctctgtgcctcagctgctttatctgtgaaagggggtcactgatgatacctttcacatagggtcacttcaagggtgaaatgagttaatccaccccaaagtacttagaatggggcttagtgcacaggataaatgttggctgtcaccctgtcgttatcatcatggactctagagcctacagcaatccactaggcatggagagggaatacttgaactttcattacattttattttcctcggatctttttaaaatttattttggattgctttgtagcgctctcgtcataatcatctcatggtgcatgtgtgtgtatatgtggataatttaaataactgttcccatatttaatatgatttttaatcacttattgatcatgattgtttaataataaaagatgattaattttttaccaatggggcctgcgatgaaaactgtggggcccacagggctaaccagaggcccctttgcgattccccgaaaagtgcacgctggctcactctccagctttgcttccatttagcgacagtgacgacctggtagtggctttgggaaaggactccctggccatcctcctctacgtgacacagtggtcctgctatttgggatctctttgaagcacatgaacttcccagatctgtacagcccaagtgcagagtccactggggagcagcatgcattcgctgacccgaaatgctgcaccgtgcgccccccagttcctcagccacactcgctgtccaggcagcatgtattggaggatgtcaagtgactggaattggtgtgggactgtgttttgcaggaaccagctgttgcacgtgcccttgaggcagcaccaggtgagctgctgtgactggctgctgaagatcatctgcggggtggtcaccatccgcaccgtgtatgcctcccacaagcaggccaaggcctgcctcgtctctcgcgttagctgtgagcgcacaggcactcgcttccacacccgtggcgtgaacgacgacggccatgtgtccaacttcgtggagacagagcaggtgagtgcccaggcccatctgtggcaccaaatggttccgagtggctgcagctcttgcacacgcgctttccacttggggcagaagaggccctggcatatttgtggtggtctctgatcaggcttcctcctataagtggagaacagcacgtgtaattgggctccacatgaaaatgtgacctctggtttgcatggtgtatcctttctgattcacatctcatgcagggaaaatgctgcttagccgaggtatttttaactaaacggaatcaggaatttttgccttggtcgtgaaaggtgggcagtattccaagggcagctgacagatgaacaaatagggtttgaaaatccttttaggacaaaaataagtagatcaatacaatggggggatgaaagaagaaaccattattttttattctaaatcaactgaagcttctcctggaagccagtattagcaccctagagttgtgaaatctgcgtgttaagcagccgctcccatccgttcccctagaatatagccatctgcttaatttctcctggagttttttctaaattctgttctcatttccagagatgctgtcttacaacttttaaatttttgctctaacttgcaatctgtaagataaggccaccatgtggaatacagtggtgtaagccataagaaaggaaaaccccggaaggcacatgtatggtactcctgccagggcaagctgggtgccctgtgtttactgagtgaaggcgtggctgtgcgtcccccctcgctgccgctgctgggagaggaatgccttatgcggactttggccactttaatccagcagaggtttgagcacctacctttactgtgtgggactttctgcctgctccagtgttcagagggtagatacagagatgaggaggctgctctcagagtccccactcagaggaacatcagagcagggggccctaagatgggcaacagctgcaattacttgcctgtaattctgctcagaatgtctgcactgtggatatcaacgaggctattatgtaaatatctggttaaaaatgagattgcttttggcatcttcgtcatgaagtctttgactgttcctatgtctagaatggtattgcctaggttgtcttccagggtttttatagttttgcgttttacattcaagtctttaacccatcttgagttgatttttgtatatggtgtaaggaaggggtccagtttcagtcttctgcatatggctagccagttatcccagcaccgtttattgaatagagaatccttttcccattgcttgtttttgtcaggttggtcaaagatcacatggttgtaggtgtacggccttatttctgggttgtctattctgtttcattggtctacgtatctgtttttgtaccagtgccatgctgctttggttactatagccctgtagtatagtttgaattcaggtagtgtgatgcctccagctttgttctttttgcttaggattgccttggctattcgggctcttttttggttccatatgaattttaaaatagttttttctagttctatgaagaatgtcaatggtagtttaataggaatagctttgaatccataaattgctttgggcattatgtccattttaatgatattgattcttcttatccatgaggatggaatgtttttccatttgtgtcatctccaatttctttgagcagtgttttgtaattctcattgtagagatctttcacctccctcataaactgtattgctaggtattttattcttctcacagcagttgtgaatgggattgtgttcctgatttggctctctgcttgactgttgttggtgtattgattttgtattctgagactttgccgaagctgtttatcaggttaaggagattttgggctgagactgtggtgttttctagatatagaatcatgttgattgcaaacagggatagtttgattcctctcttcctattgggatgccctttatttctttctcttgcctgattgccctggtcaaaacttctaatactattaatatgttaaataggagtgatgagagagggcatccttgtcctgtgctggttttcagcttttgcccattcagtatggtgttggctgtgggtttgtcataaatggctcttattagtttgaggattgtggatctaaccaaactaaagagcttctgcacagctgaagaaactatcaacagagtgaacagacaacctacagaatgggagaaaatttttgcaaactatgcatctgacaaaggtctaacacccagcatctataaggaacttaaacaaatttccaagaaaaaaaaaaaaaacattaaaaactgggcaaaggacataaacagacacttttcaaaagaaggcatacatgcggccagcaatcatggaaaaaagcttaacatcactgatcattagagaaatgagaatcaaagccacaatgagctaccccctcacacaagtcaaatggctattatttgacttgcgtgaatattatttgacttgtgtgaaatgagatacctcctcacacaataatggctattgttaaaaagtcgaaaaataacagatgctggcaagattgcagagaaaaaggaacgctcatacgctgttggtgggagtgtaaattagttccaccattgtggagaacaatgtggtgattcctcagagacctactcacaatagcaaagacatggaatcaacctgaatgtccatcagtggtagactggataaagaaaatgtggtttgtatacagcatggaatagtatgcagtcatgaaaaggaatgagatcgtgtcctttccagcaacacggatgcagctggaggccattaccctaagcagactaaggcaggagcagaaaaccaaacactgcatgttcttacttacaagtgggagctaaatgatgagaacacatggatacaaagaggggaacaacacacactggggcctatcagagggtggaggatggagggtaggaggagggagaggatcaggaaaaataactagtgggtgctgctaggcttaatactgggtgacaaaataacctgtacaacaaacccacataacatgcgtttacctatgtaacaaacctgcacaagtacccctgaacttaaaataaaagttaaattttgaaaaattttaaaaaggggacagagagagaagctgtggccccagctgggtggttttctcttttctggctgatgtctgcatggggagagtcccttccctgccccacttcattaccacttgccttttattttgaacttttatttaaaatactggttttggatttcaaccagaattttaagacttggggtctcatctattgcatctcatcgagtaagtccctgggaaccactgtcctttagcggcactgtccttcctaaaacaccggacattctaacacggaaaatgaaagcaaaccgcatgcatttcctaggactgcccataataaagtaccccaaactgggtggccgaaaacaacacacttattctctcccagttctggagactggaagtctgcaatccaggtgtcggcaggactgctgtctcttcgaagtccccctggggagaacccttctgagcccacctgaccgccccggctcttggtggcctctggcaagcatctctccatcatctctgcctccgttttcgtgtggccttctctgcctgtgtctctccaggactccagtcgtactggatcagggacccaccctactccagttgtcctcatctttttttttttttttttttttttggagacggagtctcgctctgtcacccaggctggagttcagtggcacaatctcggctcactgcaagctctgcctcccgggttcacaccattctcctgcctcagcctcctgagtagctgggactacaggcgcccgccaccacgcccggctaattttttgtatttttagtagagacagggtttcaccgtgttagccaggatggtctcgatctcctgacctcgtgatctgcccgcctcggcctcccaaagtgctgagattacaggtgtgagccactgagcccggcccagttgtcctcatcttaacccattacttctgcaaagaccctatttccaaacaagttctaaagtactgggagttggtacttcaccatatggtttttggaagggacacagctcaccctgggccgcggatgctggctcctgttagaggactgtgctttacaggcagcgcctgtggccctgtgagaactctcgctgcaatctttgctttctcacttttacagaccaacaagccctaccacagaccaggctgggccttggctctggccacttcgtgtcgcgtgcctgctgactggcgcttgtctcctgtgttgccagcctgcttgttagagctcagaggcagggaccttgcccccacatctcctgaatctcccacctcgggcagcctggcaaggccatagagatatgaacagcctgcccttccagctcctggggtacagcccgatgctgttgacattgcctccacctcccttccttgtctgggctggggagggatttcttggctcccataagcctttgtgggcttcctagtgtgtgtgttttgtcctttttcgtcctctgggctctgggctagacagggctgctctccatgaagtccccacccaaccttactcccaggagcagcagtgattgacagacagctgggcaccaccacctgccgctcacccacagatgcagccgctgcggatgctgcccccgccccacctagagagcataacttaacccatggtgacttcgttaacaagagatgcgtagcggggccccaggaaggtggccctacacccacggtagctcttggttgggtgcctatgtttcaggctgtgctgatgagcctggaggtgcctgggctggcccatccatcggagaccccggacagcctggctacccaggaaggggagctgtcacttcccagcgctgagtccagggctgtccacgtgggccttcagcaaagcctgactgagcctgccaggtcccaggctctgctcccaggtgggtgggcatggggggcagtggtggtgtgatgggggcgcagcaggagtggggcaggtgaagctggtcctgccaggtgctgccgcccttccaggtggtacttccgataggtggtgttcggctggtgacccctgcctggaggtcgaggggaagcacagcagagccagggcagggagaagcgtgtgctgcgcgcagcccaggaaggaggggcatggagcagaaagaaacacaggtgaactggaagagcagagagggttcatgcttcctgcacacatgggaccagggcatgggcctggtggggggtcagcattcctccaagccaaaaaacacaggtgtgacagccctttttctaaaacattacagaggcagcaggagcagagatggctgcagggaagtgactgttccccaaaagcaagaaactcctgttgcctcacttgtataagccaagggaacccctcgctttctgtctctggagtcactgaatatttttgtttatgtcactttatagcagtactgatgcttaggttgatctaagttcgtttttaaaacacaaaacacgtctttaagtggaatgcacagttagtatgctcctgcgaagcggcagccccgctgccctccctggctctgcggccacctgctgaggatggttcttctaggatgagtcaggagagtggagcaaggctcagcctgcaggtccttgctgagctcagggaagcagccccgtgaggatcgggctgcgtggtctgcaggggaacctgcctgggaccctgtgagatttacctgtttcaggtgccagtgatctgctgggattctggagaggtccctcaggggagcctggcacttcctacccccttgagtttgcaaagtcacaactgagggcctgggttgggacggtgtcatgagctctccataagctttgcttatggtacaggagcagactgtctggcgcttctggagacctttaacgtgctcctgtagacagtcgggtaaactcattctggtagcacagcgtggcctccccctccacctgacatctgaggaagtggcaggctctgcatctcggtggcctcgccccagggtgcagggagcaactttgaggaaattagtgctgagcctccagtaggaaggaggactgtgctggggaggcaggtgagccccttgtggtgccgcccgcaggtcacactggcctggggcccctgcagaggaaacaggatccaccagccaggcccgctgaccatttggaagtggagcgtgcacacaggcggctgtgcggtcatcgtctggcaaacaaatgatgggaccggaccaggagcagactggctccctgtggtggggactcttggcaggcggatcggcttcttaggaattaattctcaggaagccagaaaagggggaccctgtggcaccctctgcttttgctttggtttaacatgtttcctaggaggaataattaatcttctttctctgaggcaggctgtgtgtgtgtgtgcatgggttgtatgtgtacagtgtgtgcgtggtatgtgcatttatgtgttgtacatgtgtgtatgtgtcatgtgcattgtgcatttgtgtatacatgtgtgtggtgtgtgccttgtgtttgtttttcatgtgtatgtgtgcagtgtgtgcatttatgtgttgtatacacacatgtgtgggttgtgtatgtgttgtgtgtctgcacgcacagatgttgtgtacatgtgtagatagcatctcagcctccttgggaggaagccttgagtctttttggctctgctgagtcatagctggtgggcttgctctgcagtgctgactcaccacacagaaaaacaagaccaaaaagtcccctcagtctattaaaaaaaaaaaaggatgtaagataaatatttggctattcaagataaggccactgactcattcagggaggttaggatgactgcgtgtcctcccaggaatgcagacttggagataattggtttgcagacactgtatctggaagcagaaggtcagcagaaagcctttcagatagaaggtatatgtacccctgtgagttttcctgcctcgtcgctctctttttatggccattaggtttctgttcccactgctctggaagcgtcactcagggtcaccctctggctgctcagtctggggtctaggcactggcctggcctcatcacagcactggcacgggcctccgacttttcttcccgtggccccactgctcttcccggtctccctcccacctcttgtctgttccttcttgaatccctccttcttcccacgacccaagctctctccgacgccagcaggtgcccgcatgcatagtcatagcgtcagggcctctggggaagagaaaacagcttcgttttttgagtggctactctgtgctgggactatgcccccctcatttacccctcacagctcatcccttctccgtgcacaaggtgacatgaacttcatgtagagagagggaaactgaggctgaagatggctaagtaatgtgcccagggccaccctgctgggcaaatgggggtttgcaaagtggcagtcggacccagcttgtgctaataccagagcccactttctctcctgcttccactctctgtcctgcctccccaccccaagagccctctgtcaaaatgacatctgtgaggtgagacatcacttcttccactggtattaaaagttgttcgcccagaatggaaatgtatcacttgcatttgatggtcaagttatgcctactgccttgggaacaaagaagacagtcttgatgataggacggggtgcagatgtcacctacttcccctgtggaggacggggtggagaactttggagctcctttcttcttgctccagtgtgggcagggaggggcatacagagcactgacagccctcagtgttttcagaagctggttctgggcaagtctcagcttgggaggagcctgatgtgatgctggaagtggcattgttagcacttggggcagtcagagtgaagggagacattcctcagcctaaatagatcgtgggcattggtttttgttgttgtttggtgttttttggatttcactctgttgccccagctggagtgtcgtggtgcaatcatagctcactgcagcctcatcctcctgggctcaggtgatcttcttgcctcagcctcccgaatagctgggactacaggcatacgccaccatacccggctaactttttaaaaattgttttggtagagatgggggtctcactttattacccaggctggtctcgaacttttgggctcaagcccgcctcccaccttggcctcccaaagtggtgggaccgtaggcgtgagtcactgcgcccagcctggatattgttaaacattgtctttatatacaaatgttggtttcttttccaggaacctgggcctatgcagggcacacacgccggtcagttgtatgtacctaatttcacggtagccaactatgtctgtagccgttcaagcaacctgctattttattctgttttgtcagtagaagaatggagttcaacagctttgctggtactcaatacctggaaacagacagcatgttcttaacacatgcttttttcctttggagattaagggatagtattccagtttgacaatgtagataccaggctcctgtaattcctgtggcccagggaaacagtggctctaattttcaggtcctccactgcagaatgactcattcctgtgtgatctgagcgtgatctcgcctgcatgggaagctgccctgtaagggagcaggccggtgaggaacggaaaggaccacagcagcgcccaggccgccctgccaggccaggctagagttgcgcagataaaggccccgcatctgataagtgtcgccgcctgcccctgctggcgctcaggatcgtacttcttgtccttttaaaactcccttgcactccagcgtcattggaggcctgcaggcacactctgaaatagctgtacgtgtgggtggggaggagatagatgtcttaaagtgtacggatcatttgtttctctgcaggtgctgtcacactggaagcttgtggaaaccgtgttgtgtgtgttaactgagtggtgtgtttaagaacacagtggggcagggccaggaccaagtacaagatcaggttgagtcttgcattgtcaagtacgagactggctagcacagtcagtgaggtcttttggctatcactggtgcattttatttcattcagtccttttctcatagctctcttctgcttagctatttgccaaggcaaaaatagagggctctaaggaaggttacattccagagcacactgacttctattcatacagggggtggcggaaggctgttcctgggattttcagggtgacgataaggttgtgccctagaagtgtgaattcccggagaccctgggaggccccaacccccagcagaccccctcctcagaggctggtcgacagccagcatgcccgcccagtacctggcgagaggtcagggcgcattgccggatgggggagcagaggacgccggagtccaccgtccgcccttcattctggctggtgtcgggtcacaggtggccggatcccgttacccagcccaggacgttcggtttcattgaagaatacctttccctttctgtttccttgtgccgcagatgatttacatggacgatggagtgtcatcttttgtccagatcagaggctccgttccgctgttctgggaacagccagggcttcaggtaggtcatgaaaaaacttaaatgtccccttgtgatgttgtccgccctgcccttcccttcaatagctggggaagatttcttttaacacgttcgtttcatggcatagtttccagtctttttcctcagccctgttgtgttgagctgagctatcaaagtgtgcacacgtgtgtgcatatgcatgcgtgcgtgtgtgtagaaaacacagacaccactttgttgtttctggtcgtcaaggatgctgtgtgaaaccgctgactcgggaacggtgctgcggtgcctgctgagtgggtgccgcgaagtgagcttgggtagcactgcctgaggttagaccctgagttcggtgtactgaggacgtttctgagagccgtgactctcatggagggcttcaggaaggctctgtggctgcgtaggtctggcacccggtgctgcctgttgtggggccacgtgagacttccacagctttcccgtgatttctaaaaataccccttgccgtccaaagcagggctctggtcagtgccagagagatttggcattctttctcctgggaagagcacagggtggggacacattccaagaacacatttgtcatcaatggaatgacacgctgcctccttcaccagggtctctaacaaggtgggttatgggccgagtggactggaggggcctccccactaatccccagtcttcctccctgtcccgaggggacgtgggtgccttcagaaagcctctcggccagggcctttgtcgcctctcggctcttcttaccgacagctctcgtgtgactgcatttgggttgccagccagtggctactggggcccagagtgccatctgccaggttccagggggtgtttgacaacgctggactcagggtccccagtcctgggttcagcttcttagcctgctctttgacttgaccctgtgccaggagcaggtgagtcacttgtccccatggctgtcagcacgctgcagtgacagcattggtgaagtactgggtcagcctgggcagtgagatggatgaatggaggtctcgtttaggctccacgctctggtagggagaaaagtcttgattaacattcagtctttccactagaaagagcgcaccacactgcacgagataactgacggcctccggagaaacgctcctgaggacagctgtgacacagaaactcctccggagggctgcccaaaggaggtttttggaaaaatagctttattgcaatataacttacacacccatttaaagcctacaacttggtgcttttactgcatttatggtgttgtgtaaccaccactgtaatctaatcttagaacactttgatcaccactaacaaactttgtccccgttagtggtctctcccatcccctccaagtctccctcatgccaactcgactgttcatctactgtctttatctatagatttcccattccagacgttccaaaagagtttttgaaacctgattttctttaaatatggagttttcctttggaaacttctcgagctctgtattcaggagaatgaaagaattgatcgaatgagtgtggcacaaaaaggagacagggagaccatgttgtgtttcatgtaaagtttgtaagaaatccaaaaattaggggaatggtcatgatggattcttagaatttgggaaggaccctttggaattcatccattccaccctctgactctcagttcaggggtcctccttttttttttttttttttttttttttgagatggagtctcactctgttgcccaggctggagtgcagtggtgcaatctcggctcactgcaacatctgccccccgagttcaagccgttcccctgcctcagcctccagagtagctgggactacaggtgcccactaccacacccagctaattttgttgtatttttagtagagatggagtttcaccatgttagccaggctggtcttgaactcctgacttcaggtgatccacccgcctcagcctcccaaagtgctgggattacaggcgtgagccaccgcacctagccaggccagttcagggatcctcttccaggacctccaaagggctgtcctccaggccacaactggggatggttccgactcttggtgaattcctcctgtattgcactgctgcgtctgtctgactctagagtgttttggggtgcgcgggaatatgtctgattcttctccgtgtgactcaccctcagtccttggagggagtcttctggttccctcttcttcttcttgtcttctctaaggcctcccaactcctttggccaccttccctcctgtttctaagactctcaatgttctcatctccctcatctaaaaaatgacactcctttttgtcgttcccctcttggatttcattttggtcttaccttgaccttcgtgctgcctcagatgtagcctgacccgccatgggatgactgtgccttggtctgtcaggggccaggagcagtggaccacgaggtctcagctttttggcagctgtttcaccctcttaaattcctaacatttgtgctgcaccaaagcctccatggtttcttctttttgtttcttgttttgttttgtttttgagacgaagtcttgctctgtcacccaggctggagtgcaatggcacgacctcggctcactgcaacctccacttcctgggttcaagagattctcctgcctcagcctcacaagtagctggggttacaggcacccaccaccatgcctggctaattttttttgtatttttagtagagacagggtttcatcacgttgaccaggctggttttgaacttctgacctcaagtgatctgcccgcttcggcctcccaaagtgctaggattacaggcgtgagccatcgcgcccggcctccatggtttcttcaaaacaagcttcagaaggctttagtgttccttaatcttattaaggatttgctaacctttgggcctaatacctgtgtttagcctctaaacaccactcatcatcaaagagagatgaagtcatcttcacagggggccgggagggcagcgggaagtggtggtctaccgtcaccggcttcaactgtcactatcaagcctaaggctgcttctagttatttttctccactttgaactttttctcttccttcttcacttaggttagatgaaaagtgcaaagattttggggtgagtagcttgcgtattaagctgcaatgctgtccctccttgacagctgccgttggaaacccaggagcccagcaggatggggcagggttgagcagagggtggtggatgaggatttgtctgcatgttttcctgggtaatggtaccttcagtacttcctgactccatttctagcagccgtgtacctttaataaatttcatttttacgtgtaaatgggctgtgcagtttataaatagccacattgaaattttacccttaaagcaaattgctccctggtctagctagttcgagaatctatttatgatctgtgtctgtggttgacaacgttgctcccctggtcattgtagaagcttccccccaccatatgctgtatagacacgtgccttgccaaggtgagttcagtctgtcttttgaagtttcctgcaactgaaccaaacggaggaggatgccgagtagagcagacccctcttgccctgccttcacctctttcctgccagaaaccattcgccaagtttgtggagtcgtggcgctggatgggcttgtgtgtttgtaccttccttttttccaaaaaggacatgagatggcttgtagtataacagtttcaaaaataagtgaagaggtctggatgaagggcaagagtcgaattggtgcccacgatgcatgccgtgggtcttgtgtgcgtgggaaggaacctgacgcttgttccaagcttccccgcagccaccacagaaagaaccacagtcacagccagtttatgacagtgactcagagcacgagctttgaagcgacgcagacctgggctcaaattcaaactctggctcccctgctttgtaaccatgtgtgtttgagaggctcttatctgtgccaagccttagtttacttttctgtaaaatggggctaacaatatctaccttatagggttctagagaggattaaagagcgtaatataagaatttggctgggcatgggggctcatgcctgtaattccagcaatttgggaggccaaggtggacagatcacttgaggtcaggagtttaagacaagcctggccaacatggtgaaatcccatccccactaaaaatgcaaaaattagctaggcatggtgtcaggttcctgtaatcccagttattcaggaggctgaggcaggagaatcacttgaacctgggaggcggagtttgcagtgagccaagattgtgccaccacactctagcctggtcaacagagtgcgactctgtcaaaaaaaaaaaaaaaaaaaaaaaaaaaaaaacacacagtatctagaatctaaaaagtactcaaaatggcagctgctattgatactaatagaaacaaatgataacacccaggagataaaaccaatggtggcatccggagaagcatggctcttggtgccgtgagcactgaatgagagtttcctcctgtctttgtgggaacagggacaccgtgagatgtagtaacatgaagacagcttcctggtcagtcttatctctcatccctcatagactcatcgtaaatcagtaaccccagatgaagccatttattcatcccaaacattcactgagcgtctgccaggtgtagggggcaccagggatcagatacagtccctaccttcagggagcagggggcaggacagggcagtgttgtgtggacagagccgtgttggaggaagggagaggccccgggggcagctctcccaggctgagggtgtggggtgggctgcccaagggtccagactggggaggggtgaggaagaagcactcaggcaaggaaggcagcccttgcagagggaacaatgggtgcaggtgttgtaacccttgggttcacagtgatcagggagcatcactgggtagcttcttgaagtcctgggcatgaagggaatgggaggagagataggagaggtgagcagggctgggtcaagggcctgtggatgctacactgaggagtttgcatcccataccagcagctactcaagcagtactccccaaccttgctgcacagaagactctgctggggagcttgaaaaatcctgatacccaggttgataccatacacattaagtcacaatatctggggccgggagccaggcagctgtcctttttgaagacccatgggtgactccagcatgcagaaaagtatgggaaccactcggtgctgtgctggcaagccagttttattggggtcagactgggtgcagggagctttggtttgtagcatttgtccattgctgtggtgtaaattctcccatcgtggccactttcaacctacggctccacatcactgagctcagagttgggaccagctgggtgcaccgtcggcttgcaagcctgcaggagctggctccagcacacactggacactgctaaagggctgcaagaggtagagacacaattagacagaagtctaggcagtgttagtttggaggggaaagacaccagatgggaggccgttgagctgagatttttgggtaaccttaagtctggtaggagtggtggggtgcaggggagaggtgtggaggtgctgtctgcgatccttgggcctcttgagtgctgatcgagactctgctgtgacttccacagccaggagttctccagaccccgactatcctcatggctgtctacctaacctcagtctctggtctataggattgaactaatgactttaggggccagataagagtagctggccctttgcccagaggggaggaggacggggtggaggagaagggaagctacaaaatgcagtgacagtggtggaccctgccctcagctgcagctcgtgcctgtggcttctgacacccagtgacctcaggactaggtgacaccagagaaggtggatgctggtcagtgttggagcaggacagaggggaggaaggatatgaggcccaaggaacccagacaaaagggatgcacggggtgacggaggaagggtgagctccggagagaacatgaggcccagctcccctccacttttgcccaaggcctcactgtgttgcctttccacctgcctaaaagaagcagcttagggtcatgactcagggagaggcggttagacaagtccaataatacaacttgtttaggggatggagggacccagcctgaggccctgagcccagggaagcagctgtggcaagctgagcctggtcatggtgtccctgaggctctgcaggtatggacatgcctctgctggtatgcatgcagctctgcaggtggggacacggctctgcaggtggggacatggctctgcaggtatgcacgtggctctgcaggtggggacacggctctgcaggtgtcgacatgcctctgctggtatgcacgcagctctgcaggtggggacatggctctgcaggtatgcctgcggctctgcaggtatgcatgtggctctgcaggtatgcatgcagctctgcaggtggggacatgcctctaccagtatgcacgtggctctgcaggtgtgcacccggctctgcaggtatggacacagctctgcaggtgtggacagggctctgcaggtgtgcatgcagctctacaggtgtggatgcacctctgcaggtatgcacatggcccctggagctgtcttccctgctgtaccttctaccccttcatgggcatttgtaacctaagccaaaagggaacataacaagtccagaaataagtcaaaaccagaaccactcaaacttcagcgtgtgtgcataggaatctcctggagggcttgttaaaacacagattcctggccccacccctggagagtctggctcagcagatctggggtaggcccaggacttcgcatttctaacaagcacccagaggaggtccaacgaccacactttgagaatcactggtctagacttaacagatcccttctgcccattccctgctctggtcattcacccgggggtggtgctcccgactgacttcttagttctctgaggtagaaaggtttgttgtcagcagtgatactggactagccactgcaggggatattgaccatgcctgtgacagttgggcccagccctgtccacacggaatgcttgtctaagtccaggataaaagacaagcctgaggcctcagaaaagatgtgctgcagcccctgtccgggctgtgccttctgctgtgggcagctgattccagggcatgccgtctgcttgccacattccctgagtcctcactgatgtggttgaggggaccccatcaaaggcaggtgcctcatttcactctgctttgctgcctgttccctggactcaagggcatagtttctgtcaaattaatttgcgcttgtgcaggtttgttagaaactttgaagtgcttctgggtctttccatgggggttctggttaggtgtcatcacccaggccaggggattcagcgtctcccatccctttcccacctttccatgacacccaatggcatgttgactcggagggcgctcagtaactgaggaatggactgctgactgatagtgcagtttatatgttaacgggggagctatagagcctggggattggagcctgctaggtccagtcatctgtgcaacagtttctacctaattaaaagaagaggctttgggttatgactcagggagagacagttagacatgtccagtaatagaacttgtcacgacggagagccaggctcctcagccctggcctgccaagcttgtattctcttcttactgtgctcccatggggaatgatcctataaacagctgctgctcattgctgaagccaggggagacttcttgtgatgactgacagaccacacagagcaggtagcccctctcttgggttttagccctggtgacctctcatagggtggctgaggggtacacagacccgctgacttccctcagggtgcaggatggagcaggcctctcggtagtgcgaccatgaaagttttcaaattttctgagatttcctcagccaggtgttgatggccatggggataaaggacagaggtaacacaggcagcaaaaaaataaaaaaaaaccccacaggtttggggtgagcctttcttaggagctccattagccaacagccaagatacctcattttgcactcacttcacttgtgaccaaagtgtcttaggaccaaaagggatttgggactttgttgaactgtttatataaaaaaaacctggctgggcacaatggctcaagcctgtaatctcagtactttgagaggccgaggtgggtggatcacttgagcccgggagttcaagaccaacgtgggcagcatggcgaaaccccatctctacaaaaaatacaaaaaaaaaaaaaaatagccaggtgttgtagtgtacacttgtagtcccggcaacttgggaggctgaggtgggaggatcatttgaacctgggaggttgaggctgcagtgagccgtgattgcacctctgcactccagcctgggcaacagagtaagaccctgtctcaaagaaaaaaaaaagaagaagaagaataagaagaaaagaaaaggaaaccttctccaaggaatctctaaatctattgctgagaaaccagttctctagatgggtgcttttcttgagtctgagatggggcagttgtatcacctgcagtgttgagttatacaatgatacatgaagcatgataatagctgcatctcataacatagataaatctgctgacactacttttacaacactcaaacagaagcaaaactaaagagtgcatttttaactacttattcttgaaataatttcagacctacagaaaaaatttaagaacaagagggaactcccatatgtccttcacccagattcactaaattttaaccttttgccacacacgttattattttttgaaccgtttgaaagtaggttgcatatatcacattggtgttagtccatttgccttgctgtaaaggaatacctgaggctgggtaatttataaaggaaagaggtttatttggctcatggtaatgcaggctatacaggaagcatggtgccagcatctgcttctggggagggtctcaggaagcttacagtcatggtggaagaggaaggggagccagtgtgtcatatgacgagggagggagcaaggagagggaggttgcaggttcctttaaacaactagctctcctgtgaaccagcaagaactcattcattaccatggggttgtcaccaagctattaatgagggatctgtccatatgatccaaacacctcccaccaggccccacctccaacactggaggtcacgttttaacgtgagatttggaagggacacacatccaaaccatgtcagtgtctctttacttaagtgcaaaatatactctcctacatatagtataggtacacatttgggaaatttaacattgacaaagtacttcaatctcatctgcaggcaatattccaaattgatcacttgtctcagtgatgtcctttgaagcaccttttcccgccttgagtccagcatcacatactgcattttgtggtcatgtctctagtctttaatctgcatctgttcctcggctttctgtctttcaaaacattgatgtttttggagaatgcaagccttcttttccgttaagtcgagggtccctacatttacattttcctagtgtttcattataatcacattcagtgtatggatccctgaccagaatgctacctacatgatgctgtatccttctcagggtaccagatctggaggcacacggtgtccatctgccactcattagtgatgttaattttggtcacccagtcaaggtgctattcagtttctccacttacggttaatatgttttcccttccaactaatcagcaatctatgggagacactttgagaccatgctggtatcctgccttttactaaactttcccttaagatttatcatcaacagataatttttgctgaacaattatttactatgacagttgtaaaatgatgatttttaaatgatttcctccaaatttattacttggtattctactatagagccctctcttgttccccattcacccacctacacacccacccacctgcacactgattcacccatctgtcacccattaacccatccattcatccactcattcacccacccacctgcttatccactcatcgtccacctatctacccacccatccacctatccacctacttgtctacccaccatccacccatccctctatccagccagtcatccatccatccatccagtcatccacccatccagccatccagccatcaatccacccagccatccacccatccatgcacccacccatccacccacccactcacccacttacctatccatccatctttcatctacccattcacccacccatccatccatcctcacattcacctgtccatccatccacccacctatccatctactgatgcatccactgatccatccatccacccacccattctcttatccacctttctatccatccactgatccatccatccacccacccatccatccatccccattcacccatccatctatccattcagtcgcccatctattcacccatccatccatccccccattcatccatccacccattatccatccattcagccatccacccatccattcacccattcatctatccatccactcacccacccacctacccatccacccatctatccatccactcagctatccatccatccatccatccatccatccactcagctattcatccatccacccacccatccatccactcagctattcattcatccatccatccacccacctacccatccacccagctattcatccatccatccatccatccatctattcacccatctagttatcatcagtatagattcatgaatttctattttattcatcaggttaaaatccattactgtcctcatacagtttgatgttcagattgtcccagatttggccagcaaaagcccctttaagctgactccagcaacaatatgtattttaaatgtattttctaaaactaagttgagtattaaataaatagttctattaataataaggcaaatttgatcattaggatgactcagattttagggggaaattagtgtccctgaaggcataaaggagactgagaagcagtttctgtctccacagttggcttcactgacctctgtgctatcagagagcttaggatctgacatctgcctcatcatcttaccctggatctgctttctgtggaagaattgcagcagacatggtcatgttgcagttaggactgggcagcagcaaagccagactcgaggcacctagacctgagtttggagcactgttagctgaatccagagagctcagccttcctgagccctgaagagaccacagaggtggcctagtgggtgccacatgcagcctggctgggagtaagaaggagaatgggaaaaaccatggcctccttggtttgcagagaaggaagaatcactgttgttacttctctttgctttaggttggctcccatcatctgagactccacagaggcctggaagccaatgcccctgctttcgacaggtagggattgtctgacaccatccaagcctgtcattgtcatccttagacatcgtcctcccatcagacacaagggagagggtgcgacgggaaagggaggaccctgaaccctgaatctaaagctgctttccctttggtcctttatgcaagaagggagatgacaaaatgggatttttactttgttctcaggtttcctgaaggtaaattaatgaatgttcaggtagcactttataattattttatcttaccagtttcttgcaatgaggcttcatctctatgccctggttaataagggatcatctaaattatagggtatttgaagctgtttgttttttgttttaaaatagtatagaataaataagtatagaaaagtataaagtgaaaaataaaagtaccctaactcccagttaccactctcagagattttactattagtagttttttacatgtcattctagaaaacaattttggcattttatgtgtgtgtgtgtgtgtgtgtgtgtgtgtgtgtatttaaatacacaaattggatcataacacatttactgttttgcagcttgcttaagaaaagcagagctatttgtaatatgtatttgaaataataacttcttaaaagatttgcttatttaatctaaatactcaatattttaaaattgtcttagtgaatatagtaaaaattctcgaaagtaatgtgttaagtgtttgagtccttctacctgaagctttattgagactttaagtttgctttcatactcttgttaaacataacacttgtcatatgagcccagatgtgaccccctgattagtgtgtgacatgtaatgcgcttttctttaaagagatatttaatcaagtatagacaagaccattgccttttccaaagcattattagtatgtcctgagcattctacatgcggagttctttcagttgacattcatctatactaaattgtcttaggaaatagtgaacatgaaaccaaaaatgaaattctacctggctgagcttggtggctcagattttagggagtcgcctgggtgacagagcgagactccatctcaaaacaaaacaaacaaaaaacaaaaatattctacccgaagctgacccaaaacaagagggaagggtcatgctgggttcgcttcaggaaccctcctatgtttaggagctttcttaaaaaatacatacatctagttgaagcacatttgcatagactgcagatttttcagaatcctggtaatgcatggtgtgtgagatgcacgtaccacagagaatgagacctctctctcctcacaagcaccccagaggctgccttgccccttggaaagcattcctaaatctaggggtacacttagatctcttctgcaaatcacaggacaagctgctgaaagggtgagtctttctacaggcagctactgtgtccaggaagtctcttctgtaatgcttcttacccacgacactgggctggaggcaggagccctcccgttcccttgcaggtgctgctgctctcatgttcattgctgactatgttcccctcattcgagataacgtttggaaataaggtgacatgtctgcctagtggccgcagtggggcaccatgagggtccccctgaaagcaggctgcccaggtcctcgatggtgtccctggactggcatgtgcattgtcctgggtgcttcccaattctcttctctgggacatcactgcgctctcaagagtcgtgcggttctctgatggatttggttgagagtaaatcctttctttcccccgcaaaaccttcacacagatagtttttcttcccagaggatgcttgttttcctttgcctgagctgcatgggtgcccctgttagccgaggcggtctcttgtcactttgctgtatgtgctccatatctgaggagggctgtttgcagcaggactttggctgcagctgcttgagtggggtcttgtcatctctgtgtggtgatgagtctcggtggaagggctaacctttcacagggcggtgcctcagtctctcctcacaggacagtgaaccccttctccttccccgccccgctctgcgggcggctgctgtgcacatcagggtgtgctgaactcttggtccctccctgaagatgagaaggatgcttccaaatagaaatctgattcctcagtatcccgaaaaactcctgttttccaagtagcttcctatgacagtaacccgtaggcaggggaagtgaaaaaaaggcctcaatgctcagggtgacccaggaagaagcccgtatgatgccgaccttgctgctggtgccagcgtccccttacaccctgggtgccagcgctcccttacgccctgggtgccagcgtccccttacctgggtcggcagggcctctcagctcaccaataacgagcattttacaataaagggaaagaaagaggaaccacgtcccacacaccaagcactgtccttgtcactttcactcgtctttttagtcctcctcgccattctccgcaggaggggaagtagccctgctttatagatgaaggagccagctcagaagggatgaatcatttgatcaggggagtttggggttgggacagcatagagtaatgatgaggaatagtgagtaataatgagtaataacagtttccttctggccctgggctttggaaccagttgagaagaaggggcaggaatgagacaggaagagatcagaaatgggccttgtttccgatgagccacattgtgggatgtggcttggatgtgtggccacggtgggctccctcaccaggggcagagcctgcacgtgtcagagccaggcctgggatgcaccttctctgggcagaccgacccaaaagaaagcccacaagggctgaagccacacacagcccgccccagggctgcctggagctgcacgtggagacgggagcccgggagggtgctcacccttaacttaggccagggagtcagcaggcaagcctaacttcaaactacccacttcaaaatccatagtctttctactactgtcccttaaagttagtaagagaatgttttctcttactgcataagaaaaaaaatgatttttttcatgatcattattagctctttaaaaaattccgctcataaacgtgtcattacagaaatttggaaaagagaaagatgtttaaagaagaaaagaacttgaatcatataaaattctaccacttttgacatcatgtgtttttttccagtgttttttctatatttaggcataaagattgcaccattaaacgataatttgacatctgctttttgcattgtatcatttccctatgttttgtttaaggcactacatttcaacatgtttaaatttgaaatgtaaggttacttttaaaaagctggtgatcaagaataaatattggaatctctccctaatatttcaacatacatatttagaaagtagcttccaatacaaggaatgttttccaaagcctgcaagttttgttgtatctccttgttgtggtttagcataatgcaatatagcacttagattgaaaacaggatgctgagttgagagctctcctgccagttgttctagttttgagaagggtgggtaccaagcaaaataagagattcatttcacacatgtgcacattgcatgtgtaagactctagcccaggcacggtggcttatgcctgtaatcccaacactttgggacgctgaggtgggaagattgcttgaagccaggagttcgagaccagcctggacaacatagcaagaccctgtctctatgaaaaataaataagtaaaaattagccagatgtggtggcatgtacctgtagtccctattacttgagaggctgagttgggaggattgcttgagcccaggagtttgaggcagcagtgagctatgatcgtgccactgcactctagcctggacaacagactgagatcctgtctcaaaaacaaaattctcttgatgaaagttcaggttaggtgttctagatataatgggaggtgctttatagaaagtgcagcatatcctttgtctgattccaaacaaatgtagaagtctacgctagctagtagtaatggcagctgggaggtgcaggttctctccacccaggctcattttccacgatttgggtcatgggggacagctggtcctggactgtggcactaagaaagcatgactcctgccccgtcttcccttgaggggcagaacagggcagagtctgcacctgtgcccgtgcccagcatcacgcccaccccgctgcctttgcaggcacatggtgcttctgaaggagcagtacgggcagcaggtggtcgtgaaccttctgggaagcagaggcggagaggaggtgctcaacagagccttcaaggtaaggccaggctgtcctctccacagctgggctgggcggcaggtggccatggtggagcgttgagcctggaggtggctcctgcagggactggagctgacagcccagccctccttcgtttcctgaggcttccccagcattccgaccagtgaacacggcagtgctcagtgactcgggccctgatacagtgagtgtaatttcttttctttttttttttttttaagacagagtttggctcttgtcgctcaggctggagtgcagtggtgcaatctcagctcactgcaacctctgcctcccaggtacaagcgattctcctccctcagcctcccaagtagctgggattacaggcacttgccaccacgcctggctaatttttgtatttttagtagagacggggtttcaccatgttgttcaggttggtcaagaactcctgacctcaggtgatccacccgcctcggcctcccaaagtgctgggattgcaggcgtgagccaccgcgcccggcctagtgagtgtaatttctgtgagctgatggttgggagaatttgctcaaagtcaaaacagagcatctcctgtccccagccatgtgtcccttaccctccctccccatggggctttgagggcttctgacctctcaaggtacccctctcagtccagcccaggagcaggcaggcctccctttctgccctggctggtcctgggtgagttcccccagccccacagggcacccggcctcagggctcaggagcaggaacttcggtggctttcccctgcttgccggctggccttcaaatttccccctcttcctcgggctttcatggcagctcctggacctgctgctgctgggctcctgtgggctccttcagacttccttggggggcttttcctggcgatcctggacagatcctgggcagggtcacagagccatgggagcaagagactccgcccacctggccaggttccatccctgtgcatggatcccaggcctccagcccccgccccctgctccccaggcgcaagacgttccagaatgagccaagggggctcctggttcctttaccagcctccatgtcccctgcctaatagagaaaaactctctcgttgtttaaagctcaaagatatcccttacagagagagctcgaagtcgcctcactgggggttagggcgatctctgagcctttccctctgcttagaaatgccacatgctcatgctcagagcaggcacttcctggttgccaattttatttggccagacctgcttttcctgtttggtttgttgctattagcaacgagctccagaagtgtctcccttcccacccatcttggcagtgatgctggctgctaatttgaaggaagaagggaaaaagctaaaaaataagaataataataagaaccccaagtccttttcccctatgccttgagttgtggcccctcccatcagtgggtgaagtgcccggcactggtgagggcgggagatggcagccacctcatgcggagaagggtgggctcctggccaggtagggaggggcactctagctgaagacctgggtcatggccctgcctggggtggccctcagccacctcagcttggcccagaccaaggttggtggcccagcagcctgcccagggcccaatggccatcagtcctgctggatgaagcactggctgtggaatgcatggcactgagccaccaggcactgttgggctcctccagcttcttgctcacagcaagacttgcttgatgatggcagcactgcattgatttacactcagacctatcacaggcagtcacgttgagtcagatcagatgacccccaagtccttctagtccttccaagtcactaggacctctggctgggcagaggctggactggcctgacctgaagccacaagggcatgagtgcagggtgagaggcctggaagccgggtggcctgggtaggcggacagtttgtccttcatcacggggctctctggggagaaagccagggagctgccaggccagggaagcctcatatggaagggagggctcttaagggcatcctggtgccatcagcagccccttgacctgcttcccatctctccccaccttcactctggctccctgaccatcctgaagtagcatcgggatgtgggacccacctgggccacttcagctggagacttggtctcttcagagcataaccaccagaggctggtgcgcggcaggatggggccggttactagggcaacggggagctggattatgagctctgcagcctgccacagcactgctatctctgagggcctggggcacctgggccacctgaagaacagagcagtccttcctcacctgtgccctccagagagccctgcaggagtatgatctctccaggaagccacacctgctctggcttcttttgctcaagggggaccttctgccctttcgcaggcaagacgctctttgtgggttttgtccccccagggcctttgtcctgtgcttcccttttggggtcaactgcctggtccactgtgagcttccagctaaagcacgtcctgcggcgagtcaccttggttagccgcacgggtcaagctctgcaaggagcttgccctcctcgtcctcccttccctctgctcccctcaccgccctcccctcctcttttcagaagctgctctgggcttcttgccacgcgggcgacacgcctatgatcaattttgacttccatcagtttgccaaaggtgggaagctagagaaattggagaccctcttgaggccacagttaaagctgcactgggaagacttcgatgtgttcacaaagggggagaacgtcagtccacggtgaggctcgctgcgcactgtgccgcgtcttctgctggggggaagcgtcagtccacggtgaggctcgctgcgtgctgtgccttctgctgggtgaggcggcagaggggccgtgcagtctaggacatgtgccctatgaggggcagcttccttccctccccactcagttacccttgtggtttcctgggtcataagcttggcgacgccgggaatcttgggttccagcttctcatctcccttcccacagtaggagacaggcttttgaatttctcaaggagatgttgagtcatggttttctggcaaaagaaatgaactaataaaggggactgcattaaaacatggagctccacgctgtgacgcaggcctgtgagagtgagggccacagtcgcggtgggatggctggcatagacttagattgctagaggagaccagggtatggacttccctgttcagagcagaggagaaaaacgaagtccagttccctcaggtcacatgcagggggtcctcattatttgcagattccatattggcttatttacctattccttaaaactgatttgtaaccccaaaatcagtatgcaaagcatttttgctggacagagtatttaaatactctggcctcgtatttaaacaagtttcctttttaagatttagtaccatgtttttcacatttgtggggtctttgttaggggcccccctttgttcggggatctcatggtttagaatggccccaagcagtgtgccgaagagctgtctagtacttctgagcacaagaaggtggtgatgtgctttatggagaaaatgcacgttaagataagctttgtccctatatgagctacagtgctgtcggccatggttcagtgttaattaagcaaatatatatattcaataaggtgtctttatatggaaacatacataaaacaaggttgtatattgatcagttgataaaaatgtgaccagaggtttgcaggaacctaaccctatatttcccctaggagcgatggttctgtgttcatgtccaattcagtgttcctacaactttatagaacataattgctataaataacaaggaccagctaaacgtaaaacgacccagctgtgcaggctactgagtgtgtgtcctggggtctgcctgcctttccagaagtgctggggatccactgggatggacactggggtttctggggggctattgatgaggaggtgaggggttccctcttagaacatgtttcctcacacctgtaatcccagcactttggaaggctgaggtgggcggatcacaaggtcaggagttcgagactagcctggccaatgtggtgaaacaccgtctgtactaaaaatacaaaaattagccgggtgtgatggcacgcacctgtagtcccagctacttggaaggctgagacaggagaatcgcttgaacctgggaggcggaggttgcagtgagccgagatcatgccattgcactccagcttgggtgacagaggtgagactctgtctcaaaaaaaaaaaaaaaaaaaaaaaaaaaaccatgtttccttgggagtcaaaagtcagacatgggcctctgtgctatggccactgcttctttgaaaacaacatataaccgtttacatttcttcttgtcctaagttttcagaaaggcactttgcggatgaactgtcttgactgcctggaccgaaccaacactgtgcagagcttcatcgcgctcgaggtgcgtccctgccacagccttttcggccatctgtgccaggtcctgtgacttcagctggacaggctgggctgagcacctttagcggcaagatgagggtggcatcaccaagacaaccttctgactatggggaaggtggacagggagacactgggacatccagagaggggttccccaggaggctgctttctctgtcgtaggcacactttctcacaccctcttgtgaaatgcatcccagacatggtggcccaaaggctcctcgggaacagcggttgtgtgtaatgattatcgtggatgcaccattggccaaaggcagtgtgaggctggccggatgaggctggatttgaagctggggagggagacctctgtctgcatggccccagctgcctggttgacctctcctggctggggggtcgggggtagggtcagcaccccctgctgtccaccactgtttgctcttcagcctctggcattgggtggggcggggcggggtgcaggggcgttcaagtgtcagggggtgagaggagggcttgggtggaccctatagataaagttggggtgagatgtgggaagtcactctctgctgtgggaactcctcatcctctggaggggcacagagtcttccaggcacagcgtaatccacaggctgccgaataaggaacctcctgggacagtggctgcagggcctctgtgggagaagccagtgacagccatcccttattcctccccaggtcctgcatctgcagctcaagaccctggggctgagttcaaaacccatcgttgaccgctttgtggagtccttcaaagccatgtggtctctgaatggccacagcctgagcaaggtgttcacaggcagcagagccctggaagggaaggccaaggtagggccccgccgagggggcagggtgggggccccagggacccccctcacggcctgcggtctgggctctcggcaggtggggaagctgaaggatggagcccggtccatgtctcgaaccatccagtccaacttcttcgacggggtgaagcaggaggccatcaagctgctgctggttggggacgtctacggcgaggaggtggcagacaaagggggcatgctgctggacagcacggcgctcctgggtagggcctgccgcagacagggcgaggcagggaggtagggtgctccccagccccactttcccaccgcctctgtgctatccagagagcgggtggcagaggtgcctgggatctgagcagtggaccatgcaccttgcagctgtcactgcctctcccgggagcctgcgctttcctgctgggcctgggagctcacgcttcctctgtctcggcttggactgtgtttccatcctgagaagcgagagacaccaggcgggctcagggagaggccgtgcactctcctaaacgcatcggtgcctttgaatgctaacctactccagaaacttcctcagctgcctgggctcttgggcccttagcaggtgatgttggctttgggagcttcccatgaagttggcctccagtttctctcttggcccctgaggaccctgtggaaacagagaaggaggatgctgtcatcccctggggatgagggcaccttctggtctcagcggcaagacatcgtgattccatcaatataggaggttcctttgggagcattttaatggcatcttcttacctgagcctgtaaacatattaggtctcatggaaaagtaggggaaacccagcatttttccttataagaatgtacaagtgtcgctttagaaatgtcaggtgtttttgaaaaatgaactagcccatgcttaaaatgggttcacggccctttccactgtcaccgtcactgggggagctctaatttggcttccgataggcacggtgatatttagtttctactctggaaacagtgccagctctgtaaaaggtgaacaaagctccatgttgtacagtgagtcttatttaatagcaaggcagacatgagttagatttggtttacatcacaagtttaaaaggtggaatatattttggctatctagaggatggagatggttctgtgtagtgactttttaaagccttaagtctggggcaaaacaaaacatatcagacatgcaggctggaaggcagaggatgcaaaggtggctgtctgggttggtaagacacttaaagttatagtgtatatggatgcatacacaatgaaatatggtcagttacagcatgcccttgcttaaattcctatgccttgttcttctacggagtggacagtgaggacacggcttttccctgtcgtgtctgagatccctatggtctgatgtcccatggcttctggcttcagttgggggtaggggtatagacggtgagtttccagtcttatgttttcacgcctttattgtagaaatgccataagggaagccccgtggtgaccgtggttttaagccttttgtcgtcttcgtaaggagcataccctggtttatctctagaggctcatgaaccatctgtctctgccaggcagcctcatctgtctttttggaggatgcctgagctttggaactgcaaagaaatgtgcctttttatgcctcctgacagtgactcccaggatcctgaaagctatgactgagcgtcagtccgaattcacaaatttcaagcggatccggattgctatggggacctggaacgtgaacggaggaaagcagttccggagcaacgtgctcaggacggcggagctgacagactggctgctcgactcgccccagctctcgggagctaccgactcccagggtgagggcagtgactttgggggagacaaaatccaattgcacctaacctgacatgtcacgcttgaccctttagtggccatcgtcttgtggaatttcatctttggtgtcttccctcctcacaatgtctaaatagcaccatggcctgactctcaagaatgctgcctcgcaagtaactgacctttttcctttcccagatgtttagaagagttagcagatggtgaatctctcttttctctttgcatacagtgatggtgaagtcttaggctttcatcagtagaatcttttcgtcaccagtcggaaggtgtggcttctagggccatgtaactgtgaggttgggagccaagcctgtcttcggtgttttgtttttgttttttgttttgttttgttgttgtttgtttttttgagacaagtttcactcttgtcacccaggctagagtgtagtgacgcaatctcggctcactgcaacctccatctccaaggttcaagcagttatcctgcctcagcctctcgagtagctgggattacaggcgcccaccaccacgcccagctaatttttgtatttttagtagagatggggttttgccatgttggccaggctggtctcgaactcctgacctcaggggatccctctgccacagcctcccaaagtgctgggattacaggcatgcgccaccgcgcctggccctgccttcagtgttgatattgaccaagaacagctcatccctgcctctccccaaaccaactctattaatctgctttaagtagtcacaaaataattcatgtgctatttaggaataaaataattccaaatgttgacacctcaccttttatcttgtaagatacactattgaatggccccagaacttaggatctgaagaataaatgattcttgtccagtcatcgttgactcgggccttggtttttttgtttgtttgttttttgttttgcttccttggactcaggaacttgtggttcaggtttttgcctgttggtgagtgacctgtggtctagctgacatgcagtggcccatgtgtacattcatgccccacagctgcctgggtagtacccagagccccatcccttcctgtgcacacgccatctgtgccctcctttctgcctccaagggcaggtggcagagtccaccattcagagccactggggctctctgtgtcggtggtagcctgggttgctgcttgtggtgcctccagaagccattcaggatgccctccggagcaggcccatcaacgcggagagtgcttagcacagccgtggctcaagagccgtccctccagtccccacgaaagtgactcctcttcttttccctgcaggaaggtgtgcatgtttttagcaagctggctggtattttgcaggggatttttttggggtagacactagcaggctccccagcagtcccactagagatacaccaggttccagagaaggaaaggagtgctgtggttactattgccaagcagatcagacagagggacggggattgttttatttaaaaaaaaaaaaaaaaaaagctttaagtcagggaaactagaggttccaggttgcccattgtggtcaacagagaaggaagtggacagcctgcctggcctctcgctgacagaggccatgtcctcctagctggaccctggggaggtgggcctctcactttctgcagcagctggagccatgactgggcttggtggctcaggagcctgggtgtcagaggtcgtgactggacagattcagtcaatggctacaaggttatattaaattctgacctcccctgttcaaagggaacagaatggacggagggcccctctgggctcactgctgcatttaagccaggcatttctgaagcagattccactggggcagttcccagcagcaatggtagccacctggagttggactcaggcagtggacagcatgactcgggagagggcactggggagccccatgaactcgtaatgcgggacttggcggttctgacttctgtcatctcttgccttgctccccagatgacagcagcccagctgacatatttgctgtggggtttgaagagatggtggaattgagcgcagggaatattgtcaatgccaggtaaggggccaggtgtgcggggccaggcagggacttcctgagtcaggtgcctggctgggagcagagcctctgaggctctccgggagccagcctgcttctgagccagctaagctgtggccctggctgtgtggggatctctcacctggctccttctctatgctcacacatgcaccaccccgttctttccctgccacttaggaagtaaaaggaagggctgatgattgctgtgttttctttgcagtgctctggcctcggccacttgttgaggctaatttatgctgccagagggagctggcatgtgtggacctggggtgcaggggaagggcggggctgggactgggagagtagttgactcatgtttcctggggagaggtgagaaagtgtgagtgccgggaagaggtactttcttcctgcacaccagggcaaaggaaaaaatcaaaaccactggatttttatttgatatgaagtcaactgggcgctccccccaccccctgctgaactggcctggctttattttaaggaactttctgaacaaataaataagactgactctgtggggctgctatgagaaggtcctgggttgggtttcaggggtcaagggagggctgctgtgatgtgtgaggcatagccaaaaacacgcagatcctccagtagaagaaggaaagcatgacactaatattggggtgcgggcagcgtgaaatcagttctgcaaacagaataggcttgggctggagcatttggtaggtgggagatagatgtacttccagctacctgtaaacagcatcctttccttttctcttccagtactaccaacaagaagatgtggggtgaacagcttcagaaagccatctcacgctctcatagatacattctgttgacttcggcacagctggtgggcgtctgtctttatatctttgtacgtccataccatgtcccgttcatcaggtaagaacattctgtttacacacatctggggaacaaggggttgttagtctttgtgttttgttctttgacttccccctcctcccccatccccttctgtaacaggaatcgggacttaccattattttagataaatgtagcaaaaccccctaggaaaaactgggctgaatagaatattgtctagcgattaaaaagccaccatgggaaatgtctaactcaggaagctgaactcttagaaagggtgttacatgattgtcatcgcccaagctcaattccgtgcaaggaaaagggttccttgttggaatcttatagacaggaaacctctgaccaagagctgagtaactcactgggaaatgccaactcttttgtccctttttaaaagaattctaagtagaacgtgtgggcctctacaactgtgacctcattgtcatttctccaaggtgcatgctttgtgagcatcagaaagggggcgagcttaggggcggcattcctcttggggtgtgggtgtgggtgtttggatgctggaggaactcatcttttccccagcttgtggttggcctcatcttaccacctgggcctacccatggcttttgaatttccaccagcctttcggcgagctggcagcaggcgttgaactgacctccccactgagcccctgggtcccgggaagggcctgtgcctgccaagagcaccacgggtacacccctgcagcacctggagactaagactatgctgcccataatcctcttctctgactttcagaagatgtttaggtccctgtccctctgcttgtgttatttgggcagcttggcagtgtcctaggttagcaggtgaaggttagtaaaggttaaaggcaagggcggggtgagggtgagaggtaaagcatttgagaaagggctcagagctgaggagagccaggtttcccaggctcggtgtcctcggcttcacgtgcctttagccctgagctgcttccgcctacaccccttccatcagtcatccccccaaccttgtctgccagcgagcagatgcaccagcaggcctcagtctgtggcatggctgtggaaatgttaacaaatgtgcttgcagcccatgtttctatggactcgtatgcgtcataagtgggcatttgaatgcaagcaggctctggcattgcataaaagtccttttgagaccatctacccagcccagcataaacagctgcccgtttcctagctcttaaagtgcattgattgctgggcacggtgtctcacgcctgtaatcccagcactttgggaggccaaggcaggcagatcacttgaggtgaggagtttgagaccagcctggccaacatggtgaaaccctgcctctactaaaaatacaaaaattagccgggcgtgatggtgggtacctgtaatcccagctactcaggaggctgaggcaggagaattgcttgaacctgggaggccacagttgcagtgagctgagatcacgctgctgcactccagccagggtgacagagcgagactctgtctcaaaataacaataataatttaaaaatgtgttgattgcccagatgacacctcccatgcatggcgtgatggctgcacaggagtggggtgggctggtgggtggtgatgtttgcgccgctcctggtggcagtgagaacctgggcggatgcaggcattgccctgtcttcggcttcatggggtgctcaagtgagcacttcccagttggggtgtgggggagatgtccaggcagcggtgggcagcaggagggaggcggcctcctgaccaggagtcgatgacggccacggtggccactgtgttgagctgatgattttggagcactcagagcagcaggtcccgagctgctgctgggcccttctctgtggcaaagcagggtcacacttctccttcaggagccgacggcatgcgcgtatccttctgttccagggacgtagccatcgacacagtgaagacgggcatggggggcaaggcggggaacaagggcgccgtcggcatccgcttccagttccacagcaccagcttctgcttcatatgtagtcacctgacggccgggcagtcccaggtgaaggagcggaatgaagactacaaggagatcacccagaaactctgcttcccaatggtgagcggcgccgtggggacagccagcacctccctgctcagctcagtcccaaatgtgactgttgataggagccaggcactggggacacaaccacagggagggccccttcctggagggctcagcgctgggggagggggagagggctatgtccctccatggaattgacctggggcagggttagccacgtgcctggagggggccagctttgcagcatttcccgttgctgtacaattcctgagtgtctgcatggccactggatttgtatgtgtgcattttctgattttctaattcaagtgatggtttagggaagaaaaccatgtccgtggtctgtcctgtccaagtggtgaagagcatttgaatccacacagcctaagactccaccttcctgagattcacagtcttcacatgtactttctgtggtggctcagataagcacacccttttgaccaatggaccctttttgactgggaacacggttcttgaccaaggagcccagatcctgcctgtcctcgaagctgcaatagcgaaccgcacactgccctctcgtggcggtccggagggtttcattctgaaactggaggattttcttttcaaatctagacgttaatgttctccttcctgtgcaaagatgctacccgggcacagcttcagcgtgtggtgcacacgaggccacctctttcccgaagtgaggatagacttcagctgttcttgctgcaaaatcttccactgtctgagaggtggtgggttgggtcacagagctcttccctgtgagaacagatccttaaaactgacgtcccattttactgggctgcacgagcactcaaggcaaaatttccttagttttcattaagaagtgccataattcctgttggtgttgtttatttcttatgttaaaaaagaaacaaacgagttgggaaattttcaattgtgtgtaatgcagtttaaaaaaaaaaaaaaagtctaacccttctcacttaaaaatagctactttggggccaggcacagtggctcacacctgtaatcccagcactttgggaggccgaggcgggcagatcacctgaggtcaggagttcgagaccagcctggccaacgtggtgaaaccccacctctactgaaaatacaaaaaaaattagccaggcgtggtggcgtgcacctgtaatcccagctactcaggaggctgagacaggagaatcgcaacccaggaggcagaggttgcagtgagctgagatcgcaccactacactccagcctcggcaacagagcgagactctgtctaaaaaaaaaaaaaaaaaagtgctactttgacatagtaaacttgggatacataataagtaattaaaggatttataatgtagttcttttctaccataaaaatattgagggacctattaacattctacacttcttttttgtttttgttttttgagacagtctcactctgccacccaggctggagtgcagtgacatgatcttggctcactgcaaactccacctcccaggttcaagcaattctcctggctcagcctcctgagtaattgggattacaggcgtgcaccaccaggcccaactagtttttgtatttttagtagagatggggtttcgccatgttggcccagctggtctcgaactcctgacatcaagtgatccgcctgcctcagcctcccaaagtgctgggattacaggtgtgagccatggtgcctggccaacattctacactggtattgtctgataccgtagtcactggccataagtagctatttaaattcaaattaattaaaattaaataacatttaaaattccttgctttccctggccacagtgagaatgatcagtatccatgtggccagtggctcctgcatcagatggcacagagaacatttcggtgctgcagaaagttctgtctgacagtgttatctagacgctcattttatttggttgggacctgtttaatcttctgtccagattattgtcatggggggagtggaaatgcctcatattataggagaagagaagctgattttggagctaggcagatgggagctgaaatcccatctgtatcatgcactagctgtgtaaccttcagcaagtgcctttgtttctgaactttgtttttttttttttaatctaagagagagctggtggtatctgctttgtgcacctggaacagagcccagcccttgcggacagttgatccacagcagctgctgtcattgtcttccccattcctggggaagagacagggaaggcaaggaccccaggagctctccggggctgagctggggccgtgggctcctaggactgggcaggatgggaaggaggagctagagagagatggagaagggtcacctgaaaaagctcaggaagttacatgaaggtgcacatcctgagcatttctgctgctcttttgaaacctcattctcctaacgctgaatgctgggtgctgcttcctaggagtagacactccaaagtaagggtgcatctgatcttcaggacggggatccatggaggaaatgctggccatgttgctctcctccgtttagggtaggagctttcttcaaaccactcagaaacactattatttaaaattaaaaagtaagccatgtaaaaattcctccatagttttatgggatgagtgaaaaacaagatgctctagtagaaaactttcttttttctaagtagcattttgagaaaaatactcctgcttgccccagtgagcttgtttttctcctctctcccaccttccttgcaagatgggctgaatgattatgattttcttttcaacttaggggagaaatgttttttctcatgattatgtattttggtgtggcgatttcaactaccgcattgatcttacttatgaagaagtcttctattttgttaaacgccaagactggaagaaacttctggaatttgatcaactacagctacagaaatcaagtggaaaagtaagttgtgctttaaaaacattttttagcagctgctccaagatggaatgacatctgtgagatgtagaggctggtgcagcaaattcagttccgtgagtggatttcactgtttccaacattgtagaaaataggtgggcttgtgaggttacagtacaggagtggctgttcatacacacttcctgtccttttttttttttttttttttttcctgaggcagagtcttgttcagtcacccaggttggagtgcagtggcactatcttggctcactgcaacctccaccttcctgggttcaagcaattcttgtgcctcagcctcccgagtagctgcgattacaggcgtgtgccgccacacctggctaatttttgtatttttagtagagacgggttttcaccatgttggccaggctggtctcgaactcctggcctcaggtgatctgtgcgcctcagcctcccaaagtgctgggattacaggcatgagccaccatgcccggtcaaccttctcctccttctaatactgttattttagtgcctgtttgcttcctagcaggatgagctgagatgaggggagtacatttaaattccatttagctgtggagaaagaagctggttaaatgtgtgagggccaggtgcggtggctcttgcctgtaatcccaacactttgggaggccgaggcgtgcggatcacctgaggtcaggagttcaagaccagcctggccaacttggtgaaaccccatctctactaaaaatccaaaaattacctgggtatggtggcaggcgcctgtaatcccagctactcaggagactgaggcaggagaatcgcttgaacctgggaggcggaagttgcagtgagccgagatcgcaccactgcactccagcttgggcaacgagcaaaactccgtctcaaaaaaaaaaaaagtgtgaggacagggcagataaggtctctcctgaagcaagatttggacgttcttcgaggtttactgtgtcttcccacttgagccccttccagtgtgtggagtgctaaagggttgccacatggacgagtgaccataccaacctcctaaagcaggttttcctgtcttcctactaccaacattctcacatcctctcagtgatgcccaagagaatgagggtgtgtttgtcccttggtcttcccatggaagcaaggccaatccaaagttaaacaaagaaaagaaggatgtgcatgggggctcacgactgtaaggcgggaggattgcctaagcccaggagtttgagaccagcctgggaaacatagggagactttgtctctacaaaaactaaaattagccaggcatgatggtgcgtgcctgtagtcccagctacttgggaggctgaggcaggaggatcacatgagccccagaggttgaagctgtagggagccgtgattgcaccactgtattccagcctgagcaaaagagcgagaccccagggggagagagagaggagagctgactttcactctgcgcttctatgtacttagctcatttagtcaacaaacatagatgaaggcctcggggttgtgggccagacatggcaccaggcatgggactgtaaagatgagcacccctgacccgtgcctcggaccctgcactgtctcctggggggcaacaagaagacagtggatagatcagacactggagtgggtggggagatagatacgagggcggtgtgtccctcagtgggatgagctgaggatcacttttttgctaattaaaagacacatatgtccagtttcagatagattttcaaatgtaatggagcttctgttactttttaagaatagggacttgccttttcagttttcgtttttgtatataacattcaggatcgaaaactacggtggcctgatgacttctttttctcccaatagatttttaaggactttcacgaaggagccattaactttggacccacctacaagtatgacgttggctcagccgcctacgatacaagcgacaaatgccgcacccccgcctggacagacagggtgctgtggtggaggaagaaacatccctttgataaaacaggtgagggggccgtgcccgttcgagagtcggcagagggtgaataagaaatgcgacggagggagagtcacgtttacccaaccccaggcgctgctacataaatcagtttcttcataaccccagatgacacaaaagtcatcccagaccttaacccctaagctttggaagttgtgctttcagggagcctctgcttctggcctctttttttcccttttcttttcttttttttttttgagacagagtttcactcttgttgcctaggctggagtgcaatggcgtgatcttggctcaccacaacctccacctcctgggttcaagccattctcctgcctcagcctcctgagtgctgggattacaggcatgcaccaccatgcccagctaattttgtatttttagtagagacggggtttctccatgttggtcaggctggtcttgaactcccaacctcaggtgatctgcccacctcggcctcccaaagtgctgggattacaggcataagccactgcgcctggcctcctctggtctctttctagagacagtgacctgcaaataggtctgctcctggctgcccctgcatcacagctcccccccacacccccacctcttttttctgggtggggaaagcggggagcatgaggttgcctccctccctgccccccatgctccccctcatgttctttatccagcctattttgttcagctacacttttacctgcttaaagccctaattccaaattggacacgagcaagacacagtaaaaataagttagtttttaaaaatcattagttgtcaagtatgttttgaacaactgttgatgcccagctaggaagtgaagtttatttaaagttagaatatctgtaactagtacataaaaaaaaaaaaaaaaaaaaaaaaaaaaacctaattacacattttgggagagcatagccaggaaattgcatctgtgaaaagaaaatacaaattttcctctagatgagtttgtgaacgcctgtgtggccccagcgctctgagaggtggggcctagaatgagtccgagtgttctcagctcctgcagctcttaccataccaattgctgccaaccttctgggattcctgggtcctggaaacccggcagcaacactgtgtctcaagcctacagtgggcatgctggcgaggactgcaggcatggtcatgactgttttgaataattatatgcgaggaatgtttttaaccaaaggccccaaagcatccgaagcctggcttcaaggtggaggagtaaggaggatgagtctgggacgtggggtggttcgtggcgcagacctctattgtggaagtgtcatgatcgcattcttggatattgactcgattctatatgggtctctcgaatggaacccctgcgagtagctggagaactcaaccttctagacagtgatctagatgttgacaccaaagtcagacacacctggtctcctggtgccctgcagtattatggtcgtgcggagctacaagcgtctgatcacaggtgaggtcctgacttccatggcgttttctcctgtgctgggcaaggcagcgtctccctctccgcaggaaaatgcatgctgtccccataaggatgtgtgtgcgcatgggggtgcattttgtgtgcatacaatacaccaaagaacaaagaatgatggaacaaatatctgtggacccaccaccaagaactaattaatcttagcattttgtcattgctccatatcataaatgacatcattgatgaaaatttcaaagatgcaacctacgtattttgcggcttttatattttcttaaatgatgtcgttgtgcaacttgcttttttgttactatttttgagatacagtcgtgttgatgttatagctctggtgcattcatttgtaactgctgtctagtatcccactaaatatatccatggaacttacaacttatccattcacctcttgatgaatattttctccaggattttatgattacaaacaccatagggtttttaaatctgggatgcacaaccttttttttttttttttttttgagagagagtcttgctctattgcccaggctggatggagtacagtggtgtgatctcagctcactgcaacctccgcttcccagattcaagcaattctcctgcctcagcctcccaagtagctgggattacaggtgcacaccaccatgcctggctaatttttgtgtttttagtagagacggggtttcaccatgttggccaggctggtcccaaactcctgacctcaggtgatccacccaccttggcctctcaaagtgttgggattacaggcgtgagccaccgcacctggccctaggatgcagggctttttaaatgctctgtgcatcatgcattgaggatagagtggaggcagcagagggagcaccgtgggcaccagcacctgtgccctgggattgctgcagaggaagactcaccctgcagaatgtctttaaagacagaggagaggatggcatttgagggtgtgaagaaggaagagtttgatcttcccaaggctgctgcctaaactctcatgtcagtttcaacttttacaaggaaagaaattaagaaaaagaaggtaagaacagttggtcaacgaaaaaaccttttttttttttttttgagacagagtcctccaggctggagtgcagtggcacaatcttggctcattgcaacctccacccccagggtataagtgattttcatgtctcagcctcccaagtagctgggattacaggcgtgcatcaccatgcccggctgatttttgtatttttagtagaaacggttttgccatgttagccaggctggtctccaactcctggcctccagtgatgtgcccgcctcagcctcccaaagtactgggattacagccaccatgcccagactctttagataattctaaattctttagatacctgttgttgcaaatacatacctagaagtgaatcttgaggaatcttcagatatgtgacatcaaggtttgctagctcaatgtattttgaaaccttaatttaaccaatatttcttgaggggcccttacatgccaggccctgttgctggcctggagaaaagcagtgaacaaaacagatgagaccatgttatcatggaattttctggacaggacaaacagacaataaacaaacatgtgtgctatgaaagaaaaaaagtagtggcaggagtcaggccagagtcgagctattttcaatcaggctgtgtaattttcgtaatataggattttctggaaatttatagagtaataaccatccctgtgtcagaattactataaggattgcattatttcctgctggactgttttcctgtttacatcattgatttttacattattcctaatgattgcgtagggtattatatgaaggtatcataactgtttccccattaagaaacagagcattttggcctggcgcagtggctcacgcctttatcccagcactttgggaggctgaggcaggcggatcacctaaagtcaggagttcaagaccagcctagccaacacagcaaaaccccatctctaccaaaaatacaaaaattagctggtcatggtggcatgcgcctgtaatcccagctacttgggaagctgaggcaggagaatcacttgagcccgggaggcggaggttgcagtgggctgagatcgcgccactgcactccagcctggacaacagagggagactctgactcaaaataaaaaaaaaaaaaaaaacgagcatttccctttttttttttccctcccatcaacaatgctgtaaatgatcaacctcatgcattaaatctttgtctgaatccctggtcatttccttagaataaagtccgagaagtgagattactaggtgaaaagagatgaacaattttggaactctatgtaaagtgccaagtaaccctctgaaaagcttgcacccctggcagagtgcagatgtctgcttccccacctccttggtggtactgaggattaccatgtcaaaaatctttgctgatggctggttattttccaagtactgggatacatctttaaggtgccacactttattttccccctcaaatcatctttagataaaaatgccaaggtgatactaaggttaaggaaagatgaagactaaaacgccagagcagggcagctagcctgcaaatccacctgctggtggccacattcgcaggttgttggtgttgactgtttacaaagagccctgggggacagcagctggggactgggggctggagatgtggacatctgtcccaggctaactggggactggaggccgggggtgtggacatctgtcccaggctaactgtcatccctcttttgttcctaacgcagacctgtgctggcgatcgtggaggtggaagttcaggaagtcgatgtgggtgctcgggagagggttttccaggaagtgtcctccttccagggccccctggatgccactgttgtagtaaaccttcaatcaccgaccttagaagagaaaaacgagtttccagaggacctgcgtactgagctcatgcagaccttggggagttatgggacaattgttcttgtcaggtaactgctcccctggctgatgtgggttccaggggatgtcgatgaggatccgtgagagctgccaggtcgttcttggcattgacttggagtatcatttattccaggatcaaccaagggcagatgctggtaacttttgcagacagtcactcggctctcagtgtcctggacgtggacggtatgaaggtacgctgtacttggccacactgtggagtggggtctgatcaatccctctttttatgtgggcatgtcttcttcctcctcttgccctgacctgtgccttttttttttttttttttttttttttttttctctgagacaaagtcttgctctgtcattcaggctggagtacagtgatgcaatcacagctcgctgcagccttgaactgccaggcttaagcaatcctcccatctcagcctctcaagtagctgggaccgcaggcacgtgccaccacacccagctaattttttttaaattttgtagagagaggatctcactctgtagccccggctggagtgcattgggacagttatggctcactataacctcagcctcctgggctccagtcatcctcccgcctcgatctcccaatgtgttgagattacaggcatgagctttcatgcctggccaccaatctgtctttatgtatagacgccatatgcgggagggagtttggattcttcactctggttctaaggtgtctcatgtccggctctagctacttgcaaaatagggcggaagggtgggcgcagtggcttatgcttgtaatcccagcactttgtcaggccaaggtgggtagatcacccaaggtcaggagatcgagaccagcctggccaacatggcgaaatcctgtctctagtaaaaatacaaaaattagccaggcgctgggtgtggtggcttacgcctgtaatctcagcactttgggaggctgaggcgggcaaatcatgaggtcaggatttcgagaccagcctggccaacatagtgaaaccccgtctccactaaaaatacaaaaattagccaggcatagtggcaggtgcctgtaatcccagctacttgggaggctgaggcaggagaatcacttgaacccaggaggcagaggttgcagtgagccgagatcgcaccattgcactccagcccaggcaacagtgtgagactctgtctccaaaaaaaaaaaaaaaaaaaaaaaaagtagccagacatggtggcgcgtgcctctagtcccagctactcaggaggctgaggcaggagaatcgcttgaacctgggaggcagaggttacagtgagctgagatcgtgccactgcactccagcctgggtgacagcgagaccctgtctcaaaaaaccaaacaaacagaatggggaagaagaatgtcagtcactgtgtctcacgttggaaggcagctctaccagcagctgcagaagcttagccattttagagtgtagttctcgccgagttacttgatctgtctatgctcgcactgccatgtgggagactattccagtctttctctttagagtatgtattttggaatatgcagctcatagatagtttccgtttttatagtatcttggctttaaatctgtaccaaatgggaagctggagatgagagttcaactttttttttttgagatggagtcttgctctgtcacccaggctagagtgcagtggtgcaatctcggctcaccgcagcttccgcctcccgggttcaagcgattctcctgcctcagcctcccgagtagctgggactacaggcactcaccaccacgcccagctaatttttgtatttttagtacagatcgggtttcaccgtgttggtcaggctggtctcaaactcctgacctcaagggatctacctgcttcggcctctcagagtgctgggattacaggtgtgagccaccgcacccagcccagttcaacttttttttcaaccagatgtatgagagctatgctgagcctgcgtgaggtctggacccttggtttttcagggccaaatgtgaagattggcctcgttgagggtgtgtgggttttggtgagaagcattgtgtgatcaacaggaggggtcatgatttttatttattcctgggtggccgctctgccctcccaggtgaaaggcagagcagtgaagattagaccgaagaccaaggactggctgaaaggtttgcgagaggagatcattcggaaacgagacagcatggcccccgtgtctcccactgccaactcctgtttgctggaggaaaactttgacttcacaagtttggactatgagtcagaaggttagtgaccctgcagggagggacaggcaagccgttcttctagcaacaggcctgagcttttaaggacaccttctaaagcctcccttgcagaagtgacggaggacacccgcagggcaagccctctgtcccatgtgatgagcatgtttgtatgtgtggacgatgcatgccatgctacctgggacccgaggcatctaaacttaggctacaaacagtggctatgaggatatcagggctgtgggtgtaccaatggaactggggtaccgtccctgctggcgcctgggccctgagctgcagggcagggtgcacgtgtcctgacaggagctgaaccagtcccactgatggaagactgagcctttccccctcattgttttcacccgaattgtgattcatttccttctcccaggggatattcttgaagacgatgaagactacttggtggatgaattcaatcagcctggagtctcggacagtgaactcgggggagacgacctctctgatgtccccggccccacagcactggctcctcccagcaagtcacctgctctcaccaaaaagaagcagcatccaacgtacaaaggtagcctgacccttcttttctcaggagcctcagcgatggagtcagctcaggacagactttcctttttctcttggcgattgggcactgtgtgatatcaagtatgcaggtcccaggagagacctcaaccaggcaggccaagcgaggggtagggactgagccctgtggaggtagccagctccaacaccctgatttcctctagttagtttatttttttacaataacttgtacccccataacacaccagattcaagataattcttgtttcctctggatattcaaatggccacggttctgaactggttttatatttgtgattatttcccgcccccaacaaaactcttctgattcagccaggcgcagtggctcacacctgtaatcccagcactttgggaggccgagacaggtggatcgcttgaggtcaggagttcaagaccagcctggccaacatggtgaaaccctgtctctactaaaaatattttttaaaaattagcagggcatggtggtgcacatgcctgtaatcccagctactcaggcagctgaagcaggagaatcgcttgagctggggaggcagaggctgcaatgagccgagatcgccccactgcactccagcttgggtgacaaagtgagattccgtctcaaaacaacaacaacaaaaaaacttctgattcaatcccagtagagacataatccatatctatgtcctgagttgctggcgtatggtcacactcatataatatcatattattatacagtcatgtcatacggtcacatcattgtatcattttagacttcagagggcctgaaaggtcacattgattgaaccacacacccctagattaaataatacaactttttttttttttttgagacagggtctcactgtctcccaggctggagtgcagtgatacaatctcagctcactacaacctccacctcccaggctcaagcgatcctcccacctcagcctcccaagtagctgggactacagggttgcaccagcatgcccagataattttttgcattttttgtagagacggggtctcactatgttgtccaagctggtctcaaactcctgggctcaagtgaccctcctacctgggccttccaaagtgctggaattacaggtgtgagccactgcacctggccagtaactgttctgagtaacttttaaagcccactaatgttacgtggctggcaggtcgggtagttcatatttcagtgtcagctacattgatgctgttccattcatagccaggcatgcctctagctgtcaatcactgagccccgagttggaggacagctctaaccagcaggtgcaggagcttagccacgcattgggcccattgagctggggctgagagcagagaacagggctgaaagtggattaaggtacctctcagggagggtactttgccaggagaggtgcgaagacctccagcccctttgaccgtacgctggtccccacactgaccaataacttgcctataccgaaccatcaggagcttttaaacttatggatttttgagccctaccagagagatctggatttggtaggagtggggtgggaccctggaagcctggcgtttcaccaagctttccaggtggtttggacgcacagctaggtttggggacttctgcccaagactgaagcagcagccttcgccggagcatcgcggtggttctgcacgttgctcctgccaggggcttctgcccgggagttgaagtggctggagggatgtgctcttcccataagaaggacctttctctcgggggtggccgtcatgcaggcagcctctggagccagtgcaggtgtgagcacagatagtcctttgcatgaagaaactaatagcgcaacacgggacacgtgggtggctttgctacctggtgggcattgagagggtcttcccttgatgctgtggggtgttcagccattcccatttgtaggagaaatcctgcctttccccagttccagtctccaggacacatggggtggagctgctggatacagggagtggggtgtccgagtcaaagtgaaatggagaggcccatgctttgggggagccctagggctgctgatttgatctgtaaggaagcaggagctgtgcctcctgaaaacccctcagcctcttggaaggtgtgtgcagggcaggtcaagatggcagacccttgggagtggccagccatgggcccacagggtttgggatttgatgagtaagcaggacccctggtcctaggcactggccactgcatgccaggctgctctgctgccagagcagggagaacaccctcagcttcctcagccggtgtgttctagagtgaggaattgaaaacatctgcctgtggttggttgactcttcaggggttgtggtttgcaactgtctcctccactctgcccaagtcttgttatttcacaagaaaacacactttgaaatagctcagtgtctctcaagtctgagaaatacccattatccatggttcactgaatgttccaagcaggcactgggtgccctccccgcccccgcccctcgccggcagacagaggtgcccgtttctggacagagccttcagctgtcctacagcaggtccccctgccacagtcggcctcctgtgctcagatccgtgtctgacacgctcacgtgtggaacagaccattgtactcatgccccgccatgtcctccagatgacgcggacctggtggagctcaagcgggagctggaagccgtcggggagttccgccaccgttctccgagcaggtctctgtcggtccccaaccggcctcggccacctcaacccccgcagagacccccccctccaagtaagactctgcttgctttcagctccctggatgcctgccaggaataaccgcgcgttccctgctactgaaaacacggctccgaatccacttctccccggccttccggtccccacagggcttcctcctccttgggctccgttttgttgtaaatccaggacacgtttgacgtcagggctggttgtgtttctcatggcaaatgaaacagctcctttccaaagctgctgttcaccctggagctgtgctattgcttcaaactgctctcccttggcagcattagaggatgcagaatgggggccgcagccatgggagggacgtgagcttcgcagtcagaagtctctcctcagctgctgcgcagtcctccctctacttatgaggaatctggtaaacccaggtccccgcctggaggtgacctggtcacccctctgcgctccgaggcctcctgttccccgggctcagaacaagttcccccttatcctgggtctaggagcattttcttctggttgttcgaacaatgctttgcccgtccagccctccctgataacctgaaagcaaagctgtgatgtgcttttgatccacttgcattttcccaccactgtttacttctccctccccaaaaatgtgatgatcagacttagatcttcctaagccattacttcagaattgggctcattgtggctgacgtcacggtagtaaccaatggcaagccacaagcccagatataaaaataacccaaaaataaaatgcataagtcatgtattattgtggaaattttagagataactacaaagaagaacaaaattttaaaattcacttgatgacctacgggtcgtagttgttgaaaaaagtcttctttttccagtttcatgtatctgcagcaacatacttctttttttttttttttttttttaacaaaattaatacccattttctctggtcggttgtcttttatttaatgggttatattttctgtgtcattgaatgttctaaaacatgatttgttaatggctgcaagtatattttaacagtttattcccctgctttggaattttttttttttttttttttttttttttttttttttttttttttgagatgggctcattctgtcccccgggctggagtgcagtggtgcgatctcacagctcactgaagccttgacctccccggctcaagcaatcctcctacctcagcctgccaagtagctaggactgcaggcgcataccatcaggcttggctatttttattcatttatttatttattttgtagagacagtttcaccatgttgcccaggctggggtttttgaaatattttttccccacaaaatattttaaaaatctggtcctagcaatagtatttggaaccatttcaaaatcaattggtttttatatcagatctgaatcccccaagatgttaaagtttgcattctagtaatgcttctccagatcatctacatcaataggcacacactggtagaatcaagtgagaaactccatagagagtcaaggaaggcctggctctgtgggtgccctaagtgccgagtgagtattcggacctcaccgtgtctgtggtctgctgggacatccgcctcgtctagtcgggctcctggcagctggctgggaagttgtgccttcactgagattgagactctgccctcgttggtttttacagccggtttaatggtgaaaaagtcggcttcagatgcgtccatctcctccggcacccatggacagtattcaattttgcagacggcaagacttctaccaggagcacctcagcaacctgtgagttcttctgcatacatttcaatcccaagggttttgcccccgggatagtgtgggatctctcttctcagtgaatatatagatttatgtgtctttaaccctctacctgctcaccatctcatctccaaaccccataacccttcctcgcaggttctccatagggcttttaaatggttgtatggagtgttctagaaaaaatcacttttgatgaaaaatcccaaatatccctggtttccatactcatccatttctctctcttcccattcttgcctctgaccttggtgtatgggcagagcattgcttggtagaagggaaaatgggagaaattcaggtcgttatcctgggaaaatgatgatcattctggagtgaagttggttgagagaagaggctaaaaagagatgtaaataatgaagttggggaacacactaggaagactaacagccatagaggattgtggaggtgcctggttccagagggtaccatgagctgctggggaagagagcaggtgaaggcagggaagtagaggggagagaggggaagctgggtgctagggggaaactgctatgaccgggatgcctctggggtgctgggcctggttaagtgcttgtgcggccccagcaggtggaggggtgactggaggccagtgttttacagtagtgaaaataggtcataaaggcaacccacatgctggatgaagaagtagaaaaagcacttttaaggaaggtattttaacaactcagtgggaccatggaaagagtggcctcttattttgctttctggctttcacctggttattgaaaaaaagatagaagatggtctgtccatatgcaaaaatctgtaatcacccggctttgaagaccagggtcatcaggtctaagtagtatggcactcttggaaagagagtgctccaggaaagagaggattgcagggtgtggtggaggctggagtttgtatggtcagcatcgtctggtggagccctgcacagacccacgggtagcagatacgtcccacgaggctgtcctcctgcctctgctgatactctgctcttcctcattctgtcctcaagcccaaggctcggactggaataagtaaaccttataatgtcaagcagatcaaaaccaccaatgcccaggaggcagaagcagcaatccggtgtctcctggaagccagaggaggtaggtgctttcctgggggcaggggaaaaaccaattctccttttctttttatctccctgctaaaagtggtctagaaacgaaaataactctttttattcttctggaggcaatattaaatgaaaacatcctttagcaacattttatataaatctatactaagattgattgtccatttaaaattgtattttcaaaattctgtgtgtatataaaaatcatcccagggctattaagcggcgtctaaaggagagaccgaaaaaaaccaaaatgagtttttaaaaaaggtgaatgctgccggcatgaagtcattctgccagctttataaaacagagacagttatagtgtttgtcctaatttcaggcacaaaatctaccttttaaatcactggcaagtggaaataaagtttttagtgtcaggcagggacctctttcccaccacattattttttgttgctgttacttaaaccatgttagttaccttgggctctcccccacttcattcataatcactggtaatttacacttttagttttggccttggctttgttccagaatctcctagagaggagagtgtgcccttttcttcgtttttttttttgttttttttttttttttttttgagactgagtttcactctgtcagctggactggagtgcagtggctcaatcccaggtcactgcaacctccgcctcccaggttcgagcaaatatcctgcctcagccacctgagtagctgggactacaggcgcgcagccccatgatgagccaatttttgtattttcagtagaaatgtgatttcaccttgttggccaggctggtcttgaactcccaacctcaggtgattcatccaccttggcctcctaaagtgctgggattacaggtgtgagccaccacacctggccgagtgtgccctttaaaaaggtgtaagcaatcaccattgcttaatttgcttttagtcagcatgtttaacttctttggaattctcaaataatatttaagggggcatgagttaaaaggtcgagaactactgttgtagaatgatactgctacaaatgctttctttttttttattttagaaaaataactaaaactaggccgggcgcggtggctcacgcctgtaatcccagcactttgggaggccgaggcgggtggatcacgaggacaggaaatcgagaccatcctggctaacacggtgaaaccccgtctctactaaaaattcaaaaaattagccgggcgtgatggtgggtgactgtagtcccagctactcaggaggctgaggcaggagaatggctgagcccgggaggtggagctcgcagtgagccaagattgcaccactgcactccagcctgggtgacagagcaagactccgtctcaaaaaaagaaaaaagaaagaaagaaaaataactaaaactatggatataacaacccaagtgtccatcagctgataaatggataaacaaaatgtggtccatcaatacaatggaatattgttcagccattaaaaaaaaaaagaatggggctgggtgctgggtgcagtggctcacgcctgtaatcccagcgctttgggaggccgaggcaggcggatcacgaggttaggagatcgagaccatcctggctaacacagtgaaactccatctctactaaaaatacaaaaaaattagccgggcgtggtggcacgtgcctgttagtcccagttactcgggaggctgaggcagaagaatcatttgaactggggaggcagaggttgcagtgagccaagataacgccactgcactccagcctgggtgacagagctagactctgtctcataaaaaaaaaaaaaaaaaagaatgaagtgctgatacatactacttcatgggtggaccttgaagacattatactatgtgaaagaaagaaaaaaaggccacatagtgtaccatcccctttttatgaaatggccacagcaggcaaattcatggggtcagaaagttcttcttgctttccagggactggaaggaggcctgggtggggagtggctgctgtggggcttcttttttgtgtgatgaaaatgttctggagttagatcgtatgatggttgcacaactttgtggatacactaaaaaccaatgaatggtacacttagaaagggtgaatttcacggtatgtgaattatgtcacaataaagctgttaaaaaaaaaaaaactctagagatgtagatgttagtagaggtgtgaaatctacctacagtcccggcttaacactttcttggtcatggccccattgagattttgatgtctttggatcatctaccctgagaaatgtaagtacacatgagactttgcatatgatctcagggtctcactggtccccatccaaactatccacagaccccagatcaccacccctgctaggaaggtgagcatgaaaacctgggactgctgggtgcggtggctcacgcctgtaatcccagcactttgagaggctggggtgggaggactgcttgaatccaggagttcgagacaagtctgggcaacacaatgagaccctctctctacaaaaattttaaaattagctgggcgtggtggttcacaccagtagtcccagctacttgggaggctgaggtaagaagactgcatgagcccaggagttccagcaacatagtgagacctcatctctactaaaaattttaaaaattggctgggcgtggcagtgctcacccgtaatcccagctactcaggaggctgaaatgggagaattgctcgagcctgggaggttgagactgcagtgagccgtgatcatgccgctgcactccggcctgggtggcagagcgagagcccatcttaaaaaagaaaaaagaaaaaaagctgggactgaatctagctgattactcctgaaatacactgaattagtaaatgacacttaggatgtagactgtctaaggacgaaatcatgcagtgaattgccttgtcctttacacttcagccatgtggttttatgtttcaggtgcctccgaagaagccctaagtgccgtggccccaagggaccttgaagcatcctctgaaccagagcccacaccgggggcagccaaaccagagaccccacaggcgcccccactccttccccgtcggcccccacccagagttcctgccatcaagaagccaaccttgagaaggacaggaaaggtaaaagcctggtaacataaaacctcttactcctaagttgctcacatgtctcgatagataagaattgtggcctgtaatcccagcacattgggaagccgaggcaagcggattacgaggtcaggagttcaagaccagcctgaccaacatggtgtgaaaccccgtctctactaaaaatagaaaaattagccgggcgtggtggcgcacgcctgtaatcccagctactcaggaggctgaggcaggagaatcacttgaacccaggaggcagaggttgcagtgagccgaaatcgtgccactgcattccagcctgagcagcagagtgagactccacctcaaaaaaaaaaaaaacaaaaaaaaaaaaacagatttgcgtctcacagataaaaatctagttccctatggacatcctcacgttcatgagtcccagacacaggtgtgtctttggggaaagttttgacaactgcttcttccctcatatcctactgtgtcttggctctggctgagaagaacgaggagtccactgtctggcacattgtcagtgtgacaagaacagcttggaggtattatgcttcctaagtgttttgagtccccaaagggaaccatagtgggatgcttttccccacatgttctggtaccgcccatttgcgtgtggtctgtgttgtgtgcacatggtttcaaggcattaggaaaacagtaaccgattcccagattcgccatgttcacatgctctcgtgcggcctcagcctacgagaggttccacagcttttcttgccacttttcttgcagattgttttttgctctcgctctcaagcttctcagccttgcttactgcttcaaagacatgagtttgtaaggacagtagcagcccaaagactggcatctgtagacacatctggatcttccgtttgatctcagtgttctagtaacggaccctcggtaatcccttcctggtcatcacagcacgatgctgcttcccccctagagagtgtgaggggggcttcggcttcactcttgacagcgcttgctttgtatttcctgggttgctcttctgtcccacattggctcattgccatctgtcccttgtccatgcttccttacaaagtggacctttttcctccgcctcttccctgttcagatgctcctcgacttaaaatggggttacatcccaataaacccatcgtaagttgaaaaatagcgtgtcaaaactgcatctcatacacctaacctagcaaacgtcatggtctcgctgaccttgaacacactcagaacacatacattagcctacggctgggcaaaaaaaaaaaaaaaaaaaaaaaacacccatccaacacagcctattttataataataaaaataaaagatcaaacttcaaaattcaaagtatgctttctacggaacattagcgcttgtgcactagggtgaagtggaaaatcctcagtggaaccattgtaagtcggggaccacctgctttgtcttttcttgtaaaagccaggagcagaaatagattcattttcccaacctgctgcacaggagttaaagttaaaaggctgttggaaaagagactttcttttttcttagtttgttttctaaagaaccaaagaaacactgaaacgccaatgtttaactagtctactgatcaccttttctgaacaaaagcttcattttgcctgtgcagttgttttctttgtgatagatttgaaccagaaccaaggcagtgtcaaggtttccattctttaccaatatttaagtataaaagattcaaggagctgggcgcggtggctcacgcgtgtaatcccagcactttgggaggctgaggcgggtggatcacctgaggtcaggagttcaagaccagcctgaccaacatggtgaaaccccgtctctactacaaaatacaaaaattagccaggcatggtggcaggcgcctgcaatcccagctactcgggaggctgaagcaggagaatcgcttgaacctgggaggcggaggttacagtgagctgagattgcgccattgcactccagcctgggggacagggcgagactccttctcaaaaaaaaaaaaaaaattcaaggagaaagtatatccattagtccttcagttagcttatgttgtaacacaagttacttcaccctagcatgatacagaccatgaatcagcccaggttgcaacttcaaagagtgttttcagagaggtttgctttcaataaaggaagagcatcaccccatttcctaaaggacaaagaaaagtttgtctgattatttactgtggccctggttttaaattttccactcgttttcattggacctgctggtgtgacctgataaatgtgtgtttctcctgtgctttttctttttcttacctggtcacatttctggcaccccacacattctcaaatctcgaatttgctagaatgtcagcttggtctcatctctgttctctgctggccactctgttctctgatcctcagctgcctagttattggcttcttatttacactctttgtcccaccttttctcagcccctgtcaccggaagaacagtttgagcaacagactgtccattttacaatcgggcccccggagacaagcgttgaggcccctcctgtcgtgacagcccctcgagtccctcctgttcccaaaccaagaacatttcagcctgggaaagctgcagagaggccaagccacaggaagccagcatcagacgaagcccctcctggggcaggagcctctgtgccaccacctctggaggcgccgcctcttgtgcccaaggtacccccgaggaggaagaagtcagcccccgcagccttccacctgcaggtcctgcagagcaacagccagcttctccagggcctcacttacaatagcagtgacagcccctctgggcacccacctgccgcgggcaccgtcttcccacaaggggactttctcagcacttcatctgctacaagccccgacagcgatggcaccaaagcgatgaagccagaggcagccccacttcttggtgattatcaggaccccttctggaaccttcttcaccaccctaaactgttgaataacacttggctttctaagagctcagaccctttggactcaggaaccaggagccccaaaagagatcccatagacccagtgtcagctggcgcttcagctgccaaggcagagctgccaccagatcatgaacacaaaaccttaggtcactgggtgacaatcagtgaccaagaaaagaggacagcactgcaggtgtttgacccactggcaaaaacatgactgagcagctttgaaggctgcagtcctatagaatgcataccttcctccctctagacatccctccaccagaagagacatctatttaaaggcacactggccaaaacgtttgtgcatctgtcactctcgtgtagtttacaaaaatcgtgtctcttattcagtaagatggttactcagccaccaaaatatatttcactcaaggcttgtacatctgaagtttgctcttcaaggaatgggaaccttcctgttaaattcggtgtatggattttaagaaaggaatctagccaatgaggtccaagaagttctcacccattgaatttttaaatggctgttcagttcatgttgtacgtgatggagatttgtcttttgttttatttgcattttacagatttggtataacattttggggagccacctgaaggttgatgtataaagtaaggattagagaaagaggtcgttgtgaccattagtagctgtcctggcccacttaaacaaggttacaaaaaatcagagtcggaagcagccaaataggtcaacctaatgactagactgtacattcccatgagccttcatgtttaagtgtgtacatgtgcgttaaccttgatgatgcgtgaatcccgagggagccggtggcatacaccgttagcttaaccttagcttaaactagctgaaggctcctgtgccatgtcttagacattgcatgccctatcaattactataatcctgagccatggtgtgctactgaaaccaatttttatccaccatctagtccttattaaatgaaacctcacggatcctttgttccgcttatattccatgcataccacataaaagcacacagtgcgaaaactcttgctgatacgcgatattgattctcattgttagaatatggagagtgtttcagcctcgtctgtccggctggagcttcgggatggaaagtgctatgtgtccctgcatataagaatcaccaggccagtgtttctgggtttgcttgtctatatgtttgtctatattttttgcctatacatttttcccacgtttccaacagcacttctcacctattcaataactgaaaaagacattaccatagtgctttacatttttaaagtaatgttacaaggtctggaatccatttggagcagataccgtgttttcgctatttaataagaagttcagtagtgaaatcttactgtaccgcctgttgtatctgggagcctcgtacagaggctcgcacagcagtgatcaagtgtcatcccttacgtgactgggggatgtctgtcctaaaagctgactgctaggatagtaaggatcatcttgcctgggctatgccactgtcttgttaccaattagacatctggaatttcataattagttttcattgtcactgtcaagatatattgcagattacttaaatatggccatcaaaacaaaagttacaacacgtatctcttttcatctgaaaactaatacctggaaaaggataaaaaaaaaaaggaatccgtgacccacagagctagacagataagatgcatagttgaccagtcataaaaggcggtgtttaggtgatcaggatgccgttggtggcatttacgtgctttatatgatttttacctctgtaacaaacacaagaaataaacagaatggtccttaacagagtttgggggagagagcaagatgggttccttggagaagctgatttgccaagatgcacatcgctattaacagccagagtcataaatgaaatgaaattgaagaattcattcaaatgctcttttccctataacctcttttctcaccaaaaaggagataaatttgaaaacagataaatgtaacaaccagtcaaagaagcaggggaaaagtaagctcctccaaagttgcttgcagtgctggaaatagatctcatttttaggttttctcttcgttccagataccaaataaatgggacagagaataaaatttttgttaaaatatgtgctcatctcctaagtagctcttcagagtctgaccgtaagtaaaaacacacagaattgtgttgactgggggaggtgaatcacaaaaaagttacgaggagtttaagagttaaatattatttgatcgtggctgtcaaatttagtgaacaacatagattggatttggagttggtagtaggtatggttctcataccagaattctcttaaaaaaaaaaaaaaggacaattggaattgccttatttatttttaaaatcaatgcttactagttggtaggattcccaggtcagcagcagggttgattaaataatcttgacaatgagcagctgccatcttgggggatttcattctgtggttttttaaatgtttcgtctttgatgctaccatccagggcttcttattgtgaccttgtagcctattttgttcctgctgttctctaacatggtgcagttcacgcagactggtttaggtacttcacaactcaccattgtctctctgaccccaagcctagtcccttttacatcaccatcttctcagacttcttgcctattcttaaaatatgttttggttaatgattgaattaggacatcagcttaagcaattcctgtaacggtttaacgttgttgaagttcaaggcacataataaaattctccctgatgtgtgtgtaagtataaacaaagtaatgtatcaaagcgttcactttataatgaagtcatttcattgggaaggaaagctgcaaagattattgggggactagtgattaataaaatcctgtaatatttttgaagtgaaaatttgtactgaaattgtacatgatacctattaaatgtttttgtctttttttaaa

SEQ ID NO: 2 - DNA, 117290 bp, homo sapien
gcggggagctgtcgcgggcagcgcgccctcgggaggacgtggccccggcccccgcccgcagtgggcccgaccctcatggccctgagcaaagggctgcggctgctggggcgcctgggggccgagggggactgtagcgtgctgctggaggcgcgcggccgcgacgactgcctgctgttcgaggccggcacggtggccacgctgggtgagtccgggccgggggcagcgacgcccggaggagagggcgcccgcattcgcccagcctcgggaagacgggtacccccccttcccgaggggatcgggcggcgctgggactgccggggcgtaggggtcgcgcgcagaggggtggctgtttgaatgaagtggggctggggactcgagagagcactctggctgggccggtggcgcacggaggccggccgcccctccacggggatctccggcccgcgccgccagaggatatggttgctggatagccggctggggcgctttgcgtattcatgaggatcccggggtgttgacttagccggcctgaccctgtgcctggcgccttttcttttgcacctgttgatgaaaccagtgattgccctttggtttgtgttggcggctccactcctggaggggatggggacaaaagccccaaaacataggctctggagaaaccgctgatggcagctttgtcccacgtaaatacttggtcgagaagcatcggtgctgcaatttatagaacttgtaaacaaatataccatgacctttatgctgtcttagatcctgccaagccagcgaatttcacctatggacttcagaagccttgaaactggcattgtgctatgggctttagtacgtgatctcacttaattctcacaattctactaaaaagctattattatccccattttgcagatgagcaaactgaggcctaaagaggctaagttgcttgccaaggtgacaccatccacctgacttttatgtggctgtttctgtaatgtcattcaatgcgtggcttcctggtatcgctaacctgggtcccttgttttgtgctaaccattataaggaaaacctgggtagcaccaaagcacgtgaaaccaggtctgattgttagacttctaaatgtggacacccgagagtaagctcccaccactaggcgtccagctgtaacagcttgccggtattcctagctaaacaaggtgcagcccggggtcactcgtacttgaggtggtcggcaaatgcagcaggctgctttgtagatgcctctagccttcttttcacatttgtatgcatcgactcccgttcggatgtatgtcttggaaggactatctctcctatttctgattccctaatcatttcattaacatgcatttatccagcttgttctcagtcacttgattactgaaacaatgtctccagtagtctcagcatcaaaagtaggcagtgaattgtagattaagttattttcagattattctaatttgtgctgtcctgaagagtctaagtttggttatttggtgtggccttgaaagaaaaccacagaaatttccattccattttagatgtctacttcataccacatttatcggaagcgaaagagtaatttcatggtacaaaactactgtttcatcaaatagattaagatgtccttcagttggattacggcaacattaagccaagttcatacacattcaagaattctttgaagcagcttagaaaaaagagattttattttttcaaaagtgaagatttaaaaatgattaatgacagtaatgtagtgcatattttccattaggagctgaagtttgggctgagatttacatcacggtggtgattacagtataccagattttagaacagagaatgtaagaaaggaattggctcgtcttcaactcatgtgcagtccacatttgtcatttaacctagcattcatttttcagtagaaaactttaaaaaaaaattttttttttttttgaaacagaggcttcttctgttgcctaggctggcatgcagtggcacagtcacagcccactgcagcctcaacttcccaggctcaagcgatcctcctgcctcagcctcctgagtagctgggactataggcacatgccaccatgcctggctaattttttctctttcttttcttttctttaaatggggtcttactatgttgcccatgctgttctcgaactcctgggctcaggtgatctttccgccttggtctcccaaagcgttgggattacaggcatgaactacttactgcgtctggcctgaaaacttaattttttaaagtctgttttgtttaagtaaaatgaaactatttttaaaaagcagctgtagcatttggtggctttgctggtccagagaatgtttgtaggagagagccttatgttactagaaagttgtttaaaactttcaacatatcctaaatgcatcactattggataaaatggctctgaacttgaaattcccatctgtgttccaaagggctagctagctgttttactttcctttcttggagaaagtgattattttttttcttttttttgagatggagcttcactcttgttgcctgggctgagtgaagtggtgcgatctcggctgactgcaacaccgccacccccacctgggttcaagtgattctcctgcctcagcctccccagtagctaggattacaggcacacgccaccatgcctggctaatttttgtatttttagtagagacggggttgtgccattttggccaggctggtttctaactcctgaccttaggtgatccacctgctttggcctcccaaagtgctaggattacaggcgtgagccaccatgcctgacccgagaaggtgaatttttacaagaaggctgcaatctctacttttgtatgggacaggacacttgtggcagctggcgtagctggtggtctaatcttgacctttcaccctttccttgaagttccgagtcgggggtagcttccttttctcttcagctgtggttttcctgtgatgggtccctcccagttagtaatgagaggctgagagacaagttgtggtaactgcttcgtgggtagtggtctttcccggcccctgagtcattctaacttccatcctgctccgctgcggatgcacatagctgctttcctaggggcgagaggagatgaggggactgggcctgatgaccacaggcgctgccccttcatagcacattttattcctgaaggatcctaactggaaaagtacctctcgccgaaggggctgggtggttccttatcattttcaggcaaggcctggagacaggggcttgaactgggctgtgatttttaggaaaaacggatggtccatgattcgttgattgctggactccttccatctgaacctgtcttcacctgtcttcacctgtcctgctctaaagtgaactcttcacactagggccgtctggcctgctagcaggattgttgaaggaattattccaaattcctgcttctaaaagaattaggttgtgaatctgaatctgtcttttatgtagatcagtgggagcaaaaattccagaggcagaaaagccaaatgtattggcactgatttaaaaggcttttatgaagtctcgggaacctgactggagggtagttggccttctcagcacaggtgggaagccccgtagccggtaccagcatccctggccaacaatccacaattagcaaacagatgttcccttatttgcacatgggacaaaaacctactcctgtctctgtctcttgtaaacttagggaaaagtgcttattgttaatggattttgtgtattttcagagtaagcgacagtgttccaaagttcaaggttctccaacagcccccaggaacggtggcctgagggtgtccctgtgtttacagtgcgggctgtgtgtgagtgaagtcgtgcctcctgccgcccagccgggctttgctgtgaacaggcagcagggagggagggatggggcaggagacttgagagaattaattaagttaattaatgggtgtccctaaattactttgagcttctggggggaaaggttctgtgtaaacaccaggcccccaggttctcccagctgctgttgtgcgaggccacgtctcgagtttagctttgtttgaattggtgagatggccccagggcagagcaggactcagtctgaactgttgcctagatcactttctcaggcattatggccagatttatttggtcacggactggaattttgatctgctagagaggaccacagagaatcaaataagtcaccattttataaatgctcctggacaaagttgaaaccacaggtaggaccaggcaagggttccggggcccctgacgtgggaaactagagcagcgacccgggaccacagtggtctgtctgcctccaggactggtttgcctcctctcctagccggaggctgtctcagcgtagtgtttacaaaatgcaaactgggatggtgtgtgcttctcccgcgggttttgtttgtttgtttgagacggagtctctttgcccagctggagtacaatggcgcaatcttggctcactgcaacccccacctcccaggttcaagggattctcctgcctcagcctcctgagtagctgggactacaggcatgcaccaccacgcccggctaatatttgtatttttagtagagaaagggtttcaccatgttggtcaggctggtctcaaactcctggccccaaatgatccgcccacctcagcctctcaaagtgctggggttacaggcaatgagccaccacgcccggccttccccgtgatttttgtagctcacctgtaagatcgtgatttcaaatcttaccaggctcttgttttggctgtgtggctcaaaaacttggcttttagcctttaattgtgaaaacgttcaaacacaaaagtagagggaaaaatgaatcccctgtctacctgccattcggcattgatagttatcaatatattgccatttttatttcacttgtccctgtattttttatgaaaacgtgaatctcctaaaatttagtttgtttaaagccaggcaagattctaatttgttgggttggcttttttttttttcttgcatttagatatagaagttcaatttttttattttattgtaaattgacaaattattatcgttttttgagatggagtcttgctctgtcgcccagctgggagtgctgtggcacaatcttggctcactgcaacctcttcctcccaggttcaagtgattctctggccttagccttccgagtagctgggactataggcgtgtgctaccatgcccggctaatttttgtatttttagtagagatggcatttcaccatgttggccaggctggtatcgaactcctgacctcaggtgatccacccgcttcggcctcccaaagtgctgggattataggcgtgagccactgtgctaggccaacagattaggatgattatatattatggggtacaaagtgatcatatatgtgtacaatgtggaatgattaaatcaagatgattaacgtagtcatcacctcaaatacttctttgtttatttatttatcttttttgaggtggagtcttgctctctcacccaggctggagtgcagtggcaccgatctcggctcactgtaacctccgcctcccaggttcaagcgattctcctgcctcagcctcctgagtagctgggactacaggcgcgtgccatcacgtccggctaatttttgtatttttagtagagatggggtttcgccgtgttggccaagctggtctcgaactcccgacttcaagtgatccgcccgcctcggcctcccaaagttctgggattacagacatgagccactgcacctgaccacttattgtttgtagggagaacatttgaaatttactctcctagtgatttcaaaatatacattattattaactatagttaccatgctatgcaagttcgaaatttacttggcagatttcgtgtggccagcagcagaaatggtggcatgaactgattctgagacattggccaagacacatttttaaaagcaaagtgaccacttgggaagggtcgaccacagcctggaagaacagggagcgctctgctggtgagctcgggaggacaaggtctgggccctttgacggcactgcctctagccccgggcaagagagggccgagggccatgtccagtggcctctatgctcccctttctggatgacattcctgctgctcaagaccctggagtgtgctaagcacatagtgcagcctgctcagccccctcccagggcctcaggcagactgtgcagccgtatgtccaccccaggcccgggcgttggggtggcaagggcatgttacaggtggaacgggacacacactagctgggactctgtgtgtgtatgcacgaggctccctgcaggggggcatgaagcccaggttgggggaagtcggggaagagaaggtggccaggctggggccgccctccccactggctacataatgaacctggccttccaggtagtcacggtggcatacttttcgaggtaggaggatagaacctattttatttaactgttttactttgttagcttgatttaaaactgaacagctgggcatacaggatgcagcactcatgagtactctggccccagaccctacagatattcaggcagacctgtcctgggcacctgtcccagcccgtgtctctctgctgtgtctctcttctggagcgttcctggggtgctcagggtcccctttgtgctttttggctgctcagtttctgttgctgggctccttcggccttcccactgaggatggggaatatattctgttttctttccacttgaattgtccttaaagccatgggtaaactggttcaggctcagacttgggaatttgatcggagtccctttagcatttctgggcagggcttgttggtgtcgtggccagctgtcctcgaggtgaaaaccaaattgggttgggagggtgcacatgacacttaaagcccccagagctgcggtgccatcaaccacacactgtggacaggaggctgtcagtgggcaggctgggtccaccttcaaagtccatacgaaacagcccagttcagggtactgcttcttttcgagatctcacgggcaggagccttctggcactgttcatgtaaatgtaagtcattctttctttctggaggaaaatttagacaggtgaatgaaaaccggagtgcttaatccgctattctacatgcagaagtttatctcaggaaacagtcccagatcactagcccattttgcttcaaggatgtttattgcaagctgttttgagtttttcactggttaaataaactttggttcaacttgatcataatagctgacatttgtggagtgtttaccacctaccaggtgctgatccaagtggcttacatttattaacttacttcatccttatgtcaaggcaagtactagtaatctccattttacacttggggaaactgaggcacaagaggcctaagaacttgcctgaggctccacctccagtggaaggctccagagaacaatatgttcagtaaaatctcattcttatgtaaaaaaatgaatatatatatatatatatatatatatatatatatatatatggaaaaagactccctttgtcctgttgtctctggcttatcttggttttcatctgacatacacttttgatcgaggaggttcccacttggggaaacagccagcccctttctgagcagagggtgtggcactacaaaggcgaaacatgatctctgggagaaacatgcttgaagcccacgctcctgggggcagagctgagcctccacacggatgtccccagcactgggagcagaggctgatgcggccccaaggtgctgaggaaagcgccccccagtgcaaaggatgctacaaggccccaggcttctcaaggaacatgaggcctggcaaacccagaacgttagactgggtcggctccaagatgcccctgggtttgagctggagaagtgtctgcgggagggtgctggggctcctgcagagcgtgtcttcctgaccctgtgggcttttctggggggggctaggaggctctgccaagttggtggatggaactgtgccttgccctggggggcactgggcaggagccagcctcggagcgtgagtctggtgctgcgccaggaggctagatgccccggccaggtcctgtgggctgcgccgtctgtgcccttggcccgcgaggctggcaggctgccactgcttgtaaagcctcctggttgaaaaggtgactcggagcagcggattcactgggagcctgtcagccacagaggcagcttgtgcttgtgccccaggcccaggccgcaggaggtgggatcaagttccacggctccagtgtgaaggtctgagcttcattctaaggcaggtttttgaggacatggggctctcgggcacctggttgtattcctgcatcctgcattcctgcccactctctcatgccttcttctccttctccttggcgtgccccaagccagccttccatctcagatggagttgtatgtttctagtgagcccacacaccccctgaggacagggcctcacccccgcacgcctgcttcttcatcacagctgctaggaaatgaagatgcatgtccccaggggcctttgaagtaggatgtggtctccctgagcagaactctgactctaaattctctcctggctctggcgttggtgtgggacttgggttgcgggagggggcaggttgttagcggctgctgctcagacccagcggaggaccgggctgctccaggctcccggcaagatgtagcaaaatggccaaatttaaggtgagcacgggctcccgtgcagtttctcacccctgctgctggctgcctgtctgtgaatgctaaggcgaggggtggttctacattgacttcttactgactggtgggagtaacccggaagaggcaaatgacttccttccctttcctcggctctgaacctagaatgttccagtgataccacctccaaactcttcacctgtgtcctttcccacctctctgccctgaggacagggaactaagtggctgctcacggtgggtaaaactgaggcttacaagacaccttgggcaatgggacagccagaccccagccctcagcacctgcctctcccatcccaagccccagtctctttttcagctgcaaagtcaatacctttgctttcagagagggagcatgtgccacgccaggagaggctttgggtgtatcacagatggagactgcctagcttaactgcgcagtctgaatgattctaaggaggggaagagagaacgcctctggagcctggcacacacatttgccggagaaagtggtgagtgcgttgtgaccaggtgagggacgaaccttaaggagattggctgagaacacctcccctcctcaccctaagtcaacaaacccgggacagcagtgcttccaaaaccttacttgttcacctcacctggagattcttgtggcaaatcgggttctgcctcgttggctctggggtgggccttgagagcctgccgttctaacaagctcccaggtgatgctgatccacgaccagctttgagtaccaggatctcgcatcagcttgggagggaggagacctgaatcctagccctggctccttaggctcttctttccagtgggggaatttagttaattgctccgggccccaatatcttaatctgaaaacaaatgaatgacaacctagttgcctttcctactctgagattccatgcgtcttaaaagaaaaaagttttaaaactaaacatgtcactgtcaccatcgctacttcacacacctaaagttataaaccactcagaaatgcaaatcttaggagatgtgatctggcaaggcaaaatcgaagatggctttgaggtctcagctaaacctagaaatctgatgctctgcattttttatgggatgcttatgaattaatgaaaatacatgagattatctctccttgagtctaccctttcctacaaccagcaaaaacttataaaaagcaattgaagcttactttgggaatgccaagggattgattaaaatatggactgtgctgattccatggctggtttagcagattcttgttaaaagccatgactttttaaaatttttatttttttgtgggtacatagtaggtatatatatttatgggatacatgagatattttgatacaggcatgtaatatgtaataaccacatcatggaaaatggggcatccatcccctcacacatttatcttttgtgttacaaaccaattatactcttttagttattttaaaatgtacagttaaattatatagtcaccatgttgtgctatcaagtaccagttcttattcattccttttatttttatttgttttactcattaaccatccccacctcctccctagacccccgccgctacccttcctagcctctggtaaccatccttctattctctatgtccatgaattcaattttgatttttagatcccacaaataagtgagaatatgcaatgtttgtctttctgtgcctggcttgtttcttttttttttttttttttttttccttttttttgagacagagtcttgctctgtcacacaggctatagtgcagtggtgcgatctcggctcactgcaacctccgccttctgggttcaacctcccaagtagctgggattacaggcacccaccaccacacccagctaatttttgtatttttagtagagacagggtttcaccatgttgtccaggctggtctcgaactcctgacctctggtgatccacccgcctcggcctcccaaagtgctgggattacaggtgtgagcaggtgtgagcaactgcactcggctggctggcttgtttcacttaacataatgacctccagttccatccatgttgttgcagataacaggatctcattcttttttatggataaatagtactccactgtgtatatgtaccacattttctttatccattcatctgttggtggacactgaagttgcttccaaatcttggctattgtgaacagtgctgcaactaacatgggagtgcagacatctgtttgatatactgatttcctttcttttgggtagatacccagcagtgggattgctggatcatatgatagctctacttttagttttttgaggaacctccaaactgttctccatagtggttgtacgcatatagtacatatagtacataacatatataacatatgtatactacataacgtatatatagtatataacatatatagtggttgtactaatttacattcccaccaactgtgtacaagggtttccttttctccacatccttgccagcatttgttattgcctgacttttggatgtaagccattgtaattggggtaagatgatctctcagtgtagttttgatttgcctttttctgacgatcagtgatgttgggcaccttttcatatgcctgtttaccatttgtatttcttcttttgagaaatgtacattcaaatcttttgctcattttttggatcggattattagatttttttcctgtagaattgtttgagctccttatatattctggttattagtcccttgtcagatgagtagtttgcaaatatttcctcccattctgtgggctgtctcttcactttgttgattgtttcctttgctgtgcaaaagctttttaacttaatatgatctcttttgttcatttttgctttggttgcctgtgcttgtggggtattgctcaatacatttttgcccaggtcaatgtcctggaggtctttcccaatgttttactgtagtagcttcatagtttgagggccatggcattcttaaaaacataactttcattcttgcatagctttttttttttttttttttttttttaagtaacctggactgtcatttggctagggaggcgggagtttgtgtgtctggaaatgtgtgggtttttttttttttttttttttttttttttttttttgggacggagtctcgctctgtctcccaggctagagggcagtgcagctacctcggctcactgcaagctccgcctccctggttcacaccattctcctgcctcagcctcccgagtagctgggactacaggcgcccgccaccacccctggctaattttttgtattttttagtagagacggggtttcaccatgttagtcaggatggtctcgatctcctgacctcttgatccacctgcctcggacttcaaaagtgctgggattacattacaggtgtgagccaccacacccagccagtgtgtggtcttttttatctgtctgtttaaactgtctgttttatatgttgcatgaaccccgctttaccatgaatcttatcaatgactgataaatattgtctattggaaacattctctttaagaggaggtgaccgttctctgcagctcctgctgattcagcaaagctgaggtcaggtggagccagctgggcagagatgagtgattagaaggctgtcattggcagtgacagtccacgtgctccagggcagaacagtgacactggctgtcacagtctgtttcaacaatggaggttgtttgtttttatttaaatgatgtgaataaagaaaggaatctttgaaaactaaggctggagctgcccaggcctgagccaggcccttgtaacaaggcccttctgggtgggggatctacctctcttttcacctggaccacctggcggtgggtggcgagctcagaaccacagctgggtggaaaatgggagggccagcagcgagctgcaggaatggaattccatgatggcatctcatgtctccagagtggacagacagaagcttgtgttttcatttcaaagggcatctgccacgccctccaggcagaccacataaatggcatttttgggtgaggcctgagggattctgtctggtggtatacactgattttaaccagaagcaatagatttgggctctttttgacaggggaagatagatgagagatgatttccatgaggatgttgaaaatattggcaattctgaagaccccagggagtcccagggggttcctggccaggagcgcccctccattgccctgcaccaccttgctttccaggctgtggctctgtgtgtctgaatctacggcatgggggcacttccttctaattcacagaagaagctatatgggccccagactgcccatcctgctaaaactggcaaaggccttttcccagcctgagcagtagtgattcagccaactgcagaagtggtatttggagctcacccttaaaacataaagcaagttatgttcagactatatacaggcaatgagaaaatatgatgaaatatgactgcgtatgaatagccccagacttctagggcctttgagtgtgtctgtcttgggcaacatcttctccttttagatttcaggctcagtttactgtcaggaaaattaagttgctgagagcgtctcagaattttggcttaaaagagaatatacacctggaacaaagacttgaggggtgctcctattaaaaaaaacaaacagtgtgtgagcttcaaaggagcaccttgaaactccagttccatcacatccactctgctgggccctcctagggcagctctctgtcttgtgcctgccttctcttctgaacactcctaaggtattttgagtgcggaggataggatcatacactcaggaaagccgagagctagatcctgaagcatctgtgcgggaaatgcccatctgtgcctgaaatgagcatctgcgagtgaagtgatcccatgtctgggatctgtttggcatgcgccaggagacagacggacacatgcatgcacacacacacacagcggggacggggagttggtgaaataagagtgggaaaatgttgacacctccgtgagccgggtgacaagctgcttggtggttcactagacagggagttataaattggccgcttttcaacatcgacagataaaaatcaatagccttcccataatgccatcattaatcaattagagtctgtaatgggaaaagcagatgccactcccccacagtttctgggaataaacaaagtgtataaggcctaaaagaagaaagctatagaacttgactgaagcccacatctgtggaaaggcatactatgttcttggagtgggaacatcaacactatagagatggcagttcctctccagttaatctggaagtgcaagtatcgatgagtaaatccaattaaattccaacaagagcctggtctaaaatgtaacaggctgattttacagtatcgctggactccccctacatccaaacttattcttaccttcattctcccattttggttaatggcagcaccatccaggcaactgctggtaataaaagcctgtgagtcattctgacgccccttccttcctcagcctcatccaacctaccagccagtagtgaatcccaccccaaaacagatggtgaatcccaccgagtcccccggtcatcgctgctacatcgagagtgcagccactgccccatgtgctttcctggtctgcttggcagcctttgaactagtctcctggctcttcccctgctccccgtatagggatctgcttgaagctcacacagccctgatcctctgcccctgcctctttctccaccctcatctcccaccatttcctgggctcactgcattcagcagcctgacttgttcctgctcagaatcttcacacccacctgcatgcactccctcccccaggtgatatggtttggatctatgtccccacccgaagtcatgttgaaatgtgatccccagtgctggagatggggcccagtgggaggtgattgggccatggtggtggtttctcatgaatggtcagcaccatcccccctggggttatcattgaaacagtgagttgccacaagatctgattgtttaaaagtgtgtggcacccaccccaccctcttggtcctgctcctgccatgtaagttgcctattcccactttgccttccaccatgagtaaaagctccccgaggcctcccaagaagcagatgctgccatgcttcccgtatagcctacagatccatgagccaattaaatctcttttctttataaattacccagtctcaggcatttctttatagcagtgcgagaacagactagtacaccaagggttggcctggctgctgctgcctccgtctccccacctaccccacccccccccacccagcttcgtgactgcagtaagaccttcctgacctctgtggctggaataccctgcagcccccacacaccaactgtctctgccccatgcctggtttcctttcttcttggcgcttatcactgaaattaccttatgtgtgtgcttacgtgggtgtcttctgttgtccactgcaaaaatgcattgtgggttgaattgtgtgccccagaaaaatacattgctgtcctaacccgggataccttgcaatcttatttggaaatagggtctttgcagatgcaatgaagatgtaagttaagatgggatcgtactggtatagggtgggcctttaatccaatgtgactggcgtgcttataagaagaaaagggacacaggcagacgcacgcagaggggagaatgtcatgtgaaggcatacagggaagacagccatatgacaatggaggcagagatgggagtgacactgccgcaagacaaggaatgcctgggctaccggaagctgggaaaagcaagggaagatgctcctacccacgccttgatcttggatttctggcctccagaactgtgagacaataaatttgctgttgtttcaagccccccgttctgtggttctttgttacagcaacccaggcaactaatacaggggcattgtgtatctcgttcagtaccttaccttaacactgcctcgtgtggaaatgaggcttggcaaacactgttgaatgacgaagcattggcaaaaaaaatagccaagattttgaaaaggatccacaaaggcagactgatttaccaggtagcacaacatattataaagccctaaccatcgtggaggctgtcagcacaaatcaacacagtagagtggaaggctcagaacagacttgtgtgagcagtgttactttgtttcttataaaggtgatgacttcagtcggtcagagaaaggtaagggtggatactcagcacctgctgttggtgtggttgatcagtgcagtgcctaagaggcccctgctctattcaactggatattcacgcaggcattcttggccggtctgctcagagaggaataggctggaaaccttggctggccagcaggatgctgcctgctgctctgcttccaggggagcgaggggtcttcctggcagtttttgcaggtggctgcacacacaggtgtccttctggtcactagctcctcccccagctgaaagccctgggtccccaaggagccagaatcccttataaaattaccagccttacagagccctgggttcacacaggggacattccatgttacaagagttaccacacctaaggaagccccatgcctggcttcttcaccgagtggttataattcacttatgggtcttccctgtccagaggcaatttgccaatcaaggtcatttttaccatgagcaatgttgtctagtaacagagttgacgttccatcttgattggggttcccagggaaccagagctagataattaaacattaaattctcatacagaagggaagaaagattttgctaagcctgtgaccaaaactatccatatggacactaagtgagagtcctatgttttatcatacattaaaataaatgcagaggtatttaagtgtaaaaaaacaactttcaaagtattagaaaatataggctatgctttcacagtgttacggcatggaaagatgttctaaggaaagcatcaaactcagaagtcataaaggaaataaacctgacagatatgggaaatatttttattgaagtgtaacatacatagagaaaaatgaacactcgaagcgttgagctccatgaatcttcacccggtgaacacacccacatagccagcacgcagatctggagagagaacgtttgcatcccaggcccctgcctcccattccccctgtgtccgaatgatagatttgactacataaaaatggaaaactccaatatatcaaaagacaccataaacgtagtgaaaagacaagcaacagcccagaaggaaagtttgtgcagccttttgtaacatagaaaaaggccaaagatggatgatcatagcatataaaagaactcctatagatcatgaagaaggaaggacaaatagttcaacagagaaattggcaaaggatataatgagcaattctccaaaggaaaaatacaaatggccaaaacaatggagccagtgtggccggctctgcccagcacggccatttgcacatggtcactgttgggtgggtgagggctgcttgccccgtctccagccccactctcttgccatctccccctgtgattcaggcatggagctcaggccccaggtggcaggcagcgcactctgctcagttaccccagctcctggcttcatggccatgccctggcacctgtctccctccccttagcctgctgacctttttcccatgggatgtttggttgtctctgtacaggctctgagccagccctatgggcttccagacaagcccagattgagcctgccctctagggagcacccctgtgtagcttccagctgagaccttgagacacaggcccagggagccaagctcagggctgcgaacatccaggtggggctcaggtgctagaggcggacaacagcctggtgcaggacccagagtggcaagaggtagtaagggggccatggtgttcagaaattggggcaggtggtgggtaaggcatcctgcttctaaaggccagggtttgggagggcaaggaagcccctggcaggagtcagggcaggcatcccgggcaggtagtgtttgttctgtgaagcaggcttccccaaggaggggccctagagcagaaagctgtgatgtgcaggtgactgggagacctgttctctttcgagccgtgcagctccggttagtggatgacagatttcaagccctgctcaggccagtgtttttcacacatgtttgacctttggttgacagcacacagcacaacagcttatggtccaggagtgcacctcggcatgcagtttgggaatcctggcgatgatgtgattaagattctggggccaggttctcaccaagccaaaaggctttttttttttttttttttttttttttttttttttttttgaggcaggagctaattctaggtacagaaaaccattggtaaagtggaggaagctgtttccagatccacggaatgacggtaactagcgtcacttgagaaggtttcttctgcccttttgtaaaaaatagcatgatgttttctcctagagacagacagggcctctgtacgtaagagcagacatgtatggagaaggccggagagtcacaaaccatatgtgcctgagatgtgccacctgtggggctgtgaataaggaagcagaatcagtgtgtttactcagagctggatttggctgtttgttccttcatctttcttggaccacatgtcctggtgttcagaggctcctgagagcctggggctcagtaggccgtctcatagcccctctaacccaggtgagaaaacaggtcccagtgggtccctaacccatgctcattttctcaccaaacaacaacaaataaatcagaaagtcctgaaaaatgaattctggcctgttttctggattctggattgaaatatggagtaggagctgacttttagtaagttgatcaactctccccgccgaccccctagccttctggaactgtcttcagtaaacacaacccccgccacccctgacctacccgcaccgcactgtggtcggcctcttctttgaacacttcccttacagcacgcactccccccaagctgggccttctcttgagaccactctgctgtccttgggagtgatggtggaggcggggtagacaccctgtccctgtctcgtggtgctcctttgggccacagctcctcttccctccgtaggaggaaccatctggaatccatcttcctccttgaccacacctctccattcattaaggagcagagttccctgctcactgggtctctctacccctcctctgtccctgtctgggtgaggtcacagagagcctcacccgcctgctctgttggcctctcacttgccgtcctctctgccccgttccctctcctgcacacctgtccgcggtatccccagctcttagtgactctagccctctcccgccctcctctgcaccctgcatgcacttgtgttgctgaatgtggccggagaaaaacacagccatcttgactgttctgtgagacttcatagatttccagggctgtgtggaatcaaaaaatcttcctgcatttccccagccatctggcctccaggatgactatttcacatctcctcaagcctttcacatctcatccccctcacacctgatgattttgcttcctatttcactgggaaaatgaaggtaactgagaactttcttttttttttttttttttttttgagatggagcctggctcttgttccccaggctggagtgcaatggcacgatctcggctcactgcaacctccacctcccgggttcaagcgatttctcctgcctcagcctcccaagtagctgggactacaggcgcccgccaccacgcccggctaattgtcttgtatttttagtagggacggggtttcatcatgttggccaggctggtctcgatcttctgacctcgtgagccgcccacctcggcctcccaaagtgctgggattacaggcatgagccaccgcgcccagccaaaggtagctaagaactttcaacagcccccacacctactccctggctctggtacagtggacagcctcccaggcttgtcccccctcccagtgaccttgacccctctcatcttttaagacctcgctcccgcagtttccccctttccttcctaactcggtcatttgcaccattataccaacaggctgtcttagctcacattaagaaaccctctcttgaccctggcctccacctcatctcttggttttcccatgtgacgaggttgtctacacaccttccctcttcccagtctcccttgaagtgactccagccaggctgtgtcctcccactcttctgaaacctttcctcaaggcctccaggaccttctggctgctcagtcttccatccaacagcatcccacagatgaagcattttcttcgcttggcttgtcttacgctctgttctcatcatattgcttcctcactgcctgtttttctccagagcctagaaccatgcctggcacacagtgggcatctagaccctttgctgagtggtgaaattgaagtgtgacagggtgcgtggcttccctgctcaggctccctcagttcccaaagctgtgctccttcctggatctcatgtagcacaaggaggcatacgtgggtcgcaccacatggcttctagaggagtatgtgtgaatgtgttcatgttcatgtggaaatagggaggcggccctgaggtgggaggagccttcccaagaggaatcacctgagagaggaagaagagctggggaggaactgaggaactggtttggcaggttcaggaagaagtgagtgaagaaactggaaaagtagccaggatcagaacctgcagccgcaggacctaatttgttaggacctaaattaggggtgattgtgcaaggcagtgcagcatggaagccccactccaggacttccctggtggctaggaaacattgctccacccctccctcttgtcaaaagagaaggtgcacacacttggagagaaatgggtatttttgtgcttctttcttgttccctgttgggtaggagggttggggctaccaggcccagtgaaaacacagcccagccctgcctgttctgcctcatcctgaagtgtaaggaactgcccattgtgctgggccctgtcactggctctgcccgaggcatatgtggcactgggcctggctttttaaggtgcaaactgttttcctggatttcttatttttaagggaaaaaactcaactacaaagactcagtggccagtcctgtgctcaagccctcttgttaggggagctggcctccctctttggccacagctcagtgcagggaggggtgtggaaagtgctgggaaagggggaggacccaccctccccacagatgggtttctggctctgactgcgggaaaccaaactttcttgtattttctctggctgggttgtgatgtgggcatggcgttggtgggaggtagtgcatttgctgagggtgatgtgttctgagtgtcccctcactccgtggctctttcatgtggcctggactttttggtgagggagcagcagtgggaggacaggagagtcctttagcgggctgatgtgtgtctgccagttggctttgtcttggctcgcagcttctgtttcagacagatgttttgatccccctttgcttagcaaccagctgcagccttgctctgtcttgggaaaaccactgtctttctgaagagctgcttcagctccaaaactgataactctggaaggagcagccaccagccagtcagaactgagctccgaattaacacatgaaggagactttggcctcttgagaccctgaaatacaagaagatggtttcatgtcatgtccaagaatgtggccacaaatgctacctcagtctcgggctgtctgcattgttaaatgacctcattatttttctgacctgtgggccgggcttcgttggcatttccttaaggcgtgttattgtggaaaatattattcaagaaagaagagcatgtggtttgcttggttactgagctggactggctctgggtgaccagagctctcacgcagagccgtgcacggctgctcccactgagaccagcctctgcctcgggatggtctgacccacggagctctgttttgggacccagaatccatgagccacatttgctgtgctgtgggacaaaccacagtttccttgggagcctctcaagtggtggctgtttctcttccacacccttggaccactggagctggagacggggagttccccagcctcctccaaggataagcctattccttaagcctccacaagccccatcattatttatttttatcataagccatgttgttcgagtagcatagacaactttctcaggttaccagggaaacaggactagaaagtcaacccacgatcaaattaccttgccgaaagggaaccagctcagttaacccttgaccccacacgccattgtcagaggcgtatgaaccagagcgactccatcttgaataggggctgggtcaaatggatctgagacctgctgggctgcattcccaggaggttaggcattcttagccacaggatgagataagaggtcagcacaagatacaggtcataaaggccttactgctaaaacaggttgtggtacagaagccggccaaaacccaccaaaaccaagatggcaatgaaagtgacgtctggccatactcactgctcattatatgctaattataatgtattagcatgctaagagacactcgcaccagcaccatgacagtttacaaatgccatggcaaaatcaggaagttaccctatatcctctaaaaaggggaagaaccctcagttcctgggaattgcctacccctttcccagaaatgaataatccaccccttatttagcatataatccagaaataatcataaaaatgggcaaccagcagctcatactgctgccctgcctatagagtagccattcttttattcctttactttcctaataaacttgctttcacttcatggacttgctctgaattctctcttgcatgagatctaagaactctctctcggggtctggatcgggacccctttccagtaacaccacgagactgcgtccctcactgcccctcgtttcggcagttgcatggttccttggtgagttttccctctttctcgctgtcatttctcactatgagtctggcgcttgttcttggtgtcttcagtggccacgtcgttgatccttcggaggaccttcgccccctctcctccagtgagtgaccccacccctgcagccactcgctcccatgccacgcccctgacccctccagcatctgtctcaaacatcccactcttctttctccactttcaccttgtcactcatcctgcaattcccagtcccagtgactattttttttttttttttttgagacagtcttgctctgtcgcccaggctggagtgcagtggcgcgatctcggctcactgcaagctccgcctcccaggttcacgccattctcctgcctcagcctccctagtagctgggactacaggtgcccgcaacgacgcccggctaattttttgtatttttaatagagatggggtttcaccatgttagccaggatggtctcaatctcctgacctcgtgatccacccaccttggccttccaaagtgctgggattagaggcgtgagccaccacgcccggcccccagtgatctttgaccccactgggatggcgacccttagaacccatggccttctcatagcccctcgtctgtctcacaccttccctctttgccatgtcagtgaccagccctgccttgcactcaccctccccacccgcccttctcttgctgggtcctccccacctggtgaaacctcagccctggttacatcctgcactccacgcctgcaccagccgctgtgcagtagaagtttctggagatgccccactaccacacgggcagagcgtaccatcaggtcacaacccctgagccaaggtgggcctttcacttgcgtttctgtgctctgtccactcccccaccctcccagatggcaactccaccctcccctttcctcagctctgctgtgcctcctccccatcctcactttcagctgatggcctcgtcctctcattccaccccaaaatttgaagccactggaagagaattcccgctggctccctgcaccaccactacccgcccaccatgtctgtcccccttcccacctgctccccgcagtgcactgtctacgtttcaggccagtgctacctgtgtgcagatgcccatggcctgctccagggcatggctttcggagctccctgctcctgccacatccgtcctgacctctctcttggctgcattggtcctgacctctctctcagctgcatcaatcctcgcctctctcctggctcatttctgccagcacaaaacttggtataaatgcccctggcctaaggaaatcttttgacccactttcctctccagcagcctctccatttctctgcttgccacacgcacacatgcacatatgcacacgcacacacatgcgcacacacgtatgcacacacgcacgatccatgcacacaatgcatgcacacacaggaaattcctcagagaatcatctgttgccactgtgtccattttctctctccctccccattcaggtttttgcccctcaactttactgaaactgttcctctctgagctgccaaagtctgactaaagtgacaaattctcagtcctcaaattatagatgcagaagcagcctctgacctgctgactgctcttgcttccttgaaacgctttcttctcctggccgccaggatgccacgcactgctgggttttcttgtatcttcttggtcacttctcagtccaccctgaccttcaaacatgctgcacccagaagtcggtgccctccactcactccctaggggatcccctccaacctcatgattattttattttattgctgggattacaggtgcacgccacactcccagctaatttttgttgttgtttgagacggagtctcactctgtcgccctggctggagtgcagtggtgcgatctcggctcactgcaaccttcgcctcctgggttcaagtgattctcctgcctcagcctcccaagtagctgtgattacaggcgcatgccaccacacccagctaaattttgtatttttagtggaggtggggtttcaccatgctggccaggctggtctcgaactcctaaacctcaggtgatgcactcacctcagcctcccaaagtgctgggattacaggcatgagccatggcaccgatctgtttatttttgagacagtgtctcactctgtcaaccaggctggattgcagtggcgcaatcatggctaactgcagcctccacctcccaggttcagatgatcctctcacctcagcctcccaagaagctgagaccacaagtgtgcacaccttgcccgactaatattttttattatatttttagagatggagtctccctatgttgtccaggttgttctcgaactcctgagctcaagtgatcctcccatctcagcctcccaaagtgctgggattagaggcaggagcccccacacccggccccatgattgtaaataactctgtctgctgggacgcctgggggatctttccaaccagatttctctgctggactccagacttgcagagctctgtcgtcagcctccacgcagggctgcaacagagccacggcatgtctggtcccagcagatgctctgcccagacctgtcctctcactctttgctgtcacgccagggccactccatcccttccattgttcaggcaatgaacatggcatcatccttgaaccctgtctttctctcatatcccataaccaatctgtccatggttccaccttcaaaatatacccagatatgtgatgatgttgaggaattgctggtaacttttttagatgtgataatggtattgtggttgtgttttgtttttgtttttgttttagacggagtttcgctcttgtcacgcaggctggagtgcagtggcgtgaccatggctccctgcaacctccgcctcccaggtttaagcaattttcctgcctcagcctcctgagtagctggagttacaggcatgcaccacctcacctggctaatttttatatttttagtagagactaaaaacagggtagtggtgtgatcacagctcactgtagtcttgacctcttgggctcaagcaatcctcctgtctcagcctcccaaatagctgggaccacaggctcccaccaccacgcctggctaatttctttaaaaatttttatagggatggagtctcactgtgttgcccagtcttgtcttgaactcctgggctcaaccagtcctcctgccctagcctcctaaagtgctgggattacagggagggagataccatgcctagctagtttcatttgttatctgcctggtgtgtgtagacatctgctttgtcacctccattgtggagagcaacatggggtttcaccatgttggccaggctggtcttgaactcctgacctcaggtgatccattccccttggcctcccaaagtgctgggattacaggcatgagccaccacgtctggccatggttgtgttttttaaaagagctcttatcttttccagagtcttactgaaatatttacagatgaaataatgtgatgcctggaatttactcacaataacctagggcagtgcttctcaagctttggcatgcattcaagtcccctggagggctcattaaaacagctttctaggtcttgagcccagagcttctgattcacaaggcctggggtgggccccgtaacttgtatttctaatgcttggtgctgctggtgtgaggagcccattttgagttgagctgatctaaggtataggagagtgagtagcctgggcgtgggctgggagaaggtggagttggtgggtacgtggtaatgaaggtacaagggtttattatgtcaccctacttttctttggaaagttccgcaactaaaaagcacgtgtgtatgtgtgtctgtctgtctgtctgtctccagaactcttcagctcttccattgctgcgccctggctcaggctagagtcctctcttgcccaggtggcgctagcacctcctaactggtctccttccttctctccttgttcctctagagtcagttctcaacagagcagccaggatgatccccagacccctgcagtggcattcagagtgacacccaaaacctttctgtgacctcaaacttctgtgcagtgtgttccccacgccacttcctccatgaaccttcagtcacactgcccgcctcgctggggcttgcactggtcaggagtgcttctgccgcaggacctttgcacgtgcccttctgggcctacagtgctcctccactctccatgcagccatctccctggcttccttcaggtccgcattcagatgtcacccttgcaatgaggccttctcagacaacaccatctaaaatgtcacttccccttccccactattcataccctactccctgctcactttttctgctccgcgcctgtcactgtctatattgcactgattgatcttatctatcctggcaggaagcttgagcagcgcccgcctcttcctggctgtgcctcggcagtgcccccaccccagcctcccaccccatcttggtctgcaccccctgccatcactcatccattcattcctcctgttcctcttggccactgccaaatgctggatggtttccatctgtttggttcactgcagtgtctctgtccctggaagggtgccagccacagaaggcacgaagatgtgtccacagagccggcggctaaaaatctggcaagaggagtcgaggaagggggctgaggaggtagaagggagggctttcctcaaaaggccagaaattcgtggccagccaaaggggaagcaaagacattctcgtgtaggaaaacttacaggaaatgcaactaaaagcgtgggagacaggaagtctgtgtatttctgagattatcaagaaaggggggatggaagttgcaaatgggcaggccaaattcagtctgcagaggtgtgcttccagcccacgcagtgcttaaaataaatggagatggttgccagcactggaaggtctggagtgccctgtctcctggaaacactgcagcctggcaccactgggctggtgagcatgactggcacctgcagcagctgcccaccagctaatggcaggtgtgtgttctctgctcacccaaagcccttccagcagcttcattttgttgttgttgtttatttgtttttgagacagggtagtggtacaatcacagctcactgcagtcttgacctcctgggctcaagcaatcctgctatctcagcctcccaagtagctgggaccacaggttcccaccaccatgcctggttactttttaaaagaatttttgtagggatggagtctccctgtgttgcccaggctggtcttgaactcctgggctgaagcaatcctcctgccttggcctcccaaagtgctaggattacaggtgcgagccaccatgcctggctagcttcatttgctaccttcctggtgtgtgtagacatctgtgttgtcacccccattttggaaagcaaaaaggatttcttcttccttcaaacataattgagaaaatacagttgtttgggggcaagtagggtgcggggtgactctggaaccggcctcctgggaagccaggcctctgggagtgctggaggtgggcagtgaggctgccaggcccctgccttctggggtggggtgcatgctcctgatacatcagaagggtcgggggaggacttggaggtgaaggcactgcaggaagagaggaagcttactggagagacggtcaatggggcagtcaggctttaaaaggacgggcccaccaagtgaaaagatgaccaagtgtttgggctgaattgtgtcctccaaaaggatatgttgaagcctaacccccgggacctcagaaggggatcttatttggagatagggttgttgcagttgtaacgctgagatgagattatactggagtagggtgggcccttaatccagtgtgactgctgttctaagaagatgtacatgtgacaatggagggacatgtggggagaggatggccacgtgatgatgggggggacatgttgggagaggatggccacgtgacaacagggggatgtcaggagaggatggcctcgtgacaacgggggaatgccaaaagaggatggcctcgtgatgacaggggacacatggggagaggatggccttgggacgacaggacatgtggggaggggatggccacgtgatgatgggaggacatgtggggagagaatggccacataatggtggggggacacatggagagagaatggccacataatggtggggggacacgtgaggagaggatggccacatgacaattgggggacaccacgcggggagaggatgaccacataacaacgaggggacatattgggagagaattgccacgcgacgacggaggaagggactggagtgaggaagtcataagccaaggaacgccaaggacagaacaagcagcaaacacaagacacagggccaggtaaggaggtccccggctccaggtatcagaggagccctgctgatgcctggatttcagctctctggcctctggacccagaagagaatgaagttctgctgcctggggcctcccagtttgtttacagtgagtctgttcatggtggagaaacatcttctcatgatgttcctgtccaccatggtcaaggcctgttttgagctcctgcattgtataagtcagaggtaaagctgtttttctcaaggggtcaataattgacagtattggaatgggacaaggtggtcctgttgggttttgtttgtttgtttttaaaatatgaggatagaaagtcgtgtttttacttgtgcttaattttcccagtggccgggatgtgaagtccacgcctgtcatctgtaatgcaccagggcaaggggtcggttttgtttgcatagcttctcccagggtcatcttcccacccctcaagtgtggatttggccacaacactttgtttagcaatttcctgtcattttgttttatttggtcaggcttatttgcatagttgaatgaatgcacaaaggcctccttgtttcgtggttcacaaagtcagaactggatcataccagggcatacgggttctgtttcaaggctttgtaaagctgctagggttgaaataggtatttttggtgtcggaattattttattgcatttatcagtttatctactgagatggggcactgagaactgctgatttacctctctgcagccctgtctgcagtacctgaaggctgggggaagacatttggcaatgcacacagcctttgtttctgcctgtgaagttggggtttcataggccacctcccaggagggccctgtaggtgcatgacagagtccgagtgtctcaccagtgggtttgtcattgcacccaagcagatgctgggggagaggatgcctcttccttctcctcacccttcatcagtgtgtgagcagaggcaggagacctgtgggctggaaggcccacatcctccttcccaggctgtgaatgtggcacttacatggcaggaggactttgcagatgtgatcaggaccttgagaggagacattatcctggatgatctggatgagtccatcgcactcacagggtccttaaaagctggaagagggaggccaggcagggagagggaaggaaatgtgaggatggaagagggatccgattgccgcgattgctgaatttgaagaggaaggaaggagccgagagccaaggaatccaggcagcctctgggagctgcaagagacaaggaaatggattctgtcctgcagccttcggagcagatgcagacctgccgacaccttcctttcagctcagcgaaatccatttcagaattttgagtgcagagctgtaaggtcatgagtctgtgctgcttaagccactgtgtttgtagtcatttgttgcggtagtaaaggagactaatatgctattttgccaccattctgagcttttgaggcaggctgatggaaaaggtgggacagtggcttcccagtacttttctaagtgatcctgagaacctaatatcccaggcacttcatattctgtaactctccaccctgctgccagcagagtctgcttagtcctcaagacgaggggcttctgctgctctcagaggttgatgatttcattgattatctttaagttactctgcactctcccttccccagagaaaacccagtcaaattcaagtgagcattggggaaaatattcattcattaaactggttcttaacctttttgggggcaccgacctcttgagattctgttgaaaatgggattttagcccccataaaaatacatatcaaaaatttgcataaattttggccaggcacagtggctcatgcctgtaatctcagcactttggaaggccgagacgggcagatcacttgaggtcgggagttcaagaccagcctggcaacatggcgaaatgccgtccctactaaaactacaaaaaatagctgggtatggtggcgcatgcctgtaatcccagctgcttgagtggctgaggtggaagaatagcttgaacccaggaggtggaggttacagtgagctgagattgcaccattgtactccagcctgggcaacacagcgagactgtcaaaaaaaaaattgcatacaattttgagaacaattttgtatacagattcccccaggagctaccccttgaagctccaatggctgccaggggcttcctctgtcaccccaggtgaagagcccttgtacagattaattcacattaaggctgatgaatggctgttttcctagtaagtctcctcgctgtttgccaggtccttaaaccatgttcagtggatcctcattatttgtggattccatatttgtaaattcatctgctctctaaaatttgtttgtcaccccaagatcagaactcatagacaccagcagagtggcaaaaatgtgttacccgatgagccttttcccagctgaaaaggatgctctgccatcttgtttcagcctcttggtgtaaacgagtgtggttttcatgatttgtttagtgccatgttttccatgttttgtgctttttcttcgtggtttcactgtttgaaagtgctgtctagtgtttccaagtgcaagaaggcagtgatgtgccttatggggaaaatatgtgtgtttgataaactgtattcaggtttgtgttctagtgccactggttgagtgttaacgaatcaacactgtacagatgattcctgacttacgatggttccttccagtatgatttttcaacttaacaacggtgtgaaagtgatatgcattcagtagaaaccatctttcaaatacccatgggaccattctgtttttcactttctgttcaatagtctatacatttcataagatattcaacactctttcagaaagtaggctttgtatttcatgatttcgcccagctgtaggctaacatgtgttctggataggtggaaggcaagctgagacatacctctatggtgtttggtaggttaggtgtattgaatgcattgtcaacttaggatattttcaacctatcctgggtttatcaggacatgaccccattgtaagttgaggaccatctgtttattcagtaaggtgtcttttaaatagaaaaacacataaaacaaggttagtattgattgattgacaaaaatgttgcaacctgaggctttcaggaacctaactctgcatttcctttaggagtgctgcttgcttcagtatctgctggtaactctatggaacatagctactgtgaataatgggaattgggtgtattttcaagttgttggcctctccagctacgctggtaatcagtcctgctctttaaatcacatcttaccatcagtcctgcctagagaaatggaggaagggaggtctctggtcttacggtctgtcccgactctctgccccaccagctatgtcccgagaaccagcctggacagagccatctgattggtgggtggtagcaggtcatgttgtgaggccctggtgcctgccttctgtcctcctgtgctcgctagaggagcatgaggcaggagcagccagtccatgctggttcctgagcgaggcagctctagggctgccagcacccactcctggggggctgagggtaggaagagcttcagaggggaggatgggactgtctgactatatttccaatcccagttgggacttgtggtctgatgaggacaagtgcctttgagggggatggggatggttgagaggaaccaggaaatcaaatgtgtcctggaaaacatggcatggtggatcttttgagttggggtgttgggatgtgtgtcagctaaaacaaggttcacctgcttcaaatgttcatccaagctttctacacttgcctctctgagtcaacatctttagagacagcaggaagtgatgaaagtcagttcacagtccatctgctctgagggcttggtatgaggggctgatgttttctgtcattttctcctttctaacatttcactttgggatcttagttcctggttgaaattccttaactgtgggagtcgaggcagcctaagaatccttttcggctgcttaagacaatgaggtagagagggaaccagctgccccttttcttacctccatgatgatgtagaaatggttccatccagcccaagggttcttctggggcccaaagctccttaagggctctgtgcataggatttgaggattagttttcttgggtggggagaaaagtccatctttatttgtataaatcctacctgaaatgcagcatttcctctaattaggaaggcaggcaacaaaaccagtatgttagctatgtctgtgactctgtcaccaatgtgggttagccgtatctctgaccctgtcaccaattgcaattacagatgtctccacatcacattagtgttgtttcagatatcttgaagtagcatttgttcccattactagttttaaattaaggtggttaatagaccccactggattttgcgatttaatatgctgataaggcacatatattgctatactataacattttttaaaaattttgataactagatttcaataagattggtatttgtgatcatatttattttcttgatttttataacagctttattgagatataatttctgtaccatacaactcattaaagtgtacaactccatgtttctaaaatatattttcaggattgtacaaccatcaccaccaccatcaccataatctaattttagaatatttttgtcccccttaaaagaagcttcgtactcattagcaattatctacattccccctttccccactctcagcccctggcaaccacaacatctactttctgtctctagaaatgtgcctgttctggacgtttcatagaaatggaatcttgtttttgagacagagtctcactctgtcacccaggctggagtgcagtggcgtaatttcggctcactgcaacctccacctcctgggttcaagccatccccctgcctcagcctcccgagtagctgggattacaggcgtgtgccaccacgcccagctaatttttgtatttttagtagagatagggttccgccacgttgcccaggctggtctcgaacttctgacctcaggtgatccgcctgccttcgcctcccaaagtgctggggttacaggttacaaagtaagccactgtgcccggccaaaatggaatctttctgaggtcttttgtgactggcttttttcatgtagcataatgttttcaaggttcatctatctttgtattttgttctgtgcacttaaaacattattctgagaaggggtctaaaggcctgccccgtcgtcaccagaagggcctgtggcatcattggagaggttagggctctccctaaccattagggctccctgcgcctaatggttcttccctctgtcagttttgcattaaaactagtacatgccttggcttgagcatggacaatcactgccaaagacccggcaggcaaggcacgcagctttccttgggatgggttggacccaggccctgggcactcagaggtactcaggcacctctgcccagtctccccaagttaccacctccacaggtgccttggagtgagtgggcatctccaggatgtagcctctagtggatgatacccgtggacagggacagcaaggtagagtaatagggccaggccccaggtgcctgcccccagctggacgccagaaccccaggaccccagctcctccagcccccaggaagctgtcagcacttccagagagcaagctgttggtgggaaggtctgaatccttgcagctctgtgattcgcgaatcataaaacctccacacttggtgtttgtgggccgagctattgtctttgtgttttctttttggagtgggtgggaagccagcttggcaagccgggtgtgaatgaacaggaatgagcaggagacgctcgctgatgccttctgtgatgtgtttcttccccagctccagaagaaaaggaagtcattaaaggacagtatggcaagctcacggacgcgtacggctgcctgggggagctgaggctgaaatctggtgagtagccgctcgctggaggagcaggcgccaggctccccggtgggcaggagcctctgtgtcggaaggggcctcagtgcaggcattctgtttgaccgctcttctctctctcttcttttttttttttttttttttttctccgagatagagttttgctcttgttgcccaggctgcagtgcaatggcgcgatctcggctcaccacaacctctgcctcctgggttcaagcaattctcctgcctcagcctcctgaatagctgggactacaggcatgcgccaccatgcccaactaattttttgtatttttagtagagacggggtttttccatgttggtcaggctggtctgaaactccgacctcaggtggtccacctgcctcagcctcccaaagtgctgggattacagccatgagccgccgtgcctggccgacctctcttctcttttaagatgtttctttatgcctcgggacctgctcctcctgcaggaacctgcaggtggggaggtcaaggtgagtgttgcctggttgtcacctgcctgtgctggggagcagggtggggttcccggcacccccaccctgtgccagagaagctcgtcttcattgcttgccggcgagggcctaagtgtttcgaaagtctggtctggggcttctgcccagggcattgtccgcagtactctagaagatgtgccaaggcgggtgggtgaagacagggaccagggcagatgcccgggggcacaggggtgggctgggagttgtcagcaggagaaaaaagccaccgatgtgtgactgtcagtcagtgtggggtgggagggagaggaagggacagtgtgatctgacttgcgaagggagagaacttccaggaagacgtgggcatctctgctcactctgagagactcgggggtgagccggtggtggccggctgtcgtgggtggtcacagccaaactcagctgttcctggagctcaggagccctggggctttaggttctaccacctgtttccttccttctctctcaggggcccccttgccatctgtgacctggctgtggacatctcttgtgacttctggggccgagacaagagcccagtcgcattcctgattctcagtcccacctggctgagatgggaatggctgtatgggttgtctgaattcgggctttccctgagccaagcgcacacctcccttccctgctcacagcagctgttttcagagactgaggatgtttaaagctgccagaaaagcacggttacgttggccattccttttctgggtgttttctgtcatggaatgtttttccctgaactgtgacgattacagtgatcatttccaaatagcatttaggaaaaatacacagtacccacatgacaaagtgcatttatgaaatgtctgtttcgtgccaagagctagccgtacattcacttatttaatcctaaaaataggctaccgtgataatgcatctttgtccccgtttacagtaacgggaacaggctcagaagcttcaagtaacttgccttaggtcagaaacttagttggggtttgaagcgcctccaaacgcttgctttttctgtggaaatgtgcgggctgccgagggttttctgcaggactttccgctctgcagcgatcttcctgaagttgcaggaaatccctgtggcccttgccttccatggtgatgaccgtgaggatggtggcctgcacctgcatgggactcacttaggaacttgttaacacggctcccgaggccacctgtgctgttgtcaccctgccttgcaggtgtggcagggatgccgcgttccatccatgctgatgcccttttttccacatggtagtatctgggcccccaggatgcctcacagtcttggcttccttcagttatagttggcagctgcttttggtccttacatgaatgactcattttgcatccgacagggactgggattcgacgaattacagtaaccggctcaaggtcacgccattctcaagtggtgccctgggttttaaactcatgccttgcggcaccagggactgccttcaaccaccaggctgttctcctttctgaaagggacaggtggctgggctcacaagggggacaggaagtgcagtaccttcccccatcccaggggaaaagtgttttttcccagggctgtgatctaagatcagctgtgggaaattggtcctaaccaggaagggtggcagccccaaagatgtcttattagagcgagccagggtgtctgcgggggtcagcagggtctaatggggcagggagtgctcagagaggagaccacctcccagggctgccctaggcgaggcctcctccctgccttcagccagtccccgccaccccacaccccacagaagggaatgaggcttcttgtggaccatgcgagaccagccctgctccgaggctttgggtgcccccaacccctgcccacctgtgagagtcaccccccggagtgggttggagtttctctgagtgtggtgtgtatgagtttgactttggcgatgtcaggataatttggtctgttttcaacacattctggaaagcaccctgagcatctgaaggatggaaggattgggctttaaatcaagttttttcagtccccattggttatcttaaggggcgtttgactttaacttgattttctttttcccctgtaagtccctgacccagaactgtgatctattttcttttgtgttttcagctgttgagcatgttgggcagcacggagtgtagctacaactgtgtgaccccacctgtgtgactgactccacctgtgtgactccagtcgtgtgaccaactacaactgtgtaactgactccaattgtgtgactaactccacctgtgtgactctgcgtgactccaactgtgactccatctgtatgactccaattgtgtgacccccacctgtgtgactctgactgtgtgacccccacctgcgtgactctgactgtgtgacccccacctgcgtgactctgactgtgtgacccccacctgcgtgactccgactgtgtgacccccacctgtgtgactccaactgtggactgactctaactatgtgactgactccaactgtgtgactaactccacctgtatgactccaactatatgattgacttcaaccgtgtgaccccacctgtgtgactccaattgtatgactgactctaactgtgtgactccaactgtgactccatctgtatgactgacctcaactgtgtgaccccacctgtgtgactccaactgtgtactgactctgtgtgactgactccacctgtatgactccaactatatgactgacttcaaccgtgtgaccccacctgtgtgactccagttgtgtgactgatttcactgcgtggctaactccactgttgcctgactccagctgcgtgacttcagctgtgtgactccatgtatgtgaccctagctgtgtgactccaactttcagttgccacgaagacaaccagctgagtcaggagtgggcctccctttcaatgtttttagctaagcatctctgcgcaaagaaagaggcttccccacaggctcttaatttgggacaagttttcccttagtttgggaaaaatttcaatctacctaaattagccttttacattagaaatcgagaaatatgcacaaaaattcataaaagcctttttccaggttccaaggtttttgtagaaagcccaaagttcccctttccttcctgaaatgccagcaggaaggggttggagctcccgtgattagccagggagtcaggcttcctgaagtttacacaatacaaagcatattaagtgagaatgtagcagaaaaaagaacttatcccaatgattatagcaatttaaattctcttgaacccaagctctcaaggcctacctgttttatagcctcaagggactctactcctatcccagactataatgaattgtgtctcctggagttgtgcagtacgcaacctgtgaagctgtacacgacagccctataaattctctagcagacagccctcctagattctagttcccacttcacagctgaactgcatttaatctctgtaggactgttttcctgtctgtaaacctgggaaaactgcctcatctctatgttgcctagttctcaggaggtccacatgggccatgcacatggtagccatgaagagtgtggagtgctcctccaacaccaggcgggctggccacaggcagtttcttagagtgtggcctggggacgctgccaccactgagaagctctgctgtctctcctctgtccctgggagggctggagggaggagggtgctgagtgagagggacctggcctgcagagggctgaggacgaggtgtgagctggaaggccaggctgtgtggcctcggggtgtggactcctggtggggggctcctctgcacaggctagcagagggctggtttcatgggcagtcacttcagaaggaggattccctggaggggagtttgaacccttagtcttttgggatgcagtgttctctagcaagtcttgaagcgaactggcctttgtcggtcttagattaatttaattcctgtgcccagtggtgggatattaccaagagagttgacacccagctccctgagggggaagctctgagatgatgcagcatttgccagtttccaaggtgtaaattcacgggccagggctgatttcaggttcccagcgtggagctgaggaaggatctgcaccaggggtgctcactgcgggtgttcactgctgtggccgccgctgcctacacctccctcatgttctgcagaagggaggcgggagaaggccctgagaccatgccgcatgggcacacagtgcttagggttctgaacctgcgggtggcgggggaggctcaagggtcatctggccgaagcttcccccttcacaggtgggaaagcctagacatctttagtcctttgtttggagctttaagtctgagggtgtctgtgtgagcagagggacagataggatggctggagggaccccagggttgggattggcacagaccaggctgggaaggtgaactcagagctgagacaacacaaggaaatgccagcccagagccttgtgggaggcctggggtggtcccggtaaacggggccttgggtctgcaagccacgtatcatgactccaacagccagctttcctggcctgataacaaattgcctaggacctgccactggctcttgcctccctccacctcactctggcagctccctctgacccatgcatggctttgctttggccagagccctggaaccaaagccctttacctggaggggacattgagactattggcgcatcctcctgggaggcccagagagggtaagagccttgcctgaggccacacaggccactggaggtgtgcagagcctggagccccactcttctgagttttgtttttccacttgctatgagatccttgatggtttcagggccctggtctctccctcagctgaggactgccaagaaccccgcaagcctagttagcttcttggctgtcagagaattgatcctgcctcacaaaagcaaatcctgtgctctaggtgagcatcgggaggggcctcacacacctggggcaggtggggaccagtggcttgtaccacactgacggtggcccgggagtcagcgaccccccacagcctggtgctaggctcaagccaggacactttccttaaaacccatagtacaggccaggcatggtggctcatacctgtaatcctagcacctcagggggccgaggcaggaggatcacttgagcctagaagttcaagaccagcctgggctatgtagcaagactatgtctctacatagtgatggtatctacagcaccatgccaggagcggtggtgcacgcctgtagtcccagctacttgggaggctgacatgggaggatcgctggaacccagaaaggggaagttgcagtgtactgagatcaagtcactgcactccagcctgggtgacagtgcaagaacctgtctctaaatttaaaaaaaaaaaaaaaagcaaacaaacaaaaaacccatggtatacagagaacacagtcaggcagtcaggctgtcaatgagattaggcaggacacagtgaggctgggattgaaggaggaggaagccatcagtgctgggtcaggtaggcagggcggggccttcccggagaggcagcgctccacacagcggtcctgaggtttgggtaaggctcttccacagaggcctctgtggttacacccgtcccaccaactgtctgggacacacaggcaggggcagttctaaggaaaagtgtccaggtccttggcttttttctatcatggagcacctgtgatggaggacagggttcttattcccctctgaggtctccctgcttccatgttagggtagagggtaggcccttctctcttctggaagctcaccgtgtgccgtgctccaggaagtaactccccctaggattccagacaaagggacagttcaaaagcttgtcaacagcattgcaataaggtcaggactaggtagcaatctgtttgcaagtcaggtgatgtccaattgtttgcttcccccataacggaaggagaatttaattggggtatcagctgacacattagtaaccattttatcagcaggttattgtcatttcttagcgctaactcagaaggactttgaagaatcgagtgacatttctacaatgaagccctttccattcccatctatttattaatgtgaacaggatcttttagtgtttgcgtctacaaaaatgaaaactagaattagaattggtgcagaacctactttcattccttcaataaatatttattcatctcattaagagatgcctttccaacacattatgactttttgtatcaaatgattatttgtcggccaggcacggtggctcatgcctgtaatcccagcactttgggaggccaaggtgggcggaccactagaggtcaggagttctagaccagcccagtcaacatggtgaaaccccatctctactaaaaatacaaaaattagctgggcatgggggctcatgcctgtaatcccggctactcaagaggctgaggcagtaaacctgaaaggcgaaagttgaacccgggagacagaggttgcagtgagcccagattgcgccactgcactctagcctgggcgacagagcaagactgtcacctcccagagaccccacctcctaaaaccatcgccttgggtgttcatacttcagcatattaatttgggggtgcacacattcagccaatagcaggtacactggttgaccacactcttaaagaggacagccaaaaaaaattatttggcagtgttttatttacattgtattgtttacattgtttgaccagttaggtatcactggaacgataaaccagaacgctctctttttaaacacttggagttgaggaacacacggctgcaggggctgatggggtgatcagaacaggacgtagaagcaccagatatgctacattaggatggaattctgaagaggggggactggaaatatgagtgaaagttccatgtggtaagaaagagcatgctcctgcaatcctgtaaataatagatggcaatgagtatcacgttactgtgtagattccatgggatactgacaagaggctcccagagttttagttttacgtgtcaatatttagaatacactgcacaatatattatttacaggcatttcaactaccgatgaaaaccttcaatggcagttttaaaatgggcagggaacgctgtctgagtccagtgtgttgctgtgacaaagttccgtagtagacggggtggcttatcagcaacagacatttatttcgcgcagttctggaggctggaagtccgagaccagcggcccgcagattggccggctgtgaggacctgcttcttgctccatagatgctgccttttctctgcatctgacatggtgggagaggtgcaggagatctctgggcacaaaccccattcatgagggctccactgccataacccagtcacctcccaaatacccccaccacctcaaaccatcaccttgggtgttcgtacttcagcatattaacttgggggtgcacacattcagtcaatagcaggtacactggttgaccacactcttttaaagggtacagcaggccaggtgcagtggttcacacctgtaatcccagcactttgggaggctgaggcaggtgaatcacctgaggtcaggagttcgagaccagcctggccaacatggtaacaccccatctctaccaaaaatacaaaaaattagccggtataatcccagcaactcgggaggctgaggcaagagaattacttgaacccaggaggcggaggtagcagtgagctgagattgcaccactccactccagcctgggcaacaagagtgaaactccatctcaaaaaaaaatagccgggcgtggtggctcacgcctgtaatcccagcactttgggaggccgaagcaggcagatcacctgaggtcgggagtttgagaccagcctgacctacatggagaaatcccatctctactaaaaatataaaattagctgggcgtggtgacgcatgcttgtaatctcagttactccagagggtgaggcaggagaatcactagaacctgggaggcagaggttgcggtgagccgagatcacaccattgcactccagcttgggcaacaagagtaaaactctgtctcaaaaaataaataaataaataatacatgcatacatacatacatacgtacatacatacatacatacagggtacagcaaactaatccgattggaaactgccggactttggggctagatggagcatgggcttaaagtgaaacctacattttggctccaacacagactagctgtgtgcttgggcgggtgattcagcttccctgggggacagtcatgcgtgtgtgaaatggcagtggtagccctgagacagcagcatgggtaagcaggctggcgacgtcatctgcaaggaggtgtaattaggagcaccagctctggggccagacttggcttgagaaacttgcccaggcactgtagctagaaagtggatttggatcccagctccccgctctgtcaccttgggcaggctgcttaacctctctgtgcgtctgtttccctccctgtgagatggggctggtaagggcgccccttgcggtgggcgggaggatggagtccgcgcctgtacagctcccctgcctccactgcgctcaccagataccggctctcctcactcccctgtaccactcccgaccccctcctccctggacccaggagccccagtgctctgtggcaatgttaagagcctctcctggctgctacagtgctcctctccccagcctcccccaggccgtggtcctgcccaggaaatgcacctgggactgttgccaggcactcaggccccccacacggaggctgttgaggaagtggtgctagaaatcagctcatgactctcacggagtgatgtcatgcaaaattttccatagctctggcagccacaggggccatcctcctgcctctccgagggcacttctgctgaactgtgcttttccctgcactgtgtcagaagaggcaggtgggtgccgggagcgctctgtggccaaggctctgatcacaagctgccaaccccggaaccccacctatcctacttgagaagctgaactctgacccctgtccctgtccccagtgtgataacttcctttgttcttaggtggttttgaggtggtttcctgaggatgtgtctggggagatgtgatgggatcaaccccctgccctgctggcagccccaccccatggcatagcagcaggcccctgggagccctgagcgctcattaaaggaacgcactttaatgacagccacacgccaccctgccacaagcagcgctcttctccctatgaagtgtggtgaggaaattggggtgagagggtggtggaactggttgttttgggggtctcttcctggagtgtggagagatcagaaccatctccagaccatggctgtagacagcggcccttgatcattcacagaagatctcgctgggcggatccttcagacctcagcggggtgggaagagtgtccttgggactagagccctgccagaccccaaaggccggtttctacccgatgatctcttgggccccgtcccgagagtgaggcacggcgccaccaggcacccccgatacgcagcgtgttgtgaagtgcccttgtgagcagcgggggatagggaacagaaaaccagctgtcagcgacagcgaggtggtgccccagagagggctgcctcattcgcctggctgcagggtccgaggtcgtcttggtagcatttggcaggaatttatttaggaagcattcagcaaaccgagtgtgtgtggtgctgggagatgacgacgaaatggccctgcccccaaggaagcccctcccctgggggcacaggtggggagggaggtcatgagctttgtgtgtgtgacaactgctgatgggaatgctattggcacgtacgaggggtgcctgtccgtgtcttgggagagcaaggaaggcttcccggaggagttgctgccaagttggcccagaaggatgaggaggagttcttctggtgaagtggggagggcagaggcaggatggtccctacggagcatgtggggacgctcgtgttcttccagaggatggaatgctgggcccgaagcctgaagtattgttagggtagagacaggcgggaggtgcgtggagaaggggtcatggaggtcttgttgggcagatggcggtgggacgtcccaggcagggggagcatcttgggcaagagcccagggtgcgaggtcatgcttatggcacattggaccagctggaggacagccagctcaggagagcagccggctggggtgtctgcccacgtgtggggcagggatgggctgcagcggggagggatttgggagcttctgtcgtagtcctgcagggagtgggagctgtgcaagaaggaagggcggagcggctgtgatccaatgactttgggaaggccactcctgtgatggagtgggggaagacagaccccaggccaggggtcctgagtcaaggcagtggctgaggaggcactggggaaggtgatttggagaggctttgggggtaggtcctgggggtctggaagggctctaggccaggaattttcagactcctgggggcatcagaatcacctggagggcttgttaaagcaaagcttgcccccagcctcaggcagctgggcggcaggaatctgcatgtctaacaggctcccaggggatgctgacttgctggccagggtcccacactttgagaagccccgcctgaagcttacaggacagggcaggttcttgagccattgagttgggcaatgccatgggaagttgcaggtgcacagacgttgccttcctggctgcggttgaacctgggctccatttgtccccttggagctccaggcggccgggccgacttcgaccctgagctctcctgtctgatttcctttccaggtggcacgtctctgagcttcctggtgttggtgacaggctgcacatctgtgggcagaattccagatgctgaaatctacaaaatcactgccactgacttttaccctcttcaggaagaggccaaggaggaggaacgcctcatagctttgaagaaaatcctcagctcgggggtgttctatttctcatggccaaacgatgggtctcgctttgacctgactgtccgcacgcagaagcagggggatgacagctctgaatgggggaactccttcttctggtgaggccctgggtcccctgcagagggtgggccctgggtctcctgcagagggtgggccctggttggcatctgaggccttgacctccacttgcaccacccccgggttatggggcacctctggagagttaggacccaggcctgctctcagcacaatgagaggaaaactaaccctgaactccctgccaccccccacccctggcccctggaatgtgttcagtgtagatttggaaaagaaacctgcgccaggcatggtgcctcatgcctgtaatcccagcactttgggaggctgaggcaggtggatcacttgagttaaggagtttgagacaagcctggccaacatggtgaaagcccatctctactaaaaatacaaaaaaaaaaaaaaaaaaaaattagctgggcgtggtggcaggtgcctgtaatcccagctactcgggaggctgaggcaggagaatcgcttaaacccaggaaacggaggttgctgtgagccaagatggtgctactacactccaccctgggtgacagagcaagactctgtctcaaagaaaaagaaagaaagaaaagaaaccctgttccataagtaaaaaacataagagccgacgtctgctgagtgcttcctcagtcccgggtgccgagctcagtgcttctgcctggattcactctttcagtcttctcaatcatggttacctccagtgtgcagccgtttaccgctgatgtttataaagcactgtgttcgggtcaccgtgctaagatctctctgctggtaataactcatcattattataaaaacctttataataattatgcggaggctctgttaccccattttacaggtagagagactgaggcacgaatagtcgagggaattttgccaaactccacacacttactaagtggcagagccaggatttgaacccaggctttctgggtccaggtcccaagctggtaatcacagtgctaaaccagtcccatcaatggggaagctgagtcaccctgcccaaagggacttgtagcaatgcactaagactagtcccggagcccaggcagaggcggctggatttctgtatgtttaagatctcctggtgctttttcactccacagcaggacagagacaagagtggacaaccctgtaaatacataaattatctattgccctttctggacataatgctgtttctgtcagattcgcactgatgcctagagatgccaggtagaagcaaaagtgaggaaagtcctgccatgggagtcgttccaatgaacctcatctaatgtaatgttttagaatccagtgtctgccttttgaagctatatcagttttcccagctctgagacgggactcgtgaggtttatttctgacctgtttgtgcctctctccctttgccagaggcagccacaggttttcatctggccttggtgctttggattccccagaagtgaattcctgatttctgccggaagaaggggaccctccccacatccctgtgtccatcccaacgctccccacccccacatcctctttggttctctggtattgtggatatttgggggcagtaatgtgcccagacttcaaaatgcacgtgaatcaaggctgggcatggtagctcacgcctgcaatcccagcactttgggaggccaaggcaggcggatcacctgaggtcagcagtttgagaccagcctggccaacatggtgaaaccctgtctctactaaagatacaaagaaaattagccaggtgtggtggcgcacacctgtaatcccagctacatgggaggctgaggcagaagaatcgtttgaacccaggaggctgagattgcagtgagccgagatcgcaccattgcactccagcctgggctacagggtgagactctgtctcaaaataaataaataaaataaaataaataaaatcgtgaatctcatgctgatctcattaaaaatgcagattctgggatggcaggtctgggctgggcccaggattctgcatttctaataagctgccagtgctgcttgtctgcagacggcaggttgagtagcagcctctggggatggcatttctgtgaatcgctggttacattttgcagggcccagcatgtgccccgcgagctgttacatgttcccctaaaggcagccaagcttaagcacagaggggctgtgtattatgaggtatctttgtgtggagtccgtgaaactattttaaaccagaactgtcaggggagcagaggtgagagctctcagcggcagtgagggtccttggaacccctaggggatccttggggtatctgaattgcaccagcacctttgcaagttctcttccctatgccagctctgtggctggaggtgtgaggggcactaacaaagcgaggcaaaggggattcacgaggtggaggctgagaggagctttggaaggaggagccccatggtgtccctttctgaagggagaggaggaagtaactctggaggtcgccaggctcctcagctgtaacacgccagggcgtggtgtttaggctcagggtggagcccccagaactgaccacaactgagtcaccacgggcagtgtggttctgggcttgatcgcctgaacccccactgttgtggcgcagcgtgaggtctggagacccacagcaggagtcagatggggtcaacgctcagcgtggctggcacataaggcagagtgagcagcagggtgaagtcatctttctgggctggtaattgatcacactgacaattgcctcccagtacaatcaggtcctgggaggaaactctttgggggtcctggatatgagtcatcctcctgccagagggccaggcagtgtcactagaaacagggctgatttaagggttgtccacttggcaaaaggtttcttctcctgagccctggtgatagtggccatagctgtgtagctggcagactctgaaatattctggaggctttgccttgatgccacatgcagatttcatatatgtgtttcggaagctggcttatcatctgaatttgagagagtgatttggaagagggacatgtgtttttgtacagcaatgcttcagcagcatttttcaagcagaggagcgctgtggctgtgcgacctcagatggggtcagacgcgggctgtgctccctgtgggggtgcatgtgactgtgcgacctcagacggggtcagacgcgggctgtgctccctgtgggggtgcatgtggctttggtcacagtcggggaggctggcctgagagcaggaagcttggagaagccccctgggacccatcagagctcacagcacagatttccagcaatgagaaaatgctgctgctgtctttgaggaaggctcaagggcaggaattgtgttgccttttttaaacaagtagggcactgtgatctagattcactcagtgccctgtaagagggcatctcctgatgatgggtcctcctccctgagagtgagtatgggccctcaggatggtaccactcaggtcccctgggtgctaaagcaagggggccaggccagctcagggatgagagaactacgtggtcgttcgcaggtgcacatggaaacgtagggcctcatgaagaaaggctcggcgtgaagagcgcgggctccaagctccatgacgccactggctggctctgtggcattagacaagcacttcacttctctgtgcctcagctgctttatctgtgaaagggggtcactgatgatacctttcacatagggtcacttcaagggtgaaatgagttaatccaccccaaagtacttagaatggggcttagtgcacaggataaatgttggctgtcaccctgtcgttatcatcatggactctagagcctacagcaatccactaggcatggagagggaatacttgaactttcattacattttattttcctcggatctttttaaaatttattttggattgctttgtagcgctctcgtcataatcatctcatggtgcatgtgtgtgtatatgtggataatttaaataactgttcccatatttaatatgatttttaatcacttattgatcatgattgtttaataataaaagatgattaattttttaccaatggggcctgcgatgaaaactgtggggcccacagggctaaccagaggcccctttgcgattccccgaaaagtgcacgctggctcactctccagctttgcttccatttagcgacagtgacgacctggtagtggctttgggaaaggactccctggccatcctcctctacgtgacacagtggtcctgctatttgggatctctttgaagcacatgaacttcccagatctgtacagcccaagtgcagagtccactggggagcagcatgcattcgctgacccgaaatgctgcaccgtgcgccccccagttcctcagccacactcgctgtccaggcagcatgtattggaggatgtcaagtgactggaattggtgtgggactgtgttttgcaggaaccagctgttgcacgtgcccttgaggcagcaccaggtgagctgctgtgactggctgctgaagatcatctgcggggtggtcaccatccgcaccgtgtatgcctcccacaagcaggccaaggcctgcctcgtctctcgcgttagctgtgagcgcacaggcactcgcttccacacccgtggcgtgaacgacgacggccatgtgtccaacttcgtggagacagagcaggtgagtgcccaggcccatctgtggcaccaaatggttccgagtggctgcagctcttgcacacgcgctttccacttggggcagaagaggccctggcatatttgtggtggtctctgatcaggcttcctcctataagtggagaacagcacgtgtaattgggctccacatgaaaatgtgacctctggtttgcatggtgtatcctttctgattcacatctcatgcagggaaaatgctgcttagccgaggtatttttaactaaacggaatcaggaatttttgccttggtcgtgaaaggtgggcagtattccaagggcagctgacagatgaacaaatagggtttgaaaatccttttaggacaaaaataagtagatcaatacaatggggggatgaaagaagaaaccattattttttattctaaatcaactgaagcttctcctggaagccagtattagcaccctagagttgtgaaatctgcgtgttaagcagccgctcccatccgttcccctagaatatagccatctgcttaatttctcctggagttttttctaaattctgttctcatttccagagatgctgtcttacaacttttaaatttttgctctaacttgcaatctgtaagataaggccaccatgtggaatacagtggtgtaagccataagaaaggaaaaccccggaaggcacatgtatggtactcctgccagggcaagctgggtgccctgtgtttactgagtgaaggcgtggctgtgcgtcccccctcgctgccgctgctgggagaggaatgccttatgcggactttggccactttaatccagcagaggtttgagcacctacctttactgtgtgggactttctgcctgctccagtgttcagagggtagatacagagatgaggaggctgctctcagagtccccactcagaggaacatcagagcagggggccctaagatgggcaacagctgcaattacttgcctgtaattctgctcagaatgtctgcactgtggatatcaacgaggctattatgtaaatatctggttaaaaatgagattgcttttggcatcttcgtcatgaagtctttgactgttcctatgtctagaatggtattgcctaggttgtcttccagggtttttatagttttgcgttttacattcaagtctttaacccatcttgagttgatttttgtatatggtgtaaggaaggggtccagtttcagtcttctgcatatggctagccagttatcccagcaccgtttattgaatagagaatccttttcccattgcttgtttttgtcaggttggtcaaagatcacatggttgtaggtgtacggccttatttctgggttgtctattctgtttcattggtctacgtatctgtttttgtaccagtgccatgctgctttggttactatagccctgtagtatagtttgaattcaggtagtgtgatgcctccagctttgttctttttgcttaggattgccttggctattcgggctcttttttggttccatatgaattttaaaatagttttttctagttctatgaagaatgtcaatggtagtttaataggaatagctttgaatccataaattgctttgggcattatgtccattttaatgatattgattcttcttatccatgaggatggaatgtttttccatttgtgtcatctccaatttctttgagcagtgttttgtaattctcattgtagagatctttcacctccctcataaactgtattgctaggtattttattcttctcacagcagttgtgaatgggattgtgttcctgatttggctctctgcttgactgttgttggtgtattgattttgtattctgagactttgccgaagctgtttatcaggttaaggagattttgggctgagactgtggtgttttctagatatagaatcatgttgattgcaaacagggatagtttgattcctctcttcctattgggatgccctttatttctttctcttgcctgattgccctggtcaaaacttctaatactattaatatgttaaataggagtgatgagagagggcatccttgtcctgtgctggttttcagcttttgcccattcagtatggtgttggctgtgggtttgtcataaatggctcttattagtttgaggattgtggatctaaccaaactaaagagcttctgcacagctgaagaaactatcaacagagtgaacagacaacctacagaatgggagaaaatttttgcaaactatgcatctgacaaaggtctaacacccagcatctataaggaacttaaacaaatttccaagaaaaaaaaaaaaaacattaaaaactgggcaaaggacataaacagacacttttcaaaagaaggcatacatgcggccagcaatcatggaaaaaagcttaacatcactgatcattagagaaatgagaatcaaagccacaatgagctaccccctcacacaagtcaaatggctattatttgacttgcgtgaatattatttgacttgtgtgaaatgagatacctcctcacacaataatggctattgttaaaaagtcgaaaaataacagatgctggcaagattgcagagaaaaaggaacgctcatacgctgttggtgggagtgtaaattagttccaccattgtggagaacaatgtggtgattcctcagagacctactcacaatagcaaagacatggaatcaacctgaatgtccatcagtggtagactggataaagaaaatgtggtttgtatacagcatggaatagtatgcagtcatgaaaaggaatgagatcgtgtcctttccagcaacacggatgcagctggaggccattaccctaagcagactaaggcaggagcagaaaaccaaacactgcatgttcttacttacaagtgggagctaaatgatgagaacacatggatacaaagaggggaacaacacacactggggcctatcagagggtggaggatggagggtaggaggagggagaggatcaggaaaaataactagtgggtgctgctaggcttaatactgggtgacaaaataacctgtacaacaaacccacataacatgcgtttacctatgtaacaaacctgcacaagtacccctgaacttaaaataaaagttaaattttgaaaaattttaaaaaggggacagagagagaagctgtggccccagctgggtggttttctcttttctggctgatgtctgcatggggagagtcccttccctgccccacttcattaccacttgccttttattttgaacttttatttaaaatactggttttggatttcaaccagaattttaagacttggggtctcatctattgcatctcatcgagtaagtccctgggaaccactgtcctttagcggcactgtccttcctaaaacaccggacattctaacacggaaaatgaaagcaaaccgcatgcatttcctaggactgcccataataaagtaccccaaactgggtggccgaaaacaacacacttattctctcccagttctggagactggaagtctgcaatccaggtgtcggcaggactgctgtctcttcgaagtccccctggggagaacccttctgagcccacctgaccgccccggctcttggtggcctctggcaagcatctctccatcatctctgcctccgttttcgtgtggccttctctgcctgtgtctctccaggactccagtcgtactggatcagggacccaccctactccagttgtcctcatctttttttttttttttttttttttggagacggagtctcgctctgtcacccaggctggagttcagtggcacaatctcggctcactgcaagctctgcctcccgggttcacaccattctcctgcctcagcctcctgagtagctgggactacaggcgcccgccaccacgcccggctaattttttgtatttttagtagagacagggtttcaccgtgttagccaggatggtctcgatctcctgacctcgtgatctgcccgcctcggcctcccaaagtgctgagattacaggtgtgagccactgagcccggcccagttgtcctcatcttaacccattacttctgcaaagaccctatttccaaacaagttctaaagtactgggagttggtacttcaccatatggtttttggaagggacacagctcaccctgggccgcggatgctggctcctgttagaggactgtgctttacaggcagcgcctgtggccctgtgagaactctcgctgcaatctttgctttctcacttttacagaccaacaagccctaccacagaccaggctgggccttggctctggccacttcgtgtcgcgtgcctgctgactggcgcttgtctcctgtgttgccagcctgcttgttagagctcagaggcagggaccttgcccccacatctcctgaatctcccacctcgggcagcctggcaaggccatagagatatgaacagcctgcccttccagctcctggggtacagcccgatgctgttgacattgcctccacctcccttccttgtctgggctggggagggatttcttggctcccataagcctttgtgggcttcctagtgtgtgtgttttgtcctttttcgtcctctgggctctgggctagacagggctgctctccatgaagtccccacccaaccttactcccaggagcagcagtgattgacagacagctgggcaccaccacctgccgctcacccacagatgcagccgctgcggatgctgcccccgccccacctagagagcataacttaacccatggtgacttcgttaacaagagatgcgtagcggggccccaggaaggtggccctacacccacggtagctcttggttgggtgcctatgtttcaggctgtgctgatgagcctggaggtgcctgggctggcccatccatcggagaccccggacagcctggctacccaggaaggggagctgtcacttcccagcgctgagtccagggctgtccacgtgggccttcagcaaagcctgactgagcctgccaggtcccaggctctgctcccaggtgggtgggcatggggggcagtggtggtgtgatgggggcgcagcaggagtggggcaggtgaagctggtcctgccaggtgctgccgcccttccaggtggtacttccgataggtggtgttcggctggtgacccctgcctggaggtcgaggggaagcacagcagagccagggcagggagaagcgtgtgctgcgcgcagcccaggaaggaggggcatggagcagaaagaaacacaggtgaactggaagagcagagagggttcatgcttcctgcacacatgggaccagggcatgggcctggtggggggtcagcattcctccaagccaaaaaacacaggtgtgacagccctttttctaaaacattacagaggcagcaggagcagagatggctgcagggaagtgactgttccccaaaagcaagaaactcctgttgcctcacttgtataagccaagggaacccctcgctttctgtctctggagtcactgaatatttttgtttatgtcactttatagcagtactgatgcttaggttgatctaagttcgtttttaaaacacaaaacacgtctttaagtggaatgcacagttagtatgctcctgcgaagcggcagccccgctgccctccctggctctgcggccacctgctgaggatggttcttctaggatgagtcaggagagtggagcaaggctcagcctgcaggtccttgctgagctcagggaagcagccccgtgaggatcgggctgcgtggtctgcaggggaacctgcctgggaccctgtgagatttacctgtttcaggtgccagtgatctgctgggattctggagaggtccctcaggggagcctggcacttcctacccccttgagtttgcaaagtcacaactgagggcctgggttgggacggtgtcatgagctctccataagctttgcttatggtacaggagcagactgtctggcgcttctggagacctttaacgtgctcctgtagacagtcgggtaaactcattctggtagcacagcgtggcctccccctccacctgacatctgaggaagtggcaggctctgcatctcggtggcctcgccccagggtgcagggagcaactttgaggaaattagtgctgagcctccagtaggaaggaggactgtgctggggaggcaggtgagccccttgtggtgccgcccgcaggtcacactggcctggggcccctgcagaggaaacaggatccaccagccaggcccgctgaccatttggaagtggagcgtgcacacaggcggctgtgcggtcatcgtctggcaaacaaatgatgggaccggaccaggagcagactggctccctgtggtggggactcttggcaggcggatcggcttcttaggaattaattctcaggaagccagaaaagggggaccctgtggcaccctctgcttttgctttggtttaacatgtttcctaggaggaataattaatcttctttctctgaggcaggctgtgtgtgtgtgtgcatgggttgtatgtgtacagtgtgtgcgtggtatgtgcatttatgtgttgtacatgtgtgtatgtgtcatgtgcattgtgcatttgtgtatacatgtgtgtggtgtgtgccttgtgtttgtttttcatgtgtatgtgtgcagtgtgtgcatttatgtgttgtatacacacatgtgtgggttgtgtatgtgttgtgtgtctgcacgcacagatgttgtgtacatgtgtagatagcatctcagcctccttgggaggaagccttgagtctttttggctctgctgagtcatagctggtgggcttgctctgcagtgctgactcaccacacagaaaaacaagaccaaaaagtcccctcagtctattaaaaaaaaaaaaggatgtaagataaatatttggctattcaagataaggccactgactcattcagggaggttaggatgactgcgtgtcctcccaggaatgcagacttggagataattggtttgcagacactgtatctggaagcagaaggtcagcagaaagcctttcagatagaaggtatatgtacccctgtgagttttcctgcctcgtcgctctctttttatggccattaggtttctgttcccactgctctggaagcgtcactcagggtcaccctctggctgctcagtctggggtctaggcactggcctggcctcatcacagcactggcacgggcctccgacttttcttcccgtggccccactgctcttcccggtctccctcccacctcttgtctgttccttcttgaatccctccttcttcccacgacccaagctctctccgacgccagcaggtgcccgcatgcatagtcatagcgtcagggcctctggggaagagaaaacagcttcgttttttgagtggctactctgtgctgggactatgcccccctcatttacccctcacagctcatcccttctccgtgcacaaggtgacatgaacttcatgtagagagagggaaactgaggctgaagatggctaagtaatgtgcccagggccaccctgctgggcaaatgggggtttgcaaagtggcagtcggacccagcttgtgctaataccagagcccactttctctcctgcttccactctctgtcctgcctccccaccccaagagccctctgtcaaaatgacatctgtgaggtgagacatcacttcttccactggtattaaaagttgttcgcccagaatggaaatgtatcacttgcatttgatggtcaagttatgcctactgccttgggaacaaagaagacagtcttgatgataggacggggtgcagatgtcacctacttcccctgtggaggacggggtggagaactttggagctcctttcttcttgctccagtgtgggcagggaggggcatacagagcactgacagccctcagtgttttcagaagctggttctgggcaagtctcagcttgggaggagcctgatgtgatgctggaagtggcattgttagcacttggggcagtcagagtgaagggagacattcctcagcctaaatagatcgtgggcattggtttttgttgttgtttggtgttttttggatttcactctgttgccccagctggagtgtcgtggtgcaatcatagctcactgcagcctcatcctcctgggctcaggtgatcttcttgcctcagcctcccgaatagctgggactacaggcatacgccaccatacccggctaactttttaaaaattgttttggtagagatgggggtctcactttattacccaggctggtctcgaacttttgggctcaagcccgcctcccaccttggcctcccaaagtggtgggaccgtaggcgtgagtcactgcgcccagcctggatattgttaaacattgtctttatatacaaatgttggtttcttttccaggaacctgggcctatgcagggcacacacgccggtcagttgtatgtacctaatttcacggtagccaactatgtctgtagccgttcaagcaacctgctattttattctgttttgtcagtagaagaatggagttcaacagctttgctggtactcaatacctggaaacagacagcatgttcttaacacatgcttttttcctttggagattaagggatagtattccagtttgacaatgtagataccaggctcctgtaattcctgtggcccagggaaacagtggctctaattttcaggtcctccactgcagaatgactcattcctgtgtgatctgagcgtgatctcgcctgcatgggaagctgccctgtaagggagcaggccggtgaggaacggaaaggaccacagcagcgcccaggccgccctgccaggccaggctagagttgcgcagataaaggccccgcatctgataagtgtcgccgcctgcccctgctggcgctcaggatcgtacttcttgtccttttaaaactcccttgcactccagcgtcattggaggcctgcaggcacactctgaaatagctgtacgtgtgggtggggaggagatagatgtcttaaagtgtacggatcatttgtttctctgcaggtgctgtcacactggaagcttgtggaaaccgtgttgtgtgtgttaactgagtggtgtgtttaagaacacagtggggcagggccaggaccaagtacaagatcaggttgagtcttgcattgtcaagtacgagactggctagcacagtcagtgaggtcttttggctatcactggtgcattttatttcattcagtccttttctcatagctctcttctgcttagctatttgccaaggcaaaaatagagggctctaaggaaggttacattccagagcacactgacttctattcatacagggggtggcggaaggctgttcctgggattttcagggtgacgataaggttgtgccctagaagtgtgaattcccggagaccctgggaggccccaacccccagcagaccccctcctcagaggctggtcgacagccagcatgcccgcccagtacctggcgagaggtcagggcgcattgccggatgggggagcagaggacgccggagtccaccgtccgcccttcattctggctggtgtcgggtcacaggtggccggatcccgttacccagcccaggacgttcggtttcattgaagaatacctttccctttctgtttccttgtgccgcagatgatttacatggacgatggagtgtcatcttttgtccagatcagaggctccgttccgctgttctgggaacagccagggcttcaggtaggtcatgaaaaaacttaaatgtccccttgtgatgttgtccgccctgcccttcccttcaatagctggggaagatttcttttaacacgttcgtttcatggcatagtttccagtctttttcctcagccctgttgtgttgagctgagctatcaaagtgtgcacacgtgtgtgcatatgcatgcgtgcgtgtgtgtagaaaacacagacaccactttgttgtttctggtcgtcaaggatgctgtgtgaaaccgctgactcgggaacggtgctgcggtgcctgctgagtgggtgccgcgaagtgagcttgggtagcactgcctgaggttagaccctgagttcggtgtactgaggacgtttctgagagccgtgactctcatggagggcttcaggaaggctctgtggctgcgtaggtctggcacccggtgctgcctgttgtggggccacgtgagacttccacagctttcccgtgatttctaaaaataccccttgccgtccaaagcagggctctggtcagtgccagagagatttggcattctttctcctgggaagagcacagggtggggacacattccaagaacacatttgtcatcaatggaatgacacgctgcctccttcaccagggtctctaacaaggtgggttatgggccgagtggactggaggggcctccccactaatccccagtcttcctccctgtcccgaggggacgtgggtgccttcagaaagcctctcggccagggcctttgtcgcctctcggctcttcttaccgacagctctcgtgtgactgcatttgggttgccagccagtggctactggggcccagagtgccatctgccaggttccagggggtgtttgacaacgctggactcagggtccccagtcctgggttcagcttcttagcctgctctttgacttgaccctgtgccaggagcaggtgagtcacttgtccccatggctgtcagcacgctgcagtgacagcattggtgaagtactgggtcagcctgggcagtgagatggatgaatggaggtctcgtttaggctccacgctctggtagggagaaaagtcttgattaacattcagtctttccactagaaagagcgcaccacactgcacgagataactgacggcctccggagaaacgctcctgaggacagctgtgacacagaaactcctccggagggctgcccaaaggaggtttttggaaaaatagctttattgcaatataacttacacacccatttaaagcctacaacttggtgcttttactgcatttatggtgttgtgtaaccaccactgtaatctaatcttagaacactttgatcaccactaacaaactttgtccccgttagtggtctctcccatcccctccaagtctccctcatgccaactcgactgttcatctactgtctttatctatagatttcccattccagacgttccaaaagagtttttgaaacctgattttctttaaatatggagttttcctttggaaacttctcgagctctgtattcaggagaatgaaagaattgatcgaatgagtgtggcacaaaaaggagacagggagaccatgttgtgtttcatgtaaagtttgtaagaaatccaaaaattaggggaatggtcatgatggattcttagaatttgggaaggaccctttggaattcatccattccaccctctgactctcagttcaggggtcctccttttttttttttttttttttttttttgagatggagtctcactctgttgcccaggctggagtgcagtggtgcaatctcggctcactgcaacatctgccccccgagttcaagccgttcccctgcctcagcctccagagtagctgggactacaggtgcccactaccacacccagctaattttgttgtatttttagtagagatggagtttcaccatgttagccaggctggtcttgaactcctgacttcaggtgatccacccgcctcagcctcccaaagtgctgggattacaggcgtgagccaccgcacctagccaggccagttcagggatcctcttccaggacctccaaagggctgtcctccaggccacaactggggatggttccgactcttggtgaattcctcctgtattgcactgctgcgtctgtctgactctagagtgttttggggtgcgcgggaatatgtctgattcttctccgtgtgactcaccctcagtccttggagggagtcttctggttccctcttcttcttcttgtcttctctaaggcctcccaactcctttggccaccttccctcctgtttctaagactctcaatgttctcatctccctcatctaaaaaatgacactcctttttgtcgttcccctcttggatttcattttggtcttaccttgaccttcgtgctgcctcagatgtagcctgacccgccatgggatgactgtgccttggtctgtcaggggccaggagcagtggaccacgaggtctcagctttttggcagctgtttcaccctcttaaattcctaacatttgtgctgcaccaaagcctccatggtttcttctttttgtttcttgttttgttttgtttttgagacgaagtcttgctctgtcacccaggctggagtgcaatggcacgacctcggctcactgcaacctccacttcctgggttcaagagattctcctgcctcagcctcacaagtagctggggttacaggcacccaccaccatgcctggctaattttttttgtatttttagtagagacagggtttcatcacgttgaccaggctggttttgaacttctgacctcaagtgatctgcccgcttcggcctcccaaagtgctaggattacaggcgtgagccatcgcgcccggcctccatggtttcttcaaaacaagcttcagaaggctttagtgttccttaatcttattaaggatttgctaacctttgggcctaatacctgtgtttagcctctaaacaccactcatcatcaaagagagatgaagtcatcttcacagggggccgggagggcagcgggaagtggtggtctaccgtcaccggcttcaactgtcactatcaagcctaaggctgcttctagttatttttctccactttgaactttttctcttccttcttcacttaggttagatgaaaagtgcaaagattttggggtgagtagcttgcgtattaagctgcaatgctgtccctccttgacagctgccgttggaaacccaggagcccagcaggatggggcagggttgagcagagggtggtggatgaggatttgtctgcatgttttcctgggtaatggtaccttcagtacttcctgactccatttctagcagccgtgtacctttaataaatttcatttttacgtgtaaatgggctgtgcagtttataaatagccacattgaaattttacccttaaagcaaattgctccctggtctagctagttcgagaatctatttatgatctgtgtctgtggttgacaacgttgctcccctggtcattgtagaagcttccccccaccatatgctgtatagacacgtgccttgccaaggtgagttcagtctgtcttttgaagtttcctgcaactgaaccaaacggaggaggatgccgagtagagcagacccctcttgccctgccttcacctctttcctgccagaaaccattcgccaagtttgtggagtcgtggcgctggatgggcttgtgtgtttgtaccttccttttttccaaaaaggacatgagatggcttgtagtataacagtttcaaaaataagtgaagaggtctggatgaagggcaagagtcgaattggtgcccacgatgcatgccgtgggtcttgtgtgcgtgggaaggaacctgacgcttgttccaagcttccccgcagccaccacagaaagaaccacagtcacagccagtttatgacagtgactcagagcacgagctttgaagcgacgcagacctgggctcaaattcaaactctggctcccctgctttgtaaccatgtgtgtttgagaggctcttatctgtgccaagccttagtttacttttctgtaaaatggggctaacaatatctaccttatagggttctagagaggattaaagagcgtaatataagaatttggctgggcatgggggctcatgcctgtaattccagcaatttgggaggccaaggtggacagatcacttgaggtcaggagtttaagacaagcctggccaacatggtgaaatcccatccccactaaaaatgcaaaaattagctaggcatggtgtcaggttcctgtaatcccagttattcaggaggctgaggcaggagaatcacttgaacctgggaggcggagtttgcagtgagccaagattgtgccaccacactctagcctggtcaacagagtgcgactctgtcaaaaaaaaaaaaaaaaaaaaaaaaaaaaaaacacacagtatctagaatctaaaaagtactcaaaatggcagctgctattgatactaatagaaacaaatgataacacccaggagataaaaccaatggtggcatccggagaagcatggctcttggtgccgtgagcactgaatgagagtttcctcctgtctttgtgggaacagggacaccgtgagatgtagtaacatgaagacagcttcctggtcagtcttatctctcatccctcatagactcatcgtaaatcagtaaccccagatgaagccatttattcatcccaaacattcactgagcgtctgccaggtgtagggggcaccagggatcagatacagtccctaccttcagggagcagggggcaggacagggcagtgttgtgtggacagagccgtgttggaggaagggagaggccccgggggcagctctcccaggctgagggtgtggggtgggctgcccaagggtccagactggggaggggtgaggaagaagcactcaggcaaggaaggcagcccttgcagagggaacaatgggtgcaggtgttgtaacccttgggttcacagtgatcagggagcatcactgggtagcttcttgaagtcctgggcatgaagggaatgggaggagagataggagaggtgagcagggctgggtcaagggcctgtggatgctacactgaggagtttgcatcccataccagcagctactcaagcagtactccccaaccttgctgcacagaagactctgctggggagcttgaaaaatcctgatacccaggttgataccatacacattaagtcacaatatctggggccgggagccaggcagctgtcctttttgaagacccatgggtgactccagcatgcagaaaagtatgggaaccactcggtgctgtgctggcaagccagttttattggggtcagactgggtgcagggagctttggtttgtagcatttgtccattgctgtggtgtaaattctcccatcgtggccactttcaacctacggctccacatcactgagctcagagttgggaccagctgggtgcaccgtcggcttgcaagcctgcaggagctggctccagcacacactggacactgctaaagggctgcaagaggtagagacacaattagacagaagtctaggcagtgttagtttggaggggaaagacaccagatgggaggccgttgagctgagatttttgggtaaccttaagtctggtaggagtggtggggtgcaggggagaggtgtggaggtgctgtctgcgatccttgggcctcttgagtgctgatcgagactctgctgtgacttccacagccaggagttctccagaccccgactatcctcatggctgtctacctaacctcagtctctggtctataggattgaactaatgactttaggggccagataagagtagctggccctttgcccagaggggaggaggacggggtggaggagaagggaagctacaaaatgcagtgacagtggtggaccctgccctcagctgcagctcgtgcctgtggcttctgacacccagtgacctcaggactaggtgacaccagagaaggtggatgctggtcagtgttggagcaggacagaggggaggaaggatatgaggcccaaggaacccagacaaaagggatgcacggggtgacggaggaagggtgagctccggagagaacatgaggcccagctcccctccacttttgcccaaggcctcactgtgttgcctttccacctgcctaaaagaagcagcttagggtcatgactcagggagaggcggttagacaagtccaataatacaacttgtttaggggatggagggacccagcctgaggccctgagcccagggaagcagctgtggcaagctgagcctggtcatggtgtccctgaggctctgcaggtatggacatgcctctgctggtatgcatgcagctctgcaggtggggacacggctctgcaggtggggacatggctctgcaggtatgcacgtggctctgcaggtggggacacggctctgcaggtgtcgacatgcctctgctggtatgcacgcagctctgcaggtggggacatggctctgcaggtatgcctgcggctctgcaggtatgcatgtggctctgcaggtatgcatgcagctctgcaggtggggacatgcctctaccagtatgcacgtggctctgcaggtgtgcacccggctctgcaggtatggacacagctctgcaggtgtggacagggctctgcaggtgtgcatgcagctctacaggtgtggatgcacctctgcaggtatgcacatggcccctggagctgtcttccctgctgtaccttctaccccttcatgggcatttgtaacctaagccaaaagggaacataacaagtccagaaataagtcaaaaccagaaccactcaaacttcagcgtgtgtgcataggaatctcctggagggcttgttaaaacacagattcctggccccacccctggagagtctggctcagcagatctggggtaggcccaggacttcgcatttctaacaagcacccagaggaggtccaacgaccacactttgagaatcactggtctagacttaacagatcccttctgcccattccctgctctggtcattcacccgggggtggtgctcccgactgacttcttagttctctgaggtagaaaggtttgttgtcagcagtgatactggactagccactgcaggggatattgaccatgcctgtgacagttgggcccagccctgtccacacggaatgcttgtctaagtccaggataaaagacaagcctgaggcctcagaaaagatgtgctgcagcccctgtccgggctgtgccttctgctgtgggcagctgattccagggcatgccgtctgcttgccacattccctgagtcctcactgatgtggttgaggggaccccatcaaaggcaggtgcctcatttcactctgctttgctgcctgttccctggactcaagggcatagtttctgtcaaattaatttgcgcttgtgcaggtttgttagaaactttgaagtgcttctgggtctttccatgggggttctggttaggtgtcatcacccaggccaggggattcagcgtctcccatccctttcccacctttccatgacacccaatggcatgttgactcggagggcgctcagtaactgaggaatggactgctgactgatagtgcagtttatatgttaacgggggagctatagagcctggggattggagcctgctaggtccagtcatctgtgcaacagtttctacctaattaaaagaagaggctttgggttatgactcagggagagacagttagacatgtccagtaatagaacttgtcacgacggagagccaggctcctcagccctggcctgccaagcttgtattctcttcttactgtgctcccatggggaatgatcctataaacagctgctgctcattgctgaagccaggggagacttcttgtgatgactgacagaccacacagagcaggtagcccctctcttgggttttagccctggtgacctctcatagggtggctgaggggtacacagacccgctgacttccctcagggtgcaggatggagcaggcctctcggtagtgcgaccatgaaagttttcaaattttctgagatttcctcagccaggtgttgatggccatggggataaaggacagaggtaacacaggcagcaaaaaaataaaaaaaaaccccacaggtttggggtgagcctttcttaggagctccattagccaacagccaagatacctcattttgcactcacttcacttgtgaccaaagtgtcttaggaccaaaagggatttgggactttgttgaactgtttatataaaaaaaacctggctgggcacaatggctcaagcctgtaatctcagtactttgagaggccgaggtgggtggatcacttgagcccgggagttcaagaccaacgtgggcagcatggcgaaaccccatctctacaaaaaatacaaaaaaaaaaaaaaatagccaggtgttgtagtgtacacttgtagtcccggcaacttgggaggctgaggtgggaggatcatttgaacctgggaggttgaggctgcagtgagccgtgattgcacctctgcactccagcctgggcaacagagtaagaccctgtctcaaagaaaaaaaaaagaagaagaagaataagaagaaaagaaaaggaaaccttctccaaggaatctctaaatctattgctgagaaaccagttctctagatgggtgcttttcttgagtctgagatggggcagttgtatcacctgcagtgttgagttatacaatgatacatgaagcatgataatagctgcatctcataacatagataaatctgctgacactacttttacaacactcaaacagaagcaaaactaaagagtgcatttttaactacttattcttgaaataatttcagacctacagaaaaaatttaagaacaagagggaactcccatatgtccttcacccagattcactaaattttaaccttttgccacacacgttattattttttgaaccgtttgaaagtaggttgcatatatcacattggtgttagtccatttgccttgctgtaaaggaatacctgaggctgggtaatttataaaggaaagaggtttatttggctcatggtaatgcaggctatacaggaagcatggtgccagcatctgcttctggggagggtctcaggaagcttacagtcatggtggaagaggaaggggagccagtgtgtcatatgacgagggagggagcaaggagagggaggttgcaggttcctttaaacaactagctctcctgtgaaccagcaagaactcattcattaccatggggttgtcaccaagctattaatgagggatctgtccatatgatccaaacacctcccaccaggccccacctccaacactggaggtcacgttttaacgtgagatttggaagggacacacatccaaaccatgtcagtgtctctttacttaagtgcaaaatatactctcctacatatagtataggtacacatttgggaaatttaacattgacaaagtacttcaatctcatctgcaggcaatattccaaattgatcacttgtctcagtgatgtcctttgaagcaccttttcccgccttgagtccagcatcacatactgcattttgtggtcatgtctctagtctttaatctgcatctgttcctcggctttctgtctttcaaaacattgatgtttttggagaatgcaagccttcttttccgttaagtcgagggtccctacatttacattttcctagtgtttcattataatcacattcagtgtatggatccctgaccagaatgctacctacatgatgctgtatccttctcagggtaccagatctggaggcacacggtgtccatctgccactcattagtgatgttaattttggtcacccagtcaaggtgctattcagtttctccacttacggttaatatgttttcccttccaactaatcagcaatctatgggagacactttgagaccatgctggtatcctgccttttactaaactttcccttaagatttatcatcaacagataatttttgctgaacaattatttactatgacagttgtaaaatgatgatttttaaatgatttcctccaaatttattacttggtattctactatagagccctctcttgttccccattcacccacctacacacccacccacctgcacactgattcacccatctgtcacccattaacccatccattcatccactcattcacccacccacctgcttatccactcatcgtccacctatctacccacccatccacctatccacctacttgtctacccaccatccacccatccctctatccagccagtcatccatccatccatccagtcatccacccatccagccatccagccatcaatccacccagccatccacccatccatgcacccacccatccacccacccactcacccacttacctatccatccatctttcatctacccattcacccacccatccatccatcctcacattcacctgtccatccatccacccacctatccatctactgatgcatccactgatccatccatccacccacccattctcttatccacctttctatccatccactgatccatccatccacccacccatccatccatccccattcacccatccatctatccattcagtcgcccatctattcacccatccatccatccccccattcatccatccacccattatccatccattcagccatccacccatccattcacccattcatctatccatccactcacccacccacctacccatccacccatctatccatccactcagctatccatccatccatccatccatccatccactcagctattcatccatccacccacccatccatccactcagctattcattcatccatccatccacccacctacccatccacccagctattcatccatccatccatccatccatctattcacccatctagttatcatcagtatagattcatgaatttctattttattcatcaggttaaaatccattactgtcctcatacagtttgatgttcagattgtcccagatttggccagcaaaagcccctttaagctgactccagcaacaatatgtattttaaatgtattttctaaaactaagttgagtattaaataaatagttctattaataataaggcaaatttgatcattaggatgactcagattttagggggaaattagtgtccctgaaggcataaaggagactgagaagcagtttctgtctccacagttggcttcactgacctctgtgctatcagagagcttaggatctgacatctgcctcatcatcttaccctggatctgctttctgtggaagaattgcagcagacatggtcatgttgcagttaggactgggcagcagcaaagccagactcgaggcacctagacctgagtttggagcactgttagctgaatccagagagctcagccttcctgagccctgaagagaccacagaggtggcctagtgggtgccacatgcagcctggctgggagtaagaaggagaatgggaaaaaccatggcctccttggtttgcagagaaggaagaatcactgttgttacttctctttgctttaggttggctcccatcatctgagactccacagaggcctggaagccaatgcccctgctttcgacaggtagggattgtctgacaccatccaagcctgtcattgtcatccttagacatcgtcctcccatcagacacaagggagagggtgcgacgggaaagggaggaccctgaaccctgaatctaaagctgctttccctttggtcctttatgcaagaagggagatgacaaaatgggatttttactttgttctcaggtttcctgaaggtaaattaatgaatgttcaggtagcactttataattattttatcttaccagtttcttgcaatgaggcttcatctctatgccctggttaataagggatcatctaaattatagggtatttgaagctgtttgttttttgttttaaaatagtatagaataaataagtatagaaaagtataaagtgaaaaataaaagtaccctaactcccagttaccactctcagagattttactattagtagttttttacatgtcattctagaaaacaattttggcattttatgtgtgtgtgtgtgtgtgtgtgtgtgtgtgtgtatttaaatacacaaattggatcataacacatttactgttttgcagcttgcttaagaaaagcagagctatttgtaatatgtatttgaaataataacttcttaaaagatttgcttatttaatctaaatactcaatattttaaaattgtcttagtgaatatagtaaaaattctcgaaagtaatgtgttaagtgtttgagtccttctacctgaagctttattgagactttaagtttgctttcatactcttgttaaacataacacttgtcatatgagcccagatgtgaccccctgattagtgtgtgacatgtaatgcgcttttctttaaagagatatttaatcaagtatagacaagaccattgccttttccaaagcattattagtatgtcctgagcattctacatgcggagttctttcagttgacattcatctatactaaattgtcttaggaaatagtgaacatgaaaccaaaaatgaaattctacctggctgagcttggtggctcagattttagggagtcgcctgggtgacagagcgagactccatctcaaaacaaaacaaacaaaaaacaaaaatattctacccgaagctgacccaaaacaagagggaagggtcatgctgggttcgcttcaggaaccctcctatgtttaggagctttcttaaaaaatacatacatctagttgaagcacatttgcatagactgcagatttttcagaatcctggtaatgcatggtgtgtgagatgcacgtaccacagagaatgagacctctctctcctcacaagcaccccagaggctgccttgccccttggaaagcattcctaaatctaggggtacacttagatctcttctgcaaatcacaggacaagctgctgaaagggtgagtctttctacaggcagctactgtgtccaggaagtctcttctgtaatgcttcttacccacgacactgggctggaggcaggagccctcccgttcccttgcaggtgctgctgctctcatgttcattgctgactatgttcccctcattcgagataacgtttggaaataaggtgacatgtctgcctagtggccgcagtggggcaccatgagggtccccctgaaagcaggctgcccaggtcctcgatggtgtccctggactggcatgtgcattgtcctgggtgcttcccaattctcttctctgggacatcactgcgctctcaagagtcgtgcggttctctgatggatttggttgagagtaaatcctttctttcccccgcaaaaccttcacacagatagtttttcttcccagaggatgcttgttttcctttgcctgagctgcatgggtgcccctgttagccgaggcggtctcttgtcactttgctgtatgtgctccatatctgaggagggctgtttgcagcaggactttggctgcagctgcttgagtggggtcttgtcatctctgtgtggtgatgagtctcggtggaagggctaacctttcacagggcggtgcctcagtctctcctcacaggacagtgaaccccttctccttccccgccccgctctgcgggcggctgctgtgcacatcagggtgtgctgaactcttggtccctccctgaagatgagaaggatgcttccaaatagaaatctgattcctcagtatcccgaaaaactcctgttttccaagtagcttcctatgacagtaacccgtaggcaggggaagtgaaaaaaaggcctcaatgctcagggtgacccaggaagaagcccgtatgatgccgaccttgctgctggtgccagcgtccccttacaccctgggtgccagcgctcccttacgccctgggtgccagcgtccccttacctgggtcggcagggcctctcagctcaccaataacgagcattttacaataaagggaaagaaagaggaaccacgtcccacacaccaagcactgtccttgtcactttcactcgtctttttagtcctcctcgccattctccgcaggaggggaagtagccctgctttatagatgaaggagccagctcagaagggatgaatcatttgatcaggggagtttggggttgggacagcatagagtaatgatgaggaatagtgagtaataatgagtaataacagtttccttctggccctgggctttggaaccagttgagaagaaggggcaggaatgagacaggaagagatcagaaatgggccttgtttccgatgagccacattgtgggatgtggcttggatgtgtggccacggtgggctccctcaccaggggcagagcctgcacgtgtcagagccaggcctgggatgcaccttctctgggcagaccgacccaaaagaaagcccacaagggctgaagccacacacagcccgccccagggctgcctggagctgcacgtggagacgggagcccgggagggtgctcacccttaacttaggccagggagtcagcaggcaagcctaacttcaaactacccacttcaaaatccatagtctttctactactgtcccttaaagttagtaagagaatgttttctcttactgcataagaaaaaaaatgatttttttcatgatcattattagctctttaaaaaattccgctcataaacgtgtcattacagaaatttggaaaagagaaagatgtttaaagaagaaaagaacttgaatcatataaaattctaccacttttgacatcatgtgtttttttccagtgttttttctatatttaggcataaagattgcaccattaaacgataatttgacatctgctttttgcattgtatcatttccctatgttttgtttaaggcactacatttcaacatgtttaaatttgaaatgtaaggttacttttaaaaagctggtgatcaagaataaatattggaatctctccctaatatttcaacatacatatttagaaagtagcttccaatacaaggaatgttttccaaagcctgcaagttttgttgtatctccttgttgtggtttagcataatgcaatatagcacttagattgaaaacaggatgctgagttgagagctctcctgccagttgttctagttttgagaagggtgggtaccaagcaaaataagagattcatttcacacatgtgcacattgcatgtgtaagactctagcccaggcacggtggcttatgcctgtaatcccaacactttgggacgctgaggtgggaagattgcttgaagccaggagttcgagaccagcctggacaacatagcaagaccctgtctctatgaaaaataaataagtaaaaattagccagatgtggtggcatgtacctgtagtccctattacttgagaggctgagttgggaggattgcttgagcccaggagtttgaggcagcagtgagctatgatcgtgccactgcactctagcctggacaacagactgagatcctgtctcaaaaacaaaattctcttgatgaaagttcaggttaggtgttctagatataatgggaggtgctttatagaaagtgcagcatatcctttgtctgattccaaacaaatgtagaagtctacgctagctagtagtaatggcagctgggaggtgcaggttctctccacccaggctcattttccacgatttgggtcatgggggacagctggtcctggactgtggcactaagaaagcatgactcctgccccgtcttcccttgaggggcagaacagggcagagtctgcacctgtgcccgtgcccagcatcacgcccaccccgctgcctttgcaggcacatggtgcttctgaaggagcagtacgggcagcaggtggtcgtgaaccttctgggaagcagaggcggagaggaggtgctcaacagagccttcaaggtaaggccaggctgtcctctccacagctgggctgggcggcaggtggccatggtggagcgttgagcctggaggtggctcctgcagggactggagctgacagcccagccctccttcgtttcctgaggcttccccagcattccgaccagtgaacacggcagtgctcagtgactcgggccctgatacagtgagtgtaatttcttttctttttttttttttttaagacagagtttggctcttgtcgctcaggctggagtgcagtggtgcaatctcagctcactgcaacctctgcctcccaggtacaagcgattctcctccctcagcctcccaagtagctgggattacaggcacttgccaccacgcctggctaatttttgtatttttagtagagacggggtttcaccatgttgttcaggttggtcaagaactcctgacctcaggtgatccacccgcctcggcctcccaaagtgctgggattgcaggcgtgagccaccgcgcccggcctagtgagtgtaatttctgtgagctgatggttgggagaatttgctcaaagtcaaaacagagcatctcctgtccccagccatgtgtcccttaccctccctccccatggggctttgagggcttctgacctctcaaggtacccctctcagtccagcccaggagcaggcaggcctccctttctgccctggctggtcctgggtgagttcccccagccccacagggcacccggcctcagggctcaggagcaggaacttcggtggctttcccctgcttgccggctggccttcaaatttccccctcttcctcgggctttcatggcagctcctggacctgctgctgctgggctcctgtgggctccttcagacttccttggggggcttttcctggcgatcctggacagatcctgggcagggtcacagagccatgggagcaagagactccgcccacctggccaggttccatccctgtgcatggatcccaggcctccagcccccgccccctgctccccaggcgcaagacgttccagaatgagccaagggggctcctggttcctttaccagcctccatgtcccctgcctaatagagaaaaactctctcgttgtttaaagctcaaagatatcccttacagagagagctcgaagtcgcctcactgggggttagggcgatctctgagcctttccctctgcttagaaatgccacatgctcatgctcagagcaggcacttcctggttgccaattttatttggccagacctgcttttcctgtttggtttgttgctattagcaacgagctccagaagtgtctcccttcccacccatcttggcagtgatgctggctgctaatttgaaggaagaagggaaaaagctaaaaaataagaataataataagaaccccaagtccttttcccctatgccttgagttgtggcccctcccatcagtgggtgaagtgcccggcactggtgagggcgggagatggcagccacctcatgcggagaagggtgggctcctggccaggtagggaggggcactctagctgaagacctgggtcatggccctgcctggggtggccctcagccacctcagcttggcccagaccaaggttggtggcccagcagcctgcccagggcccaatggccatcagtcctgctggatgaagcactggctgtggaatgcatggcactgagccaccaggcactgttgggctcctccagcttcttgctcacagcaagacttgcttgatgatggcagcactgcattgatttacactcagacctatcacaggcagtcacgttgagtcagatcagatgacccccaagtccttctagtccttccaagtcactaggacctctggctgggcagaggctggactggcctgacctgaagccacaagggcatgagtgcagggtgagaggcctggaagccgggtggcctgggtaggcggacagtttgtccttcatcacggggctctctggggagaaagccagggagctgccaggccagggaagcctcatatggaagggagggctcttaagggcatcctggtgccatcagcagccccttgacctgcttcccatctctccccaccttcactctggctccctgaccatcctgaagtagcatcgggatgtgggacccacctgggccacttcagctggagacttggtctcttcagagcataaccaccagaggctggtgcgcggcaggatggggccggttactagggcaacggggagctggattatgagctctgcagcctgccacagcactgctatctctgagggcctggggcacctgggccacctgaagaacagagcagtccttcctcacctgtgccctccagagagccctgcaggagtatgatctctccaggaagccacacctgctctggcttcttttgctcaagggggaccttctgccctttcgcaggcaagacgctctttgtgggttttgtccccccagggcctttgtcctgtgcttcccttttggggtcaactgcctggtccactgtgagcttccagctaaagcacgtcctgcggcgagtcaccttggttagccgcacgggtcaagctctgcaaggagcttgccctcctcgtcctcccttccctctgctcccctcaccgccctcccctcctcttttcagaagctgctctgggcttcttgccacgcgggcgacacgcctatgatcaattttgacttccatcagtttgccaaaggtgggaagctagagaaattggagaccctcttgaggccacagttaaagctgcactgggaagacttcgatgtgttcacaaagggggagaacgtcagtccacggtgaggctcgctgcgcactgtgccgcgtcttctgctggggggaagcgtcagtccacggtgaggctcgctgcgtgctgtgccttctgctgggtgaggcggcagaggggccgtgcagtctaggacatgtgccctatgaggggcagcttccttccctccccactcagttacccttgtggtttcctgggtcataagcttggcgacgccgggaatcttgggttccagcttctcatctcccttcccacagtaggagacaggcttttgaatttctcaaggagatgttgagtcatggttttctggcaaaagaaatgaactaataaaggggactgcattaaaacatggagctccacgctgtgacgcaggcctgtgagagtgagggccacagtcgcggtgggatggctggcatagacttagattgctagaggagaccagggtatggacttccctgttcagagcagaggagaaaaacgaagtccagttccctcaggtcacatgcagggggtcctcattatttgcagattccatattggcttatttacctattccttaaaactgatttgtaaccccaaaatcagtatgcaaagcatttttgctggacagagtatttaaatactctggcctcgtatttaaacaagtttcctttttaagatttagtaccatgtttttcacatttgtggggtctttgttaggggcccccctttgttcggggatctcatggtttagaatggccccaagcagtgtgccgaagagctgtctagtacttctgagcacaagaaggtggtgatgtgctttatggagaaaatgcacgttaagataagctttgtccctatatgagctacagtgctgtcggccatggttcagtgttaattaagcaaatatatatattcaataaggtgtctttatatggaaacatacataaaacaaggttgtatattgatcagttgataaaaatgtgaccagaggtttgcaggaacctaaccctatatttcccctaggagcgatggttctgtgttcatgtccaattcagtgttcctacaactttatagaacataattgctataaataacaaggaccagctaaacgtaaaacgacccagctgtgcaggctactgagtgtgtgtcctggggtctgcctgcctttccagaagtgctggggatccactgggatggacactggggtttctggggggctattgatgaggaggtgaggggttccctcttagaacatgtttcctcacacctgtaatcccagcactttggaaggctgaggtgggcggatcacaaggtcaggagttcgagactagcctggccaatgtggtgaaacaccgtctgtactaaaaatacaaaaattagccgggtgtgatggcacgcacctgtagtcccagctacttggaaggctgagacaggagaatcgcttgaacctgggaggcggaggttgcagtgagccgagatcatgccattgcactccagcttgggtgacagaggtgagactctgtctcaaaaaaaaaaaaaaaaaaaaaaaaaaaaccatgtttccttgggagtcaaaagtcagacatgggcctctgtgctatggccactgcttctttgaaaacaacatataaccgtttacatttcttcttgtcctaagttttcagaaaggcactttgcggatgaactgtcttgactgcctggaccgaaccaacactgtgcagagcttcatcgcgctcgaggtgcgtccctgccacagccttttcggccatctgtgccaggtcctgtgacttcagctggacaggctgggctgagcacctttagcggcaagatgagggtggcatcaccaagacaaccttctgactatggggaaggtggacagggagacactgggacatccagagaggggttccccaggaggctgctttctctgtcgtaggcacactttctcacaccctcttgtgaaatgcatcccagacatggtggcccaaaggctcctcgggaacagcggttgtgtgtaatgattatcgtggatgcaccattggccaaaggcagtgtgaggctggccggatgaggctggatttgaagctggggagggagacctctgtctgcatggccccagctgcctggttgacctctcctggctggggggtcgggggtagggtcagcaccccctgctgtccaccactgtttgctcttcagcctctggcattgggtggggcggggcggggtgcaggggcgttcaagtgtcagggggtgagaggagggcttgggtggaccctatagataaagttggggtgagatgtgggaagtcactctctgctgtgggaactcctcatcctctggaggggcacagagtcttccaggcacagcgtaatccacaggctgccgaataaggaacctcctgggacagtggctgcagggcctctgtgggagaagccagtgacagccatcccttattcctccccaggtcctgcatctgcagctcaagaccctggggctgagttcaaaacccatcgttgaccgctttgtggagtccttcaaagccatgtggtctctgaatggccacagcctgagcaaggtgttcacaggcagcagagccctggaagggaaggccaaggtagggccccgccgagggggcagggtgggggccccagggacccccctcacggcctgcggtctgggctctcggcaggtggggaagctgaaggatggagcccggtccatgtctcgaaccatccagtccaacttcttcgacggggtgaagcaggaggccatcaagctgctgctggttggggacgtctacggcgaggaggtggcagacaaagggggcatgctgctggacagcacggcgctcctgggtagggcctgccgcagacagggcgaggcagggaggtagggtgctccccagccccactttcccaccgcctctgtgctatccagagagcgggtggcagaggtgcctgggatctgagcagtggaccatgcaccttgcagctgtcactgcctctcccgggagcctgcgctttcctgctgggcctgggagctcacgcttcctctgtctcggcttggactgtgtttccatcctgagaagcgagagacaccaggcgggctcagggagaggccgtgcactctcctaaacgcatcggtgcctttgaatgctaacctactccagaaacttcctcagctgcctgggctcttgggcccttagcaggtgatgttggctttgggagcttcccatgaagttggcctccagtttctctcttggcccctgaggaccctgtggaaacagagaaggaggatgctgtcatcccctggggatgagggcaccttctggtctcagcggcaagacatcgtgattccatcaatataggaggttcctttgggagcattttaatggcatcttcttacctgagcctgtaaacatattaggtctcatggaaaagtaggggaaacccagcatttttccttataagaatgtacaagtgtcgctttagaaatgtcaggtgtttttgaaaaatgaactagcccatgcttaaaatgggttcacggccctttccactgtcaccgtcactgggggagctctaatttggcttccgataggcacggtgatatttagtttctactctggaaacagtgccagctctgtaaaaggtgaacaaagctccatgttgtacagtgagtcttatttaatagcaaggcagacatgagttagatttggtttacatcacaagtttaaaaggtggaatatattttggctatctagaggatggagatggttctgtgtagtgactttttaaagccttaagtctggggcaaaacaaaacatatcagacatgcaggctggaaggcagaggatgcaaaggtggctgtctgggttggtaagacacttaaagttatagtgtatatggatgcatacacaatgaaatatggtcagttacagcatgcccttgcttaaattcctatgccttgttcttctacggagtggacagtgaggacacggcttttccctgtcgtgtctgagatccctatggtctgatgtcccatggcttctggcttcagttgggggtaggggtatagacggtgagtttccagtcttatgttttcacgcctttattgtagaaatgccataagggaagccccgtggtgaccgtggttttaagccttttgtcgtcttcgtaaggagcataccctggtttatctctagaggctcatgaaccatctgtctctgccaggcagcctcatctgtctttttggaggatgcctgagctttggaactgcaaagaaatgtgcctttttatgcctcctgacagtgactcccaggatcctgaaagctatgactgagcgtcagtccgaattcacaaatttcaagcggatccggattgctatggggacctggaacgtgaacggaggaaagcagttccggagcaacgtgctcaggacggcggagctgacagactggctgctcgactcgccccagctctcgggagctaccgactcccagggtgagggcagtgactttgggggagacaaaatccaattgcacctaacctgacatgtcacgcttgaccctttagtggccatcgtcttgtggaatttcatctttggtgtcttccctcctcacaatgtctaaatagcaccatggcctgactctcaagaatgctgcctcgcaagtaactgacctttttcctttcccagatgtttagaagagttagcagatggtgaatctctcttttctctttgcatacagtgatggtgaagtcttaggctttcatcagtagaatcttttcgtcaccagtcggaaggtgtggcttctagggccatgtaactgtgaggttgggagccaagcctgtcttcggtgttttgtttttgttttttgttttgttttgttgttgtttgtttttttgagacaagtttcactcttgtcacccaggctagagtgtagtgacgcaatctcggctcactgcaacctccatctccaaggttcaagcagttatcctgcctcagcctctcgagtagctgggattacaggcgcccaccaccacgcccagctaatttttgtatttttagtagagatggggttttgccatgttggccaggctggtctcgaactcctgacctcaggggatccctctgccacagcctcccaaagtgctgggattacaggcatgcgccaccgcgcctggccctgccttcagtgttgatattgaccaagaacagctcatccctgcctctccccaaaccaactctattaatctgctttaagtagtcacaaaataattcatgtgctatttaggaataaaataattccaaatgttgacacctcaccttttatcttgtaagatacactattgaatggccccagaacttaggatctgaagaataaatgattcttgtccagtcatcgttgactcgggccttggtttttttgtttgtttgttttttgttttgcttccttggactcaggaacttgtggttcaggtttttgcctgttggtgagtgacctgtggtctagctgacatgcagtggcccatgtgtacattcatgccccacagctgcctgggtagtacccagagccccatcccttcctgtgcacacgccatctgtgccctcctttctgcctccaagggcaggtggcagagtccaccattcagagccactggggctctctgtgtcggtggtagcctgggttgctgcttgtggtgcctccagaagccattcaggatgccctccggagcaggcccatcaacgcggagagtgcttagcacagccgtggctcaagagccgtccctccagtccccacgaaagtgactcctcttcttttccctgcaggaaggtgtgcatgtttttagcaagctggctggtattttgcaggggatttttttggggtagacactagcaggctccccagcagtcccactagagatacaccaggttccagagaaggaaaggagtgctgtggttactattgccaagcagatcagacagagggacggggattgttttatttaaaaaaaaaaaaaaaaaaagctttaagtcagggaaactagaggttccaggttgcccattgtggtcaacagagaaggaagtggacagcctgcctggcctctcgctgacagaggccatgtcctcctagctggaccctggggaggtgggcctctcactttctgcagcagctggagccatgactgggcttggtggctcaggagcctgggtgtcagaggtcgtgactggacagattcagtcaatggctacaaggttatattaaattctgacctcccctgttcaaagggaacagaatggacggagggcccctctgggctcactgctgcatttaagccaggcatttctgaagcagattccactggggcagttcccagcagcaatggtagccacctggagttggactcaggcagtggacagcatgactcgggagagggcactggggagccccatgaactcgtaatgcgggacttggcggttctgacttctgtcatctcttgccttgctccccagatgacagcagcccagctgacatatttgctgtggggtttgaagagatggtggaattgagcgcagggaatattgtcaatgccaggtaaggggccaggtgtgcggggccaggcagggacttcctgagtcaggtgcctggctgggagcagagcctctgaggctctccgggagccagcctgcttctgagccagctaagctgtggccctggctgtgtggggatctctcacctggctccttctctatgctcacacatgcaccaccccgttctttccctgccacttaggaagtaaaaggaagggctgatgattgctgtgttttctttgcagtgctctggcctcggccacttgttgaggctaatttatgctgccagagggagctggcatgtgtggacctggggtgcaggggaagggcggggctgggactgggagagtagttgactcatgtttcctggggagaggtgagaaagtgtgagtgccgggaagaggtactttcttcctgcacaccagggcaaaggaaaaaatcaaaaccactggatttttatttgatatgaagtcaactgggcgctccccccaccccctgctgaactggcctggctttattttaaggaactttctgaacaaataaataagactgactctgtggggctgctatgagaaggtcctgggttgggtttcaggggtcaagggagggctgctgtgatgtgtgaggcatagccaaaaacacgcagatcctccagtagaagaaggaaagcatgacactaatattggggtgcgggcagcgtgaaatcagttctgcaaacagaataggcttgggctggagcatttggtaggtgggagatagatgtacttccagctacctgtaaacagcatcctttccttttctcttccagtactaccaacaagaagatgtggggtgaacagcttcagaaagccatctcacgctctcatagatacattctgttgacttcggcacagctggtgggcgtctgtctttatatctttgtacgtccataccatgtcccgttcatcaggtaagaacattctgtttacacacatctggggaacaaggggttgttagtctttgtgttttgttctttgacttccccctcctcccccatccccttctgtaacaggaatcgggacttaccattattttagataaatgtagcaaaaccccctaggaaaaactgggctgaatagaatattgtctagcgattaaaaagccaccatgggaaatgtctaactcaggaagctgaactcttagaaagggtgttacatgattgtcatcgcccaagctcaattccgtgcaaggaaaagggttccttgttggaatcttatagacaggaaacctctgaccaagagctgagtaactcactgggaaatgccaactcttttgtccctttttaaaagaattctaagtagaacgtgtgggcctctacaactgtgacctcattgtcatttctccaaggtgcatgctttgtgagcatcagaaagggggcgagcttaggggcggcattcctcttggggtgtgggtgtgggtgtttggatgctggaggaactcatcttttccccagcttgtggttggcctcatcttaccacctgggcctacccatggcttttgaatttccaccagcctttcggcgagctggcagcaggcgttgaactgacctccccactgagcccctgggtcccgggaagggcctgtgcctgccaagagcaccacgggtacacccctgcagcacctggagactaagactatgctgcccataatcctcttctctgactttcagaagatgtttaggtccctgtccctctgcttgtgttatttgggcagcttggcagtgtcctaggttagcaggtgaaggttagtaaaggttaaaggcaagggcggggtgagggtgagaggtaaagcatttgagaaagggctcagagctgaggagagccaggtttcccaggctcggtgtcctcggcttcacgtgcctttagccctgagctgcttccgcctacaccccttccatcagtcatccccccaaccttgtctgccagcgagcagatgcaccagcaggcctcagtctgtggcatggctgtggaaatgttaacaaatgtgcttgcagcccatgtttctatggactcgtatgcgtcataagtgggcatttgaatgcaagcaggctctggcattgcataaaagtccttttgagaccatctacccagcccagcataaacagctgcccgtttcctagctcttaaagtgcattgattgctgggcacggtgtctcacgcctgtaatcccagcactttgggaggccaaggcaggcagatcacttgaggtgaggagtttgagaccagcctggccaacatggtgaaaccctgcctctactaaaaatacaaaaattagccgggcgtgatggtgggtacctgtaatcccagctactcaggaggctgaggcaggagaattgcttgaacctgggaggccacagttgcagtgagctgagatcacgctgctgcactccagccagggtgacagagcgagactctgtctcaaaataacaataataatttaaaaatgtgttgattgcccagatgacacctcccatgcatggcgtgatggctgcacaggagtggggtgggctggtgggtggtgatgtttgcgccgctcctggtggcagtgagaacctgggcggatgcaggcattgccctgtcttcggcttcatggggtgctcaagtgagcacttcccagttggggtgtgggggagatgtccaggcagcggtgggcagcaggagggaggcggcctcctgaccaggagtcgatgacggccacggtggccactgtgttgagctgatgattttggagcactcagagcagcaggtcccgagctgctgctgggcccttctctgtggcaaagcagggtcacacttctccttcaggagccgacggcatgcgcgtatccttctgttccagggacgtagccatcgacacagtgaagatgggcatggggggcaaggcggggaacaagggcgccgtcggcatccgcttccagttccacagcaccagcttctgcttcatatgtagtcacctgacggccgggcagtcccaggtgaaggagcggaatgaagactacaaggagatcacccagaaactctgcttcccaatggtgagcggcgccgtggggacagccagcacctccctgctcagctcagtcccaaatgtgactgttgataggagccaggcactggggacacaaccacagggagggccccttcctggagggctcagcgctgggggagggggagagggctatgtccctccatggaattgacctggggcagggttagccacgtgcctggagggggccagctttgcagcatttcccgttgctgtacaattcctgagtgtctgcatggccactggatttgtatgtgtgcattttctgattttctaattcaagtgatggtttagggaagaaaaccatgtccgtggtctgtcctgtccaagtggtgaagagcatttgaatccacacagcctaagactccaccttcctgagattcacagtcttcacatgtactttctgtggtggctcagataagcacacccttttgaccaatggaccctttttgactgggaacacggttcttgaccaaggagcccagatcctgcctgtcctcgaagctgcaatagcgaaccgcacactgccctctcgtggcggtccggagggtttcattctgaaactggaggattttcttttcaaatctagacgttaatgttctccttcctgtgcaaagatgctacccgggcacagcttcagcgtgtggtgcacacgaggccacctctttcccgaagtgaggatagacttcagctgttcttgctgcaaaatcttccactgtctgagaggtggtgggttgggtcacagagctcttccctgtgagaacagatccttaaaactgacgtcccattttactgggctgcacgagcactcaaggcaaaatttccttagttttcattaagaagtgccataattcctgttggtgttgtttatttcttatgttaaaaaagaaacaaacgagttgggaaattttcaattgtgtgtaatgcagtttaaaaaaaaaaaaaaagtctaacccttctcacttaaaaatagctactttggggccaggcacagtggctcacacctgtaatcccagcactttgggaggccgaggcgggcagatcacctgaggtcaggagttcgagaccagcctggccaacgtggtgaaaccccacctctactgaaaatacaaaaaaaattagccaggcgtggtggcgtgcacctgtaatcccagctactcaggaggctgagacaggagaatcgcaacccaggaggcagaggttgcagtgagctgagatcgcaccactacactccagcctcggcaacagagcgagactctgtctaaaaaaaaaaaaaaaaaagtgctactttgacatagtaaacttgggatacataataagtaattaaaggatttataatgtagttcttttctaccataaaaatattgagggacctattaacattctacacttcttttttgtttttgttttttgagacagtctcactctgccacccaggctggagtgcagtgacatgatcttggctcactgcaaactccacctcccaggttcaagcaattctcctggctcagcctcctgagtaattgggattacaggcgtgcaccaccaggcccaactagtttttgtatttttagtagagatggggtttcgccatgttggcccagctggtctcgaactcctgacatcaagtgatccgcctgcctcagcctcccaaagtgctgggattacaggtgtgagccatggtgcctggccaacattctacactggtattgtctgataccgtagtcactggccataagtagctatttaaattcaaattaattaaaattaaataacatttaaaattccttgctttccctggccacagtgagaatgatcagtatccatgtggccagtggctcctgcatcagatggcacagagaacatttcggtgctgcagaaagttctgtctgacagtgttatctagacgctcattttatttggttgggacctgtttaatcttctgtccagattattgtcatggggggagtggaaatgcctcatattataggagaagagaagctgattttggagctaggcagatgggagctgaaatcccatctgtatcatgcactagctgtgtaaccttcagcaagtgcctttgtttctgaactttgtttttttttttttaatctaagagagagctggtggtatctgctttgtgcacctggaacagagcccagcccttgcggacagttgatccacagcagctgctgtcattgtcttccccattcctggggaagagacagggaaggcaaggaccccaggagctctccggggctgagctggggccgtgggctcctaggactgggcaggatgggaaggaggagctagagagagatggagaagggtcacctgaaaaagctcaggaagttacatgaaggtgcacatcctgagcatttctgctgctcttttgaaacctcattctcctaacgctgaatgctgggtgctgcttcctaggagtagacactccaaagtaagggtgcatctgatcttcaggacggggatccatggaggaaatgctggccatgttgctctcctccgtttagggtaggagctttcttcaaaccactcagaaacactattatttaaaattaaaaagtaagccatgtaaaaattcctccatagttttatgggatgagtgaaaaacaagatgctctagtagaaaactttcttttttctaagtagcattttgagaaaaatactcctgcttgccccagtgagcttgtttttctcctctctcccaccttccttgcaagatgggctgaatgattatgattttcttttcaacttaggggagaaatgttttttctcatgattatgtattttggtgtggcgatttcaactaccgcattgatcttacttatgaagaagtcttctattttgttaaacgccaagactggaagaaacttctggaatttgatcaactacagctacagaaatcaagtggaaaagtaagttgtgctttaaaaacattttttagcagctgctccaagatggaatgacatctgtgagatgtagaggctggtgcagcaaattcagttccgtgagtggatttcactgtttccaacattgtagaaaataggtgggcttgtgaggttacagtacaggagtggctgttcatacacacttcctgtccttttttttttttttttttttttcctgaggcagagtcttgttcagtcacccaggttggagtgcagtggcactatcttggctcactgcaacctccaccttcctgggttcaagcaattcttgtgcctcagcctcccgagtagctgcgattacaggcgtgtgccgccacacctggctaatttttgtatttttagtagagacgggttttcaccatgttggccaggctggtctcgaactcctggcctcaggtgatctgtgcgcctcagcctcccaaagtgctgggattacaggcatgagccaccatgcccggtcaaccttctcctccttctaatactgttattttagtgcctgtttgcttcctagcaggatgagctgagatgaggggagtacatttaaattccatttagctgtggagaaagaagctggttaaatgtgtgagggccaggtgcggtggctcttgcctgtaatcccaacactttgggaggccgaggcgtgcggatcacctgaggtcaggagttcaagaccagcctggccaacttggtgaaaccccatctctactaaaaatccaaaaattacctgggtatggtggcaggcgcctgtaatcccagctactcaggagactgaggcaggagaatcgcttgaacctgggaggcggaagttgcagtgagccgagatcgcaccactgcactccagcttgggcaacgagcaaaactccgtctcaaaaaaaaaaaaagtgtgaggacagggcagataaggtctctcctgaagcaagatttggacgttcttcgaggtttactgtgtcttcccacttgagccccttccagtgtgtggagtgctaaagggttgccacatggacgagtgaccataccaacctcctaaagcaggttttcctgtcttcctactaccaacattctcacatcctctcagtgatgcccaagagaatgagggtgtgtttgtcccttggtcttcccatggaagcaaggccaatccaaagttaaacaaagaaaagaaggatgtgcatgggggctcacgactgtaaggcgggaggattgcctaagcccaggagtttgagaccagcctgggaaacatagggagactttgtctctacaaaaactaaaattagccaggcatgatggtgcgtgcctgtagtcccagctacttgggaggctgaggcaggaggatcacatgagccccagaggttgaagctgtagggagccgtgattgcaccactgtattccagcctgagcaaaagagcgagaccccagggggagagagagaggagagctgactttcactctgcgcttctatgtacttagctcatttagtcaacaaacatagatgaaggcctcggggttgtgggccagacatggcaccaggcatgggactgtaaagatgagcacccctgacccgtgcctcggaccctgcactgtctcctggggggcaacaagaagacagtggatagatcagacactggagtgggtggggagatagatacgagggcggtgtgtccctcagtgggatgagctgaggatcacttttttgctaattaaaagacacatatgtccagtttcagatagattttcaaatgtaatggagcttctgttactttttaagaatagggacttgccttttcagttttcgtttttgtatataacattcaggatcgaaaactacggtggcctgatgacttctttttctcccaatagatttttaaggactttcacgaaggagccattaactttggacccacctacaagtatgacgttggctcagccgcctacgatacaagcgacaaatgccgcacccccgcctggacagacagggtgctgtggtggaggaagaaacatccctttgataaaacaggtgagggggccgtgcccgttcgagagtcggcagagggtgaataagaaatgcgacggagggagagtcacgtttacccaaccccaggcgctgctacataaatcagtttcttcataaccccagatgacacaaaagtcatcccagaccttaacccctaagctttggaagttgtgctttcagggagcctctgcttctggcctctttttttcccttttcttttcttttttttttttgagacagagtttcactcttgttgcctaggctggagtgcaatggcgtgatcttggctcaccacaacctccacctcctgggttcaagccattctcctgcctcagcctcctgagtgctgggattacaggcatgcaccaccatgcccagctaattttgtatttttagtagagacggggtttctccatgttggtcaggctggtcttgaactcccaacctcaggtgatctgcccacctcggcctcccaaagtgctgggattacaggcataagccactgcgcctggcctcctctggtctctttctagagacagtgacctgcaaataggtctgctcctggctgcccctgcatcacagctcccccccacacccccacctcttttttctgggtggggaaagcggggagcatgaggttgcctccctccctgccccccatgctccccctcatgttctttatccagcctattttgttcagctacacttttacctgcttaaagccctaattccaaattggacacgagcaagacacagtaaaaataagttagtttttaaaaatcattagttgtcaagtatgttttgaacaactgttgatgcccagctaggaagtgaagtttatttaaagttagaatatctgtaactagtacataaaaaaaaaaaaaaaaaaaaaaaaaaaaacctaattacacattttgggagagcatagccaggaaattgcatctgtgaaaagaaaatacaaattttcctctagatgagtttgtgaacgcctgtgtggccccagcgctctgagaggtggggcctagaatgagtccgagtgttctcagctcctgcagctcttaccataccaattgctgccaaccttctgggattcctgggtcctggaaacccggcagcaacactgtgtctcaagcctacagtgggcatgctggcgaggactgcaggcatggtcatgactgttttgaataattatatgcgaggaatgtttttaaccaaaggccccaaagcatccgaagcctggcttcaaggtggaggagtaaggaggatgagtctgggacgtggggtggttcgtggcgcagacctctattgtggaagtgtcatgatcgcattcttggatattgactcgattctatatgggtctctcgaatggaacccctgcgagtagctggagaactcaaccttctagacagtgatctagatgttgacaccaaagtcagacacacctggtctcctggtgccctgcagtattatggtcgtgcggagctacaagcgtctgatcacaggtgaggtcctgacttccatggcgttttctcctgtgctgggcaaggcagcgtctccctctccgcaggaaaatgcatgctgtccccataaggatgtgtgtgcgcatgggggtgcattttgtgtgcatacaatacaccaaagaacaaagaatgatggaacaaatatctgtggacccaccaccaagaactaattaatcttagcattttgtcattgctccatatcataaatgacatcattgatgaaaatttcaaagatgcaacctacgtattttgcggcttttatattttcttaaatgatgtcgttgtgcaacttgcttttttgttactatttttgagatacagtcgtgttgatgttatagctctggtgcattcatttgtaactgctgtctagtatcccactaaatatatccatggaacttacaacttatccattcacctcttgatgaatattttctccaggattttatgattacaaacaccatagggtttttaaatctgggatgcacaaccttttttttttttttttttttgagagagagtcttgctctattgcccaggctggatggagtacagtggtgtgatctcagctcactgcaacctccgcttcccagattcaagcaattctcctgcctcagcctcccaagtagctgggattacaggtgcacaccaccatgcctggctaatttttgtgtttttagtagagacggggtttcaccatgttggccaggctggtcccaaactcctgacctcaggtgatccacccaccttggcctctcaaagtgttgggattacaggcgtgagccaccgcacctggccctaggatgcagggctttttaaatgctctgtgcatcatgcattgaggatagagtggaggcagcagagggagcaccgtgggcaccagcacctgtgccctgggattgctgcagaggaagactcaccctgcagaatgtctttaaagacagaggagaggatggcatttgagggtgtgaagaaggaagagtttgatcttcccaaggctgctgcctaaactctcatgtcagtttcaacttttacaaggaaagaaattaagaaaaagaaggtaagaacagttggtcaacgaaaaaaccttttttttttttttttgagacagagtcctccaggctggagtgcagtggcacaatcttggctcattgcaacctccacccccagggtataagtgattttcatgtctcagcctcccaagtagctgggattacaggcgtgcatcaccatgcccggctgatttttgtatttttagtagaaacggttttgccatgttagccaggctggtctccaactcctggcctccagtgatgtgcccgcctcagcctcccaaagtactgggattacagccaccatgcccagactctttagataattctaaattctttagatacctgttgttgcaaatacatacctagaagtgaatcttgaggaatcttcagatatgtgacatcaaggtttgctagctcaatgtattttgaaaccttaatttaaccaatatttcttgaggggcccttacatgccaggccctgttgctggcctggagaaaagcagtgaacaaaacagatgagaccatgttatcatggaattttctggacaggacaaacagacaataaacaaacatgtgtgctatgaaagaaaaaaagtagtggcaggagtcaggccagagtcgagctattttcaatcaggctgtgtaattttcgtaatataggattttctggaaatttatagagtaataaccatccctgtgtcagaattactataaggattgcattatttcctgctggactgttttcctgtttacatcattgatttttacattattcctaatgattgcgtagggtattatatgaaggtatcataactgtttccccattaagaaacagagcattttggcctggcgcagtggctcacgcctttatcccagcactttgggaggctgaggcaggcggatcacctaaagtcaggagttcaagaccagcctagccaacacagcaaaaccccatctctaccaaaaatacaaaaattagctggtcatggtggcatgcgcctgtaatcccagctacttgggaagctgaggcaggagaatcacttgagcccgggaggcggaggttgcagtgggctgagatcgcgccactgcactccagcctggacaacagagggagactctgactcaaaataaaaaaaaaaaaaaaaacgagcatttccctttttttttttccctcccatcaacaatgctgtaaatgatcaacctcatgcattaaatctttgtctgaatccctggtcatttccttagaataaagtccgagaagtgagattactaggtgaaaagagatgaacaattttggaactctatgtaaagtgccaagtaaccctctgaaaagcttgcacccctggcagagtgcagatgtctgcttccccacctccttggtggtactgaggattaccatgtcaaaaatctttgctgatggctggttattttccaagtactgggatacatctttaaggtgccacactttattttccccctcaaatcatctttagataaaaatgccaaggtgatactaaggttaaggaaagatgaagactaaaacgccagagcagggcagctagcctgcaaatccacctgctggtggccacattcgcaggttgttggtgttgactgtttacaaagagccctgggggacagcagctggggactgggggctggagatgtggacatctgtcccaggctaactggggactggaggccgggggtgtggacatctgtcccaggctaactgtcatccctcttttgttcctaacgcagacctgtgctggcgatcgtggaggtggaagttcaggaagtcgatgtgggtgctcgggagagggttttccaggaagtgtcctccttccagggccccctggatgccactgttgtagtaaaccttcaatcaccgaccttagaagagaaaaacgagtttccagaggacctgcgtactgagctcatgcagaccttggggagttatgggacaattgttcttgtcaggtaactgctcccctggctgatgtgggttccaggggatgtcgatgaggatccgtgagagctgccaggtcgttcttggcattgacttggagtatcatttattccaggatcaaccaagggcagatgctggtaacttttgcagacagtcactcggctctcagtgtcctggacgtggacggtatgaaggtacgctgtacttggccacactgtggagtggggtctgatcaatccctctttttatgtgggcatgtcttcttcctcctcttgccctgacctgtgccttttttttttttttttttttttttttttttctctgagacaaagtcttgctctgtcattcaggctggagtacagtgatgcaatcacagctcgctgcagccttgaactgccaggcttaagcaatcctcccatctcagcctctcaagtagctgggaccgcaggcacgtgccaccacacccagctaattttttttaaattttgtagagagaggatctcactctgtagccccggctggagtgcattgggacagttatggctcactataacctcagcctcctgggctccagtcatcctcccgcctcgatctcccaatgtgttgagattacaggcatgagctttcatgcctggccaccaatctgtctttatgtatagacgccatatgcgggagggagtttggattcttcactctggttctaaggtgtctcatgtccggctctagctacttgcaaaatagggcggaagggtgggcgcagtggcttatgcttgtaatcccagcactttgtcaggccaaggtgggtagatcacccaaggtcaggagatcgagaccagcctggccaacatggcgaaatcctgtctctagtaaaaatacaaaaattagccaggcgctgggtgtggtggcttacgcctgtaatctcagcactttgggaggctgaggcgggcaaatcatgaggtcaggatttcgagaccagcctggccaacatagtgaaaccccgtctccactaaaaatacaaaaattagccaggcatagtggcaggtgcctgtaatcccagctacttgggaggctgaggcaggagaatcacttgaacccaggaggcagaggttgcagtgagccgagatcgcaccattgcactccagcccaggcaacagtgtgagactctgtctccaaaaaaaaaaaaaaaaaaaaaaaaagtagccagacatggtggcgcgtgcctctagtcccagctactcaggaggctgaggcaggagaatcgcttgaacctgggaggcagaggttacagtgagctgagatcgtgccactgcactccagcctgggtgacagcgagaccctgtctcaaaaaaccaaacaaacagaatggggaagaagaatgtcagtcactgtgtctcacgttggaaggcagctctaccagcagctgcagaagcttagccattttagagtgtagttctcgccgagttacttgatctgtctatgctcgcactgccatgtgggagactattccagtctttctctttagagtatgtattttggaatatgcagctcatagatagtttccgtttttatagtatcttggctttaaatctgtaccaaatgggaagctggagatgagagttcaactttttttttttgagatggagtcttgctctgtcacccaggctagagtgcagtggtgcaatctcggctcaccgcagcttccgcctcccgggttcaagcgattctcctgcctcagcctcccgagtagctgggactacaggcactcaccaccacgcccagctaatttttgtatttttagtacagatcgggtttcaccgtgttggtcaggctggtctcaaactcctgacctcaagggatctacctgcttcggcctctcagagtgctgggattacaggtgtgagccaccgcacccagcccagttcaacttttttttcaaccagatgtatgagagctatgctgagcctgcgtgaggtctggacccttggtttttcagggccaaatgtgaagattggcctcgttgagggtgtgtgggttttggtgagaagcattgtgtgatcaacaggaggggtcatgatttttatttattcctgggtggccgctctgccctcccaggtgaaaggcagagcagtgaagattagaccgaagaccaaggactggctgaaaggtttgcgagaggagatcattcggaaacgagacagcatggcccccgtgtctcccactgccaactcctgtttgctggaggaaaactttgacttcacaagtttggactatgagtcagaaggttagtgaccctgcagggagggacaggcaagccgttcttctagcaacaggcctgagcttttaaggacaccttctaaagcctcccttgcagaagtgacggaggacacccgcagggcaagccctctgtcccatgtgatgagcatgtttgtatgtgtggacgatgcatgccatgctacctgggacccgaggcatctaaacttaggctacaaacagtggctatgaggatatcagggctgtgggtgtaccaatggaactggggtaccgtccctgctggcgcctgggccctgagctgcagggcagggtgcacgtgtcctgacaggagctgaaccagtcccactgatggaagactgagcctttccccctcattgttttcacccgaattgtgattcatttccttctcccaggggatattcttgaagacgatgaagactacttggtggatgaattcaatcagcctggagtctcggacagtgaactcgggggagacgacctctctgatgtccccggccccacagcactggctcctcccagcaagtcacctgctctcaccaaaaagaagcagcatccaacgtacaaaggtagcctgacccttcttttctcaggagcctcagcgatggagtcagctcaggacagactttcctttttctcttggcgattgggcactgtgtgatatcaagtatgcaggtcccaggagagacctcaaccaggcaggccaagcgaggggtagggactgagccctgtggaggtagccagctccaacaccctgatttcctctagttagtttatttttttacaataacttgtacccccataacacaccagattcaagataattcttgtttcctctggatattcaaatggccacggttctgaactggttttatatttgtgattatttcccgcccccaacaaaactcttctgattcagccaggcgcagtggctcacacctgtaatcccagcactttgggaggccgagacaggtggatcgcttgaggtcaggagttcaagaccagcctggccaacatggtgaaaccctgtctctactaaaaatattttttaaaaattagcagggcatggtggtgcacatgcctgtaatcccagctactcaggcagctgaagcaggagaatcgcttgagctggggaggcagaggctgcaatgagccgagatcgccccactgcactccagcttgggtgacaaagtgagattccgtctcaaaacaacaacaacaaaaaaacttctgattcaatcccagtagagacataatccatatctatgtcctgagttgctggcgtatggtcacactcatataatatcatattattatacagtcatgtcatacggtcacatcattgtatcattttagacttcagagggcctgaaaggtcacattgattgaaccacacacccctagattaaataatacaactttttttttttttttgagacagggtctcactgtctcccaggctggagtgcagtgatacaatctcagctcactacaacctccacctcccaggctcaagcgatcctcccacctcagcctcccaagtagctgggactacagggttgcaccagcatgcccagataattttttgcattttttgtagagacggggtctcactatgttgtccaagctggtctcaaactcctgggctcaagtgaccctcctacctgggccttccaaagtgctggaattacaggtgtgagccactgcacctggccagtaactgttctgagtaacttttaaagcccactaatgttacgtggctggcaggtcgggtagttcatatttcagtgtcagctacattgatgctgttccattcatagccaggcatgcctctagctgtcaatcactgagccccgagttggaggacagctctaaccagcaggtgcaggagcttagccacgcattgggcccattgagctggggctgagagcagagaacagggctgaaagtggattaaggtacctctcagggagggtactttgccaggagaggtgcgaagacctccagcccctttgaccgtacgctggtccccacactgaccaataacttgcctataccgaaccatcaggagcttttaaacttatggatttttgagccctaccagagagatctggatttggtaggagtggggtgggaccctggaagcctggcgtttcaccaagctttccaggtggtttggacgcacagctaggtttggggacttctgcccaagactgaagcagcagccttcgccggagcatcgcggtggttctgcacgttgctcctgccaggggcttctgcccgggagttgaagtggctggagggatgtgctcttcccataagaaggacctttctctcgggggtggccgtcatgcaggcagcctctggagccagtgcaggtgtgagcacagatagtcctttgcatgaagaaactaatagcgcaacacgggacacgtgggtggctttgctacctggtgggcattgagagggtcttcccttgatgctgtggggtgttcagccattcccatttgtaggagaaatcctgcctttccccagttccagtctccaggacacatggggtggagctgctggatacagggagtggggtgtccgagtcaaagtgaaatggagaggcccatgctttgggggagccctagggctgctgatttgatctgtaaggaagcaggagctgtgcctcctgaaaacccctcagcctcttggaaggtgtgtgcagggcaggtcaagatggcagacccttgggagtggccagccatgggcccacagggtttgggatttgatgagtaagcaggacccctggtcctaggcactggccactgcatgccaggctgctctgctgccagagcagggagaacaccctcagcttcctcagccggtgtgttctagagtgaggaattgaaaacatctgcctgtggttggttgactcttcaggggttgtggtttgcaactgtctcctccactctgcccaagtcttgttatttcacaagaaaacacactttgaaatagctcagtgtctctcaagtctgagaaatacccattatccatggttcactgaatgttccaagcaggcactgggtgccctccccgcccccgcccctcgccggcagacagaggtgcccgtttctggacagagccttcagctgtcctacagcaggtccccctgccacagtcggcctcctgtgctcagatccgtgtctgacacgctcacgtgtggaacagaccattgtactcatgccccgccatgtcctccagatgacgcggacctggtggagctcaagcgggagctggaagccgtcggggagttccgccaccgttctccgagcaggtctctgtcggtccccaaccggcctcggccacctcaacccccgcagagacccccccctccaagtaagactctgcttgctttcagctccctggatgcctgccaggaataaccgcgcgttccctgctactgaaaacacggctccgaatccacttctccccggccttccggtccccacagggcttcctcctccttgggctccgttttgttgtaaatccaggacacgtttgacgtcagggctggttgtgtttctcatggcaaatgaaacagctcctttccaaagctgctgttcaccctggagctgtgctattgcttcaaactgctctcccttggcagcattagaggatgcagaatgggggccgcagccatgggagggacgtgagcttcgcagtcagaagtctctcctcagctgctgcgcagtcctccctctacttatgaggaatctggtaaacccaggtccccgcctggaggtgacctggtcacccctctgcgctccgaggcctcctgttccccgggctcagaacaagttcccccttatcctgggtctaggagcattttcttctggttgttcgaacaatgctttgcccgtccagccctccctgataacctgaaagcaaagctgtgatgtgcttttgatccacttgcattttcccaccactgtttacttctccctccccaaaaatgtgatgatcagacttagatcttcctaagccattacttcagaattgggctcattgtggctgacgtcacggtagtaaccaatggcaagccacaagcccagatataaaaataacccaaaaataaaatgcataagtcatgtattattgtggaaattttagagataactacaaagaagaacaaaattttaaaattcacttgatgacctacgggtcgtagttgttgaaaaaagtcttctttttccagtttcatgtatctgcagcaacatacttctttttttttttttttttttttaacaaaattaatacccattttctctggtcggttgtcttttatttaatgggttatattttctgtgtcattgaatgttctaaaacatgatttgttaatggctgcaagtatattttaacagtttattcccctgctttggaattttttttttttttttttttttttttttttttttttttttttttgagatgggctcattctgtcccccgggctggagtgcagtggtgcgatctcacagctcactgaagccttgacctccccggctcaagcaatcctcctacctcagcctgccaagtagctaggactgcaggcgcataccatcaggcttggctatttttattcatttatttatttattttgtagagacagtttcaccatgttgcccaggctggggtttttgaaatattttttccccacaaaatattttaaaaatctggtcctagcaatagtatttggaaccatttcaaaatcaattggtttttatatcagatctgaatcccccaagatgttaaagtttgcattctagtaatgcttctccagatcatctacatcaataggcacacactggtagaatcaagtgagaaactccatagagagtcaaggaaggcctggctctgtgggtgccctaagtgccgagtgagtattcggacctcaccgtgtctgtggtctgctgggacatccgcctcgtctagtcgggctcctggcagctggctgggaagttgtgccttcactgagattgagactctgccctcgttggtttttacagccggtttaatggtgaaaaagtcggcttcagatgcgtccatctcctccggcacccatggacagtattcaattttgcagacggcaagacttctaccaggagcacctcagcaacctgtgagttcttctgcatacatttcaatcccaagggttttgcccccgggatagtgtgggatctctcttctcagtgaatatatagatttatgtgtctttaaccctctacctgctcaccatctcatctccaaaccccataacccttcctcgcaggttctccatagggcttttaaatggttgtatggagtgttctagaaaaaatcacttttgatgaaaaatcccaaatatccctggtttccatactcatccatttctctctcttcccattcttgcctctgaccttggtgtatgggcagagcattgcttggtagaagggaaaatgggagaaattcaggtcgttatcctgggaaaatgatgatcattctggagtgaagttggttgagagaagaggctaaaaagagatgtaaataatgaagttggggaacacactaggaagactaacagccatagaggattgtggaggtgcctggttccagagggtaccatgagctgctggggaagagagcaggtgaaggcagggaagtagaggggagagaggggaagctgggtgctagggggaaactgctatgaccgggatgcctctggggtgctgggcctggttaagtgcttgtgcggccccagcaggtggaggggtgactggaggccagtgttttacagtagtgaaaataggtcataaaggcaacccacatgctggatgaagaagtagaaaaagcacttttaaggaaggtattttaacaactcagtgggaccatggaaagagtggcctcttattttgctttctggctttcacctggttattgaaaaaaagatagaagatggtctgtccatatgcaaaaatctgtaatcacccggctttgaagaccagggtcatcaggtctaagtagtatggcactcttggaaagagagtgctccaggaaagagaggattgcagggtgtggtggaggctggagtttgtatggtcagcatcgtctggtggagccctgcacagacccacgggtagcagatacgtcccacgaggctgtcctcctgcctctgctgatactctgctcttcctcattctgtcctcaagcccaaggctcggactggaataagtaaaccttataatgtcaagcagatcaaaaccaccaatgcccaggaggcagaagcagcaatccggtgtctcctggaagccagaggaggtaggtgctttcctgggggcaggggaaaaaccaattctccttttctttttatctccctgctaaaagtggtctagaaacgaaaataactctttttattcttctggaggcaatattaaatgaaaacatcctttagcaacattttatataaatctatactaagattgattgtccatttaaaattgtattttcaaaattctgtgtgtatataaaaatcatcccagggctattaagcggcgtctaaaggagagaccgaaaaaaaccaaaatgagtttttaaaaaaggtgaatgctgccggcatgaagtcattctgccagctttataaaacagagacagttatagtgtttgtcctaatttcaggcacaaaatctaccttttaaatcactggcaagtggaaataaagtttttagtgtcaggcagggacctctttcccaccacattattttttgttgctgttacttaaaccatgttagttaccttgggctctcccccacttcattcataatcactggtaatttacacttttagttttggccttggctttgttccagaatctcctagagaggagagtgtgcccttttcttcgtttttttttttgttttttttttttttttttttgagactgagtttcactctgtcagctggactggagtgcagtggctcaatcccaggtcactgcaacctccgcctcccaggttcgagcaaatatcctgcctcagccacctgagtagctgggactacaggcgcgcagccccatgatgagccaatttttgtattttcagtagaaatgtgatttcaccttgttggccaggctggtcttgaactcccaacctcaggtgattcatccaccttggcctcctaaagtgctgggattacaggtgtgagccaccacacctggccgagtgtgccctttaaaaaggtgtaagcaatcaccattgcttaatttgcttttagtcagcatgtttaacttctttggaattctcaaataatatttaagggggcatgagttaaaaggtcgagaactactgttgtagaatgatactgctacaaatgctttctttttttttattttagaaaaataactaaaactaggccgggcgcggtggctcacgcctgtaatcccagcactttgggaggccgaggcgggtggatcacgaggacaggaaatcgagaccatcctggctaacacggtgaaaccccgtctctactaaaaattcaaaaaattagccgggcgtgatggtgggtgactgtagtcccagctactcaggaggctgaggcaggagaatggctgagcccgggaggtggagctcgcagtgagccaagattgcaccactgcactccagcctgggtgacagagcaagactccgtctcaaaaaaagaaaaaagaaagaaagaaaaataactaaaactatggatataacaacccaagtgtccatcagctgataaatggataaacaaaatgtggtccatcaatacaatggaatattgttcagccattaaaaaaaaaaagaatggggctgggtgctgggtgcagtggctcacgcctgtaatcccagcgctttgggaggccgaggcaggcggatcacgaggttaggagatcgagaccatcctggctaacacagtgaaactccatctctactaaaaatacaaaaaaattagccgggcgtggtggcacgtgcctgttagtcccagttactcgggaggctgaggcagaagaatcatttgaactggggaggcagaggttgcagtgagccaagataacgccactgcactccagcctgggtgacagagctagactctgtctcataaaaaaaaaaaaaaaaaagaatgaagtgctgatacatactacttcatgggtggaccttgaagacattatactatgtgaaagaaagaaaaaaaggccacatagtgtaccatcccctttttatgaaatggccacagcaggcaaattcatggggtcagaaagttcttcttgctttccagggactggaaggaggcctgggtggggagtggctgctgtggggcttcttttttgtgtgatgaaaatgttctggagttagatcgtatgatggttgcacaactttgtggatacactaaaaaccaatgaatggtacacttagaaagggtgaatttcacggtatgtgaattatgtcacaataaagctgttaaaaaaaaaaaaactctagagatgtagatgttagtagaggtgtgaaatctacctacagtcccggcttaacactttcttggtcatggccccattgagattttgatgtctttggatcatctaccctgagaaatgtaagtacacatgagactttgcatatgatctcagggtctcactggtccccatccaaactatccacagaccccagatcaccacccctgctaggaaggtgagcatgaaaacctgggactgctgggtgcggtggctcacgcctgtaatcccagcactttgagaggctggggtgggaggactgcttgaatccaggagttcgagacaagtctgggcaacacaatgagaccctctctctacaaaaattttaaaattagctgggcgtggtggttcacaccagtagtcccagctacttgggaggctgaggtaagaagactgcatgagcccaggagttccagcaacatagtgagacctcatctctactaaaaattttaaaaattggctgggcgtggcagtgctcacccgtaatcccagctactcaggaggctgaaatgggagaattgctcgagcctgggaggttgagactgcagtgagccgtgatcatgccgctgcactccggcctgggtggcagagcgagagcccatcttaaaaaagaaaaaagaaaaaaagctgggactgaatctagctgattactcctgaaatacactgaattagtaaatgacacttaggatgtagactgtctaaggacgaaatcatgcagtgaattgccttgtcctttacacttcagccatgtggttttatgtttcaggtgcctccgaagaagccctaagtgccgtggccccaagggaccttgaagcatcctctgaaccagagcccacaccgggggcagccaaaccagagaccccacaggcgcccccactccttccccgtcggcccccacccagagttcctgccatcaagaagccaaccttgagaaggacaggaaaggtaaaagcctggtaacataaaacctcttactcctaagttgctcacatgtctcgatagataagaattgtggcctgtaatcccagcacattgggaagccgaggcaagcggattacgaggtcaggagttcaagaccagcctgaccaacatggtgtgaaaccccgtctctactaaaaatagaaaaattagccgggcgtggtggcgcacgcctgtaatcccagctactcaggaggctgaggcaggagaatcacttgaacccaggaggcagaggttgcagtgagccgaaatcgtgccactgcattccagcctgagcagcagagtgagactccacctcaaaaaaaaaaaaaacaaaaaaaaaaaaacagatttgcgtctcacagataaaaatctagttccctatggacatcctcacgttcatgagtcccagacacaggtgtgtctttggggaaagttttgacaactgcttcttccctcatatcctactgtgtcttggctctggctgagaagaacgaggagtccactgtctggcacattgtcagtgtgacaagaacagcttggaggtattatgcttcctaagtgttttgagtccccaaagggaaccatagtgggatgcttttccccacatgttctggtaccgcccatttgcgtgtggtctgtgttgtgtgcacatggtttcaaggcattaggaaaacagtaaccgattcccagattcgccatgttcacatgctctcgtgcggcctcagcctacgagaggttccacagcttttcttgccacttttcttgcagattgttttttgctctcgctctcaagcttctcagccttgcttactgcttcaaagacatgagtttgtaaggacagtagcagcccaaagactggcatctgtagacacatctggatcttccgtttgatctcagtgttctagtaacggaccctcggtaatcccttcctggtcatcacagcacgatgctgcttcccccctagagagtgtgaggggggcttcggcttcactcttgacagcgcttgctttgtatttcctgggttgctcttctgtcccacattggctcattgccatctgtcccttgtccatgcttccttacaaagtggacctttttcctccgcctcttccctgttcagatgctcctcgacttaaaatggggttacatcccaataaacccatcgtaagttgaaaaatagcgtgtcaaaactgcatctcatacacctaacctagcaaacgtcatggtctcgctgaccttgaacacactcagaacacatacattagcctacggctgggcaaaaaaaaaaaaaaaaaaaaaaaacacccatccaacacagcctattttataataataaaaataaaagatcaaacttcaaaattcaaagtatgctttctacggaacattagcgcttgtgcactagggtgaagtggaaaatcctcagtggaaccattgtaagtcggggaccacctgctttgtcttttcttgtaaaagccaggagcagaaatagattcattttcccaacctgctgcacaggagttaaagttaaaaggctgttggaaaagagactttcttttttcttagtttgttttctaaagaaccaaagaaacactgaaacgccaatgtttaactagtctactgatcaccttttctgaacaaaagcttcattttgcctgtgcagttgttttctttgtgatagatttgaaccagaaccaaggcagtgtcaaggtttccattctttaccaatatttaagtataaaagattcaaggagctgggcgcggtggctcacgcgtgtaatcccagcactttgggaggctgaggcgggtggatcacctgaggtcaggagttcaagaccagcctgaccaacatggtgaaaccccgtctctactacaaaatacaaaaattagccaggcatggtggcaggcgcctgcaatcccagctactcgggaggctgaagcaggagaatcgcttgaacctgggaggcggaggttacagtgagctgagattgcgccattgcactccagcctgggggacagggcgagactccttctcaaaaaaaaaaaaaaaattcaaggagaaagtatatccattagtccttcagttagcttatgttgtaacacaagttacttcaccctagcatgatacagaccatgaatcagcccaggttgcaacttcaaagagtgttttcagagaggtttgctttcaataaaggaagagcatcaccccatttcctaaaggacaaagaaaagtttgtctgattatttactgtggccctggttttaaattttccactcgttttcattggacctgctggtgtgacctgataaatgtgtgtttctcctgtgctttttctttttcttacctggtcacatttctggcaccccacacattctcaaatctcgaatttgctagaatgtcagcttggtctcatctctgttctctgctggccactctgttctctgatcctcagctgcctagttattggcttcttatttacactctttgtcccaccttttctcagcccctgtcaccggaagaacagtttgagcaacagactgtccattttacaatcgggcccccggagacaagcgttgaggcccctcctgtcgtgacagcccctcgagtccctcctgttcccaaaccaagaacatttcagcctgggaaagctgcagagaggccaagccacaggaagccagcatcagacgaagcccctcctggggcaggagcctctgtgccaccacctctggaggcgccgcctcttgtgcccaaggtacccccgaggaggaagaagtcagcccccgcagccttccacctgcaggtcctgcagagcaacagccagcttctccagggcctcacttacaatagcagtgacagcccctctgggcacccacctgccgcgggcaccgtcttcccacaaggggactttctcagcacttcatctgctacaagccccgacagcgatggcaccaaagcgatgaagccagaggcagccccacttcttggtgattatcaggaccccttctggaaccttcttcaccaccctaaactgttgaataacacttggctttctaagagctcagaccctttggactcaggaaccaggagccccaaaagagatcccatagacccagtgtcagctggcgcttcagctgccaaggcagagctgccaccagatcatgaacacaaaaccttaggtcactgggtgacaatcagtgaccaagaaaagaggacagcactgcaggtgtttgacccactggcaaaaacatgactgagcagctttgaaggctgcagtcctatagaatgcataccttcctccctctagacatccctccaccagaagagacatctatttaaaggcacactggccaaaacgtttgtgcatctgtcactctcgtgtagtttacaaaaatcgtgtctcttattcagtaagatggttactcagccaccaaaatatatttcactcaaggcttgtacatctgaagtttgctcttcaaggaatgggaaccttcctgttaaattcggtgtatggattttaagaaaggaatctagccaatgaggtccaagaagttctcacccattgaatttttaaatggctgttcagttcatgttgtacgtgatggagatttgtcttttgttttatttgcattttacagatttggtataacattttggggagccacctgaaggttgatgtataaagtaaggattagagaaagaggtcgttgtgaccattagtagctgtcctggcccacttaaacaaggttacaaaaaatcagagtcggaagcagccaaataggtcaacctaatgactagactgtacattcccatgagccttcatgtttaagtgtgtacatgtgcgttaaccttgatgatgcgtgaatcccgagggagccggtggcatacaccgttagcttaaccttagcttaaactagctgaaggctcctgtgccatgtcttagacattgcatgccctatcaattactataatcctgagccatggtgtgctactgaaaccaatttttatccaccatctagtccttattaaatgaaacctcacggatcctttgttccgcttatattccatgcataccacataaaagcacacagtgcgaaaactcttgctgatacgcgatattgattctcattgttagaatatggagagtgtttcagcctcgtctgtccggctggagcttcgggatggaaagtgctatgtgtccctgcatataagaatcaccaggccagtgtttctgggtttgcttgtctatatgtttgtctatattttttgcctatacatttttcccacgtttccaacagcacttctcacctattcaataactgaaaaagacattaccatagtgctttacatttttaaagtaatgttacaaggtctggaatccatttggagcagataccgtgttttcgctatttaataagaagttcagtagtgaaatcttactgtaccgcctgttgtatctgggagcctcgtacagaggctcgcacagcagtgatcaagtgtcatcccttacgtgactgggggatgtctgtcctaaaagctgactgctaggatagtaaggatcatcttgcctgggctatgccactgtcttgttaccaattagacatctggaatttcataattagttttcattgtcactgtcaagatatattgcagattacttaaatatggccatcaaaacaaaagttacaacacgtatctcttttcatctgaaaactaatacctggaaaaggataaaaaaaaaaaggaatccgtgacccacagagctagacagataagatgcatagttgaccagtcataaaaggcggtgtttaggtgatcaggatgccgttggtggcatttacgtgctttatatgatttttacctctgtaacaaacacaagaaataaacagaatggtccttaacagagtttgggggagagagcaagatgggttccttggagaagctgatttgccaagatgcacatcgctattaacagccagagtcataaatgaaatgaaattgaagaattcattcaaatgctcttttccctataacctcttttctcaccaaaaaggagataaatttgaaaacagataaatgtaacaaccagtcaaagaagcaggggaaaagtaagctcctccaaagttgcttgcagtgctggaaatagatctcatttttaggttttctcttcgttccagataccaaataaatgggacagagaataaaatttttgttaaaatatgtgctcatctcctaagtagctcttcagagtctgaccgtaagtaaaaacacacagaattgtgttgactgggggaggtgaatcacaaaaaagttacgaggagtttaagagttaaatattatttgatcgtggctgtcaaatttagtgaacaacatagattggatttggagttggtagtaggtatggttctcataccagaattctcttaaaaaaaaaaaaaaggacaattggaattgccttatttatttttaaaatcaatgcttactagttggtaggattcccaggtcagcagcagggttgattaaataatcttgacaatgagcagctgccatcttgggggatttcattctgtggttttttaaatgtttcgtctttgatgctaccatccagggcttcttattgtgaccttgtagcctattttgttcctgctgttctctaacatggtgcagttcacgcagactggtttaggtacttcacaactcaccattgtctctctgaccccaagcctagtcccttttacatcaccatcttctcagacttcttgcctattcttaaaatatgttttggttaatgattgaattaggacatcagcttaagcaattcctgtaacggtttaacgttgttgaagttcaaggcacataataaaattctccctgatgtgtgtgtaagtataaacaaagtaatgtatcaaagcgttcactttataatgaagtcatttcattgggaaggaaagctgcaaagattattgggggactagtgattaataaaatcctgtaatatttttgaagtgaaaatttgtactgaaattgtacatgatacctattaaatgtttttgtctttttttaaa

SEQ ID NO: 3 - DNA, 117290 bp, homo sapien
gcggggagctgtcgcgggcagcgcgccctcgggaggacgtggccccggcccccgcccgcagtgggcccgaccctcatggccctgagcaaagggctgcggctgctggggcgcctgggggccgagggggactgtagcgtgctgctggaggcgcgcggccgcgacgactgcctgctgttcgaggccggcacggtggccacgctgggtgagtccgggccgggggcagcgacgcccggaggagagggcgcccgcattcgcccagcctcgggaagacgggtacccccccttcccgaggggatcgggcggcgctgggactgccggggcgtaggggtcgcgcgcagaggggtggctgtttgaatgaagtggggctggggactcgagagagcactctggctgggccggtggcgcacggaggccggccgcccctccacggggatctccggcccgcgccgccagaggatatggttgctggatagccggctggggcgctttgcgtattcatgaggatcccggggtgttgacttagccggcctgaccctgtgcctggcgccttttcttttgcacctgttgatgaaaccagtgattgccctttggtttgtgttggcggctccactcctggaggggatggggacaaaagccccaaaacataggctctggagaaaccgctgatggcagctttgtcccacgtaaatacttggtcgagaagcatcggtgctgcaatttatagaacttgtaaacaaatataccatgacctttatgctgtcttagatcctgccaagccagcgaatttcacctatggacttcagaagccttgaaactggcattgtgctatgggctttagtacgtgatctcacttaattctcacaattctactaaaaagctattattatccccattttgcagatgagcaaactgaggcctaaagaggctaagttgcttgccaaggtgacaccatccacctgacttttatgtggctgtttctgtaatgtcattcaatgcgtggcttcctggtatcgctaacctgggtcccttgttttgtgctaaccattataaggaaaacctgggtagcaccaaagcacgtgaaaccaggtctgattgttagacttctaaatgtggacacccgagagtaagctcccaccactaggcgtccagctgtaacagcttgccggtattcctagctaaacaaggtgcagcccggggtcactcgtacttgaggtggtcggcaaatgcagcaggctgctttgtagatgcctctagccttcttttcacatttgtatgcatcgactcccgttcggatgtatgtcttggaaggactatctctcctatttctgattccctaatcatttcattaacatgcatttatccagcttgttctcagtcacttgattactgaaacaatgtctccagtagtctcagcatcaaaagtaggcagtgaattgtagattaagttattttcagattattctaatttgtgctgtcctgaagagtctaagtttggttatttggtgtggccttgaaagaaaaccacagaaatttccattccattttagatgtctacttcataccacatttatcggaagcgaaagagtaatttcatggtacaaaactactgtttcatcaaatagattaagatgtccttcagttggattacggcaacattaagccaagttcatacacattcaagaattctttgaagcagcttagaaaaaagagattttattttttcaaaagtgaagatttaaaaatgattaatgacagtaatgtagtgcatattttccattaggagctgaagtttgggctgagatttacatcacggtggtgattacagtataccagattttagaacagagaatgtaagaaaggaattggctcgtcttcaactcatgtgcagtccacatttgtcatttaacctagcattcatttttcagtagaaaactttaaaaaaaaattttttttttttttgaaacagaggcttcttctgttgcctaggctggcatgcagtggcacagtcacagcccactgcagcctcaacttcccaggctcaagcgatcctcctgcctcagcctcctgagtagctgggactataggcacatgccaccatgcctggctaattttttctctttcttttcttttctttaaatggggtcttactatgttgcccatgctgttctcgaactcctgggctcaggtgatctttccgccttggtctcccaaagcgttgggattacaggcatgaactacttactgcgtctggcctgaaaacttaattttttaaagtctgttttgtttaagtaaaatgaaactatttttaaaaagcagctgtagcatttggtggctttgctggtccagagaatgtttgtaggagagagccttatgttactagaaagttgtttaaaactttcaacatatcctaaatgcatcactattggataaaatggctctgaacttgaaattcccatctgtgttccaaagggctagctagctgttttactttcctttcttggagaaagtgattattttttttcttttttttgagatggagcttcactcttgttgcctgggctgagtgaagtggtgcgatctcggctgactgcaacaccgccacccccacctgggttcaagtgattctcctgcctcagcctccccagtagctaggattacaggcacacgccaccatgcctggctaatttttgtatttttagtagagacggggttgtgccattttggccaggctggtttctaactcctgaccttaggtgatccacctgctttggcctcccaaagtgctaggattacaggcgtgagccaccatgcctgacccgagaaggtgaatttttacaagaaggctgcaatctctacttttgtatgggacaggacacttgtggcagctggcgtagctggtggtctaatcttgacctttcaccctttccttgaagttccgagtcgggggtagcttccttttctcttcagctgtggttttcctgtgatgggtccctcccagttagtaatgagaggctgagagacaagttgtggtaactgcttcgtgggtagtggtctttcccggcccctgagtcattctaacttccatcctgctccgctgcggatgcacatagctgctttcctaggggcgagaggagatgaggggactgggcctgatgaccacaggcgctgccccttcatagcacattttattcctgaaggatcctaactggaaaagtacctctcgccgaaggggctgggtggttccttatcattttcaggcaaggcctggagacaggggcttgaactgggctgtgatttttaggaaaaacggatggtccatgattcgttgattgctggactccttccatctgaacctgtcttcacctgtcttcacctgtcctgctctaaagtgaactcttcacactagggccgtctggcctgctagcaggattgttgaaggaattattccaaattcctgcttctaaaagaattaggttgtgaatctgaatctgtcttttatgtagatcagtgggagcaaaaattccagaggcagaaaagccaaatgtattggcactgatttaaaaggcttttatgaagtctcgggaacctgactggagggtagttggccttctcagcacaggtgggaagccccgtagccggtaccagcatccctggccaacaatccacaattagcaaacagatgttcccttatttgcacatgggacaaaaacctactcctgtctctgtctcttgtaaacttagggaaaagtgcttattgttaatggattttgtgtattttcagagtaagcgacagtgttccaaagttcaaggttctccaacagcccccaggaacggtggcctgagggtgtccctgtgtttacagtgcgggctgtgtgtgagtgaagtcgtgcctcctgccgcccagccgggctttgctgtgaacaggcagcagggagggagggatggggcaggagacttgagagaattaattaagttaattaatgggtgtccctaaattactttgagcttctggggggaaaggttctgtgtaaacaccaggcccccaggttctcccagctgctgttgtgcgaggccacgtctcgagtttagctttgtttgaattggtgagatggccccagggcagagcaggactcagtctgaactgttgcctagatcactttctcaggcattatggccagatttatttggtcacggactggaattttgatctgctagagaggaccacagagaatcaaataagtcaccattttataaatgctcctggacaaagttgaaaccacaggtaggaccaggcaagggttccggggcccctgacgtgggaaactagagcagcgacccgggaccacagtggtctgtctgcctccaggactggtttgcctcctctcctagccggaggctgtctcagcgtagtgtttacaaaatgcaaactgggatggtgtgtgcttctcccgcgggttttgtttgtttgtttgagacggagtctctttgcccagctggagtacaatggcgcaatcttggctcactgcaacccccacctcccaggttcaagggattctcctgcctcagcctcctgagtagctgggactacaggcatgcaccaccacgcccggctaatatttgtatttttagtagagaaagggtttcaccatgttggtcaggctggtctcaaactcctggccccaaatgatccgcccacctcagcctctcaaagtgctggggttacaggcaatgagccaccacgcccggccttccccgtgatttttgtagctcacctgtaagatcgtgatttcaaatcttaccaggctcttgttttggctgtgtggctcaaaaacttggcttttagcctttaattgtgaaaacgttcaaacacaaaagtagagggaaaaatgaatcccctgtctacctgccattcggcattgatagttatcaatatattgccatttttatttcacttgtccctgtattttttatgaaaacgtgaatctcctaaaatttagtttgtttaaagccaggcaagattctaatttgttgggttggcttttttttttttcttgcatttagatatagaagttcaatttttttattttattgtaaattgacaaattattatcgttttttgagatggagtcttgctctgtcgcccagctgggagtgctgtggcacaatcttggctcactgcaacctcttcctcccaggttcaagtgattctctggccttagccttccgagtagctgggactataggcgtgtgctaccatgcccggctaatttttgtatttttagtagagatggcatttcaccatgttggccaggctggtatcgaactcctgacctcaggtgatccacccgcttcggcctcccaaagtgctgggattataggcgtgagccactgtgctaggccaacagattaggatgattatatattatggggtacaaagtgatcatatatgtgtacaatgtggaatgattaaatcaagatgattaacgtagtcatcacctcaaatacttctttgtttatttatttatcttttttgaggtggagtcttgctctctcacccaggctggagtgcagtggcaccgatctcggctcactgtaacctccgcctcccaggttcaagcgattctcctgcctcagcctcctgagtagctgggactacaggcgcgtgccatcacgtccggctaatttttgtatttttagtagagatggggtttcgccgtgttggccaagctggtctcgaactcccgacttcaagtgatccgcccgcctcggcctcccaaagttctgggattacagacatgagccactgcacctgaccacttattgtttgtagggagaacatttgaaatttactctcctagtgatttcaaaatatacattattattaactatagttaccatgctatgcaagttcgaaatttacttggcagatttcgtgtggccagcagcagaaatggtggcatgaactgattctgagacattggccaagacacatttttaaaagcaaagtgaccacttgggaagggtcgaccacagcctggaagaacagggagcgctctgctggtgagctcgggaggacaaggtctgggccctttgacggcactgcctctagccccgggcaagagagggccgagggccatgtccagtggcctctatgctcccctttctggatgacattcctgctgctcaagaccctggagtgtgctaagcacatagtgcagcctgctcagccccctcccagggcctcaggcagactgtgcagccgtatgtccaccccaggcccgggcgttggggtggcaagggcatgttacaggtggaacgggacacacactagctgggactctgtgtgtgtatgcacgaggctccctgcaggggggcatgaagcccaggttgggggaagtcggggaagagaaggtggccaggctggggccgccctccccactggctacataatgaacctggccttccaggtagtcacggtggcatacttttcgaggtaggaggatagaacctattttatttaactgttttactttgttagcttgatttaaaactgaacagctgggcatacaggatgcagcactcatgagtactctggccccagaccctacagatattcaggcagacctgtcctgggcacctgtcccagcccgtgtctctctgctgtgtctctcttctggagcgttcctggggtgctcagggtcccctttgtgctttttggctgctcagtttctgttgctgggctccttcggccttcccactgaggatggggaatatattctgttttctttccacttgaattgtccttaaagccatgggtaaactggttcaggctcagacttgggaatttgatcggagtccctttagcatttctgggcagggcttgttggtgtcgtggccagctgtcctcgaggtgaaaaccaaattgggttgggagggtgcacatgacacttaaagcccccagagctgcggtgccatcaaccacacactgtggacaggaggctgtcagtgggcaggctgggtccaccttcaaagtccatacgaaacagcccagttcagggtactgcttcttttcgagatctcacgggcaggagccttctggcactgttcatgtaaatgtaagtcattctttctttctggaggaaaatttagacaggtgaatgaaaaccggagtgcttaatccgctattctacatgcagaagtttatctcaggaaacagtcccagatcactagcccattttgcttcaaggatgtttattgcaagctgttttgagtttttcactggttaaataaactttggttcaacttgatcataatagctgacatttgtggagtgtttaccacctaccaggtgctgatccaagtggcttacatttattaacttacttcatccttatgtcaaggcaagtactagtaatctccattttacacttggggaaactgaggcacaagaggcctaagaacttgcctgaggctccacctccagtggaaggctccagagaacaatatgttcagtaaaatctcattcttatgtaaaaaaatgaatatatatatatatatatatatatatatatatatatatatggaaaaagactccctttgtcctgttgtctctggcttatcttggttttcatctgacatacacttttgatcgaggaggttcccacttggggaaacagccagcccctttctgagcagagggtgtggcactacaaaggcgaaacatgatctctgggagaaacatgcttgaagcccacgctcctgggggcagagctgagcctccacacggatgtccccagcactgggagcagaggctgatgcggccccaaggtgctgaggaaagcgccccccagtgcaaaggatgctacaaggccccaggcttctcaaggaacatgaggcctggcaaacccagaacgttagactgggtcggctccaagatgcccctgggtttgagctggagaagtgtctgcgggagggtgctggggctcctgcagagcgtgtcttcctgaccctgtgggcttttctggggggggctaggaggctctgccaagttggtggatggaactgtgccttgccctggggggcactgggcaggagccagcctcggagcgtgagtctggtgctgcgccaggaggctagatgccccggccaggtcctgtgggctgcgccgtctgtgcccttggcccgcgaggctggcaggctgccactgcttgtaaagcctcctggttgaaaaggtgactcggagcagcggattcactgggagcctgtcagccacagaggcagcttgtgcttgtgccccaggcccaggccgcaggaggtgggatcaagttccacggctccagtgtgaaggtctgagcttcattctaaggcaggtttttgaggacatggggctctcgggcacctggttgtattcctgcatcctgcattcctgcccactctctcatgccttcttctccttctccttggcgtgccccaagccagccttccatctcagatggagttgtatgtttctagtgagcccacacaccccctgaggacagggcctcacccccgcacgcctgcttcttcatcacagctgctaggaaatgaagatgcatgtccccaggggcctttgaagtaggatgtggtctccctgagcagaactctgactctaaattctctcctggctctggcgttggtgtgggacttgggttgcgggagggggcaggttgttagcggctgctgctcagacccagcggaggaccgggctgctccaggctcccggcaagatgtagcaaaatggccaaatttaaggtgagcacgggctcccgtgcagtttctcacccctgctgctggctgcctgtctgtgaatgctaaggcgaggggtggttctacattgacttcttactgactggtgggagtaacccggaagaggcaaatgacttccttccctttcctcggctctgaacctagaatgttccagtgataccacctccaaactcttcacctgtgtcctttcccacctctctgccctgaggacagggaactaagtggctgctcacggtgggtaaaactgaggcttacaagacaccttgggcaatgggacagccagaccccagccctcagcacctgcctctcccatcccaagccccagtctctttttcagctgcaaagtcaatacctttgctttcagagagggagcatgtgccacgccaggagaggctttgggtgtatcacagatggagactgcctagcttaactgcgcagtctgaatgattctaaggaggggaagagagaacgcctctggagcctggcacacacatttgccggagaaagtggtgagtgcgttgtgaccaggtgagggacgaaccttaaggagattggctgagaacacctcccctcctcaccctaagtcaacaaacccgggacagcagtgcttccaaaaccttacttgttcacctcacctggagattcttgtggcaaatcgggttctgcctcgttggctctggggtgggccttgagagcctgccgttctaacaagctcccaggtgatgctgatccacgaccagctttgagtaccaggatctcgcatcagcttgggagggaggagacctgaatcctagccctggctccttaggctcttctttccagtgggggaatttagttaattgctccgggccccaatatcttaatctgaaaacaaatgaatgacaacctagttgcctttcctactctgagattccatgcgtcttaaaagaaaaaagttttaaaactaaacatgtcactgtcaccatcgctacttcacacacctaaagttataaaccactcagaaatgcaaatcttaggagatgtgatctggcaaggcaaaatcgaagatggctttgaggtctcagctaaacctagaaatctgatgctctgcattttttatgggatgcttatgaattaatgaaaatacatgagattatctctccttgagtctaccctttcctacaaccagcaaaaacttataaaaagcaattgaagcttactttgggaatgccaagggattgattaaaatatggactgtgctgattccatggctggtttagcagattcttgttaaaagccatgactttttaaaatttttatttttttgtgggtacatagtaggtatatatatttatgggatacatgagatattttgatacaggcatgtaatatgtaataaccacatcatggaaaatggggcatccatcccctcacacatttatcttttgtgttacaaaccaattatactcttttagttattttaaaatgtacagttaaattatatagtcaccatgttgtgctatcaagtaccagttcttattcattccttttatttttatttgttttactcattaaccatccccacctcctccctagacccccgccgctacccttcctagcctctggtaaccatccttctattctctatgtccatgaattcaattttgatttttagatcccacaaataagtgagaatatgcaatgtttgtctttctgtgcctggcttgtttcttttttttttttttttttttttccttttttttgagacagagtcttgctctgtcacacaggctatagtgcagtggtgcgatctcggctcactgcaacctccgccttctgggttcaacctcccaagtagctgggattacaggcacccaccaccacacccagctaatttttgtatttttagtagagacagggtttcaccatgttgtccaggctggtctcgaactcctgacctctggtgatccacccgcctcggcctcccaaagtgctgggattacaggtgtgagcaggtgtgagcaactgcactcggctggctggcttgtttcacttaacataatgacctccagttccatccatgttgttgcagataacaggatctcattcttttttatggataaatagtactccactgtgtatatgtaccacattttctttatccattcatctgttggtggacactgaagttgcttccaaatcttggctattgtgaacagtgctgcaactaacatgggagtgcagacatctgtttgatatactgatttcctttcttttgggtagatacccagcagtgggattgctggatcatatgatagctctacttttagttttttgaggaacctccaaactgttctccatagtggttgtacgcatatagtacatatagtacataacatatataacatatgtatactacataacgtatatatagtatataacatatatagtggttgtactaatttacattcccaccaactgtgtacaagggtttccttttctccacatccttgccagcatttgttattgcctgacttttggatgtaagccattgtaattggggtaagatgatctctcagtgtagttttgatttgcctttttctgacgatcagtgatgttgggcaccttttcatatgcctgtttaccatttgtatttcttcttttgagaaatgtacattcaaatcttttgctcattttttggatcggattattagatttttttcctgtagaattgtttgagctccttatatattctggttattagtcccttgtcagatgagtagtttgcaaatatttcctcccattctgtgggctgtctcttcactttgttgattgtttcctttgctgtgcaaaagctttttaacttaatatgatctcttttgttcatttttgctttggttgcctgtgcttgtggggtattgctcaatacatttttgcccaggtcaatgtcctggaggtctttcccaatgttttactgtagtagcttcatagtttgagggccatggcattcttaaaaacataactttcattcttgcatagctttttttttttttttttttttttttaagtaacctggactgtcatttggctagggaggcgggagtttgtgtgtctggaaatgtgtgggtttttttttttttttttttttttttttttttttttgggacggagtctcgctctgtctcccaggctagagggcagtgcagctacctcggctcactgcaagctccgcctccctggttcacaccattctcctgcctcagcctcccgagtagctgggactacaggcgcccgccaccacccctggctaattttttgtattttttagtagagacggggtttcaccatgttagtcaggatggtctcgatctcctgacctcttgatccacctgcctcggacttcaaaagtgctgggattacattacaggtgtgagccaccacacccagccagtgtgtggtcttttttatctgtctgtttaaactgtctgttttatatgttgcatgaaccccgctttaccatgaatcttatcaatgactgataaatattgtctattggaaacattctctttaagaggaggtgaccgttctctgcagctcctgctgattcagcaaagctgaggtcaggtggagccagctgggcagagatgagtgattagaaggctgtcattggcagtgacagtccacgtgctccagggcagaacagtgacactggctgtcacagtctgtttcaacaatggaggttgtttgtttttatttaaatgatgtgaataaagaaaggaatctttgaaaactaaggctggagctgcccaggcctgagccaggcccttgtaacaaggcccttctgggtgggggatctacctctcttttcacctggaccacctggcggtgggtggcgagctcagaaccacagctgggtggaaaatgggagggccagcagcgagctgcaggaatggaattccatgatggcatctcatgtctccagagtggacagacagaagcttgtgttttcatttcaaagggcatctgccacgccctccaggcagaccacataaatggcatttttgggtgaggcctgagggattctgtctggtggtatacactgattttaaccagaagcaatagatttgggctctttttgacaggggaagatagatgagagatgatttccatgaggatgttgaaaatattggcaattctgaagaccccagggagtcccagggggttcctggccaggagcgcccctccattgccctgcaccaccttgctttccaggctgtggctctgtgtgtctgaatctacggcatgggggcacttccttctaattcacagaagaagctatatgggccccagactgcccatcctgctaaaactggcaaaggccttttcccagcctgagcagtagtgattcagccaactgcagaagtggtatttggagctcacccttaaaacataaagcaagttatgttcagactatatacaggcaatgagaaaatatgatgaaatatgactgcgtatgaatagccccagacttctagggcctttgagtgtgtctgtcttgggcaacatcttctccttttagatttcaggctcagtttactgtcaggaaaattaagttgctgagagcgtctcagaattttggcttaaaagagaatatacacctggaacaaagacttgaggggtgctcctattaaaaaaaacaaacagtgtgtgagcttcaaaggagcaccttgaaactccagttccatcacatccactctgctgggccctcctagggcagctctctgtcttgtgcctgccttctcttctgaacactcctaaggtattttgagtgcggaggataggatcatacactcaggaaagccgagagctagatcctgaagcatctgtgcgggaaatgcccatctgtgcctgaaatgagcatctgcgagtgaagtgatcccatgtctgggatctgtttggcatgcgccaggagacagacggacacatgcatgcacacacacacacagcggggacggggagttggtgaaataagagtgggaaaatgttgacacctccgtgagccgggtgacaagctgcttggtggttcactagacagggagttataaattggccgcttttcaacatcgacagataaaaatcaatagccttcccataatgccatcattaatcaattagagtctgtaatgggaaaagcagatgccactcccccacagtttctgggaataaacaaagtgtataaggcctaaaagaagaaagctatagaacttgactgaagcccacatctgtggaaaggcatactatgttcttggagtgggaacatcaacactatagagatggcagttcctctccagttaatctggaagtgcaagtatcgatgagtaaatccaattaaattccaacaagagcctggtctaaaatgtaacaggctgattttacagtatcgctggactccccctacatccaaacttattcttaccttcattctcccattttggttaatggcagcaccatccaggcaactgctggtaataaaagcctgtgagtcattctgacgccccttccttcctcagcctcatccaacctaccagccagtagtgaatcccaccccaaaacagatggtgaatcccaccgagtcccccggtcatcgctgctacatcgagagtgcagccactgccccatgtgctttcctggtctgcttggcagcctttgaactagtctcctggctcttcccctgctccccgtatagggatctgcttgaagctcacacagccctgatcctctgcccctgcctctttctccaccctcatctcccaccatttcctgggctcactgcattcagcagcctgacttgttcctgctcagaatcttcacacccacctgcatgcactccctcccccaggtgatatggtttggatctatgtccccacccgaagtcatgttgaaatgtgatccccagtgctggagatggggcccagtgggaggtgattgggccatggtggtggtttctcatgaatggtcagcaccatcccccctggggttatcattgaaacagtgagttgccacaagatctgattgtttaaaagtgtgtggcacccaccccaccctcttggtcctgctcctgccatgtaagttgcctattcccactttgccttccaccatgagtaaaagctccccgaggcctcccaagaagcagatgctgccatgcttcccgtatagcctacagatccatgagccaattaaatctcttttctttataaattacccagtctcaggcatttctttatagcagtgcgagaacagactagtacaccaagggttggcctggctgctgctgcctccgtctccccacctaccccacccccccccacccagcttcgtgactgcagtaagaccttcctgacctctgtggctggaataccctgcagcccccacacaccaactgtctctgccccatgcctggtttcctttcttcttggcgcttatcactgaaattaccttatgtgtgtgcttacgtgggtgtcttctgttgtccactgcaaaaatgcattgtgggttgaattgtgtgccccagaaaaatacattgctgtcctaacccgggataccttgcaatcttatttggaaatagggtctttgcagatgcaatgaagatgtaagttaagatgggatcgtactggtatagggtgggcctttaatccaatgtgactggcgtgcttataagaagaaaagggacacaggcagacgcacgcagaggggagaatgtcatgtgaaggcatacagggaagacagccatatgacaatggaggcagagatgggagtgacactgccgcaagacaaggaatgcctgggctaccggaagctgggaaaagcaagggaagatgctcctacccacgccttgatcttggatttctggcctccagaactgtgagacaataaatttgctgttgtttcaagccccccgttctgtggttctttgttacagcaacccaggcaactaatacaggggcattgtgtatctcgttcagtaccttaccttaacactgcctcgtgtggaaatgaggcttggcaaacactgttgaatgacgaagcattggcaaaaaaaatagccaagattttgaaaaggatccacaaaggcagactgatttaccaggtagcacaacatattataaagccctaaccatcgtggaggctgtcagcacaaatcaacacagtagagtggaaggctcagaacagacttgtgtgagcagtgttactttgtttcttataaaggtgatgacttcagtcggtcagagaaaggtaagggtggatactcagcacctgctgttggtgtggttgatcagtgcagtgcctaagaggcccctgctctattcaactggatattcacgcaggcattcttggccggtctgctcagagaggaataggctggaaaccttggctggccagcaggatgctgcctgctgctctgcttccaggggagcgaggggtcttcctggcagtttttgcaggtggctgcacacacaggtgtccttctggtcactagctcctcccccagctgaaagccctgggtccccaaggagccagaatcccttataaaattaccagccttacagagccctgggttcacacaggggacattccatgttacaagagttaccacacctaaggaagccccatgcctggcttcttcaccgagtggttataattcacttatgggtcttccctgtccagaggcaatttgccaatcaaggtcatttttaccatgagcaatgttgtctagtaacagagttgacgttccatcttgattggggttcccagggaaccagagctagataattaaacattaaattctcatacagaagggaagaaagattttgctaagcctgtgaccaaaactatccatatggacactaagtgagagtcctatgttttatcatacattaaaataaatgcagaggtatttaagtgtaaaaaaacaactttcaaagtattagaaaatataggctatgctttcacagtgttacggcatggaaagatgttctaaggaaagcatcaaactcagaagtcataaaggaaataaacctgacagatatgggaaatatttttattgaagtgtaacatacatagagaaaaatgaacactcgaagcgttgagctccatgaatcttcacccggtgaacacacccacatagccagcacgcagatctggagagagaacgtttgcatcccaggcccctgcctcccattccccctgtgtccgaatgatagatttgactacataaaaatggaaaactccaatatatcaaaagacaccataaacgtagtgaaaagacaagcaacagcccagaaggaaagtttgtgcagccttttgtaacatagaaaaaggccaaagatggatgatcatagcatataaaagaactcctatagatcatgaagaaggaaggacaaatagttcaacagagaaattggcaaaggatataatgagcaattctccaaaggaaaaatacaaatggccaaaacaatggagccagtgtggccggctctgcccagcacggccatttgcacatggtcactgttgggtgggtgagggctgcttgccccgtctccagccccactctcttgccatctccccctgtgattcaggcatggagctcaggccccaggtggcaggcagcgcactctgctcagttaccccagctcctggcttcatggccatgccctggcacctgtctccctccccttagcctgctgacctttttcccatgggatgtttggttgtctctgtacaggctctgagccagccctatgggcttccagacaagcccagattgagcctgccctctagggagcacccctgtgtagcttccagctgagaccttgagacacaggcccagggagccaagctcagggctgcgaacatccaggtggggctcaggtgctagaggcggacaacagcctggtgcaggacccagagtggcaagaggtagtaagggggccatggtgttcagaaattggggcaggtggtgggtaaggcatcctgcttctaaaggccagggtttgggagggcaaggaagcccctggcaggagtcagggcaggcatcccgggcaggtagtgtttgttctgtgaagcaggcttccccaaggaggggccctagagcagaaagctgtgatgtgcaggtgactgggagacctgttctctttcgagccgtgcagctccggttagtggatgacagatttcaagccctgctcaggccagtgtttttcacacatgtttgacctttggttgacagcacacagcacaacagcttatggtccaggagtgcacctcggcatgcagtttgggaatcctggcgatgatgtgattaagattctggggccaggttctcaccaagccaaaaggctttttttttttttttttttttttttttttttttttttttgaggcaggagctaattctaggtacagaaaaccattggtaaagtggaggaagctgtttccagatccacggaatgacggtaactagcgtcacttgagaaggtttcttctgcccttttgtaaaaaatagcatgatgttttctcctagagacagacagggcctctgtacgtaagagcagacatgtatggagaaggccggagagtcacaaaccatatgtgcctgagatgtgccacctgtggggctgtgaataaggaagcagaatcagtgtgtttactcagagctggatttggctgtttgttccttcatctttcttggaccacatgtcctggtgttcagaggctcctgagagcctggggctcagtaggccgtctcatagcccctctaacccaggtgagaaaacaggtcccagtgggtccctaacccatgctcattttctcaccaaacaacaacaaataaatcagaaagtcctgaaaaatgaattctggcctgttttctggattctggattgaaatatggagtaggagctgacttttagtaagttgatcaactctccccgccgaccccctagccttctggaactgtcttcagtaaacacaacccccgccacccctgacctacccgcaccgcactgtggtcggcctcttctttgaacacttcccttacagcacgcactccccccaagctgggccttctcttgagaccactctgctgtccttgggagtgatggtggaggcggggtagacaccctgtccctgtctcgtggtgctcctttgggccacagctcctcttccctccgtaggaggaaccatctggaatccatcttcctccttgaccacacctctccattcattaaggagcagagttccctgctcactgggtctctctacccctcctctgtccctgtctgggtgaggtcacagagagcctcacccgcctgctctgttggcctctcacttgccgtcctctctgccccgttccctctcctgcacacctgtccgcggtatccccagctcttagtgactctagccctctcccgccctcctctgcaccctgcatgcacttgtgttgctgaatgtggccggagaaaaacacagccatcttgactgttctgtgagacttcatagatttccagggctgtgtggaatcaaaaaatcttcctgcatttccccagccatctggcctccaggatgactatttcacatctcctcaagcctttcacatctcatccccctcacacctgatgattttgcttcctatttcactgggaaaatgaaggtaactgagaactttcttttttttttttttttttttttgagatggagcctggctcttgttccccaggctggagtgcaatggcacgatctcggctcactgcaacctccacctcccgggttcaagcgatttctcctgcctcagcctcccaagtagctgggactacaggcgcccgccaccacgcccggctaattgtcttgtatttttagtagggacggggtttcatcatgttggccaggctggtctcgatcttctgacctcgtgagccgcccacctcggcctcccaaagtgctgggattacaggcatgagccaccgcgcccagccaaaggtagctaagaactttcaacagcccccacacctactccctggctctggtacagtggacagcctcccaggcttgtcccccctcccagtgaccttgacccctctcatcttttaagacctcgctcccgcagtttccccctttccttcctaactcggtcatttgcaccattataccaacaggctgtcttagctcacattaagaaaccctctcttgaccctggcctccacctcatctcttggttttcccatgtgacgaggttgtctacacaccttccctcttcccagtctcccttgaagtgactccagccaggctgtgtcctcccactcttctgaaacctttcctcaaggcctccaggaccttctggctgctcagtcttccatccaacagcatcccacagatgaagcattttcttcgcttggcttgtcttacgctctgttctcatcatattgcttcctcactgcctgtttttctccagagcctagaaccatgcctggcacacagtgggcatctagaccctttgctgagtggtgaaattgaagtgtgacagggtgcgtggcttccctgctcaggctccctcagttcccaaagctgtgctccttcctggatctcatgtagcacaaggaggcatacgtgggtcgcaccacatggcttctagaggagtatgtgtgaatgtgttcatgttcatgtggaaatagggaggcggccctgaggtgggaggagccttcccaagaggaatcacctgagagaggaagaagagctggggaggaactgaggaactggtttggcaggttcaggaagaagtgagtgaagaaactggaaaagtagccaggatcagaacctgcagccgcaggacctaatttgttaggacctaaattaggggtgattgtgcaaggcagtgcagcatggaagccccactccaggacttccctggtggctaggaaacattgctccacccctccctcttgtcaaaagagaaggtgcacacacttggagagaaatgggtatttttgtgcttctttcttgttccctgttgggtaggagggttggggctaccaggcccagtgaaaacacagcccagccctgcctgttctgcctcatcctgaagtgtaaggaactgcccattgtgctgggccctgtcactggctctgcccgaggcatatgtggcactgggcctggctttttaaggtgcaaactgttttcctggatttcttatttttaagggaaaaaactcaactacaaagactcagtggccagtcctgtgctcaagccctcttgttaggggagctggcctccctctttggccacagctcagtgcagggaggggtgtggaaagtgctgggaaagggggaggacccaccctccccacagatgggtttctggctctgactgcgggaaaccaaactttcttgtattttctctggctgggttgtgatgtgggcatggcgttggtgggaggtagtgcatttgctgagggtgatgtgttctgagtgtcccctcactccgtggctctttcatgtggcctggactttttggtgagggagcagcagtgggaggacaggagagtcctttagcgggctgatgtgtgtctgccagttggctttgtcttggctcgcagcttctgtttcagacagatgttttgatccccctttgcttagcaaccagctgcagccttgctctgtcttgggaaaaccactgtctttctgaagagctgcttcagctccaaaactgataactctggaaggagcagccaccagccagtcagaactgagctccgaattaacacatgaaggagactttggcctcttgagaccctgaaatacaagaagatggtttcatgtcatgtccaagaatgtggccacaaatgctacctcagtctcgggctgtctgcattgttaaatgacctcattatttttctgacctgtgggccgggcttcgttggcatttccttaaggcgtgttattgtggaaaatattattcaagaaagaagagcatgtggtttgcttggttactgagctggactggctctgggtgaccagagctctcacgcagagccgtgcacggctgctcccactgagaccagcctctgcctcgggatggtctgacccacggagctctgttttgggacccagaatccatgagccacatttgctgtgctgtgggacaaaccacagtttccttgggagcctctcaagtggtggctgtttctcttccacacccttggaccactggagctggagacggggagttccccagcctcctccaaggataagcctattccttaagcctccacaagccccatcattatttatttttatcataagccatgttgttcgagtagcatagacaactttctcaggttaccagggaaacaggactagaaagtcaacccacgatcaaattaccttgccgaaagggaaccagctcagttaacccttgaccccacacgccattgtcagaggcgtatgaaccagagcgactccatcttgaataggggctgggtcaaatggatctgagacctgctgggctgcattcccaggaggttaggcattcttagccacaggatgagataagaggtcagcacaagatacaggtcataaaggccttactgctaaaacaggttgtggtacagaagccggccaaaacccaccaaaaccaagatggcaatgaaagtgacgtctggccatactcactgctcattatatgctaattataatgtattagcatgctaagagacactcgcaccagcaccatgacagtttacaaatgccatggcaaaatcaggaagttaccctatatcctctaaaaaggggaagaaccctcagttcctgggaattgcctacccctttcccagaaatgaataatccaccccttatttagcatataatccagaaataatcataaaaatgggcaaccagcagctcatactgctgccctgcctatagagtagccattcttttattcctttactttcctaataaacttgctttcacttcatggacttgctctgaattctctcttgcatgagatctaagaactctctctcggggtctggatcgggacccctttccagtaacaccacgagactgcgtccctcactgcccctcgtttcggcagttgcatggttccttggtgagttttccctctttctcgctgtcatttctcactatgagtctggcgcttgttcttggtgtcttcagtggccacgtcgttgatccttcggaggaccttcgccccctctcctccagtgagtgaccccacccctgcagccactcgctcccatgccacgcccctgacccctccagcatctgtctcaaacatcccactcttctttctccactttcaccttgtcactcatcctgcaattcccagtcccagtgactattttttttttttttttttgagacagtcttgctctgtcgcccaggctggagtgcagtggcgcgatctcggctcactgcaagctccgcctcccaggttcacgccattctcctgcctcagcctccctagtagctgggactacaggtgcccgcaacgacgcccggctaattttttgtatttttaatagagatggggtttcaccatgttagccaggatggtctcaatctcctgacctcgtgatccacccaccttggccttccaaagtgctgggattagaggcgtgagccaccacgcccggcccccagtgatctttgaccccactgggatggcgacccttagaacccatggccttctcatagcccctcgtctgtctcacaccttccctctttgccatgtcagtgaccagccctgccttgcactcaccctccccacccgcccttctcttgctgggtcctccccacctggtgaaacctcagccctggttacatcctgcactccacgcctgcaccagccgctgtgcagtagaagtttctggagatgccccactaccacacgggcagagcgtaccatcaggtcacaacccctgagccaaggtgggcctttcacttgcgtttctgtgctctgtccactcccccaccctcccagatggcaactccaccctcccctttcctcagctctgctgtgcctcctccccatcctcactttcagctgatggcctcgtcctctcattccaccccaaaatttgaagccactggaagagaattcccgctggctccctgcaccaccactacccgcccaccatgtctgtcccccttcccacctgctccccgcagtgcactgtctacgtttcaggccagtgctacctgtgtgcagatgcccatggcctgctccagggcatggctttcggagctccctgctcctgccacatccgtcctgacctctctcttggctgcattggtcctgacctctctctcagctgcatcaatcctcgcctctctcctggctcatttctgccagcacaaaacttggtataaatgcccctggcctaaggaaatcttttgacccactttcctctccagcagcctctccatttctctgcttgccacacgcacacatgcacatatgcacacgcacacacatgcgcacacacgtatgcacacacgcacgatccatgcacacaatgcatgcacacacaggaaattcctcagagaatcatctgttgccactgtgtccattttctctctccctccccattcaggtttttgcccctcaactttactgaaactgttcctctctgagctgccaaagtctgactaaagtgacaaattctcagtcctcaaattatagatgcagaagcagcctctgacctgctgactgctcttgcttccttgaaacgctttcttctcctggccgccaggatgccacgcactgctgggttttcttgtatcttcttggtcacttctcagtccaccctgaccttcaaacatgctgcacccagaagtcggtgccctccactcactccctaggggatcccctccaacctcatgattattttattttattgctgggattacaggtgcacgccacactcccagctaatttttgttgttgtttgagacggagtctcactctgtcgccctggctggagtgcagtggtgcgatctcggctcactgcaaccttcgcctcctgggttcaagtgattctcctgcctcagcctcccaagtagctgtgattacaggcgcatgccaccacacccagctaaattttgtatttttagtggaggtggggtttcaccatgctggccaggctggtctcgaactcctaaacctcaggtgatgcactcacctcagcctcccaaagtgctgggattacaggcatgagccatggcaccgatctgtttatttttgagacagtgtctcactctgtcaaccaggctggattgcagtggcgcaatcatggctaactgcagcctccacctcccaggttcagatgatcctctcacctcagcctcccaagaagctgagaccacaagtgtgcacaccttgcccgactaatattttttattatatttttagagatggagtctccctatgttgtccaggttgttctcgaactcctgagctcaagtgatcctcccatctcagcctcccaaagtgctgggattagaggcaggagcccccacacccggccccatgattgtaaataactctgtctgctgggacgcctgggggatctttccaaccagatttctctgctggactccagacttgcagagctctgtcgtcagcctccacgcagggctgcaacagagccacggcatgtctggtcccagcagatgctctgcccagacctgtcctctcactctttgctgtcacgccagggccactccatcccttccattgttcaggcaatgaacatggcatcatccttgaaccctgtctttctctcatatcccataaccaatctgtccatggttccaccttcaaaatatacccagatatgtgatgatgttgaggaattgctggtaacttttttagatgtgataatggtattgtggttgtgttttgtttttgtttttgttttagacggagtttcgctcttgtcacgcaggctggagtgcagtggcgtgaccatggctccctgcaacctccgcctcccaggtttaagcaattttcctgcctcagcctcctgagtagctggagttacaggcatgcaccacctcacctggctaatttttatatttttagtagagactaaaaacagggtagtggtgtgatcacagctcactgtagtcttgacctcttgggctcaagcaatcctcctgtctcagcctcccaaatagctgggaccacaggctcccaccaccacgcctggctaatttctttaaaaatttttatagggatggagtctcactgtgttgcccagtcttgtcttgaactcctgggctcaaccagtcctcctgccctagcctcctaaagtgctgggattacagggagggagataccatgcctagctagtttcatttgttatctgcctggtgtgtgtagacatctgctttgtcacctccattgtggagagcaacatggggtttcaccatgttggccaggctggtcttgaactcctgacctcaggtgatccattccccttggcctcccaaagtgctgggattacaggcatgagccaccacgtctggccatggttgtgttttttaaaagagctcttatcttttccagagtcttactgaaatatttacagatgaaataatgtgatgcctggaatttactcacaataacctagggcagtgcttctcaagctttggcatgcattcaagtcccctggagggctcattaaaacagctttctaggtcttgagcccagagcttctgattcacaaggcctggggtgggccccgtaacttgtatttctaatgcttggtgctgctggtgtgaggagcccattttgagttgagctgatctaaggtataggagagtgagtagcctgggcgtgggctgggagaaggtggagttggtgggtacgtggtaatgaaggtacaagggtttattatgtcaccctacttttctttggaaagttccgcaactaaaaagcacgtgtgtatgtgtgtctgtctgtctgtctgtctccagaactcttcagctcttccattgctgcgccctggctcaggctagagtcctctcttgcccaggtggcgctagcacctcctaactggtctccttccttctctccttgttcctctagagtcagttctcaacagagcagccaggatgatccccagacccctgcagtggcattcagagtgacacccaaaacctttctgtgacctcaaacttctgtgcagtgtgttccccacgccacttcctccatgaaccttcagtcacactgcccgcctcgctggggcttgcactggtcaggagtgcttctgccgcaggacctttgcacgtgcccttctgggcctacagtgctcctccactctccatgcagccatctccctggcttccttcaggtccgcattcagatgtcacccttgcaatgaggccttctcagacaacaccatctaaaatgtcacttccccttccccactattcataccctactccctgctcactttttctgctccgcgcctgtcactgtctatattgcactgattgatcttatctatcctggcaggaagcttgagcagcgcccgcctcttcctggctgtgcctcggcagtgcccccaccccagcctcccaccccatcttggtctgcaccccctgccatcactcatccattcattcctcctgttcctcttggccactgccaaatgctggatggtttccatctgtttggttcactgcagtgtctctgtccctggaagggtgccagccacagaaggcacgaagatgtgtccacagagccggcggctaaaaatctggcaagaggagtcgaggaagggggctgaggaggtagaagggagggctttcctcaaaaggccagaaattcgtggccagccaaaggggaagcaaagacattctcgtgtaggaaaacttacaggaaatgcaactaaaagcgtgggagacaggaagtctgtgtatttctgagattatcaagaaaggggggatggaagttgcaaatgggcaggccaaattcagtctgcagaggtgtgcttccagcccacgcagtgcttaaaataaatggagatggttgccagcactggaaggtctggagtgccctgtctcctggaaacactgcagcctggcaccactgggctggtgagcatgactggcacctgcagcagctgcccaccagctaatggcaggtgtgtgttctctgctcacccaaagcccttccagcagcttcattttgttgttgttgtttatttgtttttgagacagggtagtggtacaatcacagctcactgcagtcttgacctcctgggctcaagcaatcctgctatctcagcctcccaagtagctgggaccacaggttcccaccaccatgcctggttactttttaaaagaatttttgtagggatggagtctccctgtgttgcccaggctggtcttgaactcctgggctgaagcaatcctcctgccttggcctcccaaagtgctaggattacaggtgcgagccaccatgcctggctagcttcatttgctaccttcctggtgtgtgtagacatctgtgttgtcacccccattttggaaagcaaaaaggatttcttcttccttcaaacataattgagaaaatacagttgtttgggggcaagtagggtgcggggtgactctggaaccggcctcctgggaagccaggcctctgggagtgctggaggtgggcagtgaggctgccaggcccctgccttctggggtggggtgcatgctcctgatacatcagaagggtcgggggaggacttggaggtgaaggcactgcaggaagagaggaagcttactggagagacggtcaatggggcagtcaggctttaaaaggacgggcccaccaagtgaaaagatgaccaagtgtttgggctgaattgtgtcctccaaaaggatatgttgaagcctaacccccgggacctcagaaggggatcttatttggagatagggttgttgcagttgtaacgctgagatgagattatactggagtagggtgggcccttaatccagtgtgactgctgttctaagaagatgtacatgtgacaatggagggacatgtggggagaggatggccacgtgatgatgggggggacatgttgggagaggatggccacgtgacaacagggggatgtcaggagaggatggcctcgtgacaacgggggaatgccaaaagaggatggcctcgtgatgacaggggacacatggggagaggatggccttgggacgacaggacatgtggggaggggatggccacgtgatgatgggaggacatgtggggagagaatggccacataatggtggggggacacatggagagagaatggccacataatggtggggggacacgtgaggagaggatggccacatgacaattgggggacaccacgcggggagaggatgaccacataacaacgaggggacatattgggagagaattgccacgcgacgacggaggaagggactggagtgaggaagtcataagccaaggaacgccaaggacagaacaagcagcaaacacaagacacagggccaggtaaggaggtccccggctccaggtatcagaggagccctgctgatgcctggatttcagctctctggcctctggacccagaagagaatgaagttctgctgcctggggcctcccagtttgtttacagtgagtctgttcatggtggagaaacatcttctcatgatgttcctgtccaccatggtcaaggcctgttttgagctcctgcattgtataagtcagaggtaaagctgtttttctcaaggggtcaataattgacagtattggaatgggacaaggtggtcctgttgggttttgtttgtttgtttttaaaatatgaggatagaaagtcgtgtttttacttgtgcttaattttcccagtggccgggatgtgaagtccacgcctgtcatctgtaatgcaccagggcaaggggtcggttttgtttgcatagcttctcccagggtcatcttcccacccctcaagtgtggatttggccacaacactttgtttagcaatttcctgtcattttgttttatttggtcaggcttatttgcatagttgaatgaatgcacaaaggcctccttgtttcgtggttcacaaagtcagaactggatcataccagggcatacgggttctgtttcaaggctttgtaaagctgctagggttgaaataggtatttttggtgtcggaattattttattgcatttatcagtttatctactgagatggggcactgagaactgctgatttacctctctgcagccctgtctgcagtacctgaaggctgggggaagacatttggcaatgcacacagcctttgtttctgcctgtgaagttggggtttcataggccacctcccaggagggccctgtaggtgcatgacagagtccgagtgtctcaccagtgggtttgtcattgcacccaagcagatgctgggggagaggatgcctcttccttctcctcacccttcatcagtgtgtgagcagaggcaggagacctgtgggctggaaggcccacatcctccttcccaggctgtgaatgtggcacttacatggcaggaggactttgcagatgtgatcaggaccttgagaggagacattatcctggatgatctggatgagtccatcgcactcacagggtccttaaaagctggaagagggaggccaggcagggagagggaaggaaatgtgaggatggaagagggatccgattgccgcgattgctgaatttgaagaggaaggaaggagccgagagccaaggaatccaggcagcctctgggagctgcaagagacaaggaaatggattctgtcctgcagccttcggagcagatgcagacctgccgacaccttcctttcagctcagcgaaatccatttcagaattttgagtgcagagctgtaaggtcatgagtctgtgctgcttaagccactgtgtttgtagtcatttgttgcggtagtaaaggagactaatatgctattttgccaccattctgagcttttgaggcaggctgatggaaaaggtgggacagtggcttcccagtacttttctaagtgatcctgagaacctaatatcccaggcacttcatattctgtaactctccaccctgctgccagcagagtctgcttagtcctcaagacgaggggcttctgctgctctcagaggttgatgatttcattgattatctttaagttactctgcactctcccttccccagagaaaacccagtcaaattcaagtgagcattggggaaaatattcattcattaaactggttcttaacctttttgggggcaccgacctcttgagattctgttgaaaatgggattttagcccccataaaaatacatatcaaaaatttgcataaattttggccaggcacagtggctcatgcctgtaatctcagcactttggaaggccgagacgggcagatcacttgaggtcgggagttcaagaccagcctggcaacatggcgaaatgccgtccctactaaaactacaaaaaatagctgggtatggtggcgcatgcctgtaatcccagctgcttgagtggctgaggtggaagaatagcttgaacccaggaggtggaggttacagtgagctgagattgcaccattgtactccagcctgggcaacacagcgagactgtcaaaaaaaaaattgcatacaattttgagaacaattttgtatacagattcccccaggagctaccccttgaagctccaatggctgccaggggcttcctctgtcaccccaggtgaagagcccttgtacagattaattcacattaaggctgatgaatggctgttttcctagtaagtctcctcgctgtttgccaggtccttaaaccatgttcagtggatcctcattatttgtggattccatatttgtaaattcatctgctctctaaaatttgtttgtcaccccaagatcagaactcatagacaccagcagagtggcaaaaatgtgttacccgatgagccttttcccagctgaaaaggatgctctgccatcttgtttcagcctcttggtgtaaacgagtgtggttttcatgatttgtttagtgccatgttttccatgttttgtgctttttcttcgtggtttcactgtttgaaagtgctgtctagtgtttccaagtgcaagaaggcagtgatgtgccttatggggaaaatatgtgtgtttgataaactgtattcaggtttgtgttctagtgccactggttgagtgttaacgaatcaacactgtacagatgattcctgacttacgatggttccttccagtatgatttttcaacttaacaacggtgtgaaagtgatatgcattcagtagaaaccatctttcaaatacccatgggaccattctgtttttcactttctgttcaatagtctatacatttcataagatattcaacactctttcagaaagtaggctttgtatttcatgatttcgcccagctgtaggctaacatgtgttctggataggtggaaggcaagctgagacatacctctatggtgtttggtaggttaggtgtattgaatgcattgtcaacttaggatattttcaacctatcctgggtttatcaggacatgaccccattgtaagttgaggaccatctgtttattcagtaaggtgtcttttaaatagaaaaacacataaaacaaggttagtattgattgattgacaaaaatgttgcaacctgaggctttcaggaacctaactctgcatttcctttaggagtgctgcttgcttcagtatctgctggtaactctatggaacatagctactgtgaataatgggaattgggtgtattttcaagttgttggcctctccagctacgctggtaatcagtcctgctctttaaatcacatcttaccatcagtcctgcctagagaaatggaggaagggaggtctctggtcttacggtctgtcccgactctctgccccaccagctatgtcccgagaaccagcctggacagagccatctgattggtgggtggtagcaggtcatgttgtgaggccctggtgcctgccttctgtcctcctgtgctcgctagaggagcatgaggcaggagcagccagtccatgctggttcctgagcgaggcagctctagggctgccagcacccactcctggggggctgagggtaggaagagcttcagaggggaggatgggactgtctgactatatttccaatcccagttgggacttgtggtctgatgaggacaagtgcctttgagggggatggggatggttgagaggaaccaggaaatcaaatgtgtcctggaaaacatggcatggtggatcttttgagttggggtgttgggatgtgtgtcagctaaaacaaggttcacctgcttcaaatgttcatccaagctttctacacttgcctctctgagtcaacatctttagagacagcaggaagtgatgaaagtcagttcacagtccatctgctctgagggcttggtatgaggggctgatgttttctgtcattttctcctttctaacatttcactttgggatcttagttcctggttgaaattccttaactgtgggagtcgaggcagcctaagaatccttttcggctgcttaagacaatgaggtagagagggaaccagctgccccttttcttacctccatgatgatgtagaaatggttccatccagcccaagggttcttctggggcccaaagctccttaagggctctgtgcataggatttgaggattagttttcttgggtggggagaaaagtccatctttatttgtataaatcctacctgaaatgcagcatttcctctaattaggaaggcaggcaacaaaaccagtatgttagctatgtctgtgactctgtcaccaatgtgggttagccgtatctctgaccctgtcaccaattgcaattacagatgtctccacatcacattagtgttgtttcagatatcttgaagtagcatttgttcccattactagttttaaattaaggtggttaatagaccccactggattttgcgatttaatatgctgataaggcacatatattgctatactataacattttttaaaaattttgataactagatttcaataagattggtatttgtgatcatatttattttcttgatttttataacagctttattgagatataatttctgtaccatacaactcattaaagtgtacaactccatgtttctaaaatatattttcaggattgtacaaccatcaccaccaccatcaccataatctaattttagaatatttttgtcccccttaaaagaagcttcgtactcattagcaattatctacattccccctttccccactctcagcccctggcaaccacaacatctactttctgtctctagaaatgtgcctgttctggacgtttcatagaaatggaatcttgtttttgagacagagtctcactctgtcacccaggctggagtgcagtggcgtaatttcggctcactgcaacctccacctcctgggttcaagccatccccctgcctcagcctcccgagtagctgggattacaggcgtgtgccaccacgcccagctaatttttgtatttttagtagagatagggttccgccacgttgcccaggctggtctcgaacttctgacctcaggtgatccgcctgccttcgcctcccaaagtgctggggttacaggttacaaagtaagccactgtgcccggccaaaatggaatctttctgaggtcttttgtgactggcttttttcatgtagcataatgttttcaaggttcatctatctttgtattttgttctgtgcacttaaaacattattctgagaaggggtctaaaggcctgccccgtcgtcaccagaagggcctgtggcatcattggagaggttagggctctccctaaccattagggctccctgcgcctaatggttcttccctctgtcagttttgcattaaaactagtacatgccttggcttgagcatggacaatcactgccaaagacccggcaggcaaggcacgcagctttccttgggatgggttggacccaggccctgggcactcagaggtactcaggcacctctgcccagtctccccaagttaccacctccacaggtgccttggagtgagtgggcatctccaggatgtagcctctagtggatgatacccgtggacagggacagcaaggtagagtaatagggccaggccccaggtgcctgcccccagctggacgccagaaccccaggaccccagctcctccagcccccaggaagctgtcagcacttccagagagcaagctgttggtgggaaggtctgaatccttgcagctctgtgattcgcgaatcataaaacctccacacttggtgtttgtgggccgagctattgtctttgtgttttctttttggagtgggtgggaagccagcttggcaagccgggtgtgaatgaacaggaatgagcaggagacgctcgctgatgccttctgtgatgtgtttcttccccagctccagaagaaaaggaagtcattaaaggacagtatggcaagctcacggacgcgtacggctgcctgggggagctgaggctgaaatctggtgagtagccgctcgctggaggagcaggcgccaggctccccggtgggcaggagcctctgtgtcggaaggggcctcagtgcaggcattctgtttgaccgctcttctctctctcttcttttttttttttttttttttttctccgagatagagttttgctcttgttgcccaggctgcagtgcaatggcgcgatctcggctcaccacaacctctgcctcctgggttcaagcaattctcctgcctcagcctcctgaatagctgggactacaggcatgcgccaccatgcccaactaattttttgtatttttagtagagacggggtttttccatgttggtcaggctggtctgaaactccgacctcaggtggtccacctgcctcagcctcccaaagtgctgggattacagccatgagccgccgtgcctggccgacctctcttctcttttaagatgtttctttatgcctcgggacctgctcctcctgcaggaacctgcaggtggggaggtcaaggtgagtgttgcctggttgtcacctgcctgtgctggggagcagggtggggttcccggcacccccaccctgtgccagagaagctcgtcttcattgcttgccggcgagggcctaagtgtttcgaaagtctggtctggggcttctgcccagggcattgtccgcagtactctagaagatgtgccaaggcgggtgggtgaagacagggaccagggcagatgcccgggggcacaggggtgggctgggagttgtcagcaggagaaaaaagccaccgatgtgtgactgtcagtcagtgtggggtgggagggagaggaagggacagtgtgatctgacttgcgaagggagagaacttccaggaagacgtgggcatctctgctcactctgagagactcgggggtgagccggtggtggccggctgtcgtgggtggtcacagccaaactcagctgttcctggagctcaggagccctggggctttaggttctaccacctgtttccttccttctctctcaggggcccccttgccatctgtgacctggctgtggacatctcttgtgacttctggggccgagacaagagcccagtcgcattcctgattctcagtcccacctggctgagatgggaatggctgtatgggttgtctgaattcgggctttccctgagccaagcgcacacctcccttccctgctcacagcagctgttttcagagactgaggatgtttaaagctgccagaaaagcacggttacgttggccattccttttctgggtgttttctgtcatggaatgtttttccctgaactgtgacgattacagtgatcatttccaaatagcatttaggaaaaatacacagtacccacatgacaaagtgcatttatgaaatgtctgtttcgtgccaagagctagccgtacattcacttatttaatcctaaaaataggctaccgtgataatgcatctttgtccccgtttacagtaacgggaacaggctcagaagcttcaagtaacttgccttaggtcagaaacttagttggggtttgaagcgcctccaaacgcttgctttttctgtggaaatgtgcgggctgccgagggttttctgcaggactttccgctctgcagcgatcttcctgaagttgcaggaaatccctgtggcccttgccttccatggtgatgaccgtgaggatggtggcctgcacctgcatgggactcacttaggaacttgttaacacggctcccgaggccacctgtgctgttgtcaccctgccttgcaggtgtggcagggatgccgcgttccatccatgctgatgcccttttttccacatggtagtatctgggcccccaggatgcctcacagtcttggcttccttcagttatagttggcagctgcttttggtccttacatgaatgactcattttgcatccgacagggactgggattcgacgaattacagtaaccggctcaaggtcacgccattctcaagtggtgccctgggttttaaactcatgccttgcggcaccagggactgccttcaaccaccaggctgttctcctttctgaaagggacaggtggctgggctcacaagggggacaggaagtgcagtaccttcccccatcccaggggaaaagtgttttttcccagggctgtgatctaagatcagctgtgggaaattggtcctaaccaggaagggtggcagccccaaagatgtcttattagagcgagccagggtgtctgcgggggtcagcagggtctaatggggcagggagtgctcagagaggagaccacctcccagggctgccctaggcgaggcctcctccctgccttcagccagtccccgccaccccacaccccacagaagggaatgaggcttcttgtggaccatgcgagaccagccctgctccgaggctttgggtgcccccaacccctgcccacctgtgagagtcaccccccggagtgggttggagtttctctgagtgtggtgtgtatgagtttgactttggcgatgtcaggataatttggtctgttttcaacacattctggaaagcaccctgagcatctgaaggatggaaggattgggctttaaatcaagttttttcagtccccattggttatcttaaggggcgtttgactttaacttgattttctttttcccctgtaagtccctgacccagaactgtgatctattttcttttgtgttttcagctgttgagcatgttgggcagcacggagtgtagctacaactgtgtgaccccacctgtgtgactgactccacctgtgtgactccagtcgtgtgaccaactacaactgtgtaactgactccaattgtgtgactaactccacctgtgtgactctgcgtgactccaactgtgactccatctgtatgactccaattgtgtgacccccacctgtgtgactctgactgtgtgacccccacctgcgtgactctgactgtgtgacccccacctgcgtgactctgactgtgtgacccccacctgcgtgactccgactgtgtgacccccacctgtgtgactccaactgtggactgactctaactatgtgactgactccaactgtgtgactaactccacctgtatgactccaactatatgattgacttcaaccgtgtgaccccacctgtgtgactccaattgtatgactgactctaactgtgtgactccaactgtgactccatctgtatgactgacctcaactgtgtgaccccacctgtgtgactccaactgtgtactgactctgtgtgactgactccacctgtatgactccaactatatgactgacttcaaccgtgtgaccccacctgtgtgactccagttgtgtgactgatttcactgcgtggctaactccactgttgcctgactccagctgcgtgacttcagctgtgtgactccatgtatgtgaccctagctgtgtgactccaactttcagttgccacgaagacaaccagctgagtcaggagtgggcctccctttcaatgtttttagctaagcatctctgcgcaaagaaagaggcttccccacaggctcttaatttgggacaagttttcccttagtttgggaaaaatttcaatctacctaaattagccttttacattagaaatcgagaaatatgcacaaaaattcataaaagcctttttccaggttccaaggtttttgtagaaagcccaaagttcccctttccttcctgaaatgccagcaggaaggggttggagctcccgtgattagccagggagtcaggcttcctgaagtttacacaatacaaagcatattaagtgagaatgtagcagaaaaaagaacttatcccaatgattatagcaatttaaattctcttgaacccaagctctcaaggcctacctgttttatagcctcaagggactctactcctatcccagactataatgaattgtgtctcctggagttgtgcagtacgcaacctgtgaagctgtacacgacagccctataaattctctagcagacagccctcctagattctagttcccacttcacagctgaactgcatttaatctctgtaggactgttttcctgtctgtaaacctgggaaaactgcctcatctctatgttgcctagttctcaggaggtccacatgggccatgcacatggtagccatgaagagtgtggagtgctcctccaacaccaggcgggctggccacaggcagtttcttagagtgtggcctggggacgctgccaccactgagaagctctgctgtctctcctctgtccctgggagggctggagggaggagggtgctgagtgagagggacctggcctgcagagggctgaggacgaggtgtgagctggaaggccaggctgtgtggcctcggggtgtggactcctggtggggggctcctctgcacaggctagcagagggctggtttcatgggcagtcacttcagaaggaggattccctggaggggagtttgaacccttagtcttttgggatgcagtgttctctagcaagtcttgaagcgaactggcctttgtcggtcttagattaatttaattcctgtgcccagtggtgggatattaccaagagagttgacacccagctccctgagggggaagctctgagatgatgcagcatttgccagtttccaaggtgtaaattcacgggccagggctgatttcaggttcccagcgtggagctgaggaaggatctgcaccaggggtgctcactgcgggtgttcactgctgtggccgccgctgcctacacctccctcatgttctgcagaagggaggcgggagaaggccctgagaccatgccgcatgggcacacagtgcttagggttctgaacctgcgggtggcgggggaggctcaagggtcatctggccgaagcttcccccttcacaggtgggaaagcctagacatctttagtcctttgtttggagctttaagtctgagggtgtctgtgtgagcagagggacagataggatggctggagggaccccagggttgggattggcacagaccaggctgggaaggtgaactcagagctgagacaacacaaggaaatgccagcccagagccttgtgggaggcctggggtggtcccggtaaacggggccttgggtctgcaagccacgtatcatgactccaacagccagctttcctggcctgataacaaattgcctaggacctgccactggctcttgcctccctccacctcactctggcagctccctctgacccatgcatggctttgctttggccagagccctggaaccaaagccctttacctggaggggacattgagactattggcgcatcctcctgggaggcccagagagggtaagagccttgcctgaggccacacaggccactggaggtgtgcagagcctggagccccactcttctgagttttgtttttccacttgctatgagatccttgatggtttcagggccctggtctctccctcagctgaggactgccaagaaccccgcaagcctagttagcttcttggctgtcagagaattgatcctgcctcacaaaagcaaatcctgtgctctaggtgagcatcgggaggggcctcacacacctggggcaggtggggaccagtggcttgtaccacactgacggtggcccgggagtcagcgaccccccacagcctggtgctaggctcaagccaggacactttccttaaaacccatagtacaggccaggcatggtggctcatacctgtaatcctagcacctcagggggccgaggcaggaggatcacttgagcctagaagttcaagaccagcctgggctatgtagcaagactatgtctctacatagtgatggtatctacagcaccatgccaggagcggtggtgcacgcctgtagtcccagctacttgggaggctgacatgggaggatcgctggaacccagaaaggggaagttgcagtgtactgagatcaagtcactgcactccagcctgggtgacagtgcaagaacctgtctctaaatttaaaaaaaaaaaaaaaagcaaacaaacaaaaaacccatggtatacagagaacacagtcaggcagtcaggctgtcaatgagattaggcaggacacagtgaggctgggattgaaggaggaggaagccatcagtgctgggtcaggtaggcagggcggggccttcccggagaggcagcgctccacacagcggtcctgaggtttgggtaaggctcttccacagaggcctctgtggttacacccgtcccaccaactgtctgggacacacaggcaggggcagttctaaggaaaagtgtccaggtccttggcttttttctatcatggagcacctgtgatggaggacagggttcttattcccctctgaggtctccctgcttccatgttagggtagagggtaggcccttctctcttctggaagctcaccgtgtgccgtgctccaggaagtaactccccctaggattccagacaaagggacagttcaaaagcttgtcaacagcattgcaataaggtcaggactaggtagcaatctgtttgcaagtcaggtgatgtccaattgtttgcttcccccataacggaaggagaatttaattggggtatcagctgacacattagtaaccattttatcagcaggttattgtcatttcttagcgctaactcagaaggactttgaagaatcgagtgacatttctacaatgaagccctttccattcccatctatttattaatgtgaacaggatcttttagtgtttgcgtctacaaaaatgaaaactagaattagaattggtgcagaacctactttcattccttcaataaatatttattcatctcattaagagatgcctttccaacacattatgactttttgtatcaaatgattatttgtcggccaggcacggtggctcatgcctgtaatcccagcactttgggaggccaaggtgggcggaccactagaggtcaggagttctagaccagcccagtcaacatggtgaaaccccatctctactaaaaatacaaaaattagctgggcatgggggctcatgcctgtaatcccggctactcaagaggctgaggcagtaaacctgaaaggcgaaagttgaacccgggagacagaggttgcagtgagcccagattgcgccactgcactctagcctgggcgacagagcaagactgtcacctcccagagaccccacctcctaaaaccatcgccttgggtgttcatacttcagcatattaatttgggggtgcacacattcagccaatagcaggtacactggttgaccacactcttaaagaggacagccaaaaaaaattatttggcagtgttttatttacattgtattgtttacattgtttgaccagttaggtatcactggaacgataaaccagaacgctctctttttaaacacttggagttgaggaacacacggctgcaggggctgatggggtgatcagaacaggacgtagaagcaccagatatgctacattaggatggaattctgaagaggggggactggaaatatgagtgaaagttccatgtggtaagaaagagcatgctcctgcaatcctgtaaataatagatggcaatgagtatcacgttactgtgtagattccatgggatactgacaagaggctcccagagttttagttttacgtgtcaatatttagaatacactgcacaatatattatttacaggcatttcaactaccgatgaaaaccttcaatggcagttttaaaatgggcagggaacgctgtctgagtccagtgtgttgctgtgacaaagttccgtagtagacggggtggcttatcagcaacagacatttatttcgcgcagttctggaggctggaagtccgagaccagcggcccgcagattggccggctgtgaggacctgcttcttgctccatagatgctgccttttctctgcatctgacatggtgggagaggtgcaggagatctctgggcacaaaccccattcatgagggctccactgccataacccagtcacctcccaaatacccccaccacctcaaaccatcaccttgggtgttcgtacttcagcatattaacttgggggtgcacacattcagtcaatagcaggtacactggttgaccacactcttttaaagggtacagcaggccaggtgcagtggttcacacctgtaatcccagcactttgggaggctgaggcaggtgaatcacctgaggtcaggagttcgagaccagcctggccaacatggtaacaccccatctctaccaaaaatacaaaaaattagccggtataatcccagcaactcgggaggctgaggcaagagaattacttgaacccaggaggcggaggtagcagtgagctgagattgcaccactccactccagcctgggcaacaagagtgaaactccatctcaaaaaaaaatagccgggcgtggtggctcacgcctgtaatcccagcactttgggaggccgaagcaggcagatcacctgaggtcgggagtttgagaccagcctgacctacatggagaaatcccatctctactaaaaatataaaattagctgggcgtggtgacgcatgcttgtaatctcagttactccagagggtgaggcaggagaatcactagaacctgggaggcagaggttgcggtgagccgagatcacaccattgcactccagcttgggcaacaagagtaaaactctgtctcaaaaaataaataaataaataatacatgcatacatacatacatacgtacatacatacatacatacagggtacagcaaactaatccgattggaaactgccggactttggggctagatggagcatgggcttaaagtgaaacctacattttggctccaacacagactagctgtgtgcttgggcgggtgattcagcttccctgggggacagtcatgcgtgtgtgaaatggcagtggtagccctgagacagcagcatgggtaagcaggctggcgacgtcatctgcaaggaggtgtaattaggagcaccagctctggggccagacttggcttgagaaacttgcccaggcactgtagctagaaagtggatttggatcccagctccccgctctgtcaccttgggcaggctgcttaacctctctgtgcgtctgtttccctccctgtgagatggggctggtaagggcgccccttgcggtgggcgggaggatggagtccgcgcctgtacagctcccctgcctccactgcgctcaccagataccggctctcctcactcccctgtaccactcccgaccccctcctccctggacccaggagccccagtgctctgtggcaatgttaagagcctctcctggctgctacagtgctcctctccccagcctcccccaggccgtggtcctgcccaggaaatgcacctgggactgttgccaggcactcaggccccccacacggaggctgttgaggaagtggtgctagaaatcagctcatgactctcacggagtgatgtcatgcaaaattttccatagctctggcagccacaggggccatcctcctgcctctccgagggcacttctgctgaactgtgcttttccctgcactgtgtcagaagaggcaggtgggtgccgggagcgctctgtggccaaggctctgatcacaagctgccaaccccggaaccccacctatcctacttgagaagctgaactctgacccctgtccctgtccccagtgtgataacttcctttgttcttaggtggttttgaggtggtttcctgaggatgtgtctggggagatgtgatgggatcaaccccctgccctgctggcagccccaccccatggcatagcagcaggcccctgggagccctgagcgctcattaaaggaacgcactttaatgacagccacacgccaccctgccacaagcagcgctcttctccctatgaagtgtggtgaggaaattggggtgagagggtggtggaactggttgttttgggggtctcttcctggagtgtggagagatcagaaccatctccagaccatggctgtagacagcggcccttgatcattcacagaagatctcgctgggcggatccttcagacctcagcggggtgggaagagtgtccttgggactagagccctgccagaccccaaaggccggtttctacccgatgatctcttgggccccgtcccgagagtgaggcacggcgccaccaggcacccccgatacgcagcgtgttgtgaagtgcccttgtgagcagcgggggatagggaacagaaaaccagctgtcagcgacagcgaggtggtgccccagagagggctgcctcattcgcctggctgcagggtccgaggtcgtcttggtagcatttggcaggaatttatttaggaagcattcagcaaaccgagtgtgtgtggtgctgggagatgacgacgaaatggccctgcccccaaggaagcccctcccctgggggcacaggtggggagggaggtcatgagctttgtgtgtgtgacaactgctgatgggaatgctattggcacgtacgaggggtgcctgtccgtgtcttgggagagcaaggaaggcttcccggaggagttgctgccaagttggcccagaaggatgaggaggagttcttctggtgaagtggggagggcagaggcaggatggtccctacggagcatgtggggacgctcgtgttcttccagaggatggaatgctgggcccgaagcctgaagtattgttagggtagagacaggcgggaggtgcgtggagaaggggtcatggaggtcttgttgggcagatggcggtgggacgtcccaggcagggggagcatcttgggcaagagcccagggtgcgaggtcatgcttatggcacattggaccagctggaggacagccagctcaggagagcagccggctggggtgtctgcccacgtgtggggcagggatgggctgcagcggggagggatttgggagcttctgtcgtagtcctgcagggagtgggagctgtgcaagaaggaagggcggagcggctgtgatccaatgactttgggaaggccactcctgtgatggagtgggggaagacagaccccaggccaggggtcctgagtcaaggcagtggctgaggaggcactggggaaggtgatttggagaggctttgggggtaggtcctgggggtctggaagggctctaggccaggaattttcagactcctgggggcatcagaatcacctggagggcttgttaaagcaaagcttgcccccagcctcaggcagctgggcggcaggaatctgcatgtctaacaggctcccaggggatgctgacttgctggccagggtcccacactttgagaagccccgcctgaagcttacaggacagggcaggttcttgagccattgagttgggcaatgccatgggaagttgcaggtgcacagacgttgccttcctggctgcggttgaacctgggctccatttgtccccttggagctccaggcggccgggccgacttcgaccctgagctctcctgtctgatttcctttccaggtggcacgtctctgagcttcctggtgttggtgacaggctgcacatctgtgggcagaattccagatgctgaaatctacaaaatcactgccactgacttttaccctcttcaggaagaggccaaggaggaggaacgcctcatagctttgaagaaaatcctcagctcgggggtgttctatttctcatggccaaacgatgggtctcgctttgacctgactgtccgcacgcagaagcagggggatgacagctctgaatgggggaactccttcttctggtgaggccctgggtcccctgcagagggtgggccctgggtctcctgcagagggtgggccctggttggcatctgaggccttgacctccacttgcaccacccccgggttatggggcacctctggagagttaggacccaggcctgctctcagcacaatgagaggaaaactaaccctgaactccctgccaccccccacccctggcccctggaatgtgttcagtgtagatttggaaaagaaacctgcgccaggcatggtgcctcatgcctgtaatcccagcactttgggaggctgaggcaggtggatcacttgagttaaggagtttgagacaagcctggccaacatggtgaaagcccatctctactaaaaatacaaaaaaaaaaaaaaaaaaaaattagctgggcgtggtggcaggtgcctgtaatcccagctactcgggaggctgaggcaggagaatcgcttaaacccaggaaacggaggttgctgtgagccaagatggtgctactacactccaccctgggtgacagagcaagactctgtctcaaagaaaaagaaagaaagaaaagaaaccctgttccataagtaaaaaacataagagccgacgtctgctgagtgcttcctcagtcccgggtgccgagctcagtgcttctgcctggattcactctttcagtcttctcaatcatggttacctccagtgtgcagccgtttaccgctgatgtttataaagcactgtgttcgggtcaccgtgctaagatctctctgctggtaataactcatcattattataaaaacctttataataattatgcggaggctctgttaccccattttacaggtagagagactgaggcacgaatagtcgagggaattttgccaaactccacacacttactaagtggcagagccaggatttgaacccaggctttctgggtccaggtcccaagctggtaatcacagtgctaaaccagtcccatcaatggggaagctgagtcaccctgcccaaagggacttgtagcaatgcactaagactagtcccggagcccaggcagaggcggctggatttctgtatgtttaagatctcctggtgctttttcactccacagcaggacagagacaagagtggacaaccctgtaaatacataaattatctattgccctttctggacataatgctgtttctgtcagattcgcactgatgcctagagatgccaggtagaagcaaaagtgaggaaagtcctgccatgggagtcgttccaatgaacctcatctaatgtaatgttttagaatccagtgtctgccttttgaagctatatcagttttcccagctctgagacgggactcgtgaggtttatttctgacctgtttgtgcctctctccctttgccagaggcagccacaggttttcatctggccttggtgctttggattccccagaagtgaattcctgatttctgccggaagaaggggaccctccccacatccctgtgtccatcccaacgctccccacccccacatcctctttggttctctggtattgtggatatttgggggcagtaatgtgcccagacttcaaaatgcacgtgaatcaaggctgggcatggtagctcacgcctgcaatcccagcactttgggaggccaaggcaggcggatcacctgaggtcagcagtttgagaccagcctggccaacatggtgaaaccctgtctctactaaagatacaaagaaaattagccaggtgtggtggcgcacacctgtaatcccagctacatgggaggctgaggcagaagaatcgtttgaacccaggaggctgagattgcagtgagccgagatcgcaccattgcactccagcctgggctacagggtgagactctgtctcaaaataaataaataaaataaaataaataaaatcgtgaatctcatgctgatctcattaaaaatgcagattctgggatggcaggtctgggctgggcccaggattctgcatttctaataagctgccagtgctgcttgtctgcagacggcaggttgagtagcagcctctggggatggcatttctgtgaatcgctggttacattttgcagggcccagcatgtgccccgcgagctgttacatgttcccctaaaggcagccaagcttaagcacagaggggctgtgtattatgaggtatctttgtgtggagtccgtgaaactattttaaaccagaactgtcaggggagcagaggtgagagctctcagcggcagtgagggtccttggaacccctaggggatccttggggtatctgaattgcaccagcacctttgcaagttctcttccctatgccagctctgtggctggaggtgtgaggggcactaacaaagcgaggcaaaggggattcacgaggtggaggctgagaggagctttggaaggaggagccccatggtgtccctttctgaagggagaggaggaagtaactctggaggtcgccaggctcctcagctgtaacacgccagggcgtggtgtttaggctcagggtggagcccccagaactgaccacaactgagtcaccacgggcagtgtggttctgggcttgatcgcctgaacccccactgttgtggcgcagcgtgaggtctggagacccacagcaggagtcagatggggtcaacgctcagcgtggctggcacataaggcagagtgagcagcagggtgaagtcatctttctgggctggtaattgatcacactgacaattgcctcccagtacaatcaggtcctgggaggaaactctttgggggtcctggatatgagtcatcctcctgccagagggccaggcagtgtcactagaaacagggctgatttaagggttgtccacttggcaaaaggtttcttctcctgagccctggtgatagtggccatagctgtgtagctggcagactctgaaatattctggaggctttgccttgatgccacatgcagatttcatatatgtgtttcggaagctggcttatcatctgaatttgagagagtgatttggaagagggacatgtgtttttgtacagcaatgcttcagcagcatttttcaagcagaggagcgctgtggctgtgcgacctcagatggggtcagacgcgggctgtgctccctgtgggggtgcatgtgactgtgcgacctcagacggggtcagacgcgggctgtgctccctgtgggggtgcatgtggctttggtcacagtcggggaggctggcctgagagcaggaagcttggagaagccccctgggacccatcagagctcacagcacagatttccagcaatgagaaaatgctgctgctgtctttgaggaaggctcaagggcaggaattgtgttgccttttttaaacaagtagggcactgtgatctagattcactcagtgccctgtaagagggcatctcctgatgatgggtcctcctccctgagagtgagtatgggccctcaggatggtaccactcaggtcccctgggtgctaaagcaagggggccaggccagctcagggatgagagaactacgtggtcgttcgcaggtgcacatggaaacgtagggcctcatgaagaaaggctcggcgtgaagagcgcgggctccaagctccatgacgccactggctggctctgtggcattagacaagcacttcacttctctgtgcctcagctgctttatctgtgaaagggggtcactgatgatacctttcacatagggtcacttcaagggtgaaatgagttaatccaccccaaagtacttagaatggggcttagtgcacaggataaatgttggctgtcaccctgtcgttatcatcatggactctagagcctacagcaatccactaggcatggagagggaatacttgaactttcattacattttattttcctcggatctttttaaaatttattttggattgctttgtagcgctctcgtcataatcatctcatggtgcatgtgtgtgtatatgtggataatttaaataactgttcccatatttaatatgatttttaatcacttattgatcatgattgtttaataataaaagatgattaattttttaccaatggggcctgcgatgaaaactgtggggcccacagggctaaccagaggcccctttgcgattccccgaaaagtgcacgctggctcactctccagctttgcttccatttagcgacagtgacgacctggtagtggctttgggaaaggactccctggccatcctcctctacgtgacacagtggtcctgctatttgggatctctttgaagcacatgaacttcccagatctgtacagcccaagtgcagagtccactggggagcagcatgcattcgctgacccgaaatgctgcaccgtgcgccccccagttcctcagccacactcgctgtccaggcagcatgtattggaggatgtcaagtgactggaattggtgtgggactgtgttttgcaggaaccagctgttgcacgtgcccttgaggcagcaccaggtgagctgctgtgactggctgctgaagatcatctgcggggtggtcaccatccgcaccgtgtatgcctcccacaagcaggccaaggcctgcctcgtctctcgcgttagctgtgagcgcacaggcactcgcttccacacccgtggcgtgaacgacgacggccatgtgtccaacttcgtggagacagagcaggtgagtgcccaggcccatctgtggcaccaaatggttccgagtggctgcagctcttgcacacgcgctttccacttggggcagaagaggccctggcatatttgtggtggtctctgatcaggcttcctcctataagtggagaacagcacgtgtaattgggctccacatgaaaatgtgacctctggtttgcatggtgtatcctttctgattcacatctcatgcagggaaaatgctgcttagccgaggtatttttaactaaacggaatcaggaatttttgccttggtcgtgaaaggtgggcagtattccaagggcagctgacagatgaacaaatagggtttgaaaatccttttaggacaaaaataagtagatcaatacaatggggggatgaaagaagaaaccattattttttattctaaatcaactgaagcttctcctggaagccagtattagcaccctagagttgtgaaatctgcgtgttaagcagccgctcccatccgttcccctagaatatagccatctgcttaatttctcctggagttttttctaaattctgttctcatttccagagatgctgtcttacaacttttaaatttttgctctaacttgcaatctgtaagataaggccaccatgtggaatacagtggtgtaagccataagaaaggaaaaccccggaaggcacatgtatggtactcctgccagggcaagctgggtgccctgtgtttactgagtgaaggcgtggctgtgcgtcccccctcgctgccgctgctgggagaggaatgccttatgcggactttggccactttaatccagcagaggtttgagcacctacctttactgtgtgggactttctgcctgctccagtgttcagagggtagatacagagatgaggaggctgctctcagagtccccactcagaggaacatcagagcagggggccctaagatgggcaacagctgcaattacttgcctgtaattctgctcagaatgtctgcactgtggatatcaacgaggctattatgtaaatatctggttaaaaatgagattgcttttggcatcttcgtcatgaagtctttgactgttcctatgtctagaatggtattgcctaggttgtcttccagggtttttatagttttgcgttttacattcaagtctttaacccatcttgagttgatttttgtatatggtgtaaggaaggggtccagtttcagtcttctgcatatggctagccagttatcccagcaccgtttattgaatagagaatccttttcccattgcttgtttttgtcaggttggtcaaagatcacatggttgtaggtgtacggccttatttctgggttgtctattctgtttcattggtctacgtatctgtttttgtaccagtgccatgctgctttggttactatagccctgtagtatagtttgaattcaggtagtgtgatgcctccagctttgttctttttgcttaggattgccttggctattcgggctcttttttggttccatatgaattttaaaatagttttttctagttctatgaagaatgtcaatggtagtttaataggaatagctttgaatccataaattgctttgggcattatgtccattttaatgatattgattcttcttatccatgaggatggaatgtttttccatttgtgtcatctccaatttctttgagcagtgttttgtaattctcattgtagagatctttcacctccctcataaactgtattgctaggtattttattcttctcacagcagttgtgaatgggattgtgttcctgatttggctctctgcttgactgttgttggtgtattgattttgtattctgagactttgccgaagctgtttatcaggttaaggagattttgggctgagactgtggtgttttctagatatagaatcatgttgattgcaaacagggatagtttgattcctctcttcctattgggatgccctttatttctttctcttgcctgattgccctggtcaaaacttctaatactattaatatgttaaataggagtgatgagagagggcatccttgtcctgtgctggttttcagcttttgcccattcagtatggtgttggctgtgggtttgtcataaatggctcttattagtttgaggattgtggatctaaccaaactaaagagcttctgcacagctgaagaaactatcaacagagtgaacagacaacctacagaatgggagaaaatttttgcaaactatgcatctgacaaaggtctaacacccagcatctataaggaacttaaacaaatttccaagaaaaaaaaaaaaaacattaaaaactgggcaaaggacataaacagacacttttcaaaagaaggcatacatgcggccagcaatcatggaaaaaagcttaacatcactgatcattagagaaatgagaatcaaagccacaatgagctaccccctcacacaagtcaaatggctattatttgacttgcgtgaatattatttgacttgtgtgaaatgagatacctcctcacacaataatggctattgttaaaaagtcgaaaaataacagatgctggcaagattgcagagaaaaaggaacgctcatacgctgttggtgggagtgtaaattagttccaccattgtggagaacaatgtggtgattcctcagagacctactcacaatagcaaagacatggaatcaacctgaatgtccatcagtggtagactggataaagaaaatgtggtttgtatacagcatggaatagtatgcagtcatgaaaaggaatgagatcgtgtcctttccagcaacacggatgcagctggaggccattaccctaagcagactaaggcaggagcagaaaaccaaacactgcatgttcttacttacaagtgggagctaaatgatgagaacacatggatacaaagaggggaacaacacacactggggcctatcagagggtggaggatggagggtaggaggagggagaggatcaggaaaaataactagtgggtgctgctaggcttaatactgggtgacaaaataacctgtacaacaaacccacataacatgcgtttacctatgtaacaaacctgcacaagtacccctgaacttaaaataaaagttaaattttgaaaaattttaaaaaggggacagagagagaagctgtggccccagctgggtggttttctcttttctggctgatgtctgcatggggagagtcccttccctgccccacttcattaccacttgccttttattttgaacttttatttaaaatactggttttggatttcaaccagaattttaagacttggggtctcatctattgcatctcatcgagtaagtccctgggaaccactgtcctttagcggcactgtccttcctaaaacaccggacattctaacacggaaaatgaaagcaaaccgcatgcatttcctaggactgcccataataaagtaccccaaactgggtggccgaaaacaacacacttattctctcccagttctggagactggaagtctgcaatccaggtgtcggcaggactgctgtctcttcgaagtccccctggggagaacccttctgagcccacctgaccgccccggctcttggtggcctctggcaagcatctctccatcatctctgcctccgttttcgtgtggccttctctgcctgtgtctctccaggactccagtcgtactggatcagggacccaccctactccagttgtcctcatctttttttttttttttttttttttggagacggagtctcgctctgtcacccaggctggagttcagtggcacaatctcggctcactgcaagctctgcctcccgggttcacaccattctcctgcctcagcctcctgagtagctgggactacaggcgcccgccaccacgcccggctaattttttgtatttttagtagagacagggtttcaccgtgttagccaggatggtctcgatctcctgacctcgtgatctgcccgcctcggcctcccaaagtgctgagattacaggtgtgagccactgagcccggcccagttgtcctcatcttaacccattacttctgcaaagaccctatttccaaacaagttctaaagtactgggagttggtacttcaccatatggtttttggaagggacacagctcaccctgggccgcggatgctggctcctgttagaggactgtgctttacaggcagcgcctgtggccctgtgagaactctcgctgcaatctttgctttctcacttttacagaccaacaagccctaccacagaccaggctgggccttggctctggccacttcgtgtcgcgtgcctgctgactggcgcttgtctcctgtgttgccagcctgcttgttagagctcagaggcagggaccttgcccccacatctcctgaatctcccacctcgggcagcctggcaaggccatagagatatgaacagcctgcccttccagctcctggggtacagcccgatgctgttgacattgcctccacctcccttccttgtctgggctggggagggatttcttggctcccataagcctttgtgggcttcctagtgtgtgtgttttgtcctttttcgtcctctgggctctgggctagacagggctgctctccatgaagtccccacccaaccttactcccaggagcagcagtgattgacagacagctgggcaccaccacctgccgctcacccacagatgcagccgctgcggatgctgcccccgccccacctagagagcataacttaacccatggtgacttcgttaacaagagatgcgtagcggggccccaggaaggtggccctacacccacggtagctcttggttgggtgcctatgtttcaggctgtgctgatgagcctggaggtgcctgggctggcccatccatcggagaccccggacagcctggctacccaggaaggggagctgtcacttcccagcgctgagtccagggctgtccacgtgggccttcagcaaagcctgactgagcctgccaggtcccaggctctgctcccaggtgggtgggcatggggggcagtggtggtgtgatgggggcgcagcaggagtggggcaggtgaagctggtcctgccaggtgctgccgcccttccaggtggtacttccgataggtggtgttcggctggtgacccctgcctggaggtcgaggggaagcacagcagagccagggcagggagaagcgtgtgctgcgcgcagcccaggaaggaggggcatggagcagaaagaaacacaggtgaactggaagagcagagagggttcatgcttcctgcacacatgggaccagggcatgggcctggtggggggtcagcattcctccaagccaaaaaacacaggtgtgacagccctttttctaaaacattacagaggcagcaggagcagagatggctgcagggaagtgactgttccccaaaagcaagaaactcctgttgcctcacttgtataagccaagggaacccctcgctttctgtctctggagtcactgaatatttttgtttatgtcactttatagcagtactgatgcttaggttgatctaagttcgtttttaaaacacaaaacacgtctttaagtggaatgcacagttagtatgctcctgcgaagcggcagccccgctgccctccctggctctgcggccacctgctgaggatggttcttctaggatgagtcaggagagtggagcaaggctcagcctgcaggtccttgctgagctcagggaagcagccccgtgaggatcgggctgcgtggtctgcaggggaacctgcctgggaccctgtgagatttacctgtttcaggtgccagtgatctgctgggattctggagaggtccctcaggggagcctggcacttcctacccccttgagtttgcaaagtcacaactgagggcctgggttgggacggtgtcatgagctctccataagctttgcttatggtacaggagcagactgtctggcgcttctggagacctttaacgtgctcctgtagacagtcgggtaaactcattctggtagcacagcgtggcctccccctccacctgacatctgaggaagtggcaggctctgcatctcggtggcctcgccccagggtgcagggagcaactttgaggaaattagtgctgagcctccagtaggaaggaggactgtgctggggaggcaggtgagccccttgtggtgccgcccgcaggtcacactggcctggggcccctgcagaggaaacaggatccaccagccaggcccgctgaccatttggaagtggagcgtgcacacaggcggctgtgcggtcatcgtctggcaaacaaatgatgggaccggaccaggagcagactggctccctgtggtggggactcttggcaggcggatcggcttcttaggaattaattctcaggaagccagaaaagggggaccctgtggcaccctctgcttttgctttggtttaacatgtttcctaggaggaataattaatcttctttctctgaggcaggctgtgtgtgtgtgtgcatgggttgtatgtgtacagtgtgtgcgtggtatgtgcatttatgtgttgtacatgtgtgtatgtgtcatgtgcattgtgcatttgtgtatacatgtgtgtggtgtgtgccttgtgtttgtttttcatgtgtatgtgtgcagtgtgtgcatttatgtgttgtatacacacatgtgtgggttgtgtatgtgttgtgtgtctgcacgcacagatgttgtgtacatgtgtagatagcatctcagcctccttgggaggaagccttgagtctttttggctctgctgagtcatagctggtgggcttgctctgcagtgctgactcaccacacagaaaaacaagaccaaaaagtcccctcagtctattaaaaaaaaaaaaggatgtaagataaatatttggctattcaagataaggccactgactcattcagggaggttaggatgactgcgtgtcctcccaggaatgcagacttggagataattggtttgcagacactgtatctggaagcagaaggtcagcagaaagcctttcagatagaaggtatatgtacccctgtgagttttcctgcctcgtcgctctctttttatggccattaggtttctgttcccactgctctggaagcgtcactcagggtcaccctctggctgctcagtctggggtctaggcactggcctggcctcatcacagcactggcacgggcctccgacttttcttcccgtggccccactgctcttcccggtctccctcccacctcttgtctgttccttcttgaatccctccttcttcccacgacccaagctctctccgacgccagcaggtgcccgcatgcatagtcatagcgtcagggcctctggggaagagaaaacagcttcgttttttgagtggctactctgtgctgggactatgcccccctcatttacccctcacagctcatcccttctccgtgcacaaggtgacatgaacttcatgtagagagagggaaactgaggctgaagatggctaagtaatgtgcccagggccaccctgctgggcaaatgggggtttgcaaagtggcagtcggacccagcttgtgctaataccagagcccactttctctcctgcttccactctctgtcctgcctccccaccccaagagccctctgtcaaaatgacatctgtgaggtgagacatcacttcttccactggtattaaaagttgttcgcccagaatggaaatgtatcacttgcatttgatggtcaagttatgcctactgccttgggaacaaagaagacagtcttgatgataggacggggtgcagatgtcacctacttcccctgtggaggacggggtggagaactttggagctcctttcttcttgctccagtgtgggcagggaggggcatacagagcactgacagccctcagtgttttcagaagctggttctgggcaagtctcagcttgggaggagcctgatgtgatgctggaagtggcattgttagcacttggggcagtcagagtgaagggagacattcctcagcctaaatagatcgtgggcattggtttttgttgttgtttggtgttttttggatttcactctgttgccccagctggagtgtcgtggtgcaatcatagctcactgcagcctcatcctcctgggctcaggtgatcttcttgcctcagcctcccgaatagctgggactacaggcatacgccaccatacccggctaactttttaaaaattgttttggtagagatgggggtctcactttattacccaggctggtctcgaacttttgggctcaagcccgcctcccaccttggcctcccaaagtggtgggaccgtaggcgtgagtcactgcgcccagcctggatattgttaaacattgtctttatatacaaatgttggtttcttttccaggaacctgggcctatgcagggcacacacgccggtcagttgtatgtacctaatttcacggtagccaactatgtctgtagccgttcaagcaacctgctattttattctgttttgtcagtagaagaatggagttcaacagctttgctggtactcaatacctggaaacagacagcatgttcttaacacatgcttttttcctttggagattaagggatagtattccagtttgacaatgtagataccaggctcctgtaattcctgtggcccagggaaacagtggctctaattttcaggtcctccactgcagaatgactcattcctgtgtgatctgagcgtgatctcgcctgcatgggaagctgccctgtaagggagcaggccggtgaggaacggaaaggaccacagcagcgcccaggccgccctgccaggccaggctagagttgcgcagataaaggccccgcatctgataagtgtcgccgcctgcccctgctggcgctcaggatcgtacttcttgtccttttaaaactcccttgcactccagcgtcattggaggcctgcaggcacactctgaaatagctgtacgtgtgggtggggaggagatagatgtcttaaagtgtacggatcatttgtttctctgcaggtgctgtcacactggaagcttgtggaaaccgtgttgtgtgtgttaactgagtggtgtgtttaagaacacagtggggcagggccaggaccaagtacaagatcaggttgagtcttgcattgtcaagtacgagactggctagcacagtcagtgaggtcttttggctatcactggtgcattttatttcattcagtccttttctcatagctctcttctgcttagctatttgccaaggcaaaaatagagggctctaaggaaggttacattccagagcacactgacttctattcatacagggggtggcggaaggctgttcctgggattttcagggtgacgataaggttgtgccctagaagtgtgaattcccggagaccctgggaggccccaacccccagcagaccccctcctcagaggctggtcgacagccagcatgcccgcccagtacctggcgagaggtcagggcgcattgccggatgggggagcagaggacgccggagtccaccgtccgcccttcattctggctggtgtcgggtcacaggtggccggatcccgttacccagcccaggacgttcggtttcattgaagaatacctttccctttctgtttccttgtgccgcagatgatttacatggacgatggagtgtcatcttttgtccagatcagaggctccgttccgctgttctgggaacagccagggcttcaggtaggtcatgaaaaaacttaaatgtccccttgtgatgttgtccgccctgcccttcccttcaatagctggggaagatttcttttaacacgttcgtttcatggcatagtttccagtctttttcctcagccctgttgtgttgagctgagctatcaaagtgtgcacacgtgtgtgcatatgcatgcgtgcgtgtgtgtagaaaacacagacaccactttgttgtttctggtcgtcaaggatgctgtgtgaaaccgctgactcgggaacggtgctgcggtgcctgctgagtgggtgccgcgaagtgagcttgggtagcactgcctgaggttagaccctgagttcggtgtactgaggacgtttctgagagccgtgactctcatggagggcttcaggaaggctctgtggctgcgtaggtctggcacccggtgctgcctgttgtggggccacgtgagacttccacagctttcccgtgatttctaaaaataccccttgccgtccaaagcagggctctggtcagtgccagagagatttggcattctttctcctgggaagagcacagggtggggacacattccaagaacacatttgtcatcaatggaatgacacgctgcctccttcaccagggtctctaacaaggtgggttatgggccgagtggactggaggggcctccccactaatccccagtcttcctccctgtcccgaggggacgtgggtgccttcagaaagcctctcggccagggcctttgtcgcctctcggctcttcttaccgacagctctcgtgtgactgcatttgggttgccagccagtggctactggggcccagagtgccatctgccaggttccagggggtgtttgacaacgctggactcagggtccccagtcctgggttcagcttcttagcctgctctttgacttgaccctgtgccaggagcaggtgagtcacttgtccccatggctgtcagcacgctgcagtgacagcattggtgaagtactgggtcagcctgggcagtgagatggatgaatggaggtctcgtttaggctccacgctctggtagggagaaaagtcttgattaacattcagtctttccactagaaagagcgcaccacactgcacgagataactgacggcctccggagaaacgctcctgaggacagctgtgacacagaaactcctccggagggctgcccaaaggaggtttttggaaaaatagctttattgcaatataacttacacacccatttaaagcctacaacttggtgcttttactgcatttatggtgttgtgtaaccaccactgtaatctaatcttagaacactttgatcaccactaacaaactttgtccccgttagtggtctctcccatcccctccaagtctccctcatgccaactcgactgttcatctactgtctttatctatagatttcccattccagacgttccaaaagagtttttgaaacctgattttctttaaatatggagttttcctttggaaacttctcgagctctgtattcaggagaatgaaagaattgatcgaatgagtgtggcacaaaaaggagacagggagaccatgttgtgtttcatgtaaagtttgtaagaaatccaaaaattaggggaatggtcatgatggattcttagaatttgggaaggaccctttggaattcatccattccaccctctgactctcagttcaggggtcctccttttttttttttttttttttttttttgagatggagtctcactctgttgcccaggctggagtgcagtggtgcaatctcggctcactgcaacatctgccccccgagttcaagccgttcccctgcctcagcctccagagtagctgggactacaggtgcccactaccacacccagctaattttgttgtatttttagtagagatggagtttcaccatgttagccaggctggtcttgaactcctgacttcaggtgatccacccgcctcagcctcccaaagtgctgggattacaggcgtgagccaccgcacctagccaggccagttcagggatcctcttccaggacctccaaagggctgtcctccaggccacaactggggatggttccgactcttggtgaattcctcctgtattgcactgctgcgtctgtctgactctagagtgttttggggtgcgcgggaatatgtctgattcttctccgtgtgactcaccctcagtccttggagggagtcttctggttccctcttcttcttcttgtcttctctaaggcctcccaactcctttggccaccttccctcctgtttctaagactctcaatgttctcatctccctcatctaaaaaatgacactcctttttgtcgttcccctcttggatttcattttggtcttaccttgaccttcgtgctgcctcagatgtagcctgacccgccatgggatgactgtgccttggtctgtcaggggccaggagcagtggaccacgaggtctcagctttttggcagctgtttcaccctcttaaattcctaacatttgtgctgcaccaaagcctccatggtttcttctttttgtttcttgttttgttttgtttttgagacgaagtcttgctctgtcacccaggctggagtgcaatggcacgacctcggctcactgcaacctccacttcctgggttcaagagattctcctgcctcagcctcacaagtagctggggttacaggcacccaccaccatgcctggctaattttttttgtatttttagtagagacagggtttcatcacgttgaccaggctggttttgaacttctgacctcaagtgatctgcccgcttcggcctcccaaagtgctaggattacaggcgtgagccatcgcgcccggcctccatggtttcttcaaaacaagcttcagaaggctttagtgttccttaatcttattaaggatttgctaacctttgggcctaatacctgtgtttagcctctaaacaccactcatcatcaaagagagatgaagtcatcttcacagggggccgggagggcagcgggaagtggtggtctaccgtcaccggcttcaactgtcactatcaagcctaaggctgcttctagttatttttctccactttgaactttttctcttccttcttcacttaggttagatgaaaagtgcaaagattttggggtgagtagcttgcgtattaagctgcaatgctgtccctccttgacagctgccgttggaaacccaggagcccagcaggatggggcagggttgagcagagggtggtggatgaggatttgtctgcatgttttcctgggtaatggtaccttcagtacttcctgactccatttctagcagccgtgtacctttaataaatttcatttttacgtgtaaatgggctgtgcagtttataaatagccacattgaaattttacccttaaagcaaattgctccctggtctagctagttcgagaatctatttatgatctgtgtctgtggttgacaacgttgctcccctggtcattgtagaagcttccccccaccatatgctgtatagacacgtgccttgccaaggtgagttcagtctgtcttttgaagtttcctgcaactgaaccaaacggaggaggatgccgagtagagcagacccctcttgccctgccttcacctctttcctgccagaaaccattcgccaagtttgtggagtcgtggcgctggatgggcttgtgtgtttgtaccttccttttttccaaaaaggacatgagatggcttgtagtataacagtttcaaaaataagtgaagaggtctggatgaagggcaagagtcgaattggtgcccacgatgcatgccgtgggtcttgtgtgcgtgggaaggaacctgacgcttgttccaagcttccccgcagccaccacagaaagaaccacagtcacagccagtttatgacagtgactcagagcacgagctttgaagcgacgcagacctgggctcaaattcaaactctggctcccctgctttgtaaccatgtgtgtttgagaggctcttatctgtgccaagccttagtttacttttctgtaaaatggggctaacaatatctaccttatagggttctagagaggattaaagagcgtaatataagaatttggctgggcatgggggctcatgcctgtaattccagcaatttgggaggccaaggtggacagatcacttgaggtcaggagtttaagacaagcctggccaacatggtgaaatcccatccccactaaaaatgcaaaaattagctaggcatggtgtcaggttcctgtaatcccagttattcaggaggctgaggcaggagaatcacttgaacctgggaggcggagtttgcagtgagccaagattgtgccaccacactctagcctggtcaacagagtgcgactctgtcaaaaaaaaaaaaaaaaaaaaaaaaaaaaaaacacacagtatctagaatctaaaaagtactcaaaatggcagctgctattgatactaatagaaacaaatgataacacccaggagataaaaccaatggtggcatccggagaagcatggctcttggtgccgtgagcactgaatgagagtttcctcctgtctttgtgggaacagggacaccgtgagatgtagtaacatgaagacagcttcctggtcagtcttatctctcatccctcatagactcatcgtaaatcagtaaccccagatgaagccatttattcatcccaaacattcactgagcgtctgccaggtgtagggggcaccagggatcagatacagtccctaccttcagggagcagggggcaggacagggcagtgttgtgtggacagagccgtgttggaggaagggagaggccccgggggcagctctcccaggctgagggtgtggggtgggctgcccaagggtccagactggggaggggtgaggaagaagcactcaggcaaggaaggcagcccttgcagagggaacaatgggtgcaggtgttgtaacccttgggttcacagtgatcagggagcatcactgggtagcttcttgaagtcctgggcatgaagggaatgggaggagagataggagaggtgagcagggctgggtcaagggcctgtggatgctacactgaggagtttgcatcccataccagcagctactcaagcagtactccccaaccttgctgcacagaagactctgctggggagcttgaaaaatcctgatacccaggttgataccatacacattaagtcacaatatctggggccgggagccaggcagctgtcctttttgaagacccatgggtgactccagcatgcagaaaagtatgggaaccactcggtgctgtgctggcaagccagttttattggggtcagactgggtgcagggagctttggtttgtagcatttgtccattgctgtggtgtaaattctcccatcgtggccactttcaacctacggctccacatcactgagctcagagttgggaccagctgggtgcaccgtcggcttgcaagcctgcaggagctggctccagcacacactggacactgctaaagggctgcaagaggtagagacacaattagacagaagtctaggcagtgttagtttggaggggaaagacaccagatgggaggccgttgagctgagatttttgggtaaccttaagtctggtaggagtggtggggtgcaggggagaggtgtggaggtgctgtctgcgatccttgggcctcttgagtgctgatcgagactctgctgtgacttccacagccaggagttctccagaccccgactatcctcatggctgtctacctaacctcagtctctggtctataggattgaactaatgactttaggggccagataagagtagctggccctttgcccagaggggaggaggacggggtggaggagaagggaagctacaaaatgcagtgacagtggtggaccctgccctcagctgcagctcgtgcctgtggcttctgacacccagtgacctcaggactaggtgacaccagagaaggtggatgctggtcagtgttggagcaggacagaggggaggaaggatatgaggcccaaggaacccagacaaaagggatgcacggggtgacggaggaagggtgagctccggagagaacatgaggcccagctcccctccacttttgcccaaggcctcactgtgttgcctttccacctgcctaaaagaagcagcttagggtcatgactcagggagaggcggttagacaagtccaataatacaacttgtttaggggatggagggacccagcctgaggccctgagcccagggaagcagctgtggcaagctgagcctggtcatggtgtccctgaggctctgcaggtatggacatgcctctgctggtatgcatgcagctctgcaggtggggacacggctctgcaggtggggacatggctctgcaggtatgcacgtggctctgcaggtggggacacggctctgcaggtgtcgacatgcctctgctggtatgcacgcagctctgcaggtggggacatggctctgcaggtatgcctgcggctctgcaggtatgcatgtggctctgcaggtatgcatgcagctctgcaggtggggacatgcctctaccagtatgcacgtggctctgcaggtgtgcacccggctctgcaggtatggacacagctctgcaggtgtggacagggctctgcaggtgtgcatgcagctctacaggtgtggatgcacctctgcaggtatgcacatggcccctggagctgtcttccctgctgtaccttctaccccttcatgggcatttgtaacctaagccaaaagggaacataacaagtccagaaataagtcaaaaccagaaccactcaaacttcagcgtgtgtgcataggaatctcctggagggcttgttaaaacacagattcctggccccacccctggagagtctggctcagcagatctggggtaggcccaggacttcgcatttctaacaagcacccagaggaggtccaacgaccacactttgagaatcactggtctagacttaacagatcccttctgcccattccctgctctggtcattcacccgggggtggtgctcccgactgacttcttagttctctgaggtagaaaggtttgttgtcagcagtgatactggactagccactgcaggggatattgaccatgcctgtgacagttgggcccagccctgtccacacggaatgcttgtctaagtccaggataaaagacaagcctgaggcctcagaaaagatgtgctgcagcccctgtccgggctgtgccttctgctgtgggcagctgattccagggcatgccgtctgcttgccacattccctgagtcctcactgatgtggttgaggggaccccatcaaaggcaggtgcctcatttcactctgctttgctgcctgttccctggactcaagggcatagtttctgtcaaattaatttgcgcttgtgcaggtttgttagaaactttgaagtgcttctgggtctttccatgggggttctggttaggtgtcatcacccaggccaggggattcagcgtctcccatccctttcccacctttccatgacacccaatggcatgttgactcggagggcgctcagtaactgaggaatggactgctgactgatagtgcagtttatatgttaacgggggagctatagagcctggggattggagcctgctaggtccagtcatctgtgcaacagtttctacctaattaaaagaagaggctttgggttatgactcagggagagacagttagacatgtccagtaatagaacttgtcacgacggagagccaggctcctcagccctggcctgccaagcttgtattctcttcttactgtgctcccatggggaatgatcctataaacagctgctgctcattgctgaagccaggggagacttcttgtgatgactgacagaccacacagagcaggtagcccctctcttgggttttagccctggtgacctctcatagggtggctgaggggtacacagacccgctgacttccctcagggtgcaggatggagcaggcctctcggtagtgcgaccatgaaagttttcaaattttctgagatttcctcagccaggtgttgatggccatggggataaaggacagaggtaacacaggcagcaaaaaaataaaaaaaaaccccacaggtttggggtgagcctttcttaggagctccattagccaacagccaagatacctcattttgcactcacttcacttgtgaccaaagtgtcttaggaccaaaagggatttgggactttgttgaactgtttatataaaaaaaacctggctgggcacaatggctcaagcctgtaatctcagtactttgagaggccgaggtgggtggatcacttgagcccgggagttcaagaccaacgtgggcagcatggcgaaaccccatctctacaaaaaatacaaaaaaaaaaaaaaatagccaggtgttgtagtgtacacttgtagtcccggcaacttgggaggctgaggtgggaggatcatttgaacctgggaggttgaggctgcagtgagccgtgattgcacctctgcactccagcctgggcaacagagtaagaccctgtctcaaagaaaaaaaaaagaagaagaagaataagaagaaaagaaaaggaaaccttctccaaggaatctctaaatctattgctgagaaaccagttctctagatgggtgcttttcttgagtctgagatggggcagttgtatcacctgcagtgttgagttatacaatgatacatgaagcatgataatagctgcatctcataacatagataaatctgctgacactacttttacaacactcaaacagaagcaaaactaaagagtgcatttttaactacttattcttgaaataatttcagacctacagaaaaaatttaagaacaagagggaactcccatatgtccttcacccagattcactaaattttaaccttttgccacacacgttattattttttgaaccgtttgaaagtaggttgcatatatcacattggtgttagtccatttgccttgctgtaaaggaatacctgaggctgggtaatttataaaggaaagaggtttatttggctcatggtaatgcaggctatacaggaagcatggtgccagcatctgcttctggggagggtctcaggaagcttacagtcatggtggaagaggaaggggagccagtgtgtcatatgacgagggagggagcaaggagagggaggttgcaggttcctttaaacaactagctctcctgtgaaccagcaagaactcattcattaccatggggttgtcaccaagctattaatgagggatctgtccatatgatccaaacacctcccaccaggccccacctccaacactggaggtcacgttttaacgtgagatttggaagggacacacatccaaaccatgtcagtgtctctttacttaagtgcaaaatatactctcctacatatagtataggtacacatttgggaaatttaacattgacaaagtacttcaatctcatctgcaggcaatattccaaattgatcacttgtctcagtgatgtcctttgaagcaccttttcccgccttgagtccagcatcacatactgcattttgtggtcatgtctctagtctttaatctgcatctgttcctcggctttctgtctttcaaaacattgatgtttttggagaatgcaagccttcttttccgttaagtcgagggtccctacatttacattttcctagtgtttcattataatcacattcagtgtatggatccctgaccagaatgctacctacatgatgctgtatccttctcagggtaccagatctggaggcacacggtgtccatctgccactcattagtgatgttaattttggtcacccagtcaaggtgctattcagtttctccacttacggttaatatgttttcccttccaactaatcagcaatctatgggagacactttgagaccatgctggtatcctgccttttactaaactttcccttaagatttatcatcaacagataatttttgctgaacaattatttactatgacagttgtaaaatgatgatttttaaatgatttcctccaaatttattacttggtattctactatagagccctctcttgttccccattcacccacctacacacccacccacctgcacactgattcacccatctgtcacccattaacccatccattcatccactcattcacccacccacctgcttatccactcatcgtccacctatctacccacccatccacctatccacctacttgtctacccaccatccacccatccctctatccagccagtcatccatccatccatccagtcatccacccatccagccatccagccatcaatccacccagccatccacccatccatgcacccacccatccacccacccactcacccacttacctatccatccatctttcatctacccattcacccacccatccatccatcctcacattcacctgtccatccatccacccacctatccatctactgatgcatccactgatccatccatccacccacccattctcttatccacctttctatccatccactgatccatccatccacccacccatccatccatccccattcacccatccatctatccattcagtcgcccatctattcacccatccatccatccccccattcatccatccacccattatccatccattcagccatccacccatccattcacccattcatctatccatccactcacccacccacctacccatccacccatctatccatccactcagctatccatccatccatccatccatccatccactcagctattcatccatccacccacccatccatccactcagctattcattcatccatccatccacccacctacccatccacccagctattcatccatccatccatccatccatctattcacccatctagttatcatcagtatagattcatgaatttctattttattcatcaggttaaaatccattactgtcctcatacagtttgatgttcagattgtcccagatttggccagcaaaagcccctttaagctgactccagcaacaatatgtattttaaatgtattttctaaaactaagttgagtattaaataaatagttctattaataataaggcaaatttgatcattaggatgactcagattttagggggaaattagtgtccctgaaggcataaaggagactgagaagcagtttctgtctccacagttggcttcactgacctctgtgctatcagagagcttaggatctgacatctgcctcatcatcttaccctggatctgctttctgtggaagaattgcagcagacatggtcatgttgcagttaggactgggcagcagcaaagccagactcgaggcacctagacctgagtttggagcactgttagctgaatccagagagctcagccttcctgagccctgaagagaccacagaggtggcctagtgggtgccacatgcagcctggctgggagtaagaaggagaatgggaaaaaccatggcctccttggtttgcagagaaggaagaatcactgttgttacttctctttgctttaggttggctcccatcatctgagactccacagaggcctggaagccaatgcccctgctttcgacaggtagggattgtctgacaccatccaagcctgtcattgtcatccttagacatcgtcctcccatcagacacaagggagagggtgcgacgggaaagggaggaccctgaaccctgaatctaaagctgctttccctttggtcctttatgcaagaagggagatgacaaaatgggatttttactttgttctcaggtttcctgaaggtaaattaatgaatgttcaggtagcactttataattattttatcttaccagtttcttgcaatgaggcttcatctctatgccctggttaataagggatcatctaaattatagggtatttgaagctgtttgttttttgttttaaaatagtatagaataaataagtatagaaaagtataaagtgaaaaataaaagtaccctaactcccagttaccactctcagagattttactattagtagttttttacatgtcattctagaaaacaattttggcattttatgtgtgtgtgtgtgtgtgtgtgtgtgtgtgtgtatttaaatacacaaattggatcataacacatttactgttttgcagcttgcttaagaaaagcagagctatttgtaatatgtatttgaaataataacttcttaaaagatttgcttatttaatctaaatactcaatattttaaaattgtcttagtgaatatagtaaaaattctcgaaagtaatgtgttaagtgtttgagtccttctacctgaagctttattgagactttaagtttgctttcatactcttgttaaacataacacttgtcatatgagcccagatgtgaccccctgattagtgtgtgacatgtaatgcgcttttctttaaagagatatttaatcaagtatagacaagaccattgccttttccaaagcattattagtatgtcctgagcattctacatgcggagttctttcagttgacattcatctatactaaattgtcttaggaaatagtgaacatgaaaccaaaaatgaaattctacctggctgagcttggtggctcagattttagggagtcgcctgggtgacagagcgagactccatctcaaaacaaaacaaacaaaaaacaaaaatattctacccgaagctgacccaaaacaagagggaagggtcatgctgggttcgcttcaggaaccctcctatgtttaggagctttcttaaaaaatacatacatctagttgaagcacatttgcatagactgcagatttttcagaatcctggtaatgcatggtgtgtgagatgcacgtaccacagagaatgagacctctctctcctcacaagcaccccagaggctgccttgccccttggaaagcattcctaaatctaggggtacacttagatctcttctgcaaatcacaggacaagctgctgaaagggtgagtctttctacaggcagctactgtgtccaggaagtctcttctgtaatgcttcttacccacgacactgggctggaggcaggagccctcccgttcccttgcaggtgctgctgctctcatgttcattgctgactatgttcccctcattcgagataacgtttggaaataaggtgacatgtctgcctagtggccgcagtggggcaccatgagggtccccctgaaagcaggctgcccaggtcctcgatggtgtccctggactggcatgtgcattgtcctgggtgcttcccaattctcttctctgggacatcactgcgctctcaagagtcgtgcggttctctgatggatttggttgagagtaaatcctttctttcccccgcaaaaccttcacacagatagtttttcttcccagaggatgcttgttttcctttgcctgagctgcatgggtgcccctgttagccgaggcggtctcttgtcactttgctgtatgtgctccatatctgaggagggctgtttgcagcaggactttggctgcagctgcttgagtggggtcttgtcatctctgtgtggtgatgagtctcggtggaagggctaacctttcacagggcggtgcctcagtctctcctcacaggacagtgaaccccttctccttccccgccccgctctgcgggcggctgctgtgcacatcagggtgtgctgaactcttggtccctccctgaagatgagaaggatgcttccaaatagaaatctgattcctcagtatcccgaaaaactcctgttttccaagtagcttcctatgacagtaacccgtaggcaggggaagtgaaaaaaaggcctcaatgctcagggtgacccaggaagaagcccgtatgatgccgaccttgctgctggtgccagcgtccccttacaccctgggtgccagcgctcccttacgccctgggtgccagcgtccccttacctgggtcggcagggcctctcagctcaccaataacgagcattttacaataaagggaaagaaagaggaaccacgtcccacacaccaagcactgtccttgtcactttcactcgtctttttagtcctcctcgccattctccgcaggaggggaagtagccctgctttatagatgaaggagccagctcagaagggatgaatcatttgatcaggggagtttggggttgggacagcatagagtaatgatgaggaatagtgagtaataatgagtaataacagtttccttctggccctgggctttggaaccagttgagaagaaggggcaggaatgagacaggaagagatcagaaatgggccttgtttccgatgagccacattgtgggatgtggcttggatgtgtggccacggtgggctccctcaccaggggcagagcctgcacgtgtcagagccaggcctgggatgcaccttctctgggcagaccgacccaaaagaaagcccacaagggctgaagccacacacagcccgccccagggctgcctggagctgcacgtggagacgggagcccgggagggtgctcacccttaacttaggccagggagtcagcaggcaagcctaacttcaaactacccacttcaaaatccatagtctttctactactgtcccttaaagttagtaagagaatgttttctcttactgcataagaaaaaaaatgatttttttcatgatcattattagctctttaaaaaattccgctcataaacgtgtcattacagaaatttggaaaagagaaagatgtttaaagaagaaaagaacttgaatcatataaaattctaccacttttgacatcatgtgtttttttccagtgttttttctatatttaggcataaagattgcaccattaaacgataatttgacatctgctttttgcattgtatcatttccctatgttttgtttaaggcactacatttcaacatgtttaaatttgaaatgtaaggttacttttaaaaagctggtgatcaagaataaatattggaatctctccctaatatttcaacatacatatttagaaagtagcttccaatacaaggaatgttttccaaagcctgcaagttttgttgtatctccttgttgtggtttagcataatgcaatatagcacttagattgaaaacaggatgctgagttgagagctctcctgccagttgttctagttttgagaagggtgggtaccaagcaaaataagagattcatttcacacatgtgcacattgcatgtgtaagactctagcccaggcacggtggcttatgcctgtaatcccaacactttgggacgctgaggtgggaagattgcttgaagccaggagttcgagaccagcctggacaacatagcaagaccctgtctctatgaaaaataaataagtaaaaattagccagatgtggtggcatgtacctgtagtccctattacttgagaggctgagttgggaggattgcttgagcccaggagtttgaggcagcagtgagctatgatcgtgccactgcactctagcctggacaacagactgagatcctgtctcaaaaacaaaattctcttgatgaaagttcaggttaggtgttctagatataatgggaggtgctttatagaaagtgcagcatatcctttgtctgattccaaacaaatgtagaagtctacgctagctagtagtaatggcagctgggaggtgcaggttctctccacccaggctcattttccacgatttgggtcatgggggacagctggtcctggactgtggcactaagaaagcatgactcctgccccgtcttcccttgaggggcagaacagggcagagtctgcacctgtgcccgtgcccagcatcacgcccaccccgctgcctttgcaggcacatggtgcttctgaaggagcagtacgggcagcaggtggtcgtgaaccttctgggaagcagaggcggagaggaggtgctcaacagagccttcaaggtaaggccaggctgtcctctccacagctgggctgggcggcaggtggccatggtggagcgttgagcctggaggtggctcctgcagggactggagctgacagcccagccctccttcgtttcctgaggcttccccagcattccgaccagtgaacacggcagtgctcagtgactcgggccctgatacagtgagtgtaatttcttttctttttttttttttttaagacagagtttggctcttgtcgctcaggctggagtgcagtggtgcaatctcagctcactgcaacctctgcctcccaggtacaagcgattctcctccctcagcctcccaagtagctgggattacaggcacttgccaccacgcctggctaatttttgtatttttagtagagacggggtttcaccatgttgttcaggttggtcaagaactcctgacctcaggtgatccacccgcctcggcctcccaaagtgctgggattgcaggcgtgagccaccgcgcccggcctagtgagtgtaatttctgtgagctgatggttgggagaatttgctcaaagtcaaaacagagcatctcctgtccccagccatgtgtcccttaccctccctccccatggggctttgagggcttctgacctctcaaggtacccctctcagtccagcccaggagcaggcaggcctccctttctgccctggctggtcctgggtgagttcccccagccccacagggcacccggcctcagggctcaggagcaggaacttcggtggctttcccctgcttgccggctggccttcaaatttccccctcttcctcgggctttcatggcagctcctggacctgctgctgctgggctcctgtgggctccttcagacttccttggggggcttttcctggcgatcctggacagatcctgggcagggtcacagagccatgggagcaagagactccgcccacctggccaggttccatccctgtgcatggatcccaggcctccagcccccgccccctgctccccaggcgcaagacgttccagaatgagccaagggggctcctggttcctttaccagcctccatgtcccctgcctaatagagaaaaactctctcgttgtttaaagctcaaagatatcccttacagagagagctcgaagtcgcctcactgggggttagggcgatctctgagcctttccctctgcttagaaatgccacatgctcatgctcagagcaggcacttcctggttgccaattttatttggccagacctgcttttcctgtttggtttgttgctattagcaacgagctccagaagtgtctcccttcccacccatcttggcagtgatgctggctgctaatttgaaggaagaagggaaaaagctaaaaaataagaataataataagaaccccaagtccttttcccctatgccttgagttgtggcccctcccatcagtgggtgaagtgcccggcactggtgagggcgggagatggcagccacctcatgcggagaagggtgggctcctggccaggtagggaggggcactctagctgaagacctgggtcatggccctgcctggggtggccctcagccacctcagcttggcccagaccaaggttggtggcccagcagcctgcccagggcccaatggccatcagtcctgctggatgaagcactggctgtggaatgcatggcactgagccaccaggcactgttgggctcctccagcttcttgctcacagcaagacttgcttgatgatggcagcactgcattgatttacactcagacctatcacaggcagtcacgttgagtcagatcagatgacccccaagtccttctagtccttccaagtcactaggacctctggctgggcagaggctggactggcctgacctgaagccacaagggcatgagtgcagggtgagaggcctggaagccgggtggcctgggtaggcggacagtttgtccttcatcacggggctctctggggagaaagccagggagctgccaggccagggaagcctcatatggaagggagggctcttaagggcatcctggtgccatcagcagccccttgacctgcttcccatctctccccaccttcactctggctccctgaccatcctgaagtagcatcgggatgtgggacccacctgggccacttcagctggagacttggtctcttcagagcataaccaccagaggctggtgcgcggcaggatggggccggttactagggcaacggggagctggattatgagctctgcagcctgccacagcactgctatctctgagggcctggggcacctgggccacctgaagaacagagcagtccttcctcacctgtgccctccagagagccctgcaggagtatgatctctccaggaagccacacctgctctggcttcttttgctcaagggggaccttctgccctttcgcaggcaagacgctctttgtgggttttgtccccccagggcctttgtcctgtgcttcccttttggggtcaactgcctggtccactgtgagcttccagctaaagcacgtcctgcggcgagtcaccttggttagccgcacgggtcaagctctgcaaggagcttgccctcctcgtcctcccttccctctgctcccctcaccgccctcccctcctcttttcagaagctgctctgggcttcttgccacgcgggcgacacgcctatgatcaattttgacttccatcagtttgccaaaggtgggaagctagagaaattggagaccctcttgaggccacagttaaagctgcactgggaagacttcgatgtgttcacaaagggggagaacgtcagtccacggtgaggctcgctgcgcactgtgccgcgtcttctgctggggggaagcgtcagtccacggtgaggctcgctgcgtgctgtgccttctgctgggtgaggcggcagaggggccgtgcagtctaggacatgtgccctatgaggggcagcttccttccctccccactcagttacccttgtggtttcctgggtcataagcttggcgacgccgggaatcttgggttccagcttctcatctcccttcccacagtaggagacaggcttttgaatttctcaaggagatgttgagtcatggttttctggcaaaagaaatgaactaataaaggggactgcattaaaacatggagctccacgctgtgacgcaggcctgtgagagtgagggccacagtcgcggtgggatggctggcatagacttagattgctagaggagaccagggtatggacttccctgttcagagcagaggagaaaaacgaagtccagttccctcaggtcacatgcagggggtcctcattatttgcagattccatattggcttatttacctattccttaaaactgatttgtaaccccaaaatcagtatgcaaagcatttttgctggacagagtatttaaatactctggcctcgtatttaaacaagtttcctttttaagatttagtaccatgtttttcacatttgtggggtctttgttaggggcccccctttgttcggggatctcatggtttagaatggccccaagcagtgtgccgaagagctgtctagtacttctgagcacaagaaggtggtgatgtgctttatggagaaaatgcacgttaagataagctttgtccctatatgagctacagtgctgtcggccatggttcagtgttaattaagcaaatatatatattcaataaggtgtctttatatggaaacatacataaaacaaggttgtatattgatcagttgataaaaatgtgaccagaggtttgcaggaacctaaccctatatttcccctaggagcgatggttctgtgttcatgtccaattcagtgttcctacaactttatagaacataattgctataaataacaaggaccagctaaacgtaaaacgacccagctgtgcaggctactgagtgtgtgtcctggggtctgcctgcctttccagaagtgctggggatccactgggatggacactggggtttctggggggctattgatgaggaggtgaggggttccctcttagaacatgtttcctcacacctgtaatcccagcactttggaaggctgaggtgggcggatcacaaggtcaggagttcgagactagcctggccaatgtggtgaaacaccgtctgtactaaaaatacaaaaattagccgggtgtgatggcacgcacctgtagtcccagctacttggaaggctgagacaggagaatcgcttgaacctgggaggcggaggttgcagtgagccgagatcatgccattgcactccagcttgggtgacagaggtgagactctgtctcaaaaaaaaaaaaaaaaaaaaaaaaaaaaccatgtttccttgggagtcaaaagtcagacatgggcctctgtgctatggccactgcttctttgaaaacaacatataaccgtttacatttcttcttgtcctaagttttcagaaaggcactttgcggatgaactgtcttgactgcctggaccgaaccaacactgtgcagagcttcatcgcgctcgaggtgcgtccctgccacagccttttcggccatctgtgccaggtcctgtgacttcagctggacaggctgggctgagcacctttagcggcaagatgagggtggcatcaccaagacaaccttctgactatggggaaggtggacagggagacactgggacatccagagaggggttccccaggaggctgctttctctgtcgtaggcacactttctcacaccctcttgtgaaatgcatcccagacatggtggcccaaaggctcctcgggaacagcggttgtgtgtaatgattatcgtggatgcaccattggccaaaggcagtgtgaggctggccggatgaggctggatttgaagctggggagggagacctctgtctgcatggccccagctgcctggttgacctctcctggctggggggtcgggggtagggtcagcaccccctgctgtccaccactgtttgctcttcagcctctggcattgggtggggcggggcggggtgcaggggcgttcaagtgtcagggggtgagaggagggcttgggtggaccctatagataaagttggggtgagatgtgggaagtcactctctgctgtgggaactcctcatcctctggaggggcacagagtcttccaggcacagcgtaatccacaggctgccgaataaggaacctcctgggacagtggctgcagggcctctgtgggagaagccagtgacagccatcccttattcctccccaggtcctgcatctgcagctcaagaccctggggctgagttcaaaacccatcgttgaccgctttgtggagtccttcaaagccatgtggtctctgaatggccacagcctgagcaaggtgttcacaggcagcagagccctggaagggaaggccaaggtagggccccgccgagggggcagggtgggggccccagggacccccctcacggcctgcggtctgggctctcggcaggtggggaagctgaaggatggagcccggtccatgtctcgaaccatccagtccaacttcttcgacggggtgaagcaggaggccatcaagctgctgctggttggggacgtctacggcgaggaggtggcagacaaagggggcatgctgctggacagcacggcgctcctgggtagggcctgccgcagacagggcgaggcagggaggtagggtgctccccagccccactttcccaccgcctctgtgctatccagagagcgggtggcagaggtgcctgggatctgagcagtggaccatgcaccttgcagctgtcactgcctctcccgggagcctgcgctttcctgctgggcctgggagctcacgcttcctctgtctcggcttggactgtgtttccatcctgagaagcgagagacaccaggcgggctcagggagaggccgtgcactctcctaaacgcatcggtgcctttgaatgctaacctactccagaaacttcctcagctgcctgggctcttgggcccttagcaggtgatgttggctttgggagcttcccatgaagttggcctccagtttctctcttggcccctgaggaccctgtggaaacagagaaggaggatgctgtcatcccctggggatgagggcaccttctggtctcagcggcaagacatcgtgattccatcaatataggaggttcctttgggagcattttaatggcatcttcttacctgagcctgtaaacatattaggtctcatggaaaagtaggggaaacccagcatttttccttataagaatgtacaagtgtcgctttagaaatgtcaggtgtttttgaaaaatgaactagcccatgcttaaaatgggttcacggccctttccactgtcaccgtcactgggggagctctaatttggcttccgataggcacggtgatatttagtttctactctggaaacagtgccagctctgtaaaaggtgaacaaagctccatgttgtacagtgagtcttatttaatagcaaggcagacatgagttagatttggtttacatcacaagtttaaaaggtggaatatattttggctatctagaggatggagatggttctgtgtagtgactttttaaagccttaagtctggggcaaaacaaaacatatcagacatgcaggctggaaggcagaggatgcaaaggtggctgtctgggttggtaagacacttaaagttatagtgtatatggatgcatacacaatgaaatatggtcagttacagcatgcccttgcttaaattcctatgccttgttcttctacggagtggacagtgaggacacggcttttccctgtcgtgtctgagatccctatggtctgatgtcccatggcttctggcttcagttgggggtaggggtatagacggtgagtttccagtcttatgttttcacgcctttattgtagaaatgccataagggaagccccgtggtgaccgtggttttaagccttttgtcgtcttcgtaaggagcataccctggtttatctctagaggctcatgaaccatctgtctctgccaggcagcctcatctgtctttttggaggatgcctgagctttggaactgcaaagaaatgtgcctttttatgcctcctgacagtgactcccaggatcctgaaagctatgactgagcgtcagtccgaattcacaaatttcaagcggatccggattgctatggggacctggaacgtgaacggaggaaagcagttccggagcaacgtgctcaggacggcggagctgacagactggctgctcgactcgccccagctctcgggagctaccgactcccagggtgagggcagtgactttgggggagacaaaatccaattgcacctaacctgacatgtcacgcttgaccctttagtggccatcgtcttgtggaatttcatctttggtgtcttccctcctcacaatgtctaaatagcaccatggcctgactctcaagaatgctgcctcgcaagtaactgacctttttcctttcccagatgtttagaagagttagcagatggtgaatctctcttttctctttgcatacagtgatggtgaagtcttaggctttcatcagtagaatcttttcgtcaccagtcggaaggtgtggcttctagggccatgtaactgtgaggttgggagccaagcctgtcttcggtgttttgtttttgttttttgttttgttttgttgttgtttgtttttttgagacaagtttcactcttgtcacccaggctagagtgtagtgacgcaatctcggctcactgcaacctccatctccaaggttcaagcagttatcctgcctcagcctctcgagtagctgggattacaggcgcccaccaccacgcccagctaatttttgtatttttagtagagatggggttttgccatgttggccaggctggtctcgaactcctgacctcaggggatccctctgccacagcctcccaaagtgctgggattacaggcatgcgccaccgcgcctggccctgccttcagtgttgatattgaccaagaacagctcatccctgcctctccccaaaccaactctattaatctgctttaagtagtcacaaaataattcatgtgctatttaggaataaaataattccaaatgttgacacctcaccttttatcttgtaagatacactattgaatggccccagaacttaggatctgaagaataaatgattcttgtccagtcatcgttgactcgggccttggtttttttgtttgtttgttttttgttttgcttccttggactcaggaacttgtggttcaggtttttgcctgttggtgagtgacctgtggtctagctgacatgcagtggcccatgtgtacattcatgccccacagctgcctgggtagtacccagagccccatcccttcctgtgcacacgccatctgtgccctcctttctgcctccaagggcaggtggcagagtccaccattcagagccactggggctctctgtgtcggtggtagcctgggttgctgcttgtggtgcctccagaagccattcaggatgccctccggagcaggcccatcaacgcggagagtgcttagcacagccgtggctcaagagccgtccctccagtccccacgaaagtgactcctcttcttttccctgcaggaaggtgtgcatgtttttagcaagctggctggtattttgcaggggatttttttggggtagacactagcaggctccccagcagtcccactagagatacaccaggttccagagaaggaaaggagtgctgtggttactattgccaagcagatcagacagagggacggggattgttttatttaaaaaaaaaaaaaaaaaaagctttaagtcagggaaactagaggttccaggttgcccattgtggtcaacagagaaggaagtggacagcctgcctggcctctcgctgacagaggccatgtcctcctagctggaccctggggaggtgggcctctcactttctgcagcagctggagccatgactgggcttggtggctcaggagcctgggtgtcagaggtcgtgactggacagattcagtcaatggctacaaggttatattaaattctgacctcccctgttcaaagggaacagaatggacggagggcccctctgggctcactgctgcatttaagccaggcatttctgaagcagattccactggggcagttcccagcagcaatggtagccacctggagttggactcaggcagtggacagcatgactcgggagagggcactggggagccccatgaactcgtaatgcgggacttggcggttctgacttctgtcatctcttgccttgctccccagatgacagcagcccagctgacatatttgctgtggggtttgaagagatggtggaattgagcgcagggaatattgtcaatgccaggtaaggggccaggtgtgcggggccaggcagggacttcctgagtcaggtgcctggctgggagcagagcctctgaggctctccgggagccagcctgcttctgagccagctaagctgtggccctggctgtgtggggatctctcacctggctccttctctatgctcacacatgcaccaccccgttctttccctgccacttaggaagtaaaaggaagggctgatgattgctgtgttttctttgcagtgctctggcctcggccacttgttgaggctaatttatgctgccagagggagctggcatgtgtggacctggggtgcaggggaagggcggggctgggactgggagagtagttgactcatgtttcctggggagaggtgagaaagtgtgagtgccgggaagaggtactttcttcctgcacaccagggcaaaggaaaaaatcaaaaccactggatttttatttgatatgaagtcaactgggcgctccccccaccccctgctgaactggcctggctttattttaaggaactttctgaacaaataaataagactgactctgtggggctgctatgagaaggtcctgggttgggtttcaggggtcaagggagggctgctgtgatgtgtgaggcatagccaaaaacacgcagatcctccagtagaagaaggaaagcatgacactaatattggggtgcgggcagcgtgaaatcagttctgcaaacagaataggcttgggctggagcatttggtaggtgggagatagatgtacttccagctacctgtaaacagcatcctttccttttctcttccagtactaccaacaagaagatgtggggtgaacagcttcagaaagccatctcacgctctcatagatacattctgttgacttcggcacagctggtgggcgtctgtctttatatctttgtacgtccataccatgtcccgttcatcaggtaagaacattctgtttacacacatctggggaacaaggggttgttagtctttgtgttttgttctttgacttccccctcctcccccatccccttctgtaacaggaatcgggacttaccattattttagataaatgtagcaaaaccccctaggaaaaactgggctgaatagaatattgtctagcgattaaaaagccaccatgggaaatgtctaactcaggaagctgaactcttagaaagggtgttacatgattgtcatcgcccaagctcaattccgtgcaaggaaaagggttccttgttggaatcttatagacaggaaacctctgaccaagagctgagtaactcactgggaaatgccaactcttttgtccctttttaaaagaattctaagtagaacgtgtgggcctctacaactgtgacctcattgtcatttctccaaggtgcatgctttgtgagcatcagaaagggggcgagcttaggggcggcattcctcttggggtgtgggtgtgggtgtttggatgctggaggaactcatcttttccccagcttgtggttggcctcatcttaccacctgggcctacccatggcttttgaatttccaccagcctttcggcgagctggcagcaggcgttgaactgacctccccactgagcccctgggtcccgggaagggcctgtgcctgccaagagcaccacgggtacacccctgcagcacctggagactaagactatgctgcccataatcctcttctctgactttcagaagatgtttaggtccctgtccctctgcttgtgttatttgggcagcttggcagtgtcctaggttagcaggtgaaggttagtaaaggttaaaggcaagggcggggtgagggtgagaggtaaagcatttgagaaagggctcagagctgaggagagccaggtttcccaggctcggtgtcctcggcttcacgtgcctttagccctgagctgcttccgcctacaccccttccatcagtcatccccccaaccttgtctgccagcgagcagatgcaccagcaggcctcagtctgtggcatggctgtggaaatgttaacaaatgtgcttgcagcccatgtttctatggactcgtatgcgtcataagtgggcatttgaatgcaagcaggctctggcattgcataaaagtccttttgagaccatctacccagcccagcataaacagctgcccgtttcctagctcttaaagtgcattgattgctgggcacggtgtctcacgcctgtaatcccagcactttgggaggccaaggcaggcagatcacttgaggtgaggagtttgagaccagcctggccaacatggtgaaaccctgcctctactaaaaatacaaaaattagccgggcgtgatggtgggtacctgtaatcccagctactcaggaggctgaggcaggagaattgcttgaacctgggaggccacagttgcagtgagctgagatcacgctgctgcactccagccagggtgacagagcgagactctgtctcaaaataacaataataatttaaaaatgtgttgattgcccagatgacacctcccatgcatggcgtgatggctgcacaggagtggggtgggctggtgggtggtgatgtttgcgccgctcctggtggcagtgagaacctgggcggatgcaggcattgccctgtcttcggcttcatggggtgctcaagtgagcacttcccagttggggtgtgggggagatgtccaggcagcggtgggcagcaggagggaggcggcctcctgaccaggagtcgatgacggccacggtggccactgtgttgagctgatgattttggagcactcagagcagcaggtcccgagctgctgctgggcccttctctgtggcaaagcagggtcacacttctccttcaggagccgacggcatgcgcgtatccttctgttccagggacgtagccatcgacacagtgaagacgggcatggggggcaaggcggggaacaagggcgccgtcggcatccgcttccagttccacagcaccagcttctgcttcatatgtagtcacctgacggccgggcagtcccaggtgaaggagcggaatgaagactacaaggagatcacccagaaactctgcttcccaatggtgagcggcgccgtggggacagccagcacctccctgctcagctcagtcccaaatgtgactgttgataggagccaggcactggggacacaaccacagggagggccccttcctggagggctcagcgctgggggagggggagagggctatgtccctccatggaattgacctggggcagggttagccacgtgcctggagggggccagctttgcagcatttcccgttgctgtacaattcctgagtgtctgcatggccactggatttgtatgtgtgcattttctgattttctaattcaagtgatggtttagggaagaaaaccatgtccgtggtctgtcctgtccaagtggtgaagagcatttgaatccacacagcctaagactccaccttcctgagattcacagtcttcacatgtactttctgtggtggctcagataagcacacccttttgaccaatggaccctttttgactgggaacacggttcttgaccaaggagcccagatcctgcctgtcctcgaagctgcaatagcgaaccgcacactgccctctcgtggcggtccggagggtttcattctgaaactggaggattttcttttcaaatctagacgttaatgttctccttcctgtgcaaagatgctacccgggcacagcttcagcgtgtggtgcacacgaggccacctctttcccgaagtgaggatagacttcagctgttcttgctgcaaaatcttccactgtctgagaggtggtgggttgggtcacagagctcttccctgtgagaacagatccttaaaactgacgtcccattttactgggctgcacgagcactcaaggcaaaatttccttagttttcattaagaagtgccataattcctgttggtgttgtttatttcttatgttaaaaaagaaacaaacgagttgggaaattttcaattgtgtgtaatgcagtttaaaaaaaaaaaaaaagtctaacccttctcacttaaaaatagctactttggggccaggcacagtggctcacacctgtaatcccagcactttgggaggccgaggcgggcagatcacctgaggtcaggagttcgagaccagcctggccaacgtggtgaaaccccacctctactgaaaatacaaaaaaaattagccaggcgtggtggcgtgcacctgtaatcccagctactcaggaggctgagacaggagaatcgcaacccaggaggcagaggttgcagtgagctgagatcgcaccactacactccagcctcggcaacagagcgagactctgtctaaaaaaaaaaaaaaaaaagtgctactttgacatagtaaacttgggatacataataagtaattaaaggatttataatgtagttcttttctaccataaaaatattgagggacctattaacattctacacttcttttttgtttttgttttttgagacagtctcactctgccacccaggctggagtgcagtgacatgatcttggctcactgcaaactccacctcccaggttcaagcaattctcctggctcagcctcctgagtaattgggattacaggcgtgcaccaccaggcccaactagtttttgtatttttagtagagatggggtttcgccatgttggcccagctggtctcgaactcctgacatcaagtgatccgcctgcctcagcctcccaaagtgctgggattacaggtgtgagccatggtgcctggccaacattctacactggtattgtctgataccgtagtcactggccataagtagctatttaaattcaaattaattaaaattaaataacatttaaaattccttgctttccctggccacagtgagaatgatcagtatccatgtggccagtggctcctgcatcagatggcacagagaacatttcggtgctgcagaaagttctgtctgacagtgttatctagacgctcattttatttggttgggacctgtttaatcttctgtccagattattgtcatggggggagtggaaatgcctcatattataggagaagagaagctgattttggagctaggcagatgggagctgaaatcccatctgtatcatgcactagctgtgtaaccttcagcaagtgcctttgtttctgaactttgtttttttttttttaatctaagagagagctggtggtatctgctttgtgcacctggaacagagcccagcccttgcggacagttgatccacagcagctgctgtcattgtcttccccattcctggggaagagacagggaaggcaaggaccccaggagctctccggggctgagctggggccgtgggctcctaggactgggcaggatgggaaggaggagctagagagagatggagaagggtcacctgaaaaagctcaggaagttacatgaaggtgcacatcctgagcatttctgctgctcttttgaaacctcattctcctaacgctgaatgctgggtgctgcttcctaggagtagacactccaaagtaagggtgcatctgatcttcaggacggggatccatggaggaaatgctggccatgttgctctcctccgtttagggtaggagctttcttcaaaccactcagaaacactattatttaaaattaaaaagtaagccatgtaaaaattcctccatagttttatgggatgagtgaaaaacaagatgctctagtagaaaactttcttttttctaagtagcattttgagaaaaatactcctgcttgccccagtgagcttgtttttctcctctctcccaccttccttgcaagatgggctgaatgattatgattttcttttcaacttaggggagaaatgttttttctcatgattatgtattttggtgtggcgatttcaactaccgcattgatcttacttatgaagaagtcttctattttgttaaacgccaagactggaagaaacttctggaatttgatcaactacagctacagaaatcaagtggaaaagtaagttgtgctttaaaaacattttttagcagctgctccaagatggaatgacatctgtgagatgtagaggctggtgcagcaaattcagttccgtgagtggatttcactgtttccaacattgtagaaaataggtgggcttgtgaggttacagtacaggagtggctgttcatacacacttcctgtccttttttttttttttttttttttcctgaggcagagtcttgttcagtcacccaggttggagtgcagtggcactatcttggctcactgcaacctccaccttcctgggttcaagcaattcttgtgcctcagcctcccgagtagctgcgattacaggcgtgtgccgccacacctggctaatttttgtatttttagtagagacgggttttcaccatgttggccaggctggtctcgaactcctggcctcaggtgatctgtgcgcctcagcctcccaaagtgctgggattacaggcatgagccaccatgcccggtcaaccttctcctccttctaatactgttattttagtgcctgtttgcttcctagcaggatgagctgagatgaggggagtacatttaaattccatttagctgtggagaaagaagctggttaaatgtgtgagggccaggtgcggtggctcttgcctgtaatcccaacactttgggaggccgaggcgtgcggatcacctgaggtcaggagttcaagaccagcctggccaacttggtgaaaccccatctctactaaaaatccaaaaattacctgggtatggtggcaggcgcctgtaatcccagctactcaggagactgaggcaggagaatcgcttgaacctgggaggcggaagttgcagtgagccgagatcgcaccactgcactccagcttgggcaacgagcaaaactccgtctcaaaaaaaaaaaaagtgtgaggacagggcagataaggtctctcctgaagcaagatttggacgttcttcgaggtttactgtgtcttcccacttgagccccttccagtgtgtggagtgctaaagggttgccacatggacgagtgaccataccaacctcctaaagcaggttttcctgtcttcctactaccaacattctcacatcctctcagtgatgcccaagagaatgagggtgtgtttgtcccttggtcttcccatggaagcaaggccaatccaaagttaaacaaagaaaagaaggatgtgcatgggggctcacgactgtaaggcgggaggattgcctaagcccaggagtttgagaccagcctgggaaacatagggagactttgtctctacaaaaactaaaattagccaggcatgatggtgcgtgcctgtagtcccagctacttgggaggctgaggcaggaggatcacatgagccccagaggttgaagctgtagggagccgtgattgcaccactgtattccagcctgagcaaaagagcgagaccccagggggagagagagaggagagctgactttcactctgcgcttctatgtacttagctcatttagtcaacaaacatagatgaaggcctcggggttgtgggccagacatggcaccaggcatgggactgtaaagatgagcacccctgacccgtgcctcggaccctgcactgtctcctggggggcaacaagaagacagtggatagatcagacactggagtgggtggggagatagatacgagggcggtgtgtccctcagtgggatgagctgaggatcacttttttgctaattaaaagacacatatgtccagtttcagatagattttcaaatgtaatggagcttctgttactttttaagaatagggacttgccttttcagttttcgtttttgtatataacattcaggatcgaaaactacggtggcctgatgacttctttttctcccaatagatttttaaggactttcacgaaggagccattaactttggacccacctacaagtatgacgttggctcagccgcctacgatacaagcgacaaatgccgcacccccgcctggacagacagggtgctgtggtggaggaagaaacatccctttgataaaacaggtgagggggccgtgcccgttcgagagtcggcagagggtgaataagaaatgcgacggagggagagtcacgtttacccaaccccaggcgctgctacataaatcagtttcttcataaccccagatgacacaaaagtcatcccagaccttaacccctaagctttggaagttgtgctttcagggagcctctgcttctggcctctttttttcccttttcttttcttttttttttttgagacagagtttcactcttgttgcctaggctggagtgcaatggcgtgatcttggctcaccacaacctccacctcctgggttcaagccattctcctgcctcagcctcctgagtgctgggattacaggcatgcaccaccatgcccagctaattttgtatttttagtagagacggggtttctccatgttggtcaggctggtcttgaactcccaacctcaggtgatctgcccacctcggcctcccaaagtgctgggattacaggcataagccactgcgcctggcctcctctggtctctttctagagacagtgacctgcaaataggtctgctcctggctgcccctgcatcacagctcccccccacacccccacctcttttttctgggtggggaaagcggggagcatgaggttgcctccctccctgccccccatgctccccctcatgttctttatccagcctattttgttcagctacacttttacctgcttaaagccctaattccaaattggacacgagcaagacacagtaaaaataagttagtttttaaaaatcattagttgtcaagtatgttttgaacaactgttgatgcccagctaggaagtgaagtttatttaaagttagaatatctgtaactagtacataaaaaaaaaaaaaaaaaaaaaaaaaaaaacctaattacacattttgggagagcatagccaggaaattgcatctgtgaaaagaaaatacaaattttcctctagatgagtttgtgaacgcctgtgtggccccagcgctctgagaggtggggcctagaatgagtccgagtgttctcagctcctgcagctcttaccataccaattgctgccaaccttctgggattcctgggtcctggaaacccggcagcaacactgtgtctcaagcctacagtgggcatgctggcgaggactgcaggcatggtcatgactgttttgaataattatatgcgaggaatgtttttaaccaaaggccccaaagcatccgaagcctggcttcaaggtggaggagtaaggaggatgagtctgggacgtggggtggttcgtggcgcagacctctattgtggaagtgtcatgatcgcattcttggatattgactcgattctatatgggtctctcgaatggaacccctgcgagtagctggagaactcaaccttctagacagtgatctagatgttgacaccaaagtcagacacacctggtctcctggtgccctgcagtattatggtcgtgcggagctacaagcgtctgatcacaggtgaggtcctgacttccatggcgttttctcctgtgctgggcaaggcagcgtctccctctccgcaggaaaatgcatgctgtccccataaggatgtgtgtgcgcatgggggtgcattttgtgtgcatacaatacaccaaagaacaaagaatgatggaacaaatatctgtggacccaccaccaagaactaattaatcttagcattttgtcattgctccatatcataaatgacatcattgatgaaaatttcaaagatgcaacctacgtattttgcggcttttatattttcttaaatgatgtcgttgtgcaacttgcttttttgttactatttttgagatacagtcgtgttgatgttatagctctggtgcattcatttgtaactgctgtctagtatcccactaaatatatccatggaacttacaacttatccattcacctcttgatgaatattttctccaggattttatgattacaaacaccatagggtttttaaatctgggatgcacaaccttttttttttttttttttttgagagagagtcttgctctattgcccaggctggatggagtacagtggtgtgatctcagctcactgcaacctccgcttcccagattcaagcaattctcctgcctcagcctcccaagtagctgggattacaggtgcacaccaccatgcctggctaatttttgtgtttttagtagagacggggtttcaccatgttggccaggctggtcccaaactcctgacctcaggtgatccacccaccttggcctctcaaagtgttgggattacaggcgtgagccaccgcacctggccctaggatgcagggctttttaaatgctctgtgcatcatgcattgaggatagagtggaggcagcagagggagcaccgtgggcaccagcacctgtgccctgggattgctgcagaggaagactcaccctgcagaatgtctttaaagacagaggagaggatggcatttgagggtgtgaagaaggaagagtttgatcttcccaaggctgctgcctaaactctcatgtcagtttcaacttttacaaggaaagaaattaagaaaaagaaggtaagaacagttggtcaacgaaaaaaccttttttttttttttttgagacagagtcctccaggctggagtgcagtggcacaatcttggctcattgcaacctccacccccagggtataagtgattttcatgtctcagcctcccaagtagctgggattacaggcgtgcatcaccatgcccggctgatttttgtatttttagtagaaacggttttgccatgttagccaggctggtctccaactcctggcctccagtgatgtgcccgcctcagcctcccaaagtactgggattacagccaccatgcccagactctttagataattctaaattctttagatacctgttgttgcaaatacatacctagaagtgaatcttgaggaatcttcagatatgtgacatcaaggtttgctagctcaatgtattttgaaaccttaatttaaccaatatttcttgaggggcccttacatgccaggccctgttgctggcctggagaaaagcagtgaacaaaacagatgagaccatgttatcatggaattttctggacaggacaaacagacaataaacaaacatgtgtgctatgaaagaaaaaaagtagtggcaggagtcaggccagagtcgagctattttcaatcaggctgtgtaattttcgtaatataggattttctggaaatttatagagtaataaccatccctgtgtcagaattactataaggattgcattatttcctgctggactgttttcctgtttacatcattgatttttacattattcctaatgattgcgtagggtattatatgaaggtatcataactgtttccccattaagaaacagagcattttggcctggcgcagtggctcacgcctttatcccagcactttgggaggctgaggcaggcggatcacctaaagtcaggagttcaagaccagcctagccaacacagcaaaaccccatctctaccaaaaatacaaaaattagctggtcatggtggcatgcgcctgtaatcccagctacttgggaagctgaggcaggagaatcacttgagcccgggaggcggaggttgcagtgggctgagatcgcgccactgcactccagcctggacaacagagggagactctgactcaaaataaaaaaaaaaaaaaaaacgagcatttccctttttttttttccctcccatcaacaatgctgtaaatgatcaacctcatgcattaaatctttgtctgaatccctggtcatttccttagaataaagtccgagaagtgagattactaggtgaaaagagatgaacaattttggaactctatgtaaagtgccaagtaaccctctgaaaagcttgcacccctggcagagtgcagatgtctgcttccccacctccttggtggtactgaggattaccatgtcaaaaatctttgctgatggctggttattttccaagtactgggatacatctttaaggtgccacactttattttccccctcaaatcatctttagataaaaatgccaaggtgatactaaggttaaggaaagatgaagactaaaacgccagagcagggcagctagcctgcaaatccacctgctggtggccacattcgcaggttgttggtgttgactgtttacaaagagccctgggggacagcagctggggactgggggctggagatgtggacatctgtcccaggctaactggggactggaggccgggggtgtggacatctgtcccaggctaactgtcatccctcttttgttcctaactcagacctgtgctggcgatcgtggaggtggaagttcaggaagtcgatgtgggtgctcgggagagggttttccaggaagtgtcctccttccagggccccctggatgccactgttgtagtaaaccttcaatcaccgaccttagaagagaaaaacgagtttccagaggacctgcgtactgagctcatgcagaccttggggagttatgggacaattgttcttgtcaggtaactgctcccctggctgatgtgggttccaggggatgtcgatgaggatccgtgagagctgccaggtcgttcttggcattgacttggagtatcatttattccaggatcaaccaagggcagatgctggtaacttttgcagacagtcactcggctctcagtgtcctggacgtggacggtatgaaggtacgctgtacttggccacactgtggagtggggtctgatcaatccctctttttatgtgggcatgtcttcttcctcctcttgccctgacctgtgccttttttttttttttttttttttttttttttctctgagacaaagtcttgctctgtcattcaggctggagtacagtgatgcaatcacagctcgctgcagccttgaactgccaggcttaagcaatcctcccatctcagcctctcaagtagctgggaccgcaggcacgtgccaccacacccagctaattttttttaaattttgtagagagaggatctcactctgtagccccggctggagtgcattgggacagttatggctcactataacctcagcctcctgggctccagtcatcctcccgcctcgatctcccaatgtgttgagattacaggcatgagctttcatgcctggccaccaatctgtctttatgtatagacgccatatgcgggagggagtttggattcttcactctggttctaaggtgtctcatgtccggctctagctacttgcaaaatagggcggaagggtgggcgcagtggcttatgcttgtaatcccagcactttgtcaggccaaggtgggtagatcacccaaggtcaggagatcgagaccagcctggccaacatggcgaaatcctgtctctagtaaaaatacaaaaattagccaggcgctgggtgtggtggcttacgcctgtaatctcagcactttgggaggctgaggcgggcaaatcatgaggtcaggatttcgagaccagcctggccaacatagtgaaaccccgtctccactaaaaatacaaaaattagccaggcatagtggcaggtgcctgtaatcccagctacttgggaggctgaggcaggagaatcacttgaacccaggaggcagaggttgcagtgagccgagatcgcaccattgcactccagcccaggcaacagtgtgagactctgtctccaaaaaaaaaaaaaaaaaaaaaaaaagtagccagacatggtggcgcgtgcctctagtcccagctactcaggaggctgaggcaggagaatcgcttgaacctgggaggcagaggttacagtgagctgagatcgtgccactgcactccagcctgggtgacagcgagaccctgtctcaaaaaaccaaacaaacagaatggggaagaagaatgtcagtcactgtgtctcacgttggaaggcagctctaccagcagctgcagaagcttagccattttagagtgtagttctcgccgagttacttgatctgtctatgctcgcactgccatgtgggagactattccagtctttctctttagagtatgtattttggaatatgcagctcatagatagtttccgtttttatagtatcttggctttaaatctgtaccaaatgggaagctggagatgagagttcaactttttttttttgagatggagtcttgctctgtcacccaggctagagtgcagtggtgcaatctcggctcaccgcagcttccgcctcccgggttcaagcgattctcctgcctcagcctcccgagtagctgggactacaggcactcaccaccacgcccagctaatttttgtatttttagtacagatcgggtttcaccgtgttggtcaggctggtctcaaactcctgacctcaagggatctacctgcttcggcctctcagagtgctgggattacaggtgtgagccaccgcacccagcccagttcaacttttttttcaaccagatgtatgagagctatgctgagcctgcgtgaggtctggacccttggtttttcagggccaaatgtgaagattggcctcgttgagggtgtgtgggttttggtgagaagcattgtgtgatcaacaggaggggtcatgatttttatttattcctgggtggccgctctgccctcccaggtgaaaggcagagcagtgaagattagaccgaagaccaaggactggctgaaaggtttgcgagaggagatcattcggaaacgagacagcatggcccccgtgtctcccactgccaactcctgtttgctggaggaaaactttgacttcacaagtttggactatgagtcagaaggttagtgaccctgcagggagggacaggcaagccgttcttctagcaacaggcctgagcttttaaggacaccttctaaagcctcccttgcagaagtgacggaggacacccgcagggcaagccctctgtcccatgtgatgagcatgtttgtatgtgtggacgatgcatgccatgctacctgggacccgaggcatctaaacttaggctacaaacagtggctatgaggatatcagggctgtgggtgtaccaatggaactggggtaccgtccctgctggcgcctgggccctgagctgcagggcagggtgcacgtgtcctgacaggagctgaaccagtcccactgatggaagactgagcctttccccctcattgttttcacccgaattgtgattcatttccttctcccaggggatattcttgaagacgatgaagactacttggtggatgaattcaatcagcctggagtctcggacagtgaactcgggggagacgacctctctgatgtccccggccccacagcactggctcctcccagcaagtcacctgctctcaccaaaaagaagcagcatccaacgtacaaaggtagcctgacccttcttttctcaggagcctcagcgatggagtcagctcaggacagactttcctttttctcttggcgattgggcactgtgtgatatcaagtatgcaggtcccaggagagacctcaaccaggcaggccaagcgaggggtagggactgagccctgtggaggtagccagctccaacaccctgatttcctctagttagtttatttttttacaataacttgtacccccataacacaccagattcaagataattcttgtttcctctggatattcaaatggccacggttctgaactggttttatatttgtgattatttcccgcccccaacaaaactcttctgattcagccaggcgcagtggctcacacctgtaatcccagcactttgggaggccgagacaggtggatcgcttgaggtcaggagttcaagaccagcctggccaacatggtgaaaccctgtctctactaaaaatattttttaaaaattagcagggcatggtggtgcacatgcctgtaatcccagctactcaggcagctgaagcaggagaatcgcttgagctggggaggcagaggctgcaatgagccgagatcgccccactgcactccagcttgggtgacaaagtgagattccgtctcaaaacaacaacaacaaaaaaacttctgattcaatcccagtagagacataatccatatctatgtcctgagttgctggcgtatggtcacactcatataatatcatattattatacagtcatgtcatacggtcacatcattgtatcattttagacttcagagggcctgaaaggtcacattgattgaaccacacacccctagattaaataatacaactttttttttttttttgagacagggtctcactgtctcccaggctggagtgcagtgatacaatctcagctcactacaacctccacctcccaggctcaagcgatcctcccacctcagcctcccaagtagctgggactacagggttgcaccagcatgcccagataattttttgcattttttgtagagacggggtctcactatgttgtccaagctggtctcaaactcctgggctcaagtgaccctcctacctgggccttccaaagtgctggaattacaggtgtgagccactgcacctggccagtaactgttctgagtaacttttaaagcccactaatgttacgtggctggcaggtcgggtagttcatatttcagtgtcagctacattgatgctgttccattcatagccaggcatgcctctagctgtcaatcactgagccccgagttggaggacagctctaaccagcaggtgcaggagcttagccacgcattgggcccattgagctggggctgagagcagagaacagggctgaaagtggattaaggtacctctcagggagggtactttgccaggagaggtgcgaagacctccagcccctttgaccgtacgctggtccccacactgaccaataacttgcctataccgaaccatcaggagcttttaaacttatggatttttgagccctaccagagagatctggatttggtaggagtggggtgggaccctggaagcctggcgtttcaccaagctttccaggtggtttggacgcacagctaggtttggggacttctgcccaagactgaagcagcagccttcgccggagcatcgcggtggttctgcacgttgctcctgccaggggcttctgcccgggagttgaagtggctggagggatgtgctcttcccataagaaggacctttctctcgggggtggccgtcatgcaggcagcctctggagccagtgcaggtgtgagcacagatagtcctttgcatgaagaaactaatagcgcaacacgggacacgtgggtggctttgctacctggtgggcattgagagggtcttcccttgatgctgtggggtgttcagccattcccatttgtaggagaaatcctgcctttccccagttccagtctccaggacacatggggtggagctgctggatacagggagtggggtgtccgagtcaaagtgaaatggagaggcccatgctttgggggagccctagggctgctgatttgatctgtaaggaagcaggagctgtgcctcctgaaaacccctcagcctcttggaaggtgtgtgcagggcaggtcaagatggcagacccttgggagtggccagccatgggcccacagggtttgggatttgatgagtaagcaggacccctggtcctaggcactggccactgcatgccaggctgctctgctgccagagcagggagaacaccctcagcttcctcagccggtgtgttctagagtgaggaattgaaaacatctgcctgtggttggttgactcttcaggggttgtggtttgcaactgtctcctccactctgcccaagtcttgttatttcacaagaaaacacactttgaaatagctcagtgtctctcaagtctgagaaatacccattatccatggttcactgaatgttccaagcaggcactgggtgccctccccgcccccgcccctcgccggcagacagaggtgcccgtttctggacagagccttcagctgtcctacagcaggtccccctgccacagtcggcctcctgtgctcagatccgtgtctgacacgctcacgtgtggaacagaccattgtactcatgccccgccatgtcctccagatgacgcggacctggtggagctcaagcgggagctggaagccgtcggggagttccgccaccgttctccgagcaggtctctgtcggtccccaaccggcctcggccacctcaacccccgcagagacccccccctccaagtaagactctgcttgctttcagctccctggatgcctgccaggaataaccgcgcgttccctgctactgaaaacacggctccgaatccacttctccccggccttccggtccccacagggcttcctcctccttgggctccgttttgttgtaaatccaggacacgtttgacgtcagggctggttgtgtttctcatggcaaatgaaacagctcctttccaaagctgctgttcaccctggagctgtgctattgcttcaaactgctctcccttggcagcattagaggatgcagaatgggggccgcagccatgggagggacgtgagcttcgcagtcagaagtctctcctcagctgctgcgcagtcctccctctacttatgaggaatctggtaaacccaggtccccgcctggaggtgacctggtcacccctctgcgctccgaggcctcctgttccccgggctcagaacaagttcccccttatcctgggtctaggagcattttcttctggttgttcgaacaatgctttgcccgtccagccctccctgataacctgaaagcaaagctgtgatgtgcttttgatccacttgcattttcccaccactgtttacttctccctccccaaaaatgtgatgatcagacttagatcttcctaagccattacttcagaattgggctcattgtggctgacgtcacggtagtaaccaatggcaagccacaagcccagatataaaaataacccaaaaataaaatgcataagtcatgtattattgtggaaattttagagataactacaaagaagaacaaaattttaaaattcacttgatgacctacgggtcgtagttgttgaaaaaagtcttctttttccagtttcatgtatctgcagcaacatacttctttttttttttttttttttttaacaaaattaatacccattttctctggtcggttgtcttttatttaatgggttatattttctgtgtcattgaatgttctaaaacatgatttgttaatggctgcaagtatattttaacagtttattcccctgctttggaattttttttttttttttttttttttttttttttttttttttttttgagatgggctcattctgtcccccgggctggagtgcagtggtgcgatctcacagctcactgaagccttgacctccccggctcaagcaatcctcctacctcagcctgccaagtagctaggactgcaggcgcataccatcaggcttggctatttttattcatttatttatttattttgtagagacagtttcaccatgttgcccaggctggggtttttgaaatattttttccccacaaaatattttaaaaatctggtcctagcaatagtatttggaaccatttcaaaatcaattggtttttatatcagatctgaatcccccaagatgttaaagtttgcattctagtaatgcttctccagatcatctacatcaataggcacacactggtagaatcaagtgagaaactccatagagagtcaaggaaggcctggctctgtgggtgccctaagtgccgagtgagtattcggacctcaccgtgtctgtggtctgctgggacatccgcctcgtctagtcgggctcctggcagctggctgggaagttgtgccttcactgagattgagactctgccctcgttggtttttacagccggtttaatggtgaaaaagtcggcttcagatgcgtccatctcctccggcacccatggacagtattcaattttgcagacggcaagacttctaccaggagcacctcagcaacctgtgagttcttctgcatacatttcaatcccaagggttttgcccccgggatagtgtgggatctctcttctcagtgaatatatagatttatgtgtctttaaccctctacctgctcaccatctcatctccaaaccccataacccttcctcgcaggttctccatagggcttttaaatggttgtatggagtgttctagaaaaaatcacttttgatgaaaaatcccaaatatccctggtttccatactcatccatttctctctcttcccattcttgcctctgaccttggtgtatgggcagagcattgcttggtagaagggaaaatgggagaaattcaggtcgttatcctgggaaaatgatgatcattctggagtgaagttggttgagagaagaggctaaaaagagatgtaaataatgaagttggggaacacactaggaagactaacagccatagaggattgtggaggtgcctggttccagagggtaccatgagctgctggggaagagagcaggtgaaggcagggaagtagaggggagagaggggaagctgggtgctagggggaaactgctatgaccgggatgcctctggggtgctgggcctggttaagtgcttgtgcggccccagcaggtggaggggtgactggaggccagtgttttacagtagtgaaaataggtcataaaggcaacccacatgctggatgaagaagtagaaaaagcacttttaaggaaggtattttaacaactcagtgggaccatggaaagagtggcctcttattttgctttctggctttcacctggttattgaaaaaaagatagaagatggtctgtccatatgcaaaaatctgtaatcacccggctttgaagaccagggtcatcaggtctaagtagtatggcactcttggaaagagagtgctccaggaaagagaggattgcagggtgtggtggaggctggagtttgtatggtcagcatcgtctggtggagccctgcacagacccacgggtagcagatacgtcccacgaggctgtcctcctgcctctgctgatactctgctcttcctcattctgtcctcaagcccaaggctcggactggaataagtaaaccttataatgtcaagcagatcaaaaccaccaatgcccaggaggcagaagcagcaatccggtgtctcctggaagccagaggaggtaggtgctttcctgggggcaggggaaaaaccaattctccttttctttttatctccctgctaaaagtggtctagaaacgaaaataactctttttattcttctggaggcaatattaaatgaaaacatcctttagcaacattttatataaatctatactaagattgattgtccatttaaaattgtattttcaaaattctgtgtgtatataaaaatcatcccagggctattaagcggcgtctaaaggagagaccgaaaaaaaccaaaatgagtttttaaaaaaggtgaatgctgccggcatgaagtcattctgccagctttataaaacagagacagttatagtgtttgtcctaatttcaggcacaaaatctaccttttaaatcactggcaagtggaaataaagtttttagtgtcaggcagggacctctttcccaccacattattttttgttgctgttacttaaaccatgttagttaccttgggctctcccccacttcattcataatcactggtaatttacacttttagttttggccttggctttgttccagaatctcctagagaggagagtgtgcccttttcttcgtttttttttttgttttttttttttttttttttgagactgagtttcactctgtcagctggactggagtgcagtggctcaatcccaggtcactgcaacctccgcctcccaggttcgagcaaatatcctgcctcagccacctgagtagctgggactacaggcgcgcagccccatgatgagccaatttttgtattttcagtagaaatgtgatttcaccttgttggccaggctggtcttgaactcccaacctcaggtgattcatccaccttggcctcctaaagtgctgggattacaggtgtgagccaccacacctggccgagtgtgccctttaaaaaggtgtaagcaatcaccattgcttaatttgcttttagtcagcatgtttaacttctttggaattctcaaataatatttaagggggcatgagttaaaaggtcgagaactactgttgtagaatgatactgctacaaatgctttctttttttttattttagaaaaataactaaaactaggccgggcgcggtggctcacgcctgtaatcccagcactttgggaggccgaggcgggtggatcacgaggacaggaaatcgagaccatcctggctaacacggtgaaaccccgtctctactaaaaattcaaaaaattagccgggcgtgatggtgggtgactgtagtcccagctactcaggaggctgaggcaggagaatggctgagcccgggaggtggagctcgcagtgagccaagattgcaccactgcactccagcctgggtgacagagcaagactccgtctcaaaaaaagaaaaaagaaagaaagaaaaataactaaaactatggatataacaacccaagtgtccatcagctgataaatggataaacaaaatgtggtccatcaatacaatggaatattgttcagccattaaaaaaaaaaagaatggggctgggtgctgggtgcagtggctcacgcctgtaatcccagcgctttgggaggccgaggcaggcggatcacgaggttaggagatcgagaccatcctggctaacacagtgaaactccatctctactaaaaatacaaaaaaattagccgggcgtggtggcacgtgcctgttagtcccagttactcgggaggctgaggcagaagaatcatttgaactggggaggcagaggttgcagtgagccaagataacgccactgcactccagcctgggtgacagagctagactctgtctcataaaaaaaaaaaaaaaaaagaatgaagtgctgatacatactacttcatgggtggaccttgaagacattatactatgtgaaagaaagaaaaaaaggccacatagtgtaccatcccctttttatgaaatggccacagcaggcaaattcatggggtcagaaagttcttcttgctttccagggactggaaggaggcctgggtggggagtggctgctgtggggcttcttttttgtgtgatgaaaatgttctggagttagatcgtatgatggttgcacaactttgtggatacactaaaaaccaatgaatggtacacttagaaagggtgaatttcacggtatgtgaattatgtcacaataaagctgttaaaaaaaaaaaaactctagagatgtagatgttagtagaggtgtgaaatctacctacagtcccggcttaacactttcttggtcatggccccattgagattttgatgtctttggatcatctaccctgagaaatgtaagtacacatgagactttgcatatgatctcagggtctcactggtccccatccaaactatccacagaccccagatcaccacccctgctaggaaggtgagcatgaaaacctgggactgctgggtgcggtggctcacgcctgtaatcccagcactttgagaggctggggtgggaggactgcttgaatccaggagttcgagacaagtctgggcaacacaatgagaccctctctctacaaaaattttaaaattagctgggcgtggtggttcacaccagtagtcccagctacttgggaggctgaggtaagaagactgcatgagcccaggagttccagcaacatagtgagacctcatctctactaaaaattttaaaaattggctgggcgtggcagtgctcacccgtaatcccagctactcaggaggctgaaatgggagaattgctcgagcctgggaggttgagactgcagtgagccgtgatcatgccgctgcactccggcctgggtggcagagcgagagcccatcttaaaaaagaaaaaagaaaaaaagctgggactgaatctagctgattactcctgaaatacactgaattagtaaatgacacttaggatgtagactgtctaaggacgaaatcatgcagtgaattgccttgtcctttacacttcagccatgtggttttatgtttcaggtgcctccgaagaagccctaagtgccgtggccccaagggaccttgaagcatcctctgaaccagagcccacaccgggggcagccaaaccagagaccccacaggcgcccccactccttccccgtcggcccccacccagagttcctgccatcaagaagccaaccttgagaaggacaggaaaggtaaaagcctggtaacataaaacctcttactcctaagttgctcacatgtctcgatagataagaattgtggcctgtaatcccagcacattgggaagccgaggcaagcggattacgaggtcaggagttcaagaccagcctgaccaacatggtgtgaaaccccgtctctactaaaaatagaaaaattagccgggcgtggtggcgcacgcctgtaatcccagctactcaggaggctgaggcaggagaatcacttgaacccaggaggcagaggttgcagtgagccgaaatcgtgccactgcattccagcctgagcagcagagtgagactccacctcaaaaaaaaaaaaaacaaaaaaaaaaaaacagatttgcgtctcacagataaaaatctagttccctatggacatcctcacgttcatgagtcccagacacaggtgtgtctttggggaaagttttgacaactgcttcttccctcatatcctactgtgtcttggctctggctgagaagaacgaggagtccactgtctggcacattgtcagtgtgacaagaacagcttggaggtattatgcttcctaagtgttttgagtccccaaagggaaccatagtgggatgcttttccccacatgttctggtaccgcccatttgcgtgtggtctgtgttgtgtgcacatggtttcaaggcattaggaaaacagtaaccgattcccagattcgccatgttcacatgctctcgtgcggcctcagcctacgagaggttccacagcttttcttgccacttttcttgcagattgttttttgctctcgctctcaagcttctcagccttgcttactgcttcaaagacatgagtttgtaaggacagtagcagcccaaagactggcatctgtagacacatctggatcttccgtttgatctcagtgttctagtaacggaccctcggtaatcccttcctggtcatcacagcacgatgctgcttcccccctagagagtgtgaggggggcttcggcttcactcttgacagcgcttgctttgtatttcctgggttgctcttctgtcccacattggctcattgccatctgtcccttgtccatgcttccttacaaagtggacctttttcctccgcctcttccctgttcagatgctcctcgacttaaaatggggttacatcccaataaacccatcgtaagttgaaaaatagcgtgtcaaaactgcatctcatacacctaacctagcaaacgtcatggtctcgctgaccttgaacacactcagaacacatacattagcctacggctgggcaaaaaaaaaaaaaaaaaaaaaaaacacccatccaacacagcctattttataataataaaaataaaagatcaaacttcaaaattcaaagtatgctttctacggaacattagcgcttgtgcactagggtgaagtggaaaatcctcagtggaaccattgtaagtcggggaccacctgctttgtcttttcttgtaaaagccaggagcagaaatagattcattttcccaacctgctgcacaggagttaaagttaaaaggctgttggaaaagagactttcttttttcttagtttgttttctaaagaaccaaagaaacactgaaacgccaatgtttaactagtctactgatcaccttttctgaacaaaagcttcattttgcctgtgcagttgttttctttgtgatagatttgaaccagaaccaaggcagtgtcaaggtttccattctttaccaatatttaagtataaaagattcaaggagctgggcgcggtggctcacgcgtgtaatcccagcactttgggaggctgaggcgggtggatcacctgaggtcaggagttcaagaccagcctgaccaacatggtgaaaccccgtctctactacaaaatacaaaaattagccaggcatggtggcaggcgcctgcaatcccagctactcgggaggctgaagcaggagaatcgcttgaacctgggaggcggaggttacagtgagctgagattgcgccattgcactccagcctgggggacagggcgagactccttctcaaaaaaaaaaaaaaaattcaaggagaaagtatatccattagtccttcagttagcttatgttgtaacacaagttacttcaccctagcatgatacagaccatgaatcagcccaggttgcaacttcaaagagtgttttcagagaggtttgctttcaataaaggaagagcatcaccccatttcctaaaggacaaagaaaagtttgtctgattatttactgtggccctggttttaaattttccactcgttttcattggacctgctggtgtgacctgataaatgtgtgtttctcctgtgctttttctttttcttacctggtcacatttctggcaccccacacattctcaaatctcgaatttgctagaatgtcagcttggtctcatctctgttctctgctggccactctgttctctgatcctcagctgcctagttattggcttcttatttacactctttgtcccaccttttctcagcccctgtcaccggaagaacagtttgagcaacagactgtccattttacaatcgggcccccggagacaagcgttgaggcccctcctgtcgtgacagcccctcgagtccctcctgttcccaaaccaagaacatttcagcctgggaaagctgcagagaggccaagccacaggaagccagcatcagacgaagcccctcctggggcaggagcctctgtgccaccacctctggaggcgccgcctcttgtgcccaaggtacccccgaggaggaagaagtcagcccccgcagccttccacctgcaggtcctgcagagcaacagccagcttctccagggcctcacttacaatagcagtgacagcccctctgggcacccacctgccgcgggcaccgtcttcccacaaggggactttctcagcacttcatctgctacaagccccgacagcgatggcaccaaagcgatgaagccagaggcagccccacttcttggtgattatcaggaccccttctggaaccttcttcaccaccctaaactgttgaataacacttggctttctaagagctcagaccctttggactcaggaaccaggagccccaaaagagatcccatagacccagtgtcagctggcgcttcagctgccaaggcagagctgccaccagatcatgaacacaaaaccttaggtcactgggtgacaatcagtgaccaagaaaagaggacagcactgcaggtgtttgacccactggcaaaaacatgactgagcagctttgaaggctgcagtcctatagaatgcataccttcctccctctagacatccctccaccagaagagacatctatttaaaggcacactggccaaaacgtttgtgcatctgtcactctcgtgtagtttacaaaaatcgtgtctcttattcagtaagatggttactcagccaccaaaatatatttcactcaaggcttgtacatctgaagtttgctcttcaaggaatgggaaccttcctgttaaattcggtgtatggattttaagaaaggaatctagccaatgaggtccaagaagttctcacccattgaatttttaaatggctgttcagttcatgttgtacgtgatggagatttgtcttttgttttatttgcattttacagatttggtataacattttggggagccacctgaaggttgatgtataaagtaaggattagagaaagaggtcgttgtgaccattagtagctgtcctggcccacttaaacaaggttacaaaaaatcagagtcggaagcagccaaataggtcaacctaatgactagactgtacattcccatgagccttcatgtttaagtgtgtacatgtgcgttaaccttgatgatgcgtgaatcccgagggagccggtggcatacaccgttagcttaaccttagcttaaactagctgaaggctcctgtgccatgtcttagacattgcatgccctatcaattactataatcctgagccatggtgtgctactgaaaccaatttttatccaccatctagtccttattaaatgaaacctcacggatcctttgttccgcttatattccatgcataccacataaaagcacacagtgcgaaaactcttgctgatacgcgatattgattctcattgttagaatatggagagtgtttcagcctcgtctgtccggctggagcttcgggatggaaagtgctatgtgtccctgcatataagaatcaccaggccagtgtttctgggtttgcttgtctatatgtttgtctatattttttgcctatacatttttcccacgtttccaacagcacttctcacctattcaataactgaaaaagacattaccatagtgctttacatttttaaagtaatgttacaaggtctggaatccatttggagcagataccgtgttttcgctatttaataagaagttcagtagtgaaatcttactgtaccgcctgttgtatctgggagcctcgtacagaggctcgcacagcagtgatcaagtgtcatcccttacgtgactgggggatgtctgtcctaaaagctgactgctaggatagtaaggatcatcttgcctgggctatgccactgtcttgttaccaattagacatctggaatttcataattagttttcattgtcactgtcaagatatattgcagattacttaaatatggccatcaaaacaaaagttacaacacgtatctcttttcatctgaaaactaatacctggaaaaggataaaaaaaaaaaggaatccgtgacccacagagctagacagataagatgcatagttgaccagtcataaaaggcggtgtttaggtgatcaggatgccgttggtggcatttacgtgctttatatgatttttacctctgtaacaaacacaagaaataaacagaatggtccttaacagagtttgggggagagagcaagatgggttccttggagaagctgatttgccaagatgcacatcgctattaacagccagagtcataaatgaaatgaaattgaagaattcattcaaatgctcttttccctataacctcttttctcaccaaaaaggagataaatttgaaaacagataaatgtaacaaccagtcaaagaagcaggggaaaagtaagctcctccaaagttgcttgcagtgctggaaatagatctcatttttaggttttctcttcgttccagataccaaataaatgggacagagaataaaatttttgttaaaatatgtgctcatctcctaagtagctcttcagagtctgaccgtaagtaaaaacacacagaattgtgttgactgggggaggtgaatcacaaaaaagttacgaggagtttaagagttaaatattatttgatcgtggctgtcaaatttagtgaacaacatagattggatttggagttggtagtaggtatggttctcataccagaattctcttaaaaaaaaaaaaaaggacaattggaattgccttatttatttttaaaatcaatgcttactagttggtaggattcccaggtcagcagcagggttgattaaataatcttgacaatgagcagctgccatcttgggggatttcattctgtggttttttaaatgtttcgtctttgatgctaccatccagggcttcttattgtgaccttgtagcctattttgttcctgctgttctctaacatggtgcagttcacgcagactggtttaggtacttcacaactcaccattgtctctctgaccccaagcctagtcccttttacatcaccatcttctcagacttcttgcctattcttaaaatatgttttggttaatgattgaattaggacatcagcttaagcaattcctgtaacggtttaacgttgttgaagttcaaggcacataataaaattctccctgatgtgtgtgtaagtataaacaaagtaatgtatcaaagcgttcactttataatgaagtcatttcattgggaaggaaagctgcaaagattattgggggactagtgattaataaaatcctgtaatatttttgaagtgaaaatttgtactgaaattgtacatgatacctattaaatgtttttgtctttttttaaa

SEQ ID NO: 4 - DNA, 6746 bp, homo sapien
ggcacgaggcggggagcugucgcgggcagcgcgcccucgggaggacguggccccggcccccgcccgcagugggcccgacccucauggcccugagcaaagggcugcggcugcuggggcgccugggggccgagggggacuguagcgugcugcuggaggcgcgcggccgcgacgacugccugcuguucgaggccggcacgguggccacgcuggcuccagaagaaaaggaagucauuaaaggacaguauggcaagcucacggacgcguacggcugccugggggagcugaggcugaaaucugguggcacgucucugagcuuccugguguuggugacaggcugcacaucugugggcagaauuccagaugcugaaaucuacaaaaucacugccacugacuuuuacccucuucaggaagaggccaaggaggaggaacgccucauagcuuugaagaaaauccucagcucggggguguucuauuucucauggccaaacgaugggucucgcuuugaccugacuguccgcacgcagaagcagggggaugacagcucugaaugggggaacuccuucuucuggaaccagcuguugcacgugcccuugaggcagcaccaggugagcugcugugacuggcugcugaagaucaucugcgggguggucaccauccgcaccguguaugccucccacaagcaggccaaggccugccucgucucucgcguuagcugugagcgcacaggcacucgcuuccacacccguggcgugaacgacgacggccauguguccaacuucguggagacagagcagaugauuuacauggacgauggagugucaucuuuuguccagaucagaggcuccguuccgcuguucugggaacagccagggcuucagguuggcucccaucaucugagacuccacagaggccuggaagccaaugccccugcuuucgacaggcacauggugcuucugaaggagcaguacgggcagcagguggucgugaaccuucugggaagcagaggcggagaggaggugcucaacagagccuucaagaagcugcucugggcuucuugccacgcgggcgacacgccuaugaucaauuuugacuuccaucaguuugccaaaggugggaagcuagagaaauuggagacccucuugaggccacaguuaaagcugcacugggaagacuucgauguguucacaaagggggagaacgucaguccacguuuucagaaaggcacuuugcggaugaacugucuugacugccuggaccgaaccaacacugugcagagcuucaucgcgcucgagguccugcaucugcagcucaagacccuggggcugaguucaaaacccaucguugaccgcuuuguggaguccuucaaagccauguggucucugaauggccacagccugagcaagguguucacaggcagcagagcccuggaagggaaggccaagguggggaagcugaaggauggagcccgguccaugucucgaaccauccaguccaacuucuucgacggggugaagcaggaggccaucaagcugcugcugguuggggacgucuacggcgaggagguggcagacaaagggggcaugcugcuggacagcacggcgcuccuggugacucccaggauccugaaagcuaugacugagcgucaguccgaauucacaaauuucaagcggauccggauugcuauggggaccuggaacgugaacggaggaaagcaguuccggagcaacgugcucaggacggcggagcugacagacuggcugcucgacucgccccagcucucgggagcuaccgacucccaggaugacagcagcccagcugacauauuugcugugggguuugaagagaugguggaauugagcgcagggaauauugucaaugccaguacuaccaacaagaagauguggggugaacagcuucagaaagccaucucacgcucucauagauacauucuguugacuucggcacagcuggugggcgucugucuuuauaucuuuguacguccauaccaugucccguucaucagggacguagccaucgacacagugaagacgggcauggggggcaaggcggggaacaagggcgccgucggcauccgcuuccaguuccacagcaccagcuucugcuucauauguagucaccugacggccgggcagucccaggugaaggagcggaaugaagacuacaaggagaucacccagaaacucugcuucccaauggggagaaauguuuuuucucaugauuauguauuuugguguggcgauuucaacuaccgcauugaucuuacuuaugaagaagucuucuauuuuguuaaacgccaagacuggaagaaacuucuggaauuugaucaacuacagcuacagaaaucaaguggaaaaauuuuuaaggacuuucacgaaggagccauuaacuuuggacccaccuacaaguaugacguuggcucagccgccuacgauacaagcgacaaaugccgcacccccgccuggacagacagggugcugugguggaggaagaaacaucccuuugauaaaacagcuggagaacucaaccuucuagacagugaucuagauguugacaccaaagucagacacaccuggucuccuggugcccugcaguauuauggucgugcggagcuacaagcgucugaucacagaccugugcuggcgaucguggagguggaaguucaggaagucgaugugggugcucgggagaggguuuuccaggaaguguccuccuuccagggcccccuggaugccacuguuguaguaaaccuucaaucaccgaccuuagaagagaaaaacgaguuuccagaggaccugcguacugagcucaugcagaccuuggggaguuaugggacaauuguucuugucaggaucaaccaagggcagaugcugguaacuuuugcagacagucacucggcucucaguguccuggacguggacgguaugaaggugaaaggcagagcagugaagauuagaccgaagaccaaggacuggcugaaagguuugcgagaggagaucauucggaaacgagacagcauggcccccgugucucccacugccaacuccuguuugcuggaggaaaacuuugacuucacaaguuuggacuaugagucagaaggggauauucuugaagacgaugaagacuacuugguggaugaauucaaucagccuggagucucggacagugaacucgggggagacgaccucucugauguccccggccccacagcacuggcuccucccagcaagucaccugcucucaccaaaaagaagcagcauccaacguacaaagaugacgcggaccugguggagcucaagcgggagcuggaagccgucggggaguuccgccaccguucuccgagcaggucucugucgguccccaaccggccucggccaccucaacccccgcagagaccccccccuccaaccgguuuaauggugaaaaagucggcuucagaugcguccaucuccuccggcacccauggacaguauucaauuuugcagacggcaagacuucuaccaggagcaccucagcaaccucccaaggcucggacuggaauaaguaaaccuuauaaugucaagcagaucaaaaccaccaaugcccaggaggcagaagcagcaauccggugucuccuggaagccagaggaggugccuccgaagaagcccuaagugccguggccccaagggaccuugaagcauccucugaaccagagcccacaccgggggcagccaaaccagagaccccacaggcgcccccacuccuuccccgucggcccccacccagaguuccugccaucaagaagccaaccuugagaaggacaggaaagccccugucaccggaagaacaguuugagcaacagacuguccauuuuacaaucgggcccccggagacaagcguugaggccccuccugucgugacagccccucgagucccuccuguucccaaaccaagaacauuucagccugggaaagcugcagagaggccaagccacaggaagccagcaucagacgaagccccuccuggggcaggagccucugugccaccaccucuggaggcgccgccucuugugcccaagguacccccgaggaggaagaagucagcccccgcagccuuccaccugcagguccugcagagcaacagccagcuucuccagggccucacuuacaauagcagugacagccccucugggcacccaccugccgcgggcaccgucuucccacaaggggacuuucucagcacuucaucugcuacaagccccgacagcgauggcaccaaagcgaugaagccagaggcagccccacuucuuggugauuaucaggaccccuucuggaaccuucuucaccacccuaaacuguugaauaacacuuggcuuucuaagagcucagacccuuuggacucaggaaccaggagccccaaaagagaucccauagacccagugucagcuggcgcuucagcugccaaggcagagcugccaccagaucaugaacacaaaaccuuaggucacugggugacaaucagugaccaagaaaagaggacagcacugcagguguuugacccacuggcaaaaacaugacugagcagcuuugaaggcugcaguccuauagaaugcauaccuuccucccucuagacaucccuccaccagaagagacaucuauuuaaaggcacacuggccaaaacguuugugcaucugucacucucguguaguuuacaaaaaucgugucucuuauucaguaagaugguuacucagccaccaaaauauauuucacucaaggcuuguacaucugaaguuugcucuucaaggaaugggaaccuuccuguuaaauucgguguauggauuuuaagaaaggaaucuagccaaugagguccaagaaguucucacccauugaauuuuuaaauggcuguucaguucauguuguacgugauggagauuugucuuuuguuuuauuugcauuuuacagauuugguauaacauuuuggggagccaccugaagguugauguauaaaguaaggauuagagaaagaggucguugugaccauuaauagcuguccuggcccacuuaaacaagguuacaaaaaaucagagucggaagcagccaaauaggucaaccuaaugacuagacuguacauucccaugagccuucauguuuaaguguguacaugugcguuaaccuugaugaugcgugaaucccgagggagccgguggcauacaccguuagcuuaaccuuagcuuaaacuagcugaaggcuccugugccaugucuuagacauugcaugcccuaucaauuacuauaauccugagccauggugugcuacugaaaccaauuuuuauccaccaucuaguccuuauuaaaugaaaccucacggauccuuuguuccgcuuauauuccaugcauaccacauaaaagcacacagugcgaaaacucuugcugauacgcgauauugauucucauuguuagaauauggagaguguuucagccucgucuguccggcuggagcuucgggauggaaagugcuaugugucccugcauauaagaaucaccaggccaguguuucuggguuugcuugucuauauguuugucuauauuuuuugccuauacauuuuucccacguuuccaacagcacuucucaccuauucaauaacugaaaaagacauuaccauagugcuuuacauuuuuaaaguaauguuacaaggucuggaauccauuuggagcagauaccguguuuucgcuauuuaauaagaaguucaguagugaaaucuuacuguaccgccuguuguaucugggagccucguacagaggcucgcacagcagugaucaagugucaucccuuacgugacugggggaugucuguccuaaaagcugacugcuaggauaguaaggaucaucuugccugggcuaugccacugucuuguuaccaauuagacaucuggaauuucauaauuaguuuucauugucacugucaagauauauugcagauuacuuaaauauggccaucaaaacaaaaguuacaacacguaucucuuuucaucugaaaacuaauaccuggaaaaggauaaaaaaaaaaaggaauccgugacccacagagcuagacagauaagaugcauaguugaccagucauaaaaggcgguguuuaagugaucaggaugccguugguggcauuuacgugcuuuauaugauuuuuaccucuguaacaaacacaagaaauaaacagaaugguccuuaacagaguuugggggagagagcaagauggguuccuuggagaagcugauuugccaagaugcacaucgcuauuaacagccagagucauaaaugaaaugaaauugaagaauucauucaaaugcucuuuucccuauaaccucuuuucucaccaaaaaggagauaaauuugaaaacagauaaauguaacaaccagucaaagaagcaggggaaaaguaagcuccuccaaaguugcuugcagugcuggaaauagaucucauuuuuagguuuucucuucguuccagauaccaaauaaaugggacagagaauaaaauuuuuguuaaaauaugugcucaucuccuaaguagcucuucagagucugaccguaaguaaaaacacacagaauuguguugacugggggaggugaaucacaaaaaaguuacgaggaguuuaagaguuaaauauuauuugaucguggcugucaaauuuagugaacaacauagauuggauuuggaguugguaguaaguaugguucucauaccagaauucucuu

SEQ ID NO: 5 - DNA, 6746 bp, homo sapien
ggcacgaggcggggagcugucgcgggcagcgcgcccucgggaggacguggccccggcccccgcccgcagugggcccgacccucauggcccugagcaaagggcugcggcugcuggggcgccugggggccgagggggacuguagcgugcugcuggaggcgcgcggccgcgacgacugccugcuguucgaggccggcacgguggccacgcuggcuccagaagaaaaggaagucauuaaaggacaguauggcaagcucacggacgcguacggcugccugggggagcugaggcugaaaucugguggcacgucucugagcuuccugguguuggugacaggcugcacaucugugggcagaauuccagaugcugaaaucuacaaaaucacugccacugacuuuuacccucuucaggaagaggccaaggaggaggaacgccucauagcuuugaagaaaauccucagcucggggguguucuauuucucauggccaaacgaugggucucgcuuugaccugacuguccgcacgcagaagcagggggaugacagcucugaaugggggaacuccuucuucuggaaccagcuguugcacgugcccuugaggcagcaccaggugagcugcugugacuggcugcugaagaucaucugcgggguggucaccauccgcaccguguaugccucccacaagcaggccaaggccugccucgucucucgcguuagcugugagcgcacaggcacucgcuuccacacccguggcgugaacgacgacggccauguguccaacuucguggagacagagcagaugauuuacauggacgauggagugucaucuuuuguccagaucagaggcuccguuccgcuguucugggaacagccagggcuucagguuggcucccaucaucugagacuccacagaggccuggaagccaaugccccugcuuucgacaggcacauggugcuucugaaggagcaguacgggcagcagguggucgugaaccuucugggaagcagaggcggagaggaggugcucaacagagccuucaagaagcugcucugggcuucuugccacgcgggcgacacgccuaugaucaauuuugacuuccaucaguuugccaaaggugggaagcuagagaaauuggagacccucuugaggccacaguuaaagcugcacugggaagacuucgauguguucacaaagggggagaacgucaguccacguuuucagaaaggcacuuugcggaugaacugucuugacugccuggaccgaaccaacacugugcagagcuucaucgcgcucgagguccugcaucugcagcucaagacccuggggcugaguucaaaacccaucguugaccgcuuuguggaguccuucaaagccauguggucucugaauggccacagccugagcaagguguucacaggcagcagagcccuggaagggaaggccaagguggggaagcugaaggauggagcccgguccaugucucgaaccauccaguccaacuucuucgacggggugaagcaggaggccaucaagcugcugcugguuggggacgucuacggcgaggagguggcagacaaagggggcaugcugcuggacagcacggcgcuccuggugacucccaggauccugaaagcuaugacugagcgucaguccgaauucacaaauuucaagcggauccggauugcuauggggaccuggaacgugaacggaggaaagcaguuccggagcaacgugcucaggacggcggagcugacagacuggcugcucgacucgccccagcucucgggagcuaccgacucccaggaugacagcagcccagcugacauauuugcugugggguuugaagagaugguggaauugagcgcagggaauauugucaaugccaguacuaccaacaagaagauguggggugaacagcuucagaaagccaucucacgcucucauagauacauucuguugacuucggcacagcuggugggcgucugucuuuauaucuuuguacguccauaccaugucccguucaucagggacguagccaucgacacagugaagaugggcauggggggcaaggcggggaacaagggcgccgucggcauccgcuuccaguuccacagcaccagcuucugcuucauauguagucaccugacggccgggcagucccaggugaaggagcggaaugaagacuacaaggagaucacccagaaacucugcuucccaauggggagaaauguuuuuucucaugauuauguauuuugguguggcgauuucaacuaccgcauugaucuuacuuaugaagaagucuucuauuuuguuaaacgccaagacuggaagaaacuucuggaauuugaucaacuacagcuacagaaaucaaguggaaaaauuuuuaaggacuuucacgaaggagccauuaacuuuggacccaccuacaaguaugacguuggcucagccgccuacgauacaagcgacaaaugccgcacccccgccuggacagacagggugcugugguggaggaagaaacaucccuuugauaaaacagcuggagaacucaaccuucuagacagugaucuagauguugacaccaaagucagacacaccuggucuccuggugcccugcaguauuauggucgugcggagcuacaagcgucugaucacagaccugugcuggcgaucguggagguggaaguucaggaagucgaugugggugcucgggagaggguuuuccaggaaguguccuccuuccagggcccccuggaugccacuguuguaguaaaccuucaaucaccgaccuuagaagagaaaaacgaguuuccagaggaccugcguacugagcucaugcagaccuuggggaguuaugggacaauuguucuugucaggaucaaccaagggcagaugcugguaacuuuugcagacagucacucggcucucaguguccuggacguggacgguaugaaggugaaaggcagagcagugaagauuagaccgaagaccaaggacuggcugaaagguuugcgagaggagaucauucggaaacgagacagcauggcccccgugucucccacugccaacuccuguuugcuggaggaaaacuuugacuucacaaguuuggacuaugagucagaaggggauauucuugaagacgaugaagacuacuugguggaugaauucaaucagccuggagucucggacagugaacucgggggagacgaccucucugauguccccggccccacagcacuggcuccucccagcaagucaccugcucucaccaaaaagaagcagcauccaacguacaaagaugacgcggaccugguggagcucaagcgggagcuggaagccgucggggaguuccgccaccguucuccgagcaggucucugucgguccccaaccggccucggccaccucaacccccgcagagaccccccccuccaaccgguuuaauggugaaaaagucggcuucagaugcguccaucuccuccggcacccauggacaguauucaauuuugcagacggcaagacuucuaccaggagcaccucagcaaccucccaaggcucggacuggaauaaguaaaccuuauaaugucaagcagaucaaaaccaccaaugcccaggaggcagaagcagcaauccggugucuccuggaagccagaggaggugccuccgaagaagcccuaagugccguggccccaagggaccuugaagcauccucugaaccagagcccacaccgggggcagccaaaccagagaccccacaggcgcccccacuccuuccccgucggcccccacccagaguuccugccaucaagaagccaaccuugagaaggacaggaaagccccugucaccggaagaacaguuugagcaacagacuguccauuuuacaaucgggcccccggagacaagcguugaggccccuccugucgugacagccccucgagucccuccuguucccaaaccaagaacauuucagccugggaaagcugcagagaggccaagccacaggaagccagcaucagacgaagccccuccuggggcaggagccucugugccaccaccucuggaggcgccgccucuugugcccaagguacccccgaggaggaagaagucagcccccgcagccuuccaccugcagguccugcagagcaacagccagcuucuccagggccucacuuacaauagcagugacagccccucugggcacccaccugccgcgggcaccgucuucccacaaggggacuuucucagcacuucaucugcuacaagccccgacagcgauggcaccaaagcgaugaagccagaggcagccccacuucuuggugauuaucaggaccccuucuggaaccuucuucaccacccuaaacuguugaauaacacuuggcuuucuaagagcucagacccuuuggacucaggaaccaggagccccaaaagagaucccauagacccagugucagcuggcgcuucagcugccaaggcagagcugccaccagaucaugaacacaaaaccuuaggucacugggugacaaucagugaccaagaaaagaggacagcacugcagguguuugacccacuggcaaaaacaugacugagcagcuuugaaggcugcaguccuauagaaugcauaccuuccucccucuagacaucccuccaccagaagagacaucuauuuaaaggcacacuggccaaaacguuugugcaucugucacucucguguaguuuacaaaaaucgugucucuuauucaguaagaugguuacucagccaccaaaauauauuucacucaaggcuuguacaucugaaguuugcucuucaaggaaugggaaccuuccuguuaaauucgguguauggauuuuaagaaaggaaucuagccaaugagguccaagaaguucucacccauugaauuuuuaaauggcuguucaguucauguuguacgugauggagauuugucuuuuguuuuauuugcauuuuacagauuugguauaacauuuuggggagccaccugaagguugauguauaaaguaaggauuagagaaagaggucguugugaccauuaauagcuguccuggcccacuuaaacaagguuacaaaaaaucagagucggaagcagccaaauaggucaaccuaaugacuagacuguacauucccaugagccuucauguuuaaguguguacaugugcguuaaccuugaugaugcgugaaucccgagggagccgguggcauacaccguuagcuuaaccuuagcuuaaacuagcugaaggcuccugugccaugucuuagacauugcaugcccuaucaauuacuauaauccugagccauggugugcuacugaaaccaauuuuuauccaccaucuaguccuuauuaaaugaaaccucacggauccuuuguuccgcuuauauuccaugcauaccacauaaaagcacacagugcgaaaacucuugcugauacgcgauauugauucucauuguuagaauauggagaguguuucagccucgucuguccggcuggagcuucgggauggaaagugcuaugugucccugcauauaagaaucaccaggccaguguuucuggguuugcuugucuauauguuugucuauauuuuuugccuauacauuuuucccacguuuccaacagcacuucucaccuauucaauaacugaaaaagacauuaccauagugcuuuacauuuuuaaaguaauguuacaaggucuggaauccauuuggagcagauaccguguuuucgcuauuuaauaagaaguucaguagugaaaucuuacuguaccgccuguuguaucugggagccucguacagaggcucgcacagcagugaucaagugucaucccuuacgugacugggggaugucuguccuaaaagcugacugcuaggauaguaaggaucaucuugccugggcuaugccacugucuuguuaccaauuagacaucuggaauuucauaauuaguuuucauugucacugucaagauauauugcagauuacuuaaauauggccaucaaaacaaaaguuacaacacguaucucuuuucaucugaaaacuaauaccuggaaaaggauaaaaaaaaaaaggaauccgugacccacagagcuagacagauaagaugcauaguugaccagucauaaaaggcgguguuuaagugaucaggaugccguugguggcauuuacgugcuuuauaugauuuuuaccucuguaacaaacacaagaaauaaacagaaugguccuuaacagaguuugggggagagagcaagauggguuccuuggagaagcugauuugccaagaugcacaucgcuauuaacagccagagucauaaaugaaaugaaauugaagaauucauucaaaugcucuuuucccuauaaccucuuuucucaccaaaaaggagauaaauuugaaaacagauaaauguaacaaccagucaaagaagcaggggaaaaguaagcuccuccaaaguugcuugcagugcuggaaauagaucucauuuuuagguuuucucuucguuccagauaccaaauaaaugggacagagaauaaaauuuuuguuaaaauaugugcucaucuccuaaguagcucuucagagucugaccguaaguaaaaacacacagaauuguguugacugggggaggugaaucacaaaaaaguuacgaggaguuuaagaguuaaauauuauuugaucguggcugucaaauuuagugaacaacauagauuggauuuggaguugguaguaaguaugguucucauaccagaauucucuu

SEQ ID NO: 6 - DNA, 6746 bp, homo sapien
ggcacgaggcggggagctgtcgcgggcagcgcgccctcgggaggacgtggccccggcccccgcccgcagtgggcccgaccctcatggccctgagcaaagggctgcggctgctggggcgcctgggggccgagggggactgtagcgtgctgctggaggcgcgcggccgcgacgactgcctgctgttcgaggccggcacggtggccacgctggctccagaagaaaaggaagtcattaaaggacagtatggcaagctcacggacgcgtacggctgcctgggggagctgaggctgaaatctggtggcacgtctctgagcttcctggtgttggtgacaggctgcacatctgtgggcagaattccagatgctgaaatctacaaaatcactgccactgacttttaccctcttcaggaagaggccaaggaggaggaacgcctcatagctttgaagaaaatcctcagctcgggggtgttctatttctcatggccaaacgatgggtctcgctttgacctgactgtccgcacgcagaagcagggggatgacagctctgaatgggggaactccttcttctggaaccagctgttgcacgtgcccttgaggcagcaccaggtgagctgctgtgactggctgctgaagatcatctgcggggtggtcaccatccgcaccgtgtatgcctcccacaagcaggccaaggcctgcctcgtctctcgcgttagctgtgagcgcacaggcactcgcttccacacccgtggcgtgaacgacgacggccatgtgtccaacttcgtggagacagagcagatgatttacatggacgatggagtgtcatcttttgtccagatcagaggctccgttccgctgttctgggaacagccagggcttcaggttggctcccatcatctgagactccacagaggcctggaagccaatgcccctgctttcgacaggcacatggtgcttctgaaggagcagtacgggcagcaggtggtcgtgaaccttctgggaagcagaggcggagaggaggtgctcaacagagccttcaagaagctgctctgggcttcttgccacgcgggcgacacgcctatgatcaattttgacttccatcagtttgccaaaggtgggaagctagagaaattggagaccctcttgaggccacagttaaagctgcactgggaagacttcgatgtgttcacaaagggggagaacgtcagtccacgttttcagaaaggcactttgcggatgaactgtcttgactgcctggaccgaaccaacactgtgcagagcttcatcgcgctcgaggtcctgcatctgcagctcaagaccctggggctgagttcaaaacccatcgttgaccgctttgtggagtccttcaaagccatgtggtctctgaatggccacagcctgagcaaggtgttcacaggcagcagagccctggaagggaaggccaaggtggggaagctgaaggatggagcccggtccatgtctcgaaccatccagtccaacttcttcgacggggtgaagcaggaggccatcaagctgctgctggttggggacgtctacggcgaggaggtggcagacaaagggggcatgctgctggacagcacggcgctcctggtgactcccaggatcctgaaagctatgactgagcgtcagtccgaattcacaaatttcaagcggatccggattgctatggggacctggaacgtgaacggaggaaagcagttccggagcaacgtgctcaggacggcggagctgacagactggctgctcgactcgccccagctctcgggagctaccgactcccaggatgacagcagcccagctgacatatttgctgtggggtttgaagagatggtggaattgagcgcagggaatattgtcaatgccagtactaccaacaagaagatgtggggtgaacagcttcagaaagccatctcacgctctcatagatacattctgttgacttcggcacagctggtgggcgtctgtctttatatctttgtacgtccataccatgtcccgttcatcagggacgtagccatcgacacagtgaagacgggcatggggggcaaggcggggaacaagggcgccgtcggcatccgcttccagttccacagcaccagcttctgcttcatatgtagtcacctgacggccgggcagtcccaggtgaaggagcggaatgaagactacaaggagatcacccagaaactctgcttcccaatggggagaaatgttttttctcatgattatgtattttggtgtggcgatttcaactaccgcattgatcttacttatgaagaagtcttctattttgttaaacgccaagactggaagaaacttctggaatttgatcaactacagctacagaaatcaagtggaaaaatttttaaggactttcacgaaggagccattaactttggacccacctacaagtatgacgttggctcagccgcctacgatacaagcgacaaatgccgcacccccgcctggacagacagggtgctgtggtggaggaagaaacatccctttgataaaacagctggagaactcaaccttctagacagtgatctagatgttgacaccaaagtcagacacacctggtctcctggtgccctgcagtattatggtcgtgcggagctacaagcgtctgatcacagacctgtgctggcgatcgtggaggtggaagttcaggaagtcgatgtgggtgctcgggagagggttttccaggaagtgtcctccttccagggccccctggatgccactgttgtagtaaaccttcaatcaccgaccttagaagagaaaaacgagtttccagaggacctgcgtactgagctcatgcagaccttggggagttatgggacaattgttcttgtcaggatcaaccaagggcagatgctggtaacttttgcagacagtcactcggctctcagtgtcctggacgtggacggtatgaaggtgaaaggcagagcagtgaagattagaccgaagaccaaggactggctgaaaggtttgcgagaggagatcattcggaaacgagacagcatggcccccgtgtctcccactgccaactcctgtttgctggaggaaaactttgacttcacaagtttggactatgagtcagaaggggatattcttgaagacgatgaagactacttggtggatgaattcaatcagcctggagtctcggacagtgaactcgggggagacgacctctctgatgtccccggccccacagcactggctcctcccagcaagtcacctgctctcaccaaaaagaagcagcatccaacgtacaaagatgacgcggacctggtggagctcaagcgggagctggaagccgtcggggagttccgccaccgttctccgagcaggtctctgtcggtccccaaccggcctcggccacctcaacccccgcagagacccccccctccaaccggtttaatggtgaaaaagtcggcttcagatgcgtccatctcctccggcacccatggacagtattcaattttgcagacggcaagacttctaccaggagcacctcagcaacctcccaaggctcggactggaataagtaaaccttataatgtcaagcagatcaaaaccaccaatgcccaggaggcagaagcagcaatccggtgtctcctggaagccagaggaggtgcctccgaagaagccctaagtgccgtggccccaagggaccttgaagcatcctctgaaccagagcccacaccgggggcagccaaaccagagaccccacaggcgcccccactccttccccgtcggcccccacccagagttcctgccatcaagaagccaaccttgagaaggacaggaaagcccctgtcaccggaagaacagtttgagcaacagactgtccattttacaatcgggcccccggagacaagcgttgaggcccctcctgtcgtgacagcccctcgagtccctcctgttcccaaaccaagaacatttcagcctgggaaagctgcagagaggccaagccacaggaagccagcatcagacgaagcccctcctggggcaggagcctctgtgccaccacctctggaggcgccgcctcttgtgcccaaggtacccccgaggaggaagaagtcagcccccgcagccttccacctgcaggtcctgcagagcaacagccagcttctccagggcctcacttacaatagcagtgacagcccctctgggcacccacctgccgcgggcaccgtcttcccacaaggggactttctcagcacttcatctgctacaagccccgacagcgatggcaccaaagcgatgaagccagaggcagccccacttcttggtgattatcaggaccccttctggaaccttcttcaccaccctaaactgttgaataacacttggctttctaagagctcagaccctttggactcaggaaccaggagccccaaaagagatcccatagacccagtgtcagctggcgcttcagctgccaaggcagagctgccaccagatcatgaacacaaaaccttaggtcactgggtgacaatcagtgaccaagaaaagaggacagcactgcaggtgtttgacccactggcaaaaacatgactgagcagctttgaaggctgcagtcctatagaatgcataccttcctccctctagacatccctccaccagaagagacatctatttaaaggcacactggccaaaacgtttgtgcatctgtcactctcgtgtagtttacaaaaatcgtgtctcttattcagtaagatggttactcagccaccaaaatatatttcactcaaggcttgtacatctgaagtttgctcttcaaggaatgggaaccttcctgttaaattcggtgtatggattttaagaaaggaatctagccaatgaggtccaagaagttctcacccattgaatttttaaatggctgttcagttcatgttgtacgtgatggagatttgtcttttgttttatttgcattttacagatttggtataacattttggggagccacctgaaggttgatgtataaagtaaggattagagaaagaggtcgttgtgaccattaatagctgtcctggcccacttaaacaaggttacaaaaaatcagagtcggaagcagccaaataggtcaacctaatgactagactgtacattcccatgagccttcatgtttaagtgtgtacatgtgcgttaaccttgatgatgcgtgaatcccgagggagccggtggcatacaccgttagcttaaccttagcttaaactagctgaaggctcctgtgccatgtcttagacattgcatgccctatcaattactataatcctgagccatggtgtgctactgaaaccaatttttatccaccatctagtccttattaaatgaaacctcacggatcctttgttccgcttatattccatgcataccacataaaagcacacagtgcgaaaactcttgctgatacgcgatattgattctcattgttagaatatggagagtgtttcagcctcgtctgtccggctggagcttcgggatggaaagtgctatgtgtccctgcatataagaatcaccaggccagtgtttctgggtttgcttgtctatatgtttgtctatattttttgcctatacatttttcccacgtttccaacagcacttctcacctattcaataactgaaaaagacattaccatagtgctttacatttttaaagtaatgttacaaggtctggaatccatttggagcagataccgtgttttcgctatttaataagaagttcagtagtgaaatcttactgtaccgcctgttgtatctgggagcctcgtacagaggctcgcacagcagtgatcaagtgtcatcccttacgtgactgggggatgtctgtcctaaaagctgactgctaggatagtaaggatcatcttgcctgggctatgccactgtcttgttaccaattagacatctggaatttcataattagttttcattgtcactgtcaagatatattgcagattacttaaatatggccatcaaaacaaaagttacaacacgtatctcttttcatctgaaaactaatacctggaaaaggataaaaaaaaaaaggaatccgtgacccacagagctagacagataagatgcatagttgaccagtcataaaaggcggtgtttaagtgatcaggatgccgttggtggcatttacgtgctttatatgatttttacctctgtaacaaacacaagaaataaacagaatggtccttaacagagtttgggggagagagcaagatgggttccttggagaagctgatttgccaagatgcacatcgctattaacagccagagtcataaatgaaatgaaattgaagaattcattcaaatgctcttttccctataacctcttttctcaccaaaaaggagataaatttgaaaacagataaatgtaacaaccagtcaaagaagcaggggaaaagtaagctcctccaaagttgcttgcagtgctggaaatagatctcatttttaggttttctcttcgttccagataccaaataaatgggacagagaataaaatttttgttaaaatatgtgctcatctcctaagtagctcttcagagtctgaccgtaagtaaaaacacacagaattgtgttgactgggggaggtgaatcacaaaaaagttacgaggagtttaagagttaaatattatttgatcgtggctgtcaaatttagtgaacaacatagattggatttggagttggtagtaagtatggttctcataccagaattctctt

SEQ ID NO: 7 - DNA, 6746 bp, homo sapien
ggcacgaggcggggagctgtcgcgggcagcgcgccctcgggaggacgtggccccggcccccgcccgcagtgggcccgaccctcatggccctgagcaaagggctgcggctgctggggcgcctgggggccgagggggactgtagcgtgctgctggaggcgcgcggccgcgacgactgcctgctgttcgaggccggcacggtggccacgctggctccagaagaaaaggaagtcattaaaggacagtatggcaagctcacggacgcgtacggctgcctgggggagctgaggctgaaatctggtggcacgtctctgagcttcctggtgttggtgacaggctgcacatctgtgggcagaattccagatgctgaaatctacaaaatcactgccactgacttttaccctcttcaggaagaggccaaggaggaggaacgcctcatagctttgaagaaaatcctcagctcgggggtgttctatttctcatggccaaacgatgggtctcgctttgacctgactgtccgcacgcagaagcagggggatgacagctctgaatgggggaactccttcttctggaaccagctgttgcacgtgcccttgaggcagcaccaggtgagctgctgtgactggctgctgaagatcatctgcggggtggtcaccatccgcaccgtgtatgcctcccacaagcaggccaaggcctgcctcgtctctcgcgttagctgtgagcgcacaggcactcgcttccacacccgtggcgtgaacgacgacggccatgtgtccaacttcgtggagacagagcagatgatttacatggacgatggagtgtcatcttttgtccagatcagaggctccgttccgctgttctgggaacagccagggcttcaggttggctcccatcatctgagactccacagaggcctggaagccaatgcccctgctttcgacaggcacatggtgcttctgaaggagcagtacgggcagcaggtggtcgtgaaccttctgggaagcagaggcggagaggaggtgctcaacagagccttcaagaagctgctctgggcttcttgccacgcgggcgacacgcctatgatcaattttgacttccatcagtttgccaaaggtgggaagctagagaaattggagaccctcttgaggccacagttaaagctgcactgggaagacttcgatgtgttcacaaagggggagaacgtcagtccacgttttcagaaaggcactttgcggatgaactgtcttgactgcctggaccgaaccaacactgtgcagagcttcatcgcgctcgaggtcctgcatctgcagctcaagaccctggggctgagttcaaaacccatcgttgaccgctttgtggagtccttcaaagccatgtggtctctgaatggccacagcctgagcaaggtgttcacaggcagcagagccctggaagggaaggccaaggtggggaagctgaaggatggagcccggtccatgtctcgaaccatccagtccaacttcttcgacggggtgaagcaggaggccatcaagctgctgctggttggggacgtctacggcgaggaggtggcagacaaagggggcatgctgctggacagcacggcgctcctggtgactcccaggatcctgaaagctatgactgagcgtcagtccgaattcacaaatttcaagcggatccggattgctatggggacctggaacgtgaacggaggaaagcagttccggagcaacgtgctcaggacggcggagctgacagactggctgctcgactcgccccagctctcgggagctaccgactcccaggatgacagcagcccagctgacatatttgctgtggggtttgaagagatggtggaattgagcgcagggaatattgtcaatgccagtactaccaacaagaagatgtggggtgaacagcttcagaaagccatctcacgctctcatagatacattctgttgacttcggcacagctggtgggcgtctgtctttatatctttgtacgtccataccatgtcccgttcatcagggacgtagccatcgacacagtgaagatgggcatggggggcaaggcggggaacaagggcgccgtcggcatccgcttccagttccacagcaccagcttctgcttcatatgtagtcacctgacggccgggcagtcccaggtgaaggagcggaatgaagactacaaggagatcacccagaaactctgcttcccaatggggagaaatgttttttctcatgattatgtattttggtgtggcgatttcaactaccgcattgatcttacttatgaagaagtcttctattttgttaaacgccaagactggaagaaacttctggaatttgatcaactacagctacagaaatcaagtggaaaaatttttaaggactttcacgaaggagccattaactttggacccacctacaagtatgacgttggctcagccgcctacgatacaagcgacaaatgccgcacccccgcctggacagacagggtgctgtggtggaggaagaaacatccctttgataaaacagctggagaactcaaccttctagacagtgatctagatgttgacaccaaagtcagacacacctggtctcctggtgccctgcagtattatggtcgtgcggagctacaagcgtctgatcacagacctgtgctggcgatcgtggaggtggaagttcaggaagtcgatgtgggtgctcgggagagggttttccaggaagtgtcctccttccagggccccctggatgccactgttgtagtaaaccttcaatcaccgaccttagaagagaaaaacgagtttccagaggacctgcgtactgagctcatgcagaccttggggagttatgggacaattgttcttgtcaggatcaaccaagggcagatgctggtaacttttgcagacagtcactcggctctcagtgtcctggacgtggacggtatgaaggtgaaaggcagagcagtgaagattagaccgaagaccaaggactggctgaaaggtttgcgagaggagatcattcggaaacgagacagcatggcccccgtgtctcccactgccaactcctgtttgctggaggaaaactttgacttcacaagtttggactatgagtcagaaggggatattcttgaagacgatgaagactacttggtggatgaattcaatcagcctggagtctcggacagtgaactcgggggagacgacctctctgatgtccccggccccacagcactggctcctcccagcaagtcacctgctctcaccaaaaagaagcagcatccaacgtacaaagatgacgcggacctggtggagctcaagcgggagctggaagccgtcggggagttccgccaccgttctccgagcaggtctctgtcggtccccaaccggcctcggccacctcaacccccgcagagacccccccctccaaccggtttaatggtgaaaaagtcggcttcagatgcgtccatctcctccggcacccatggacagtattcaattttgcagacggcaagacttctaccaggagcacctcagcaacctcccaaggctcggactggaataagtaaaccttataatgtcaagcagatcaaaaccaccaatgcccaggaggcagaagcagcaatccggtgtctcctggaagccagaggaggtgcctccgaagaagccctaagtgccgtggccccaagggaccttgaagcatcctctgaaccagagcccacaccgggggcagccaaaccagagaccccacaggcgcccccactccttccccgtcggcccccacccagagttcctgccatcaagaagccaaccttgagaaggacaggaaagcccctgtcaccggaagaacagtttgagcaacagactgtccattttacaatcgggcccccggagacaagcgttgaggcccctcctgtcgtgacagcccctcgagtccctcctgttcccaaaccaagaacatttcagcctgggaaagctgcagagaggccaagccacaggaagccagcatcagacgaagcccctcctggggcaggagcctctgtgccaccacctctggaggcgccgcctcttgtgcccaaggtacccccgaggaggaagaagtcagcccccgcagccttccacctgcaggtcctgcagagcaacagccagcttctccagggcctcacttacaatagcagtgacagcccctctgggcacccacctgccgcgggcaccgtcttcccacaaggggactttctcagcacttcatctgctacaagccccgacagcgatggcaccaaagcgatgaagccagaggcagccccacttcttggtgattatcaggaccccttctggaaccttcttcaccaccctaaactgttgaataacacttggctttctaagagctcagaccctttggactcaggaaccaggagccccaaaagagatcccatagacccagtgtcagctggcgcttcagctgccaaggcagagctgccaccagatcatgaacacaaaaccttaggtcactgggtgacaatcagtgaccaagaaaagaggacagcactgcaggtgtttgacccactggcaaaaacatgactgagcagctttgaaggctgcagtcctatagaatgcataccttcctccctctagacatccctccaccagaagagacatctatttaaaggcacactggccaaaacgtttgtgcatctgtcactctcgtgtagtttacaaaaatcgtgtctcttattcagtaagatggttactcagccaccaaaatatatttcactcaaggcttgtacatctgaagtttgctcttcaaggaatgggaaccttcctgttaaattcggtgtatggattttaagaaaggaatctagccaatgaggtccaagaagttctcacccattgaatttttaaatggctgttcagttcatgttgtacgtgatggagatttgtcttttgttttatttgcattttacagatttggtataacattttggggagccacctgaaggttgatgtataaagtaaggattagagaaagaggtcgttgtgaccattaatagctgtcctggcccacttaaacaaggttacaaaaaatcagagtcggaagcagccaaataggtcaacctaatgactagactgtacattcccatgagccttcatgtttaagtgtgtacatgtgcgttaaccttgatgatgcgtgaatcccgagggagccggtggcatacaccgttagcttaaccttagcttaaactagctgaaggctcctgtgccatgtcttagacattgcatgccctatcaattactataatcctgagccatggtgtgctactgaaaccaatttttatccaccatctagtccttattaaatgaaacctcacggatcctttgttccgcttatattccatgcataccacataaaagcacacagtgcgaaaactcttgctgatacgcgatattgattctcattgttagaatatggagagtgtttcagcctcgtctgtccggctggagcttcgggatggaaagtgctatgtgtccctgcatataagaatcaccaggccagtgtttctgggtttgcttgtctatatgtttgtctatattttttgcctatacatttttcccacgtttccaacagcacttctcacctattcaataactgaaaaagacattaccatagtgctttacatttttaaagtaatgttacaaggtctggaatccatttggagcagataccgtgttttcgctatttaataagaagttcagtagtgaaatcttactgtaccgcctgttgtatctgggagcctcgtacagaggctcgcacagcagtgatcaagtgtcatcccttacgtgactgggggatgtctgtcctaaaagctgactgctaggatagtaaggatcatcttgcctgggctatgccactgtcttgttaccaattagacatctggaatttcataattagttttcattgtcactgtcaagatatattgcagattacttaaatatggccatcaaaacaaaagttacaacacgtatctcttttcatctgaaaactaatacctggaaaaggataaaaaaaaaaaggaatccgtgacccacagagctagacagataagatgcatagttgaccagtcataaaaggcggtgtttaagtgatcaggatgccgttggtggcatttacgtgctttatatgatttttacctctgtaacaaacacaagaaataaacagaatggtccttaacagagtttgggggagagagcaagatgggttccttggagaagctgatttgccaagatgcacatcgctattaacagccagagtcataaatgaaatgaaattgaagaattcattcaaatgctcttttccctataacctcttttctcaccaaaaaggagataaatttgaaaacagataaatgtaacaaccagtcaaagaagcaggggaaaagtaagctcctccaaagttgcttgcagtgctggaaatagatctcatttttaggttttctcttcgttccagataccaaataaatgggacagagaataaaatttttgttaaaatatgtgctcatctcctaagtagctcttcagagtctgaccgtaagtaaaaacacacagaattgtgttgactgggggaggtgaatcacaaaaaagttacgaggagtttaagagttaaatattatttgatcgtggctgtcaaatttagtgaacaacatagattggatttggagttggtagtaagtatggttctcataccagaattctctt

SEQ ID NO: 8 - protein, 1496 aa, homo sapien
MALSKGLRLLGRLGAEGDCSVLLEARGRDDCLLFEAGTVATLAPEEKEVIKGQYGKLTDAYGCLGELRLKSGGTSLSFLVLVTGCTSVGRIPDAEIYKITATDFYPLQEEAKEEERLIALKKILSSGVFYFSWPNDGSRFDLTVRTQKQGDDSSEWGNSFFWNQLLHVPLRQHQVSCCDWLLKIICGVVTIRTVYASHKQAKACLVSRVSCERTGTRFHTRGVNDDGHVSNFVETEQMIYMDDGVSSFVQIRGSVPLFWEQPGLQVGSHHLRLHRGLEANAPAFDRHMVLLKEQYGQQVVVNLLGSRGGEEVLNRAFKKLLWASCHAGDTPMINFDFHQFAKGGKLEKLETLLRPQLKLHWEDFDVFTKGENVSPRFQKGTLRMNCLDCLDRTNTVQSFIALEVLHLQLKTLGLSSKPIVDRFVESFKAMWSLNGHSLSKVFTGSRALEGKAKVGKLKDGARSMSRTIQSNFFDGVKQEAIKLLLVGDVYGEEVADKGGMLLDSTALLVTPRILKAMTERQSEFTNFKRIRIAMGTWNVNGGKQFRSNVLRTAELTDWLLDSPQLSGATDSQDDSSPADIFAVGFEEMVELSAGNIVNASTTNKKMWGEQLQKAISRSHRYILLTSAQLVGVCLYIFVRPYHVPFIRDVAIDTVKTGMGGKAGNKGAVGIRFQFHSTSFCFICSHLTAGQSQVKERNEDYKEITQKLCFPMGRNVFSHDYVFWCGDFNYRIDLTYEEVFYFVKRQDWKKLLEFDQLQLQKSSGKIFKDFHEGAINFGPTYKYDVGSAAYDTSDKCRTPAWTDRVLWWRKKHPFDKTAGELNLLDSDLDVDTKVRHTWSPGALQYYGRAELQASDHRPVLAIVEVEVQEVDVGARERVFQEVSSFQGPLDATVVVNLQSPTLEEKNEFPEDLRTELMQTLGSYGTIVLVRINQGQMLVTFADSHSALSVLDVDGMKVKGRAVKIRPKTKDWLKGLREEIIRKRDSMAPVSPTANSCLLEENFDFTSLDYESEGDILEDDEDYLVDEFNQPGVSDSELGGDDLSDVPGPTALAPPSKSPALTKKKQHPTYKDDADLVELKRELEAVGEFRHRSPSRSLSVPNRPRPPQPPQRPPPPTGLMVKKSASDASISSGTHGQYSILQTARLLPGAPQQPPKARTGISKPYNVKQIKTTNAQEAEAAIRCLLEARGGASEEALSAVAPRDLEASSEPEPTPGAAKPETPQAPPLLPRRPPPRVPAIKKPTLRRTGKPLSPEEQFEQQTVHFTIGPPETSVEAPPVVTAPRVPPVPKPRTFQPGKAAERPSHRKPASDEAPPGAGASVPPPLEAPPLVPKVPPRRKKSAPAAFHLQVLQSNSQLLQGLTYNSSDSPSGHPPAAGTVFPQGDFLSTSSATSPDSDGTKAMKPEAAPLLGDYQDPFWNLLHHPKLLNNTWLSKSSDPLDSGTRSPKRDPIDPVSAGASAAKAELPPDHEHKTLGHWVTISDQEKRTALQVFDPLAKT

SEQ ID NO: 9 - protein, 1496 aa, homo sapien
MALSKGLRLLGRLGAEGDCSVLLEARGRDDCLLFEAGTVATLAPEEKEVIKGQYGKLTDAYGCLGELRLKSGGTSLSFLVLVTGCTSVGRIPDAEIYKITATDFYPLQEEAKEEERLIALKKILSSGVFYFSWPNDGSRFDLTVRTQKQGDDSSEWGNSFFWNQLLHVPLRQHQVSCCDWLLKIICGVVTIRTVYASHKQAKACLVSRVSCERTGTRFHTRGVNDDGHVSNFVETEQMIYMDDGVSSFVQIRGSVPLFWEQPGLQVGSHHLRLHRGLEANAPAFDRHMVLLKEQYGQQVVVNLLGSRGGEEVLNRAFKKLLWASCHAGDTPMINFDFHQFAKGGKLEKLETLLRPQLKLHWEDFDVFTKGENVSPRFQKGTLRMNCLDCLDRTNTVQSFIALEVLHLQLKTLGLSSKPIVDRFVESFKAMWSLNGHSLSKVFTGSRALEGKAKVGKLKDGARSMSRTIQSNFFDGVKQEAIKLLLVGDVYGEEVADKGGMLLDSTALLVTPRILKAMTERQSEFTNFKRIRIAMGTWNVNGGKQFRSNVLRTAELTDWLLDSPQLSGATDSQDDSSPADIFAVGFEEMVELSAGNIVNASTTNKKMWGEQLQKAISRSHRYILLTSAQLVGVCLYIFVRPYHVPFIRDVAIDTVKMGMGGKAGNKGAVGIRFQFHSTSFCFICSHLTAGQSQVKERNEDYKEITQKLCFPMGRNVFSHDYVFWCGDFNYRIDLTYEEVFYFVKRQDWKKLLEFDQLQLQKSSGKIFKDFHEGAINFGPTYKYDVGSAAYDTSDKCRTPAWTDRVLWWRKKHPFDKTAGELNLLDSDLDVDTKVRHTWSPGALQYYGRAELQASDHRPVLAIVEVEVQEVDVGARERVFQEVSSFQGPLDATVVVNLQSPTLEEKNEFPEDLRTELMQTLGSYGTIVLVRINQGQMLVTFADSHSALSVLDVDGMKVKGRAVKIRPKTKDWLKGLREEIIRKRDSMAPVSPTANSCLLEENFDFTSLDYESEGDILEDDEDYLVDEFNQPGVSDSELGGDDLSDVPGPTALAPPSKSPALTKKKQHPTYKDDADLVELKRELEAVGEFRHRSPSRSLSVPNRPRPPQPPQRPPPPTGLMVKKSASDASISSGTHGQYSILQTARLLPGAPQQPPKARTGISKPYNVKQIKTTNAQEAEAAIRCLLEARGGASEEALSAVAPRDLEASSEPEPTPGAAKPETPQAPPLLPRRPPPRVPAIKKPTLRRTGKPLSPEEQFEQQTVHFTIGPPETSVEAPPVVTAPRVPPVPKPRTFQPGKAAERPSHRKPASDEAPPGAGASVPPPLEAPPLVPKVPPRRKKSAPAAFHLQVLQSNSQLLQGLTYNSSDSPSGHPPAAGTVFPQGDFLSTSSATSPDSDGTKAMKPEAAPLLGDYQDPFWNLLHHPKLLNNTWLSKSSDPLDSGTRSPKRDPIDPVSAGASAAKAELPPDHEHKTLGHWVTISDQEKRTALQVFDPLAKT